(19) United States
(12) Patent Application Publication
(10) Pub. No.: US 2026/0286317 A1
(43) Pub. Date: Sep. 24, 2026

(54) REGULATABLE FUSOGENIC ONCOLYTIC HERPES SIMPLEX VIRUS TYPE 1 VIRUS AND METHODS OF USE

(71) Applicant: THE BRIGHAM AND WOMEN'S HOSPITAL, INC., Boston, MA (US)

(72) Inventor: Feng YAO, Southborough, MA (US)

(73) Assignee: THE BRIGHAM AND WOMEN'S HOSPITAL, INC., Boston, MA (US)

(21) Appl. No.: 19/556,694

(22) Filed: Mar. 4, 2026

Related U.S. Application Data
(63) Continuation of application No. 17/294,894, filed on Nov. 15, 2019, now abandoned.
(60) Provisional application No. 62/769,280, filed on Nov. 19, 2018.

(51) Int. Cl. C12N 7/00; A61K 35/763

(52) U.S. Cl. CPC C12N 7/00; A61K 35/763; C12N 2710/16621; C12N 2710/16632

(57) ABSTRACT

Malignant tumors that are resistant to conventional therapies represent significant therapeutic challenges. An embodiment of the present invention provides a regulatable fusogenic oncolytic herpes simplex virus-1 that is more effective at selective killing target cells, such as tumor cells. In various embodiments presented herein, the oncolytic virus described herein is suitable for treatment of solid tumors, as well as other cancers.

### CROSS-REFERENCE TO RELATED APPLICATIONS

This application is a Continuation of U.S. patent application Ser. No. 17/294,894, filed May 18, 2021, which is a 371 National Phase Entry of International Patent Application No. PCT/US2019/061662, filed Nov. 15, 2019, which claims benefit under 35 U.S.C. § 119 (e) of U.S. Provisional Application No. 62/769,280 filed Nov. 19, 2018, the contents of which are incorporated herein by reference in their entirety.

### SEQUENCE LISTING

The instant application contains a Sequence Listing which has been submitted electronically in XML format and is hereby incorporated by reference in its entirety. Said XML copy, created on Apr. 24, 2026, is named 043214-089130USC1_SL.xml and is 170,288 bytes in size.

### FIELD OF INVENTION

The present invention is directed compositions and methods of treating cancer using regulatable fusogenic oncolytic herpes simplex virus 1 (HSV-1) virus.

### BACKGROUND

Oncolytic viral therapy entails harnessing the ability of a virus to reproduce in and lyse human cells and directing this viral replication-dependent lysis preferentially toward cancerous cells. Advances in cancer biology, together with a detailed understanding of the roles of host factors and virus-encoded gene products in controlling virus production in infected cells, have facilitated the use of some viruses as potential therapeutic agents against cancer (Aghi and Martuza, 2005; Parato et al., 2005). Herpes simplex virus (HSV) possesses several unique properties as an oncolytic agent (Aghi and Martuza, 2005). It can infect a broad range of cell types, leading to the replication of new virus and cell death. HSV has a short replication cycle (9 to 18 h) and encodes many non-essential genes that, when deleted, greatly restrict the ability of the virus to replicate in non-dividing normal cells. Because of its large genome, multiple therapeutic genes can be packaged into the genome of oncolytic recombinants.

The use of a replication-conditional strain of HSV-1 as an oncolytic agent was first reported for the treatment of malignant gliomas (Martuza et al., 1991). Since then, various efforts have been made in an attempt to broaden their therapeutic efficacy and increase the replication specificity of the virus in tumor cells. Not surprisingly, however, deletion of genes that impair viral replication in normal cells also leads to a marked decrease in the oncolytic activity of the virus for the targeted tumor cells (Advani et al., 1998; Chung et al., 1999). Currently, no oncolytic viruses that are able to kill only tumor cells while leaving normal cells intact are available. Consequently, the therapeutic doses of existing oncolytic viruses are significantly restricted (Aghi and Martuza, 2005). The availability of an oncolytic virus whose replication can be tightly controlled and adjusted pharmacologically would offer greatly increased safety and therapeutic efficacy. Such a regulatable oncolytic virus would minimize unwanted replication in adjacent and distant tissues as well as undesirable progeny virus overload in the target area after the tumor has been eliminated. This regulatory feature would also allow the oncolytic activity of the virus to be quickly shut down should adverse effects be detected (Aghi and Martuza, 2005; Shen and Nemunaitis, 2005). Work described herein presents a regulatable fusogenic variant of a oncolytic HSV that is significantly more effective at killing cancer cells than its non-fusogenic parent.

### SUMMARY OF THE INVENTION

The invention described herein is based, in part, on an isolated fusogenic variant of a novel oncolytic HSV-1 recombinant, KTR27, whose replication can be tightly controlled and regulated by tetracycline in a dose-dependent manner (Yao et al., J Virol, 2010) (U.S. Pat. No. 8,236,941). Work described herein demonstrates that this fusogenic variant, KTR27-F, is significantly more superior to its non-fusogenic parent in lysing various tested human cancer cells. Like KTR27, replication of KTR27-F in primary human fibroblasts is markedly reduced compared with various human tumor cells. The yield of KTR27-F in human breast cancer cells (MCF-7) is 21,800-fold higher than in growth-arrested normal human breast fibroblasts. Moreover, while infection of growth-arrested human breast fibroblasts with KTR27 induced little or no cytotoxicity in the infected cells, over 99% of infected MCF7 cells were non-viable compared with the mock-infected control. Collectively, KTR27-F represents proof-of-concept advancement in the design of safer and more effective oncolytic viruses.

Accordingly, one aspect described herein provides an oncolytic Herpes Simplex Virus (HSV) comprising recombinant DNA, wherein the recombinant DNA has both ICP0 and ICP34.5 gene deleted or does not express functional ICP0 and ICP34.5

Another aspect described herein provides an oncolytic Herpes Simplex Virus (HSV) comprising recombinant DNA, wherein the recombinant DNA comprises: a gene comprising a 5′ untranslated region and a HSV-1, or HSV-2, ICP27 gene that is operably linked to an ICP27 promoter comprising a TATA element; a tetracycline operator sequence positioned between 6 and 24 nucleotides 3′ to said TATA element, wherein the ICP27 gene lies 3′ to said tetracycline operator sequence; a ribozyme sequence located in said 5′ untranslated region of said gene; a gene sequence encoding tetracycline repressor operably linked to an immediate-early promoter, wherein the gene sequence is located at the ICP0 locus; and a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant, wherein said oncolytic HSV does not encode functional ICP0 and functional ICP34.5 protein.

In one embodiment of any aspect, the variant gene is a gK variant gene that encodes an amino acid substitution selected from the group consisting of: an Ala to Val amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2. In one embodiment, the oncolytic HSV further comprises a variant UL24 gene that encodes a Ser to Asn amino acid substitution corresponding to amino acid 113 of SEQ ID NO: 3. In one embodiment of any aspect, the variant gene is a UL24 gene that encodes a Ser to Asn amino acid substitution corresponding to amino acid 113 of SEQ ID NO: 3. In one embodiment, the amino acids described herein can be substituted for any known amino acid.

In one embodiment of any aspect, the tetracycline operator sequence comprises two Op2 repressor binding sites.

In one embodiment of any aspect, the ICP27 promoter is an HSV-1 or HSV-2 ICP27 promoter.

In one embodiment of any aspect, the immediate-early promoter is an HSV-1 or HSV-2 immediate-early promoter or the HCMV immediate-early promoter.

In one embodiment of any aspect, the HSV immediate-early promoter is selected from the group consisting of: ICP0 promoter, ICP4 promoter, ICP27 promoter, and ICP22 promoter.

In one embodiment of any aspect, the recombinant DNA is part of the HSV-1 genome. In one embodiment of any aspect, the recombinant DNA is part of the HSV-2 genome.

In one embodiment of any aspect, the oncolytic HSV described herein further comprises a pharmaceutically acceptable carrier

In one embodiment of any aspect, the oncolytic HSV described herein further encodes at least one polypeptide that can increase the efficacy of the oncolytic HSV to induce an anti-tumor-specific immunity. In one embodiment, the at least one polypeptide encodes a product selected from the group consisting of: interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, CTLA-4 antibody or antibody reagent, TIM-3 antibody or antibody reagent, and TIGIT antibody or antibody reagent.

Another aspect described herein provides a composition comprising any of the oncolytic HSV described herein. In one embodiment, the composition further comprises a pharmaceutically acceptable carrier.

Another aspect described herein provides a method for treating cancer comprising administering any of the oncolytic HSV described herein or a composition thereof to a subject having cancer.

In one embodiment of any aspect, the cancer is a solid tumor.

In one embodiment of any aspect, the tumor is benign or malignant.

In one embodiment of any aspect, the subject is diagnosed or has been diagnosed as having a carcinoma, a melanoma, a sarcoma, a germ cell tumor, or a blastoma. In one embodiment of any aspect, the subject is diagnosed or has been diagnosed as having non-small-cell lung cancer, breast cancer, brain cancer, colon cancer, prostate cancer, liver cancer, lung cancer, ovarian cancer, skin cancer, and pancreatic cancer.

In one embodiment of any aspect, the cancer is metastatic.

In one embodiment of aspect, the oncolytic HSV is administered directly to the tumor.

In one embodiment of any aspect, the method further comprises administering an agent that regulates the tet operator. In one embodiment, the agent is doxycycline or tetracycline. In one embodiment, the agent is administered locally or systemically.

### Definitions

All references cited herein are incorporated by reference in their entirety as though fully set forth.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. Definitions of common terms can be found in Singleton et al., Dictionary of Microbiology and Molecular Biology 3^{rd} ed., J. Wiley & Sons New York, NY (2001); March, Advanced Organic Chemistry Reactions, Mechanisms and Structure 5^{th} ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, A D A M Kruisbeek, David H Margulies, John Wiley and Sons, Inc., (2003); each of which provide one skilled in the art with a general guide to many of the terms used in the present application.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligan-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “operably linked,” as used herein, refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed. The nucleic acid sequence elements, when operably linked, can act together to modulate the activity of one another, and ultimately may affect the level of expression of the gene of interest, including any of those encoded by the sequences described above.

The term “vector,” as used herein, refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both of which are incorporated herein by reference). Additionally, the techniques described herein and demonstrated in the referenced figures are also instructive with regard to effective vector construction.

The term “oncolytic HSV-1 vector” refers to a genetically engineered HSV-1 virus corresponding to at least a portion of the genome of HSV-1 that is capable of infecting a target cell, replicating, and being packaged into HSV-1 virions. The genetically engineered virus comprises deletions and or mutations and or insertions of nucleic acid that render the virus oncolytic such that the engineered virus replicates in- and kills-tumor cells by oncolytic activity. The virus may be attenuated or non-attenuated. The virus may or may not deliver a transgene—that differs from the HSV viral genome. In one embodiment, the oncolytic HSV-1 vector does not express a transgene to produce a protein foreign to the virus.

The term “promoter,” as used herein, refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the gene of interest. The promoter comprises a DNA regulatory sequence, wherein the regulatory sequence is derived from a gene, which is capable of binding RNA polymerase and initiating transcription of a downstream (3′-direction) coding sequence. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one can position the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter used, individual elements can function either cooperatively or independently to activate transcription. The promoters described herein may or may not be used in conjunction with an “enhancer,” which refers to a cis-acting regulatory sequence involved in the transcriptional activation of a nucleic acid sequence, such as those for the genes, or portions or functional equivalents thereof, listed herein.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include, the HCMV immediate-early promoter, the beta-lactamase (penicillinase), lactose and tryptophan (trp) promoter systems.

A “gene,” or a “sequence which encodes” a particular protein, is a nucleic acid molecule which is transcribed (in the case of DNA) and translated (in the case of mRNA) into a polypeptide in vitro or in vivo when placed under the control of one or more appropriate regulatory sequences. A gene of interest can include, but is no way limited to, cDNA from eukaryotic mRNA, genomic DNA sequences from eukaryotic DNA, and even synthetic DNA sequences. A transcription termination sequence will usually be located 3′ to the gene sequence. Typically, a polyadenylation signal is provided to terminate transcription of genes inserted into a recombinant virus.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “transgene” refers to a particular nucleic acid sequence encoding a polypeptide or a portion of a polypeptide to be expressed in a cell into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been inserted; (3) a nucleic acid sequence that serves to add additional copies of the same (i.e., homologous) or a similar nucleic acid sequence naturally occurring in the cell into which it has been inserted; or (4) a silent naturally occurring or homologous nucleic acid sequence whose expression is induced in the cell into which it has been inserted. A “mutant form” or “modified nucleic acid” or “modified nucleotide” sequence means a sequence that contains one or more nucleotides that are different from the wild-type or naturally occurring sequence, i.e., the mutant nucleic acid sequence contains one or more nucleotide substitutions, deletions, and/or insertions. In some cases, the gene of interest may also include a sequence encoding a leader peptide or signal sequence such that the transgene product may be secreted from the cell.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

The term “oncolytic activity,” as used herein, refers to cytotoxic effects in vitro and/or in vivo exerted on tumor cells without any appreciable or significant deleterious effects to normal cells under the same conditions. The cytotoxic effects under in vitro conditions are detected by various means as known in prior art, for example, by staining with a selective stain for dead cells, by inhibition of DNA synthesis, or by apoptosis. Detection of the cytotoxic effects under in vivo conditions is performed by methods known in the art.

A “biologically active” portion of a molecule, as used herein, refers to a portion of a larger molecule that can perform a similar function as the larger molecule. Merely by way of non-limiting example, a biologically active portion of a promoter is any portion of a promoter that retains the ability to influence gene expression, even if only slightly. Similarly, a biologically active portion of a protein is any portion of a protein which retains the ability to perform one or more biological functions of the full-length protein (e.g. binding with another molecule, phosphorylation, etc.), even if only slightly.

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

With the aforementioned preliminary descriptions and definitions in mind, additional background is provided herein below to provide context for the genesis and development of the inventive vectors, compositions and methods described herein.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 shows U2OS cells seeded at 1×10⁶ cells per 60 mm dish. Cells were infected with KTR27 or KTR27-F at 200 PFU/dish at 72 h post-cell seeding in the presence of tetracycline. KTR27 and KTR27-F plaques were photographed at 48 and 72 h post-infection.

FIG. 2 shows KTR27-F replication is highly regulated by tetracycline. Vero cells were seeded at 7.5×10⁵ cells per 60 mm dish. At 48 h post-seeding, triplicate dishes of cells were infected with KTR27 and KTR27-F at a MOI of 1 PFU/cell in a volume of 0.5 ml. After 1.5 h of incubation at 37° C., the inocula were removed and the cells were washed twice with acid-glycine saline (to remove membrane-bound extracellular virions) and then twice by DMEM. KTR27 infections were carried out in the presence of tetracycline at 2.5 μg/ml, and KTR27-F infections were carried out in the presence and absence of tetracycline. Infected cells were harvested at 48 and 72 h post-infection. Viral titers were determined on U2OS monolayers in the presence of tetracycline. KTR27-F production in the absence of tetracycline was not detected. Viral titers are expressed as means±standard deviation.

FIGS. 3A and 3B show KTR27-F replication is efficient and highly regulated in various human tumor cell lines. Human cancer cells H1299 (lung), U87 (glioma), MDA MB 231 (breast), and MCF7 (breast) were seeded at 7.5×10⁵, 1×10⁶, 7.5×10⁵, and 7.5×10⁵ cells per 60 mm dish, respectively. At 48, 24, 72, and 48 h post-seeding, respectively, triplicate dishes were infected. (FIG. 3A) H1299, U87, MDA-MB-231, and MCF-7 dishes were infected with KTR27 and KTR27-F at a MOI of 1 PFU/cell in a volume of 0.5 ml. After 1.5 h of incubation at 37° C., the inocula were removed and the cells were washed twice with acid-glycine saline and then twice by DMEM. Infections were then carried out in the absence or presence of tetracycline at 2.5 μg/ml. Infected cells were harvested at 48, 72, 72, and 40 h post-infection, respectively, and viral titers were determined on U2OS monolayers in the presence of tetracycline. Numbers located above the brackets indicate the fold difference in viral yield between the indicated conditions. (FIG. 3B) H1299, U87, MDA-MB-231, and MCF-7 cells were mock-infected and infected with KTR5 and KTR27 at MOIs 0.25, 1, 1, and 0.25 PFU/cell, respectively. Cells were harvested at 72, 72, 96, and 72 h post-infection. Viable cells were counted by trypan blue exclusion and graphed as a percentage of viable cells in the mock-infected controls, expressed as means±standard deviation.

FIGS. 4A-4C show cytotoxicity and replication of KTR27-F are significantly enhanced in human breast cancer cells versus in normal human breast fibroblasts. For results labeled “HF-serum free,” primary human fibroblasts (HF) were seeded at 1.5×10⁶ cells per 60 mm dish in normal growth medium. 24 h post-seeding, normal medium was removed and replaced with serum-free DMEM containing antibiotics. These cells were infected at 42 h post-serum starvation. All other cells were seeded at 7.5×10⁵ cells per 60 mm dish in normal growth medium and infected 66 h post-seeding. All cells described above were either mock infected or infected with KTR27-F at a MOI of 1 PFU/cell in the absence or presence of tetracycline at 2.5 μg/ml in DMEM containing 2% FBS. (FIG. 4A) Triplicate dishes of infected cells were harvested at 48 h post-infection and viral titers were determined on U2OS monolayers in the presence of tetracycline. (FIG. 4B) Mock-infected and infected cells in the presence of tetracycline in triplicate dishes were harvested at 48 h post-infection. Viable cells were counted by trypan blue exclusion and graphed as a percentage of viable cells in the mock-infected controls, expressed as means±standard deviation. (FIG. 4C) Selective lysis of MCF7 cells. Images cells infected with KTR27-F in the absence and presence of tetracycline, photographed at 48 h post-infection.

FIG. 5 shows KTR27-F is avirulent following intracerebral inoculation. Female CD1 mice were intracerebrally inoculated with 20 μl of DMEM or DMEM containing 1×10⁷ PFU of indicated viruses. Half of the mice injected with KTR27-F were fed a doxycycline-containing diet beginning three days prior to inoculation (T+). The mice were examined for signs of illness for 29 days.

### DESCRIPTION OF THE INVENTION

Oncolytic viruses are genetically modified viruses that preferentially replicate in host cancer cells, leading to the production of new viruses, lysis of cancer cells, and ultimately, induction of tumor-specific immunity. Using the T-REx™ (Invitrogen, CA) gene switch technology and a self-cleaving ribozyme, a novel oncolytic HSV-1 recombinant, KTR27, was constructed, whose replication can be tightly controlled and regulated by tetracycline in a dose-dependent manner. This virus is further described in Yao et al., J Virol, 2010 and U.S. Pat. No. 8,236,941, which are incorporated herein by reference in their entirety. Infection of normal replicating cells as well as multiple human cancer cell types with KTR27 in the presence of tetracycline led to 1000- to 250,000-fold higher progeny virus production than in the absence of tetracycline, while little viral replication and virus-associated cytotoxicity are observed in infected growth-arrested normal human cells. Importantly, KTR27 is very effective against pre-established Non-Small cell lung cancer in nude mice and can prevent the growth of pre-established M3 mouse melanoma in immuno-competent mice. Intratumoral inoculation of KTR27 can elicit systemic immune response that can effectively prevent the growth of a distant tumor in immuno-competent mice.

In an effort to further enhance the therapeutic efficacy of KTR27 and its effectiveness in eliciting tumor specific immunity following oncolytic virotherapy, a fusogenic variant of KTR27, KTR27-F, was isolated. Work described herein demonstrate that KTR27-F is significantly more superior to its non-fusogenic parent in lysing various tested human cancer cells. Like KTR27, replication of KTR27-F in primary human fibroblasts is markedly reduced compared with various human tumor cells. The yield of KTR27-F in human breast cancer cells (MCF-7) is 21,800-fold higher than in growth-arrested normal human breast fibroblasts. Moreover, while infection of growth-arrested human breast fibroblasts with KTR27-F induced little or no cytotoxicity in the infected cells, over 99% of infected MCF7 cells were non-viable compared with the mock-infected control. Collectively, KTR27-F represents an advancement in the design of safer and more effective oncolytic viruses.

HSV-1 is a human neurotropic virus that is capable of infecting virtually all vertebrate cells. Natural infections follow either a lytic, replicative cycle or establish latency, usually in peripheral ganglia, where the DNA is maintained indefinitely in an episomal state. HSV-1 contains a double-stranded, linear DNA genome, about 152 kilobases in length, which has been completely sequenced by McGeoch (McGeoch et al., J. Gen. Virol. 69:1531 (1988); McGeoch et al., Nucleic Acids Res 14:1727 (1986); McGeoch et al., J. Mol. Biol. 181:1 (1985); Perry and McGeoch, J. Gen. Virol. 69:2831 (1988); Szpara M L et al., J Virol. 2010, 84:5303; Macdonald S J et al., J Virol. 2012, 86:6371). DNA replication and virion assembly occurs in the nucleus of infected cells. Late in infection, concatemeric viral DNA is cleaved into genome length molecules which are packaged into virions. In the CNS, herpes simplex virus spreads transneuronally followed by intraaxonal transport to the nucleus, either retrograde or anterograde, where replication occurs.

One aspect described herein provides an oncolytic Herpes Simplex Virus (HSV) comprising recombinant DNA, wherein the recombinant DNA has both ICP0 and ICP34.5 gene product deleted or does not express functional ICP0 and ICP34.5.

Infected cell protein 34.5 (ICP34.5) is a protein (e.g., a gene product) expressed by the γ34.5 gene in viruses, such as the herpes simplex virus. IPC34.5 has been shown to block the cellar stress response to a viral infection (Agarwalla, P. K., et al. Method in Mol. Bio., 2012). Infected cell polypeptide 0 (ICP0) is a protein encoded by the HSV-1 α0 gene. ICP0 is generated during the immediate-early phase of viral gene expression. ICP0 is synthesized and transported to the nucleus of the infected host cell, where it promotes transcription from viral genes, disrupts nuclear and cytoplasmic cellular structures, such as the microtubule network, and alters the expression of host genes.

One skilled in the art can determine if the ICP0 or ICP34.5 gene products have been deleted or if the virus does not express functional forms of these gene products using PCR-based assays to detect the presence of the gene in the viral genome or the expression of the gene products, or using functional assays to assess their function, respectively.

In one embodiment, the gene that encodes these gene products contain a mutation, for example, an inactivating mutation, that inhibits proper expression of the gene product. For example, the gene may encode a mutation in the gene product that inhibits proper folding, expression, function, ect. of the gene product. As used herein, the term “inactivating mutation” is intended to broadly mean a mutation or alteration to a gene wherein the expression of that gene is significantly decreased, or wherein the gene product is rendered nonfunctional, or its ability to function is significantly decreased. The term “gene” encompasses both the regions coding the gene product as well as regulatory regions for that gene, such as a promoter or enhancer, unless otherwise indicated.

Ways to achieve such alterations include: (a) any method to disrupt the expression of the product of the gene or (b) any method to render the expressed gene nonfunctional. Numerous methods to disrupt the expression of a gene are known, including the alterations of the coding region of the gene, or its promoter sequence, by insertions, deletions and/or base changes. (See, Roizman, B. and Jenkins, F. J., Science 229:1208-1214 (1985)).

Further described herein is an oncolytic Herpes Simplex Virus (HSV) comprising recombinant DNA, wherein the recombinant DNA comprises: (a) a gene comprising a 5′ untranslated region and a HSV-1, or HSV-2, ICP27 gene that is operably linked to an ICP27 promoter comprising a TATA element; (b) a tetracycline operator sequence positioned between 6 and 24 nucleotides 3′ to said TATA element, wherein the ICP27 gene lies 3′ to said tetracycline operator sequence; (c) a ribozyme sequence located in said 5′ untranslated region of said gene; (d) a gene sequence encoding tetracycline repressor operably linked to an immediate early promoter, wherein the gene sequence is located at the ICP0 locus; and (e) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant, wherein said oncolytic HSV does not encode functional ICP0 and functional ICP34.5 protein. In one embodiment, the recombinant DNA is derived from the HSV-1 genome. In an alternative embodiment, the recombinant DNA is derived from the HSV-2 genome. In one embodiment, the genome of the HSV comprising recombinant DNA consists of, consists essentially of, or comprises the sequence of SEQ ID NO: 1. The nucleotide sequence of SEQ ID NO: 1 contains the plasmid vector sequence present in pSH-tetR (SEQ ID NO: 9).

An essential feature of the DNA of the present invention is the presence of a gene needed for virus replication that is operably linked to a promoter having a TATA element. A tet operator sequence is located between 6 and 24 nucleotides 3′ to the last nucleotide in the TATA element of the promoter and 5′ to the gene. The strength with which the tet repressor binds to the operator sequence is enhanced by using a form of operator which contains two op2 repressor binding sites (each such site having the nucleotide sequence: TCCCTATCAGTGATAGAGA (SEQ ID NO: 8)) linked by a sequence of 2-20, preferably 1-3 or 10-13, nucleotides. When repressor is bound to this operator, very little or no transcription of the associated gene will occur. If DNA with these characteristics is present in a cell that also expresses the tetracycline repressor, transcription of the gene will be blocked by the repressor binding to the operator and replication of the virus will not occur. However, if tetracycline is introduced, it will bind to the repressor, cause it to dissociate from the operator, and virus replication will proceed.

During productive infection, HSV gene expression falls into three major classes based on the temporal order of expression: immediate-early (α), early (β), and late (γ), with late genes being further divided into two groups, γ1 and γ2. The expression of immediate-early genes does not require de novo viral protein synthesis and is activated by the virion-associated protein VP16 together with cellular transcription factors when the viral DNA enters the nucleus. The protein products of the immediate-early genes are designated infected cell polypeptides ICPO, ICP4, ICP22, ICP27, and ICP47 and it is the promoters of these genes that are preferably used in directing the expression of tet repressor (tetR). The expression of a gene needed for virus replication is under the control of the tetO-containing promoters and these essential genes may be immediate-early, early or late genes, e.g., ICP4, ICP27, ICP8, UL9, gD and VP5. In one embodiment, the tetR has the sequence of SEQ ID NO: 9.

ICP0 plays a major role in enhancing the reactivation of HSV from latency and confers a significant growth advantage on the virus at low multiplicities of infection. ICP4 is the major transcriptional regulatory protein of HSV-1, which activates the expression of viral early and late genes. ICP27 is essential for productive viral infection and is required for efficient viral DNA replication and the optimal expression of subset of viral β genes and γ1 genes as well as viral γ2 genes. The function of ICP47 during HSV infection appears to be to down-regulate the expression of the major histocompatibility complex (MHC) class I on the surface of infected cells.

The recombinant DNA may also include at least one, and preferably at least two, sequences coding for the tetracycline repressor with expression of these sequences being under the control of an immediate early promoter, preferably ICP0 or ICP4. The sequence for the HSV ICP0 and ICP4 promoters and for the genes whose regulation they endogenously control are well known in the art (Perry, et al., J. Gen. Virol. 67:2365-2380 (1986); McGeoch et al., J. Gen. Virol. 72:3057-3075 (1991); McGeoch et al., Nucl. Acid Res. 14:1727-1745 (1986)) and procedures for making viral vectors containing these elements have been previously described (see US published application 2005-0266564 In one embodiment, the tetR has the sequence of SEQ ID NO: 9.

These promoters are not only very active in promoting gene expression, they are also specifically induced by VP16, a transactivator released when HSV-1 infects a cell. Thus, transcription from ICP0 promoter is particularly high when repressor is most needed to shut down virus replication. Once appropriate DNA constructs have been produced, they may be incorporated into HSV-1 virus using methods that are well known in the art. One appropriate procedure is described in US 2005-0266564 but other methods known in the art may also be employed.

In various embodiments, the variant gene comprises at least one amino acid change that deviates from the wild-type sequence of the gene. In one embodiment, an oncolytic HSV described herein can contain two or more amino acid substitutions in at least one variant gene. The at least two amino acid substitutions can be found in the same gene, for example, the gK variant gene contains at least two amino acid substitutions. Alternatively, the at least two amino acid substitutions can be found in the at least two different genes, for example, the gK variant gene and the UL24 variant gene each contains at least one amino acid substitutions.

SEQ ID NO: 2 is the amino acid sequence encoding gK (strain KOS).

(SEQ ID NO: 2)
MLAVRSLQHLSTVVLITAYGLVLVWYTVFGASPLHRCIYAVRPTGTNNDTA
LVWMKMNQTLLFLGAPTHPPNGGWRNHAHICYANLIAGRVVPFQVPPDATN
RRIMNVHEAVNCLETLWYTRVRLVVVGWFLYLAFVALHQRRCMFGVVSPAH
KMVAPATYLLNYAGRIVSSVFLQYPYTKITRLLCELSVQRQNLVQLFETDP
VTFLYHRPAIGVIVGCELMLRFVAVGLIVGTAFISRGACAITYPLFLTITT
WCFVSTIGLTELYCILRRGPAPKNADKAAAPGRSKGLSGVCGRCCSIILSG
IAMRLCYIAVVAGVVLVALHYEQEIQRRLFDV

SEQ ID NO: 3 is the amino acid sequence encoding UL24 (strain KOS).

(SEQ ID NO: 3)
MAARTRSLVERRRVLMAGVRSHTRFYKALAKEVREFHATKICGTLLTLLSG
SLQGRSVFEATRVTLICEVDLGPRRPDCICVFEFANDKTLGGVCVIIELKT
CKYISSGDTASKREQRATGMKQLRHSLKLLQSLAPPGDKIVYLCPVLVFVA
QRTLRVSRVTRLVPQKVSGNITAVVRMLQSLSTYTVPMEPRTQRARRRRGG
AARGSASRPKRSHSGARDPPEPAARQVPPADQTPASTEGGGVLKRIAALFC
VPVATKTKPRAASE

Exemplary amino acid substitutions present in the variant gene are described in Table 1.

TABLE 1
Amino acid (A.A.) substitution in variant genes.

| | SEQ ID | A.A. | Wild-type | Substitution |
|---|---|---|---|---|
| Gene | NO: | Position | A.A. | A.A. |
| gK | 2 | 40 | Ala | Val |
| gK | 2 | 40 | Ala | Val |
| gK | 2 | 99 | Asp | Asn |
| gK | 2 | 304 | Leu | Pro |
| gK | 2 | 310 | Arg | Leu |
| UL24 | 3 | 113 | Ser | Asn |

In Table 1, “X” refers to any known amino acid. It is specifically contemplated herein that any amino acid in a variant gene can be substituted for any known amino acid. The list provided in Table 1 is meant to be exemplary, and is in no way supposed to be limiting to the invention. All mutations listed in table 1 for gK are derived from the HSV-1 KOS strain.

The oncolytic HSV described herein comprises a sequence encoding a ribozyme. A ribozyme is an RNA molecule that is capable of catalyzing a biochemical reaction in a similar manner as a protein enzyme. For example, a ribozyme is commonly known to facilitate cleavage or ligation of RNA and DNA, and peptide bond formation. Ribozymes have further roles in RNA processing, such as RNA splicing, viral replication, and transfer RNA biosynthesis. In one embodiment, the oncolytic HSV described herein has a ribozyme sequence that is naturally occurring. In an alternative embodiment, the oncolytic HSV described herein has a synthetic ribozyme sequence, e.g., a non-naturally occurring ribozyme. Ribozymes are further described in, e.g., Yen et al., Nature 431:471-476, 2004, the contents of which are incorporated herein by reference in its entirety. In one embodiment, the ribozyme is N107 ribozyme. SEQ ID NO: 4 is a nucleotide sequence encoding N107 ribozyme.

(SEQ ID NO: 4)
ctgaggtgcaggtacatccagctgacgagtcccaaataggacgaaacgcgc
ttcggtgtgtcctggattccactgctatcc

In one embodiment, the oncolytic HSV described herein further comprises at least one polypeptide that encodes a product (e.g., a protein, a gene, a gene product, or an antibody or antibody reagent) that can increase the efficacy of the oncolytic HSV to induce an anti-tumor-specific immunity. Exemplary products include, but are not limited to, interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, CTLA-4 antibody or antibody reagent, TIM-3 antibody or antibody reagent, and TIGIT antibody or antibody reagent. In one embodiment, the product is a fragment of IL-2, IL-12, or IL-15, that comprises the same functionality of IL-2, IL-12, or IL-15, as described herein below. One skilled in the art can determine if an anti-tumor specific immunity is induced using stand techniques in the art, which are further described in, for example, Clay, T M, et al. Clinical Cancer Research (2001); Malyguine, A, et al. J Transl Med (2004); or Macchia I, et al. BioMed Research International (2013), each of which are incorporated herein by reference in their entireties.

Interleukin-2 (IL-2) is an interleukin, a type of cytokine signaling molecule in the immune system. IL-2 regulates the activities of white blood cells (for example, leukocytes and lymphocytes) that are responsible for immunity. IL-2 is part of the body's natural response to microbial infection, and in discriminating between foreign “non-self” and “self”. It mediates its effects by binding to IL-2 receptors, which are expressed by lymphocytes. Sequences for IL-2, also known TCGF and lympokine, are known for a number of species, e.g., human IL-2 (NCBI Gene ID: 3558) polypeptide (e.g., NCBI Ref Seq NP_000577.2) and mRNA (e.g., NCBI Ref Seq NM_000586.3). IL-2 can refer to human IL-2, including naturally occurring variants, molecules, and alleles thereof. IL-2 refers to the mammalian IL-2 of, e.g., mouse, rat, rabbit, dog, cat, cow, horse, pig, and the like. The nucleic sequence of SEQ ID NO: 5 comprises the nucleic sequence which encodes IL-2.

SEQ ID NO: 5 is the nucleotide sequence encoding IL-2.

(SEQ ID NO: 5)

| | atgta |
|---|---|
| 61 | caggatgcaa ctcctgtctt gcattgcact aagtcttgca |
| | cttgtcacaa acagtgcacc |
| 121 | tacttcaagt tctacaaaga aaacacagct acaactggag |
| | catttactgc tggatttaca |
| 181 | gatgattttg aatggaatta ataattacaa gaatcccaaa |
| | ctcaccagga tgctcacatt |
| 241 | taagttttac atgcccaaga aggccacaga actgaaacat |
| | cttcagtgtc tagaagaaga |
| 301 | actcaaacct ctggaggaag tgctaaattt agctcaaagc |
| | aaaaactttc acttaagacc |
| 361 | cagggactta atcagcaata tcaacgtaat agttctggaa |
| | ctaaagggat ctgaaacaac |
| 421 | attcatgtgt gaatatgctg atgagacagc aaccattgta |
| | gaatttctga acagatggat |
| 481 | taccttttgt caaagcatca tctcaacact gacttgataa |

Interleukin-12 (IL-12) is an interleukin naturally produced by dendritic cells, macrophages, neutrophils, and human B-lymphoblastoid cells (NC-37) in response to antigenic stimulation. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. Sequences for IL-12a, also known P35, CLMF, NFSK, and KSF1, are known for a number of species, e.g., human IL-12a (NCBI Gene ID: 3592) polypeptide (e.g., NCBI Ref Seq NP_000873.2) and mRNA (e.g., NCBI Ref Seq NM_000882.3). IL-12 can refer to human IL-12, including naturally occurring variants, molecules, and alleles thereof. IL-12 refers to the mammalian IL-12 of, e.g., mouse, rat, rabbit, dog, cat, cow, horse, pig, and the like. The nucleic sequence of SEQ ID NO: 6 comprises the nucleic sequence which encodes IL-12a.

SEQ ID NO: 6 is the nucleotide sequence encoding IL-12a.

(SEQ ID NO: 6)

| | aatgtggccc cctgggtcag |
|---|---|
| 241 | cctcccagcc accgccctca cctgccgcgg ccacaggtct |
| | gcatccagcg gctcgccctg |
| 301 | tgtccctgca gtgccggctc agcatgtgtc cagcgcgcag |
| | cctcctcctt gtggctaccc |
| 361 | tggtcctcct ggaccacctc agtttggcca gaaacctccc |
| | cgtggccact ccagacccag |
| 421 | gaatgttccc atgccttcac cactcccaaa acctgctgag |
| | ggccgtcagc aacatgctcc |
| 481 | agaaggccag acaaactcta gaattttacc cttgcacttc |
| | tgaagagatt gatcatgaag |
| 541 | atatcacaaa agataaaacc agcacagtgg aggcctgttt |
| | accattggaa ttaaccaaga |
| 601 | atgagagttg cctaaattcc agagagacct ctttcataac |
| | taatgggagt tgcctggcct |
| 661 | ccagaaagac ctcttttatg atggccctgt gccttagtag |
| | tatttatgaa gacttgaaga |
| 721 | tgtaccaggt ggagttcaag accatgaatg caaagcttct |
| | gatggatcct aagaggcaga |
| 781 | tctttctaga tcaaaacatg ctggcagtta ttgatgagct |
| | gatgcaggcc ctgaatttca |
| 841 | acagtgagac tgtgccacaa aaatcctccc ttgaagaacc |
| | ggatttttat aaaactaaaa |
| 901 | tcaagctctg catacttctt catgctttca gaattcgggc |
| | agtgactatt gatagagtga |
| 961 | tgagctatct gaatgcttcc taa |

Interleukin-15 (IL-15) is an interleukin secreted by mononuclear phagocytes (and some other cells) following infection by virus(es). This cytokine induces cell proliferation of natural killer cells; cells of the innate immune system whose principal role is to kill virally infected cells. Sequences for IL-15 are known for a number of species, e.g., human IL-15 (NCBI Gene ID: 3600) polypeptide (e.g., NCBI Ref Seq NP_000585.4) and mRNA (e.g., NCBI Ref Seq NM_000576.1). IL-15 can refer to human IL-15, including naturally occurring variants, molecules, and alleles thereof. IL-15 refers to the mammalian IL-15 of, e.g., mouse, rat, rabbit, dog, cat, cow, horse, pig, and the like. The nucleic sequence of SEQ ID NO: 7 comprises the nucleic sequence which encodes IL-15.

SEQ ID NO: 7 is the nucleotide sequence encoding IL-15.

(SEQ ID NO: 7)

| | atgaga atttcgaaac cacatttgag aagtatttcc |
|---|---|
| | atccagtgct |
| 421 | acttgtgttt acttctaaac agtcattttc taactgaagc |
| | tggcattcat gtcttcattt |
| 481 | tgggctgttt cagtgcaggg cttcctaaaa cagaagccaa |
| | ctgggtgaat gtaataagtg |
| 541 | atttgaaaaa aattgaagat cttattcaat ctatgcatat |
| | tgatgctact ttatatacgg |
| 601 | aaagtgatgt tcaccccagt tgcaaagtaa cagcaatgaa |
| | gtgctttctc ttggagttac |
| 661 | aagttatttc acttgagtcc ggagatgcaa gtattcatga |
| | tacagtagaa aatctgatca |
| 721 | tcctagcaaa caacagtttg tcttctaatg ggaatgtaac |
| | agaatctgga tgcaaagaat |
| 781 | gtgaggaact ggaggaaaaa aatattaaag aatttttgca |
| | gagttttgta catattgtcc |
| 841 | aaatgttcat caacacttct tga |

Antibodies or antibody reagents that bind to PD-1, or its ligand PD-L1, are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; which are incorporated by reference herein in their entireties. In certain embodiments the PD-1 antibodies include nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade. Also specifically contemplated herein are agents that disrupt or block the interaction between PD-1 and PD-L1, such as a high affinity PD-L1 antagonist.

Non-limiting examples of PD-1 antibodies include: pembrolizumab (Merck); nivolumab (Bristol Meyers Squibb); pidilizumab (Medivation); and AUNP12 (Aurigene). Non-limiting examples of PD-L1 antibodies can include atezolizumab (Genentech); MPDL3280A (Roche); MEDI4736 (AstraZeneca); MSB0010718C (EMD Serono); avelumab (Merck); and durvalumab (Medimmune).

Antibodies that bind to OX40 (also known as CD134), are described in U.S. Pat. Nos. 9,006,399, 9,738,723, 9,975,957, 9,969,810, 9,828,432; PCT Published Patent Application Nos: WO2015153513, WO2014148895, WO2017021791, WO2018002339; and US Application Nos: US20180273632; US20180237534; US20180230227; US20120269825; which are incorporated by reference herein in their entireties.

Antibodies that bind to CTLA-4, are described in U.S. Pat. Nos. 9,714,290, 6,984,720, 7,605,238, 6,682,736, 7,452,535; PCT Published Patent Application No: WO2009100140; and US Application Nos: US20090117132A, US20030086930, US20050226875, US20090238820; which are incorporated by reference herein in their entireties.

Non-limiting examples of CTLA-4 antibodies include: ipilimumab (Bristol-Myers Squibb)

Antibodies that bind to TIM3, are described in U.S. Pat. Nos. 8,552,156, 9,605,070, 9,163,087, 8,329,660; PCT Published Patent Application No: WO2018036561, WO2017031242, WO2017178493; and US Application Nos: US20170306016, US20150110792, US20180057591, US20160200815; which are incorporated by reference herein in their entireties.

Antibodies that bind to TIGIT (also known as CD134), are described in US Patent Nos. U.S. Pat. Nos. 10,017,572, 9,713,641; PCT Published Patent Application No: WO2017030823; and US Application Nos: US20160355589, US20160176963, US20150322119; which are incorporated by reference herein in their entireties.

One aspect of the invention described herein provides a composition comprising any of the oncolytic HSV described herein. In one embodiment, the composition is a pharmaceutical composition. As used herein, the term “pharmaceutical composition” refers to the active agent in combination with a pharmaceutically acceptable carrier e.g. a carrier commonly used in the pharmaceutical industry.

In one embodiment, the composition further comprises at least one pharmaceutically acceptable carrier. Pharmaceutically acceptable carriers are well known in the art and include aqueous solutions such as physiologically buffered saline or other solvents or vehicles such as glycols, glycerol, vegetable oils (e.g., olive oil) or injectable organic esters. A pharmaceutically acceptable carrier can be used to administer the compositions of the invention to a cell in vitro or to a subject in vivo. A pharmaceutically acceptable carrier can contain a physiologically acceptable compound that acts, for example, to stabilize the composition or to increase the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the oncolytic HSV.

The oncolytic viruses described herein or composition thereof can be administered to a subject having cancer. In one embodiment, an agent that regulates the tet operator is further administered with the oncolytic viruses described herein or composition thereof. Exemplary agents include, but are not limited to, doxycycline or tetracycline.

In one embodiment, the cancer is a solid tumor. The solid tumor can be malignant or benign. In one embodiment, the subject is diagnosed or has been diagnosed with having a carcinoma, a melanoma, a sarcoma, a germ cell tumor, and a blastoma. Exemplary cancers include, but are in no way limited to, non-small-cell lung cancer, breast cancer, brain cancer, colon cancer, prostate cancer, liver cancer, lung cancer, ovarian cancer, skin cancer, and pancreatic cancer. In one embodiment, the cancer is metastatic. These types of cancers are known in the art and can be diagnosed by a skilled clinician using standard techniques known in the art, for example blood analysis, blood cell count analysis, tissue biopsy non-invasive imaging, and review of family history.

In cases where tumors are readily accessible, e.g., tumors of the skin, mouth or which are accessible as the result of surgery, virus can be applied topically. In other cases, it can be administered by injection or infusion. The agent that regulates the tet operator, for example doxycycline or tetracycline, used prior to infection or at a time of infection can also be administered in this way or it can be administered systemically.

Although certain routes of administration are provided in the foregoing description, according to the invention, any suitable route of administration of the vectors may be adapted, and therefore the routes of administration described above are not intended to be limiting. Routes of administration may including but are not limited to, intravenous, oral, buccal, intranasal, inhalation, topical application to a mucosal membrane or injection, including intratumoral, intradermal, intrathecal, intracisternal, intralesional or any other type of injection. Administration can be effected continuously or intermittently and will vary with the subject and the condition to be treated. One of skill in the art would readily appreciate that the various routes of administration described herein would allow for the inventive vectors or compositions to be delivered on, in, or near the tumor or targeted cancer cells. One of skill in the art would also readily appreciate that various routes of administration described herein will allow for the vectors and compositions described herein to be delivered to a region in the vicinity of the tumor or individual cells to be treated. “In the vicinity” can include any tissue or bodily fluid in the subject that is in sufficiently close proximity to the tumor or individual cancer cells such that at least a portion of the vectors or compositions administered to the subject reach their intended targets and exert their therapeutic effects.

Prior to administration, the oncolytic viruses can be suspended in any pharmaceutically acceptable solution including sterile isotonic saline, water, phosphate buffered saline, 1,2-propylene glycol, polyglycols mixed with water, Ringer's solution, etc. The exact number of viruses to be administered is not crucial to the invention but should be an “effective amount,” i.e., an amount sufficient to cause cell lysis extensive enough to generate an immune response to released tumor antigens. Since virus is replicated in the cells after infection, the number initially administered will increase rapidly with time. Thus, widely different amounts of initially administered virus can give the same result by varying the time that they are allowed to replicate, i.e., the time during which cells are exposed to tetracycline. In general, it is expected that the number of viruses (PFU) initially administered will be between 1×10⁶ and 1×10¹⁰.

Tetracycline or doxycycline will be administered either locally or systemically to induce viral replication at a time of infection or 1-72 h prior to infection. The amount of tetracycline or doxycycline to be administered will depend upon the route of delivery. In vitro, 1 μg/ml of tetracycline is more than sufficient to allow viral replication in infected cells. Thus, when delivered locally, a solution containing anywhere from 0.01 μg/ml to 100 μg/ml may be administered. However, much higher doses of tetracycline or doxycycline (e.g., 10-500 mg/ml) can be employed if desired. The total amount given locally at a single time will depend on the size of the tumor or tumors undergoing treatment but in general, it is expected that between 0.5 and 200 ml of tetracycline solution would be used at a time. When given systemically, higher doses of tetracycline will be given but it is expected that the total amount needed will be significantly less than that typically used to treat bacterial infections (usually 1-2 grams per day in adults divided into 2-4 equal doses and, in children, 10-20 mg per pound of body weight per day). It is expected that 100-200 mg per day should be effective in most cases.

The effectiveness of a dosage, as well as the effectiveness of the overall treatment can be assessed by monitoring tumor size using standard imaging techniques over a period of days, weeks and/or months. A shrinkage in the size or number of tumors is an indication that the treatment has been successful. If this does not occur or continue, then the treatment can be repeated as many times as desired. In addition, treatment with virus can be combined with any other therapy typically used for solid tumors, including surgery, radiation therapy or chemotherapy. In addition, the procedure can be combined with methods or compositions designed to help induce an immune response.

As used herein, the term “therapeutically effective amount” is intended to mean the amount of vector which exerts oncolytic activity, causing attenuation or inhibition of tumor cell proliferation, leading to tumor regression. An effective amount will vary, depending upon the pathology or condition to be treated, by the patient and his or her status, and other factors well known to those of skill in the art. Effective amounts are easily determined by those of skill in the art. In some embodiments a therapeutic range is from 103 to 1012 plaque forming units introduced once. In some embodiments a therapeutic dose in the aforementioned therapeutic range is administered at an interval from every day to every month via the intratumoral, intrathecal, convection-enhanced, intravenous or intra-arterial route.

The invention provided herein can further be described in the following numbered paragraphs:
1. An oncolytic Herpes Simplex Virus (HSV) comprising recombinant DNA, wherein the recombinant DNA has both ICP0 and ICP34.5 gene product deleted or does not express functional ICP0 and ICP34.5 gene product.
2. An oncolytic Herpes Simplex Virus (HSV) comprising recombinant DNA, wherein the recombinant DNA comprises:
   a) a gene comprising a 5′ untranslated region and a HSV-1, or HSV-2, ICP27 gene that is operably linked to an ICP27 promoter comprising a TATA element;
   b) a tetracycline operator sequence positioned between 6 and 24 nucleotides 3′ to said TATA element, wherein the ICP27 gene lies 3′ to said tetracycline operator sequence;
   c) a ribozyme sequence located in said 5′ untranslated region of said gene;
   d) a gene sequence encoding tetracycline repressor operably linked to an HSV immediate-early promoter, wherein the gene sequence is located at the ICP0 locus; and
   e) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant,
wherein said oncolytic HSV does not encode functional ICP0 and functional ICP34.5 protein.

3 The oncolytic HSV of paragraph 2, wherein the variant gene is a gK variant gene that encodes an amino acid substitution selected from the group consisting of: an Ala to Val amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.

4 The oncolytic HSV of any preceding paragraph, wherein the variant gene is a UL24 gene that encodes a Ser to Asn amino acid substitution corresponding to amino acid 113 of SEQ ID NO: 3.

5 The oncolytic HSV of any preceding paragraph, further comprising a variant UL24 gene that encodes a Ser to Asn amino acid substitution corresponding to amino acid 113 of SEQ ID NO: 3.

6. The oncolytic HSV of any preceding paragraph, wherein the tetracycline operator sequence comprises two Op2 repressor binding sites.

7. The oncolytic HSV of any preceding paragraph, wherein the ICP27 promoter is an HSV-1 or HSV-2 ICP27 promoter.

8 The oncolytic HSV of any preceding paragraph, wherein the immediate-early promoter is an HSV-1 or HSV-2 immediate-early promoter.

9 The oncolytic HSV of any preceding paragraph, wherein the HSV immediate-early promoter is selected from the group consisting of: ICP0 promoter and ICP4 promoter.

10. The oncolytic HSV of any preceding paragraph, wherein the recombinant DNA is part of the HSV-1 genome.

11. The oncolytic HSV of any preceding paragraph, wherein the recombinant DNA is part of the HSV-2 genome.

12. The oncolytic HSV of any preceding paragraph, further comprising a pharmaceutically acceptable carrier.

13. The oncolytic HSV of any preceding paragraph, further encoding at least one polypeptide that can increase the efficacy of the oncolytic HSV to induce an anti-tumor-specific immunity.

14. The oncolytic HSV of any preceding paragraph, wherein the at least one polypeptide encodes a product selected from the group consisting of: interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, CTLA-4 antibody or antibody reagent, TIM-3 antibody or antibody reagent, and TIGIT antibody or antibody reagent.

15. A composition comprising an oncolytic HSV of any preceding paragraph.

16. The composition of any preceding paragraph, further comprising a pharmaceutically acceptable carrier.

17. A method for treating cancer, the method comprising administering the oncolytic HSV of any preceding paragraph or the composition of any preceding paragraph to a subject having cancer.

18. The method of any preceding paragraph, wherein the cancer is a solid tumor.

19. The method of any preceding paragraph, wherein the tumor is benign or malignant.

20 The method of any preceding paragraph, wherein the subject is diagnosed or has been diagnosed as having cancer is selected from the list consisting of: a carcinoma, a melanoma, a sarcoma, a germ cell tumor, and a blastoma.

21. The method of any preceding paragraph, wherein the subject is diagnosed or has been diagnosed as having a cancer selected from the group consisting of: non-small-cell lung cancer, breast cancer, brain cancer, colon cancer, prostate cancer, liver cancer, lung cancer, ovarian cancer, skin cancer, and pancreatic cancer.

22. The method of any preceding paragraph, wherein the cancer is metastatic.

23. The method of any preceding paragraph, further comprising administering an agent that regulates the tet operator-containing promoter.

24. The method of any preceding paragraph, wherein the agent is doxycycline or tetracycline.

25. The method of any preceding paragraph, wherein the agent is administered locally or systemically.

26. The method of any preceding paragraph, wherein the oncolytic virus is administered directly to the tumor.

### EXAMPLES

HSV replicates in epithelial cells and fibroblasts and establishes life-long latent infection in neuronal cell bodies within the sensory ganglia of infected individuals. During productive infection, HSV genes fall into three major classes based on the temporal order of their expression: immediate-early (IE), early (E), and late (L) (Roizman, 2001). The HSV-1 viral proteins directly relevant to the current study are two IE regulatory proteins, ICP27 and ICP0. ICP27 is an essential viral IE protein that modifies and transports viral transcripts to the cytoplasm (Sandri-Goldin, 2008). Although not essential for productive infection, ICP0 is required for efficient viral gene expression and replication at low multiplicities of infection in normal cells and efficient reactivation from latent infection (Cai and Schaffer, 1989; Leib et al., 1989; Yao and Schaffer, 1995). Studies have revealed that ICP0 is needed to stimulate translation of viral mRNA in quiescent cells (Walsh and Mohr, 2004) and plays a key role in blocking IFN-induced inhibition of viral infection (Eidson et al., 2002; Mossman et al., 2000). ICP0 also has E3 ubiquitin ligase activity and induces the disruption and degradation of ND10 proteins that have been implicated in controlling cell senescence and DNA repair (Everett, 2006). Given that tumor cells are impaired in various cellular pathways, such as DNA repair, interferon signaling, and translation regulation (Kastan and Bartek, 2004; Mohr, 2005), it is not surprising that ICP0 deletion mutants replicate more efficiently in cancer cells than in normal cells, in particular, quiescent cells and terminally differentiated cells. The oncolytic potential of ICP0 mutants was first illustrated by Yao and Schaffer (Yao and Schaffer, 1995), who showed that the plaque-forming efficiency of an ICP0 null mutant in human osteoscarcoma cells (U2OS) is 100- to 200-fold higher than in non tumorigenic African green monkey kidney cells (Vero). The preferential ability of ICP0 mutants to replicate in selected types of cancer cells has been further explored in the recent study of Hummel et al. with an HSV-1 virus lacking both ICP0 and HSV-1 virion-associated transactivator, VP16 (Hummel et al., 2005).

Using the T-REx™ (Invitrogen, CA) gene switch technology and a self-cleaving ribozyme, a novel regulatable oncolytic HSV-1 recombinant, KTR27, which encodes the tetR gene controlled by the ICP0 promoter at the ICP0 locus and the essential ICP27 gene under control of the tetO-bearing ICP27 promoter was constructed (Yao et al., 2010). Infection of normal replicating cells as well as multiple human cancer cell types with KTR27 in the presence of tetracycline led to 1000- to 250,000-fold higher progeny virus production than in the absence of tetracycline, while little viral replication and virus-associated cytotoxicity are observed in infected growth-arrested normal human cells. Intratumoral inoculation with KTR27 was shown to markedly inhibit tumor growth in a xenograft model of human non-small-cell lung cancer in nude mice. It was shown further that replication of KTR27 in the inoculated tumors can be efficiently controlled by local co-delivery of tetracycline to the target tumors at the time of KTR27 inoculation. Collectively, KTR27 possesses a unique pharmacological feature that can limit its replication to the targeted tumor microenvironment with localized tetracycline delivery, thus minimizing unwanted viral replication in distant tissues following local virotherapy. This regulatory mechanism would also allow the replication of the virus to be quickly shut down should adverse effects be detected.

Human cancers are heterogeneous and contain multiple barriers that limit viruses from efficiently infecting distant tumor cells following initial viral replication (McKee et al., 2006; Nagano et al., 2008; Pluen et al., 2001). In an effort to overcome the inability of oncolytic viruses or viral vectors to infect or deliver therapeutic gene to large number of tumor cells within the tumor mass, a viral fusogentic glycoprotein approach has been employed. It was specifically contemplated that a fusogenic variant of KTR27 could offer a significant immunological benefit in augmenting the anti-tumor response induced by KTR27.

HSV encodes several surface glycoproteins that involve the fusion of the viral envelope with the cell membrane as well as the fusion of an infected cell with adjacent cells, leading to syncytia. HSV variants exhibiting extensive syncytium formation consisting of as many as thousands of nuclei can be isolated by the propagation of virus in cell cultures (Pertel and Spear, 1996). Studies have shown that mutations in the cytoplasmic domain of HSV-1 glycoprotein B (gB) can lead to extensive syncytial (Baghian A et al., J Virol. 67:2396-2401, 1993; Bzik D J et al., Virology 137:185-190, 1984; Cai W H et al., J Virol 62:2596-2604, 1988; Engel J P et al., Virology 192:112-120, 1993; Diakidi-Kosta A et al., Gage P J et al., J Virol 67:2191-2201, 1993; Virus Res 93-99-108, 2003). HSV-1 syncytial mutations have also been identified in gene encoding for glycoprotein K (gK) (Bond V C et al., J Gen Virol 61:245-254, 1982; Bond V C and Person S, Virology 132:368-376, 1984; Debroy C et al., et al., Virology 145:36-48, 1985; Hutchinson et al., J Virol 66:5603-5609; Pogue-Geile K L et al., Virology 136:100-109, 1984; Pogue-Geile K L et al., Virology 157:67-74, 1987), the UL20 gene (Melancon J M et al., J Virol 78:7329-7343, 2004) and the UL24 gene (Sanders P G et al., J Gen Virol 63:277-95, 1982; Jacobson J G et al., J Virol 63:1839-1843; Jacobson J G et al., Virology 242:161-169, 1998). Notably, UL20 interacts with both gB and gK (Foster T P et al., J Virol 82:6310-6323, 2008; Chouljenko V N et al., J Virol 84:8596-8606).

During the propagation of KTR27 in U2OS cells, the presence of fusogenic forms of KTR27 was noticed in addition to the non-fusogenic regular KTR27 in passage 3 KTR27 stock. KTR27-F was a second-round plaque-purified syncytium-forming KTR27 variant (KTR27-F) with a plaque size ~12 times larger than that of parental KTR27 and exhibited similar replication efficiency as KTR27 in U2OS cells. While the replication efficiency of KTR27-F and KTR27 is comparable in the tested various human cancer cell lines, it was shown that KTR27-F exhibits more stringent tet-dependent regulation in these cells lines with regulatability ranges from ~65,000-fold to ~881,000-fold, whereas the degrees of KTR27 regulation ranged from ~785-fold to ~37,000-fold. The effectiveness of KTR27-F in killing tested human lung and breast tumor cell lines is enhanced 11 to 37-fold at a low multiplicity of infection.

Sequence analyses of KTR27-F genome confirms that KTR27-F encodes tetR at the HSV-1 ICP0 locus, and ICP27 under the control of the tetO-containing ICP27 promoter with a self-cleaving ribozyme present at the 5′ untranslated region of ICP27 gene. Using the parental wild-type HSV-1 strain KOS genome as the reference, a single amino acid substitution, Ala to Val at residue 40, is identified in the gK gene of KTR27-F, while no mutation is found in the gB gene and the UL20 gene. KTR27-F also contains a single amino acid substitution, Ser to Asn at the residue 113 in UL24 gene. Because the same Ala to Val substitution has been identified in the HSV-1 syncytial mutants, syn102, syn105 and syn 33 (Dolter K E et al., J Virol 68:8277-8281, 1994), which were isolated from KOS-infected cells in the presence of mutagens, 2-aminopurine (Bond V C et al., J Gen Virol 61:245-254, 1982) or 5-bromodeoxyuridine (Read G S et al., J Virol 35:105-113, 1980), it is specifically contemplated that the Ala to Val substitution at residue 40 of the gK gene in KTR27-F is a key factor for the observed fusogenic phenotype. Previous studies identified several additional syncytial mutations in the gK gene, which include Ala to Thr at residue 40 in syn20, Asp to Asn at residue 99 in syn31 and syn32, Leu to Pro at residue 304 in syn30, and Arg to Leu at residue 310 (Dolter K E et al., J Virol 68:8277-8281, 1994). Whether the Ser to Asn substitution at residue 113 in the UL24 gene contributes to the fusogenic activity of KTR27-F remains to be determined.

Surprisingly, sequencing analysis indicates that KTR27-F does not encode the HSV-1 ICP34.5 gene. Like ICP0, the ICP34.5 gene is located in the inverted repeat region that flanks the unique long region of the HSV-1 genome. PCR analyses with primers specific for the ICP34.5 gene indicate that the ICP34.5 gene is likely non-specifically lost during the construction of KOR27-lacZ, the parental virus of KTR27.

### Materials and Methods

### Cells and Viruses.

The osteosarcoma line U2OS and the African green monkey kidney cell line (Vero) were grown in Dulbecco's modified Eagle's medium (DMEM) supplemented with 10% fetal bovine serum (FBS) (Yao and Schaffer, 1995). U2OS cells express a cellular activity that can effectively complement the function of the HSV-1 IE regulatory protein ICP0 lacking in ICP0-mutant viruses (Yao and Schaffer, 1995). Primary human fibroblasts were grown in DMEM containing 10% FBS plus 1× non-essential amino acids (Yao and Eriksson, 1999).

Human non-small-cell lung cancer cells (H1299), human breast cancer cells (MCF7), human prostate cancer cells (PC1435), and pancreatic cancer cells (Panc 1) were cultured in DMEM containing 10% FBS. PC1435 and MCF7 were kindly provided by Dr. Sheng Xiao (Brigham and Women's Hospital). Panc 1 was the kind gift of Dr. Edward Hwang (Brigham and Women's Hospital).

7134 is an ICP0-null mutant derived from HSV-1 strain KOS, in which both copies of the ICP0 coding sequence are replaced by the LacZ gene of Escherichia coli (Cai and Schaffer, 1989). 7134 was propagated and assayed in U2OS cells (Yao and Schaffer, 1995). KOR is an HSV-1 recombinant generated by recombinational replacement of the LacZ gene in 7134 with the DNA sequence encoding tetR (Yao et al., 2006). KOR27-lacZ was derived from KOR in which the ICP27 coding sequence was replaced with the LacZ gene by homologous recombination (Yao et al., 2010). KTR27 is a 7134-derived recombinant virus that encodes tetR under the control of HSV-1 ICP0 promoter at the ICP0 locus, and the essential ICP27 gene under the control of the tetO-containing ICP27 promoter and a self-cleaving ribozyme located at the 5′ untranslated region of ICP27 coding sequence (Yao et al., J Virol, 2010) (U.S. Pat. No. 8,236,941).

### Neurovirulence of KTR27-F.

A mouse model for the evaluation of the neurovirulence of KTR27-F was established by injecting 4-6 week female CD1 outbred mice (Charles River Laboratories, Wilmington, MA) with 20 μl of medium containing 1×10⁷ PFU of KTR27-F or 7134. Intracerebral inoculation was performed with a 28½ gauge needle with a needle guard such that the distance from the guard to the needle tip was 5.5 mm, and to the beginning of the bevel of the needle was 4.5 mm. The needle was inserted at a point equidistant between the outer canthus of the eye, the front of the pinna, and midline of the head (Lynas et al., 1993). Half of the mice inoculated with KTR27-F were given a normal diet, and the other half were fed a doxycycline-containing diet at 200 mg/kg (Bio-Serv, Frenchtown, NJ), beginning 3 days prior to inoculation and lasting for the duration of the experiment. Mice were examined for signs of illness for 29 days following inoculation.

All mouse studies were conducted in accordance with the protocols set forth by the Harvard Medical Area Standing Committee on Animals and the American Veterinary Medical Association. The Harvard Medical School animal management program is accredited by the Association for Assessment and Accreditation of Laboratory Animal Care (AAALAC) and meets National Institutes of Health standards as set forth in “The Guide for the Care and Use of Laboratory Animals” (National Academy Press, 1996).

### Illumina Sequencing.

KTR27-F viral DNA was prepared from KTR27-F-infected U2OS cells with Qiagen Genomic DNA kit. Quantitative real-time PCR analysis reveals close to 55% of total DNA represents KTR27F viral DNA. The isolated DNA (2.2 μg) was used for library construction with TruSeq DNA OCR-Free Library Preparation Kits at Translational Genomics Core Facility, Partners HealthCare, Cambrige, MA, targeting 550 bp fragments, and were sequenced on a 250 bp MiSeq run. The resulting contigs were assembled and analyzed in Illumina MiSeq Reporter Resequencing workflow using HSV-1 strain KOS genome as the reference.

### Results

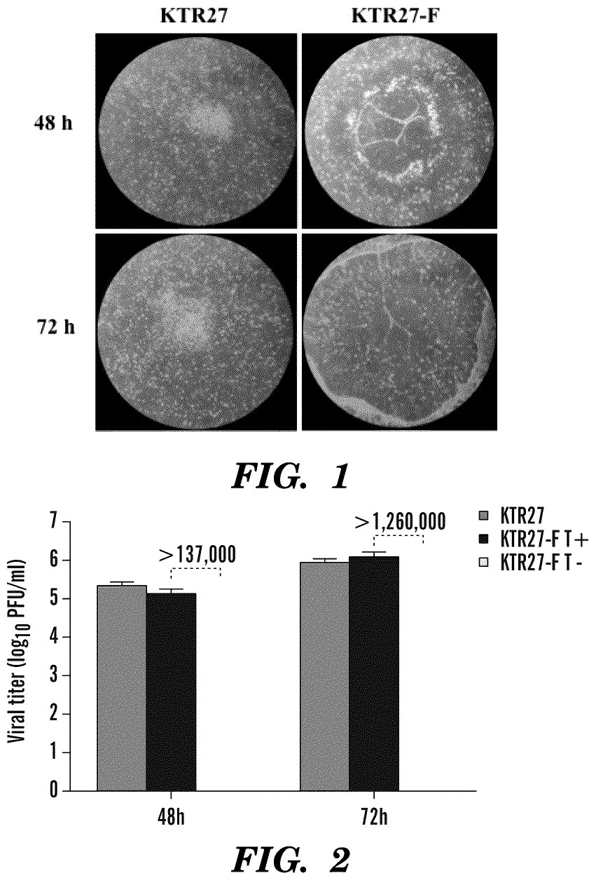

Selection of KTR27-F. During the propagation of KTR27 in U2OS cells, the presence of fusogenic forms of KTR27 was noticed in addition to the non-fusogenic regular KTR27 in passage 3 KTR27 stock. To isolate fusogenic variants of KTR27, passage 3 KTR27 was diluted with DMEM containing 10% FBS followed by plaque purification. Specifically, 10×100 mm dishes of confluent 72 h-old U2OS cells were infected with the diluted passage 3 KTR27 at either 100 PFU/dish or 200 PFU/dish. After 1 h incubation at 37 C, inoculation medium was removed and 10 ml/dish of DMEM growth medium containing tetracycline at 10 μg/ml were added to each dish. After an additional 3 h incubation at 37 C, tetracycline-containing medium was removed from individual dishes followed by addition of 1 ml/dish of fresh tetracycline-containing DMEM growth medium, Infected cells were then overlaid with 1% methylcellulose prepared in DMEM containing 5% FBS at 25 ml/dish. After incubation at 37 C for 72 h, infected dishes were stained with 10 ml/dish of 0.02% neutral-red prepared in DMEM. Individual single fusogenic plaques were picked at 20 h post neutral-red staining and suspended in 1.5 ml of DMEM growth medium followed by amplification in U2OS cells in the presence of tetracycline. KTR27-F was a second-round plaque-purified syncytium-forming KTR27 variant with a plaque size ~12-13 times larger than that of parental KTR27 at 48 and 72 h post-infection (FIG. 1), while exhibited similar replication efficiency as KTR27 in U2OS cells.

Control of KTR27-F replication by tetracycline. To assess the dependence of KTR27-F replication on the presence of tetracycline, Vero cells were infected with KTR27-F at a MOI of 1 PFU/cell in the presence and absence of tetracycline and the infected cells were harvested at 48 and 72 h post-infection (FIG. 2). While the yield of KTR27-F at 72 h post-infection was 1.26×10⁶ PFU/ml, no infectious KTR27-F was detectable in cells infected in the absence of tetracycline at either time point, indicating that the regulation of KTR27-F viral replication by tetracycline is greater than 1,260,000-fold in Vero cells.

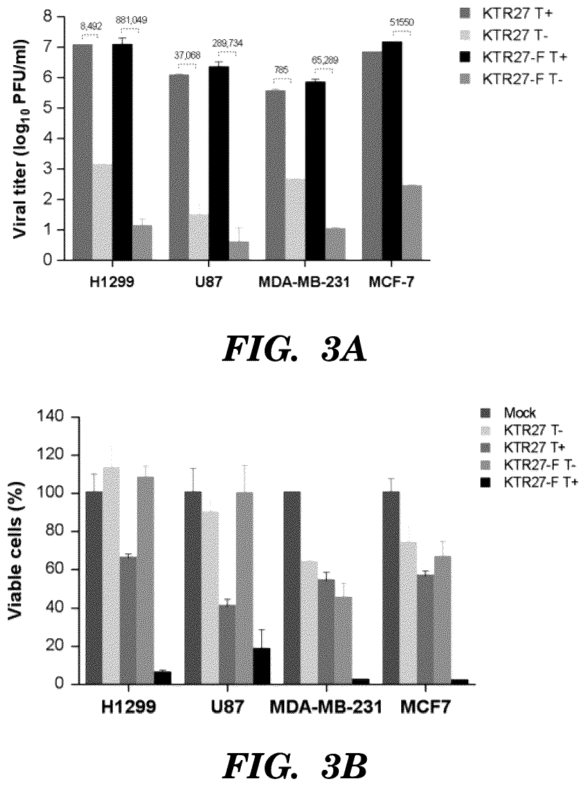

Tetracycline-dependent replication of KTR27-F in cultured human tumor cells and primary cells. Having demonstrated that the replication of KTR27-F is as productive as that of KTR27 in Vero cells, and that KTR27-F is unable to replicate in Vero cells in the absence of tetracycline, the replicative and regulative abilities of KTR27-F in various human tumor cell lines were then investigated. As a control, KTR27 was also used in these experiments. As depicted in FIG. 3A, KTR27-F infection of human lung, brain, and breast tumor cell lines demonstrated that KTR27-F regulatability ranges from ~52,000-fold to ~880,000-fold, whereas the degrees of KTR27 regulation ranged from ~785-fold to ~37,000-fold. The enhanced regulatability of KTR27-F relative to that of KTR27 is a combination of slightly increased viral yields in the presence of tetracycline and significantly reduced yields in the absence of tetracycline.

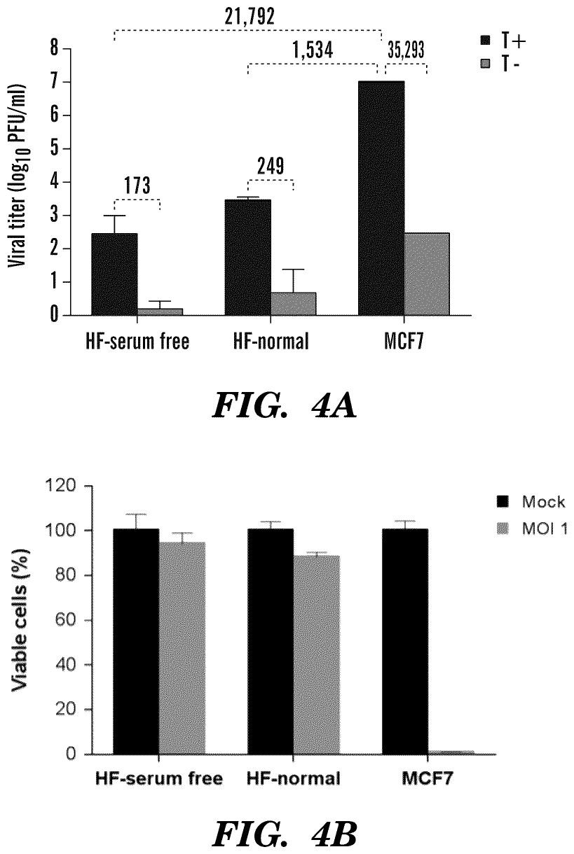

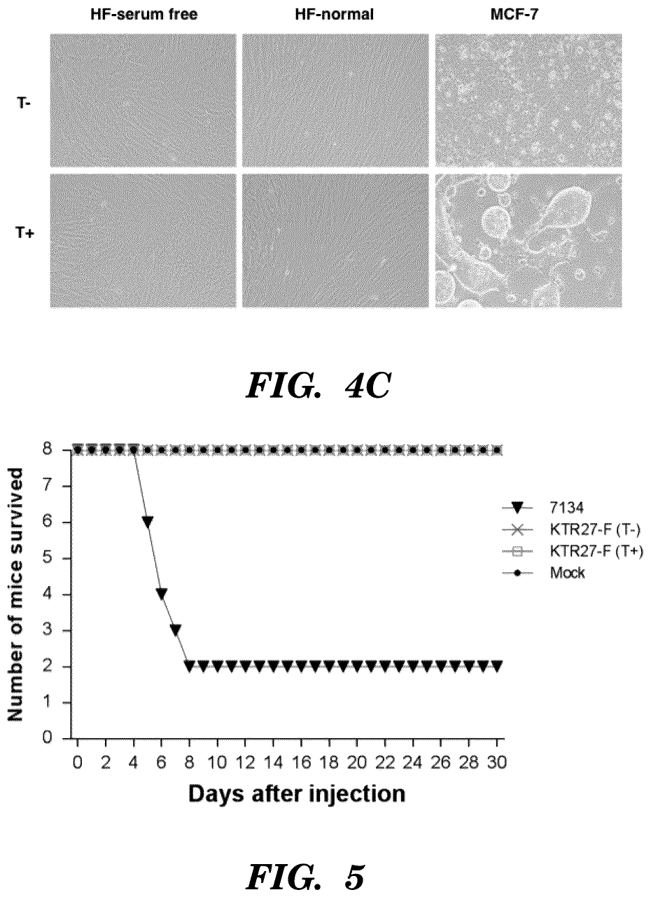

The drastic enhancement of the cytotoxic effect of KTR27-F relative to that of KTR27 is best visualized by the cytotoxicity assays depicted in FIG. 3B. In the human cancer cell lines H1299, U87, MDA-MB-231, and MCF-7, cell death following KTR27-F infection in the presence of tetracycline was ~11-fold, ~2.3-fold, ~28-fold, and ~37-fold higher, respectively, than cell death following KTR27 infection in the presence of tetracycline. To directly examine the oncoselectivity of KTR27-F in non-tumor primary human cells relative to a cancer line of similar tissue type, MCF-7 cells and dividing and non-dividing human breast fibroblasts were infected with KTR27-F in the presence and absence of tetracycline as described by Yao et al. (2010). The results of FIG. 4A demonstrate that replication of KTR27-F in primary human fibroblasts, particularly non-dividing fibroblasts, is reduced compared with replication in MCF-7. Yields of KTR27-F at 72 h post-infection in MCF7 cells were approximately 21,800-fold higher than those in the serum-starved fibroblasts, and 1,530-fold higher than in fibroblasts grown in normal growth medium. Additionally, the cytotoxic effect of KTR27-F infection in the presence of tetracycline was evaluated (FIG. 4B). The results show that KTR27-F exhibits little cytotoxic effect in non-dividing fibroblasts, modest cytotoxic effect in dividing fibroblasts (88% of infected cells remained viable), and drastic cytotoxic effect in MCF-7 cells (0.8% of infected cells remained viable). The corresponding morphological images of cells from the cytotoxicity assay (FIG. 4C) depict this cytopathic effect in MCF-7 (note the extensive formation of syncytia). In contrast, very little or no cytotoxic effects are visible among the infected or mock-infected human fibroblasts. Together, the results presented in FIGS. 4A and 4B indicate that the ability of KTR27-F to replicate in and kill normal primary human fibroblasts is markedly reduced relative to various human tumor cell lines.

Neurovirulence of KTR27-F. The ability of an oncolytic viral recombinant to replicate efficiently in tumor cells must be balanced against the potentially dangerous side effects of its replication in non-tumor tissues. HSV is highly neurotropic, and thus a clinically-relevant HSV recombinant ideally causes little to no neurovirulence. KTR27 was previously demonstrated to be avirulent following intracerebral inoculation in mice (Yao et al., 2010), herein, a similar assay was conducted with KTR27-F to investigate should the enhanced cytotoxicity of KTR27-F in the presence of tetracycline in cancer cells lead to a higher degree of neurovirulence. In brief, mice receiving a doxycycline-containing diet or normal diet were intracerebrally inoculated with KTR27-F at a dose of 1×10⁷ PFU/mouse (FIG. 5), along with control groups injected with DMEM or 7134 at a dose of 1×10⁷ PFU/mouse, and monitored the mice for 29 days. The groups injected with DMEM, KTR27-F in the presence of doxycycline (T+), and KTR27-F in the absence of doxycycline (T−) showed no signs of neurovirulence throughout the course of the experiment, whereas all of the mice injected with 7134 showed signs of central nervous system (CNS) illness commonly associated with HSV-1 infection, including roughened fur, hunched posture, ataxia, and anorexia. Six of the eight 7134-inoculated mice died by day 8 post-inoculation, and two of the eight fully recovered from CNS illness within 11 days post-inoculation. In light of the demonstration that the doxycycline concentration in the brains of mice receiving the doxycycline-containing diet can efficiently release the tetR-mediated repression of gene expression following intracerebral inoculation of the T-REX-encoding replication-defective HSV-1 recombinant virus (Yao et al., 2006), the study indicates that the observed avirulence of KTR27-F in mice receiving a doxycycline-containing diet is primarily the result of impairment in the ability of KTR27 to replicate in the mouse brain.

Sequence analyses of KTR27-F genome. As expected, sequence analysis of KTR27-F viral genome confirms that KTR27-F encodes tetR at the HSV-1 ICP0 locus, and ICP27 under the control of the tetO-containing ICP27 promoter with a self-cleaving ribozyme present at the 5′ untranslated region of ICP27 gene. Using the parental wild-type HSV-1 strain KOS genome as the reference, a total of 58 missense mutations and 2 frame shift mutations are identified in the KTR27-F genome. The UL36 gene of KTR27-F contains 16 missense mutations and 2 frame shift mutations. Other missense mutations are located in the UL5 gene, the UL8 gene, the UL12 gene, the UL13 gene, the UL16 gene, UL17 gene, UL19 gene, the UL24 gene, the UL25 gene, UL26 gene, the UL28 gene, the UL29 gene, the UL30 gene, the UL37 gene, the UL39 gene, the UL40 gene, the UL44 gene, UL47 gene, the UL52 gene, the UL53 gene (gK), the US1 gene, and the US8 gene.

A single amino acid substitution, Ala to Val at residue 40, is identified in the gK gene of KTR27-F. The same Ala to Val substitution has been identified in the HSV-1 syncytial mutants, syn 102, syn105 and syn 33 (Dolter K E et al., J Virol 68:8277-8281, 1994), which were isolated from KOS-infected cells in the presence of mutagens, 2-aminopurine (Bond V C et al., J Gen Virol 61:245-254, 1982) or 5-bromodeoxyuridine (Read G S et al., J Virol 35:105-113, 1980), indicating that the Ala to Val substitution at residue 40 of the gK gene in KTR27-F is a key factor for the observed fusogenic phenotype. Syncytial mutations in the gK gene also include Ala to Thr at residue 40 in syn20, Asp to Asn at residue 99 in syn31 and syn32, Leu to Pro at residue 304 in syn30, and Arg to Leu at residue 310 (Dolter K E et al., J Virol 68:8277-8281, 1994). In addition to the single amino acid substitution in the gK gene, KTR27-F contains a single amino acid substitution of Ser to Asn in UL24 gene at residue 113. Whether this Ser to Asn substitution contributes to the fusogenic activity of KTR27-F remains to be determined. No mutation is found in the gene encoding gB and the UL20 gene.

Unexpectedly, sequencing analysis of KTR27-F reveals that the HSV-1 ICP34.5 gene is missing from the KTR27-F genome. To date, most of HSV-1 based oncolytic viruses are based on deletion of the ICP34.5 gene or through conditional regulations of ICP34.5 expression (Aghi M and Martuza R L, Oncogen 24:7802-7816, 2005; Lawler S E et al., JAMA Oncology, 2016). The ICP35.5 deletion mutant-based HSV-1 oncolytic virus, T-Vec (Amgen) has been approved for the treatment of advanced-stage melanoma in late 2015. Like ICP0, the ICP34.5 gene is located in the inverted repeat region that flanks the unique long region of the HSV-1 genome. PCR analyses with primers specific for the ICP34.5 gene indicate that while both 7134 and KOR yield a predicated ICP34.5-specific amplified PCR fragment, no ICP34.5-specific DNA fragment was detected in PCR reactions with KTR27, KTR27-F, and KOR27-lacZ viral DNA. PCR analysis with tetR-specific primers confirm that KTR27, KTR27-F, and KOR27-lacZ encode tetR at the ICP0 locus. Collectively, these results indicate that the ICP34.5 gene was likely lost during the construction of KOR27-lacZ virus.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Other than in the operating examples, or where otherwise indicated, all numbers expressing quantities of ingredients or reaction conditions used herein should be understood as modified in all instances by the term “about.” The term “about” when used in connection with percentages can mean ±1%.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

### REFERENCES

Advani, S. J., Sibley, G. S., Song, P. Y., Hallahan, D. E., Kataoka, Y., Roizman, B., and Weichselbaum, R. R. (1998). Enhancement of replication of genetically engineered herpes simplex viruses by ionizing radiation: a new paradigm for destruction of therapeutically intractable tumors. Gene Ther 5, 160-165.
Aghi, M., and Martuza, R. L. (2005). Oncolytic viral therapies—the clinical experience. Oncogene 24, 7802-7816.
Cai, W. Z., and Schaffer, P. A. (1989). Herpes simplex virus type 1 ICP0 plays a critical role in the de novo synthesis of infectious virus following transfection of viral DNA. J Virol 63, 4579-4589.
Chung, R. Y., Saeki, Y., and Chiocca, E. A. (1999). B-myb promoter retargeting of herpes simplex virus gamma34.5 gene-mediated virulence toward tumor and cycling cells. J Virol 73, 7556-7564.
Eidson, K. M., Hobbs, W. E., Manning, B. J., Carlson, P., and DeLuca, N. A. (2002). Expression of herpes simplex virus ICP0 inhibits the induction of interferon-stimulated genes by viral infection. J Virol 76, 2180-2191.
Everett, R. D. (2006). Interactions between DNA viruses, ND10 and the DNA damage response. Cell Microbiol 8, 365-374.
Hummel, J. L., Safroneeva, E., and Mossman, K. L. (2005). The role of ICP0-Null HSV-1 and interferon signaling defects in the effective treatment of breast adenocarcinoma. Mol Ther 12, 1101-1110.
Kastan, M. B., and Bartek, J. (2004). Cell-cycle checkpoints and cancer. Nature 432, 316-323.
Leib, D. A., Coen, D. M., Bogard, C. L., Hicks, K. A., Yager, D. R., Knipe, D. M., Tyler, K. L., and Schaffer, P. A. (1989). Immediate-early regulatory gene mutants define different stages in the establishment and reactivation of herpes simplex virus latency. J Virol 63, 759-768.
Martuza, R. L., Malick, A., Markert, J. M., Ruffner, K. L., and Coen, D. M. (1991). Experimental therapy of human glioma by means of a genetically engineered virus mutant. Science 252, 854-856.
McKee, T. D., Grandi, P., Mok, W., Alexandrakis, G., Insin, N., Zimmer, J. P., Bawendi, M. G., Boucher, Y., Breakefield, X. O., and Jain, R. K. (2006). Degradation of fibrillar collagen in a human melanoma xenograft improves the efficacy of an oncolytic herpes simplex virus vector. Cancer Res 66, 2509-2513.
Mohr, I. (2005). To replicate or not to replicate: achieving selective oncolytic virus replication in cancer cells through translational control. Oncogene 24, 7697-7709.
Mossman, K. L., Saffran, H. A., and Smiley, J. R. (2000). Herpes simplex virus ICP0 mutants are hypersensitive to interferon. J Virol 74, 2052-2056.
Nagano, S., Perentes, J. Y., Jain, R. K., and Boucher, Y. (2008). Cancer cell death enhances the penetration and efficacy of oncolytic herpes simplex virus in tumors. Cancer Res 68, 3795-3802.
Parato, K. A., Senger, D., Forsyth, P. A., and Bell, J. C. (2005). Recent progress in the battle between oncolytic viruses and tumours. Nat Rev Cancer 5, 965-976.
Pluen, A., Boucher, Y., Ramanujan, S., McKee, T. D., Gohongi, T., di Tomaso, E., Brown, E. B., Izumi, Y., Campbell, R. B., Berk, D. A., et al. (2001). Role of tumor-host interactions in interstitial diffusion of macromolecules: cranial vs. subcutaneous tumors. Proc Natl Acad Sci USA 98, 4628-4633.
Roizman, B., and D. M. Knipe (2001). Herpes simplex viruses and their replication. In Fields Virology, a. P. M. H. D. M. Knipe, ed. (Philadelphia, Pa: Lippincott Williams & Wilkins), pp. 2399-2459.
Sandri-Goldin, R. M. (2008). The many roles of the regulatory protein ICP27 during herpes simplex virus infection. Front Biosci 13, 5241-5256.
Shen, Y., and Nemunaitis, J. (2005). Fighting cancer with vaccinia virus: teaching new tricks to an old dog. Mol Ther 11, 180-195.
Walsh, D., and Mohr, I. (2004). Phosphorylation of eIF4E by Mnk-1 enhances HSV-1 translation and replication in quiescent cells. Genes Dev 18, 660-672.
Yao, F., and Eriksson, E. (1999). A novel anti-herpes simplex virus type 1-specific herpes simplex virus type 1 recombinant. Hum Gene Ther 10, 1811-1818.
Yao, F., Murakami, N., Bleiziffer, O., Zhang, P., Akhrameyeva, N. V., Xu, X., and Brans, R. (2010). Development of a regulatable oncolytic herpes simplex virus type 1 recombinant virus for tumor therapy. J Virol 84, 8163-8171.
Yao, F., and Schaffer, P. A. (1995). An activity specified by the osteosarcoma line U2OS can substitute functionally for ICP0, a major regulatory protein of herpes simplex virus type 1. J Virol 69, 6249-6258.
Yao, F., Theopold, C., Hoeller, D., Bleiziffer, O., and Lu, Z. (2006). Highly efficient regulation of gene expression by tetracycline in a replication-defective herpes simplex viral vector. Mol Ther 13, 1133-1141.

Sequence Listing

SEQ ID NO: 1 is a nucleotide sequence that
encodes KTR27-F Linear Genome (147,630 bp)
CCCTAGAGGATCTGCGGCTGGAGGGTCGCTGACGGAGGGT
CCCTGGGGGTCGCAACGTAGGCTTTTCTTCTTTTTTTCTT
CTTCCCTCCCCCGCCCGAGGGGGCGCCCGAGTCTGCCTGG
CTGCTGCGTCTCGCTCCGAGTGCCGAGGTGCAAATGCGAC
CAGACCGTCGGGCCAGGGCTAACTTATACCCCACGCCTTT
CCCCTCCCCAAAGGGGCGGCAGTGACGATTCCCCCAATGG
CCGCGCGTCCCAGGGGAGGCAGGCCCACCGCGGAGCGGCC
CCGTCCCCGGGGACCAACCCGGCGCCCCCAAAGAATATCA
TTAGCATGCACGGCCCGGCCCCCGATTTGGGGGACCAACC
CGGTGTCCCCCAAAGAACCCCATTAGCATGCCCCTCCCGC
CGACGCAACAGGGGCTTGGCCTGCGTCGGTGCCCCGGGGC
TTCCCGCCTTCCCGAAGAAACTCATTACCATACCCGGAAC
CCCAGGGGACCAATGCGGGTTCATTGAGCGACCCGCGGGC
CACTGCGCGAGGGGCCGTGTGTTCCGCCAAAAAAGCAATT
AACATAACCCGGAACCCCAGGGGAGTGGTTACGCGCGGCG
CGGGAGGCGGGGAATACCGGGGTTGCCCATTAAGGGCCGC
GGGAATTGCCGGAAGCGGGAAGGGCGGCCGGGGCCGCCCA
TTAATGAGTTTCTAATTACCATCCCGGGAAGCGGAACAAG
GCCTCTGCAAGTTTTTAATTACCATACCGGGAAGTGGGCG
GCCCGGCCCACTGGGCGGGAGTTACCGCCCAGTGGGCCGG
GCCCCGACGACTCGGCGGACGCTGGTTGGCCGGGCCCCGC
CGCGCTGGCGGCCGCCGATTGGCCAGTCCCGCCCCCCGAG
GGCGGGCCCGCCTCGGGGGCGGGCCGGCCCCAAGCGAATA
TGCGCGGCTCCTGCCTTCGTCTCTCCGGAGAGCGGCTTGG
TGGCGGGGCCCGGCCACCAGCGTCCGCCGAGTCGTCGGGG
CCCGGCCCACTGGGCGGTAACTCCCGCCCAGTGGGCCGGG
CCGCCCACTTCCCGGTATGGTAATTAAAAACTTGCAGAGG
CCTTGTTCCGCTTCCCGGTATGGTAATTAGAAACTCATTA
ATGGGCGGCCCCGGCCGCCCTTCCCGCTTCCGGCAATTCC
CGCGGCCCTTAATGGGCAACCCCGGTATTCCCCGCCTCCC
GCGCCGCGCGTAACCACTCCCCTGGGGTTCCGGGTTATGT
TAATTGCTTTTTTGGCGGAACACACGGCCCCTCGCGCATT
GGCCCGCGGGTCGCTCAATGAACCCGCATTGGTCCCCTGG
GGTTCCGGGTATGGTAATGAGTTTCTTCGGGAAGGCGGGA
AGCCCCGGGGCACCGACGCAGGCCAAGCCCCTGTTGCGTC
GGCGGGAGGGGCATGCTAATGGGGTTCTTTGGGGGACACC
GGGTTGGTCCCCCAAATCGGGGGCCGGGCCGTGCATGCTA
ATGATATTCTTTGGGGGCGCCGGGTTGGTCCCCGGGGACG
GGGCCGCTCCGCGGTGGGCCTGCCTCCCCTGGGACGCGCG
GCCATTGGGGGAATCGTCACTGCCGCCCCTTTGGGGAGGG
GAAAGGCGTGGGGTATAAGTTAGCCCTGGCCCGACGGTCT
GGTCGCATTTGCACCTCGGCACTCGGAGCGAGACGCAGCA
GCCAGGCAGACTCGGGCCGCCCCCTCTCCGCATCACCACA
GAAGCCCCGCCTACGTTGCGACCCCCAGGGACCCTCCGTC
AGCGACCCTCCAGCCGCATACGACCCCCCGGGGATCCTCT
AGGGCCTCTGAGCTATTCCAGAAGTAGTGAAGAGGCTTTT
TTGGAGGCCTAGGCTTTTGCAAAAAGCTCCGGATCGATCC
TGAGAACTTCAGGGTGAGTTTGGGGACCCTTGATTGTTCT
TTCTTTTTCGCTATTGTAAAATTCATGTTATATGGAGGGG
GCAAAGTTTTCAGGGTGTTGTTTAGAATGGGAAGATGTCC
CTTGTATCACCATGGACCCTCATGATAATTTTGTTTCTTT
CACTTTCTACTCTGTTGACAACCATTGTCTCCTCTTATTT
TCTTTTCATTTTCTGTAACTTTTTCGTTAAACTTTAGCTT
GCATTTGTAACGAATTTTTAAATTCACTTTTGTTTATTTG
TCAGATTGTAAGTACTTTCTCTAATCACTTTTTTTTCAAG
GCAATCAGGGTATATTATATTGTACTTCAGCACAGTTTTA
GAGAACAATTGTTATAATTAAATGATAAGGTAGAATATTT
CTGCATATAAATTCTGGCTGGCGTGGAAATATTCTTATTG
GTAGAAACAACTACATCCTGGTCATCATCCTGCCTTTCTC
TTTATGGTTACAACGATATACACTGTTTGAGATGAGGATA
AAATACTCTGAGTCCAAACCGGGCCCCTCTGCTAACCATG
TTCATGCCTTCTTCTTTTTCCTACAGCTCCTGGGCAACGT
GCTGGTTATTGTGCTGTCTCATCATTTTGGCAAAGAATTG
TAATACGACTCACTATAGGGCGAATTGATATGTCTAGATT
AGATAAAAGTAAAGTGATTAACAGCGCATTAGAGCTGCTT
AATGAGGTCGGAATCGAAGGTTTAACAACCCGTAAACTCG
CCCAGAAGCTAGGTGTAGAGCAGCCTACATTGTATTGGCA
TGTAAAAAATAAGCGGGCTTTGCTCGACGCCTTAGCCATT
GAGATGTTAGATAGGCACCATACTCACTTTTGCCCTTTAG
AAGGGGAAAGCTGGCAAGATTTTTTACGTAATAACGCTAA
AAGTTTTAGATGTGCTTTACTAAGTCATCGCGATGGAGCA
AAAGTACATTTAGGTACACGGCCTACAGAAAAACAGTATG
AAACTCTCGAAAATCAATTAGCCTTTTTATGCCAACAAGG
TTTTTCACTAGAGAATGCATTATATGCACTCAGCGCTGTG
GGGCATTTTACTTTAGGTTGCGTATTGGAAGATCAAGAGC
ATCAAGTCGCTAAAGAAGAAAGGGAAACACCTACTACTGA
TAGTATGCCGCCATTATTACGACAAGCTATCGAATTATTT
GATCACCAAGGTGCAGAGCCAGCCTTCTTATTCGGCCTTG
AATTGATCATATGCGGATTAGAAAAACAACTTAAATGTGA
AAGTGGGTCCGCGTACAGCGGATCCCGGGAATTCAGATCT
TATTAAAGCAGAACTTGTTTATTGCAGCTTATAATGGTTA
CAAATAAAGCAATAGCATCACAAATTTCACAAATAAAGCA
TTTTTTTCACTGCATTCTAGTTGTGGTTTGTCCAAACTCA
TCAATGTATCTTATCATGTCTGGTCGACCCGGGACGAGGG
AAAACAATAAGGGACGCCCCCGTGTTTGTGGGGAGGGGGG
GGTCGGGCGCTGGGTGGTCTCTGGCCGCGCCCACTACACC
AGCCAATCCGTGTCGGGGAGGTGGAAAGTGAAAGACACGG
GCACCACACACCAGCGGGTCTTTTGTGTTGGCCCTAATAA
AAAAAACTCAGGGGATTTTTGCTGTCTGTTGGGAAATAAA
GGTTTACTTTTGTATCTTTTCCCTGTCTGTGTTGGATGTA
TCGCGGGGGTGCGTGGGAGTGGGGGCCCCCACTCCCACGC
ACCCCCACTCCCACGCACCCCCACTCCCACGCACCCCCGC
GATACATCCAACACAGACAGGGAAAAGATACAAAAGTAAA
CCTTTATTTCCCAACAGACAGCAAAAATCCCCTGAGTTTT
TTTTATTAGGGCCAACACAAAAGACCCGCTGGTGTGTGGT
GCCCGTGTCTTTCACTTTCCACCTCCCCGACACGGATTGG
CTGGTGTAGTGGGCGCGGCCAGAGACCACCCAGCGCCCGC
CCCCCCCCCCCCCACAACCCCGGGGGCGTCCCTTATTGTT
TCCCTCGTCCCGGGTCGACGTCGACCCGGGACGAGGGAAA
ACAATAAGGGACGCCCCCGTGTTTGTGGGGAGGGGGGGGT
CGGGCGCTGGGTGGTCTCTGGCCGCGCCCACTACACCAGC
CAATCCGTGTCGGGGAGGTGGAAAGTGAAAGACACGGGCA
CCACACACCAGCGGGTCTTTTGTGTTGGCCCTAATAAAAA
AAACTCAGGGGATTTTTGCTGTCTGTTGGGAAATAAAGGT
TTACTTTTGTATCTTTTCCCTGTCTGTGTTGGATGTATCG
CGGGGGTGCGTGGGAGTGGGGGTGCGTGGGAGTGGGGGTG
CGTGGGAGTGGGGGTGGGGGGGGGGGTGCGTGGGGGAGGG
GGGGCGTGGGAGTGGGGGTGCGTGGGGGTGGGGGTGCGTG
GGAGTGGCCCGGAGAGCCGCGGCCCCCGGACGCGCCCGGA
AAGTCTTTCGCCCACCGGCGATCGGCACGGCCGCACCCCC
GCTTTTATAAAGGCTCAGATGACGCAGCAAAAACAGGCCA
CAGCACCACATGGGTAGGGGATGTAATTTTATTTTCCTCG
TCTGCGGCCTAATGGATTTCCGGGCGCGGTGCCCCTGTCT
GCAGAGCACTTAACGGATTGATATCTCGCGGGCACGCGCG
CCCTTAAGGGGCCGGGGGGGGCGGGGGGCCGGATACCCAC
ACGGGCGGGGGGGGGTGTCGCGGGCCGTCTGCTGGCCCGC
GGCCACATAAACAATGACTCGGGGCCTTTCTGCCTCTGCC
GCTTGTGTGTGCGCGCGCCGGCTCTGCGGTGTCGGCGGCG
GCGGCGGCGGTGGCCGCCGTGTTCGGTCTCGGTAGCCGGC
CGGCGGGGGACTCGCGGGGGGCCGGAGGGTGGAAGGCAGG
GGGGTGTAGGATGGGTATCAGGACTTCCACTTCCCGTCCT
TCCATCCCCCGTTCCCCTCGGTTGTTCCTCGCCTCCCCCA
ACACCCCGCCGCTTTCCGTTGGGGTTGTTATTGTTGTCGG
GATCGTGCGGGCCGGGGGTCGCCGGGGCAGGGGCGGGGGC
GTGGGCGGGGGTGCTCGTCGATCGACCGGGCTCAGTGGGG
GCGTGGGGTGGGTGGGAGAAGGCGAGGAGACTGGGGTGGG
GGCGCCCCCACTGAGCCCGGTCGATCGACGAGCACCCCCG
CCCCCCCCCGCCCCTGCCCCGGCGACCCCCGGCCCGCACG
ATCCCGACAACAATAACAACCCCAACGGAAAGCGGCGGGG
TGTTGGGGGAGGCGAGGAACAACCGAGGGGAACGGGGGAT
GGAAGGACGGGAAGTGGAAGTCCTGATACCCATCCTACCC
CCCCCTGCCTTCCCCCCTCCGGCCCCCCGCGAGTCCACCC
GCCGGCCGGCTACCGAGACCGAACACGGCGGCCACCGCCG
CCGCCGCCGCCGACACCGCAGAGCCGGCGCGCGCACACAC
AAGCGGCAGAGGCAGAAAGGCCCCGAGTCATTGTTTATGT
GGCCGCGGGCCAGCAGACGGCCCGCGACACCCCCCCCCGC
CCGTGTGGGTATCCGGCCCCCCGCCCCGCGCCGGCCCCTT
AAGGGCGCGCGTGCCCGCGAGATATCAATCCGTTAAGTGC
TCTGCAGACAGGGGCACCGCGCCCGGAAATCCATTAGGCC
GCAGACGAGGAAAATAAAATTACATCACCTACCCATGTGG
GCTGTGGCCTGTTTTGCTGCGTCATCTGAGCCTTTATAAA
AGCGGGGGCGCGGTCGTTCCGATCGCCGGTGGTGCGAAAG
ACTTTCCGGGCGCTGGGGTGGGGGTGTCGGTGGGTGGTTG
TTTTTTTTTTTGTGGTTGTTTTTTGTGTCTGTTTCCGTCC
CCCGTCACCCCCCTCCCTCCGTCCCCTCCGTCCCCCCGTC
GCGGGTGTTTGTGTTTGTTTATTCCGACATCGGTTTATTT
AAAATAAACACAGCCGTTCTGCGTGTCTGTTCTTGCGTGT
GGCTGGGGGCTTATATGTGGGGTCCCGGGGGCGGGATGGG
GTTTAGCGGCGGGGGGCGGCGCGCCGGACGGGGCGCTGGA
GATAACGGCCCCCGGGGAACGGGGGACCGGGGCTGGGTAT
CCCGAGGTGGGTGGGTGGGCGGCGGTGGCCGGGCCGGGCC
GGGCCGGGCCGGGCCAGCGCCCCGCCGGCCCCCCCCCCCG
CCGCTAAACCCCATCCCGCCCCCGGGACCCCACATATAAG
CCCCCAGCCACACGCAAGAACAGACACGCAGAACGGCTGT
GTTTATTTTAAATAAACCGATGTCGGAATAAACAAACACA
AACACCCGCGACGGGGGGACGGAGGGGACGGAGGGAGGGG
GGTGACGGGGGACGGAAACAGACACAAAAAACAACCACAA
AAAAAAAAACAACCACCCACCGCACCCCCCCCCTTCTCCT
CCTCCTCCTCGTTTTCCAACCCCGCCCACCCGGCCCGGCC
CGGCCCGGCCCGGCCCCGCCGCCCACCCACCCACCTCGGG
ATACCCAGCCCCGGTCCCCCGTTCCCCGGGGGCCGTTATC
TCCAGCGGGGGTTTGGAAAAACGAGGAGGAGGAGGAGAAG
GCGGGGGGGGAGACGGGGGGAAAGCAAGGACACGGCCCGG
GGGGTGGGAGCGCGGGCCGGGCCGCTCGTAAGAGCCGCGA
CCCGGCCGCCGGGGAGCGTTGTCGCCGTCGGTCTGCCGGC
CCCCGTCCCTCCCTTTTTTGACCAACCAGCGCCCTCCCCC
CCACCACCATTCCTACTACCACCACCACCACCACCCCCAC
CACCGACACCTCCCGCGCACCCCCGCCCACATCCCCCCAC
CCCGCACCACGAGCACGGGGTGGGGGTAGCAGGGGATCAA
AGGGGGGCAAAGCCGGCGGGGCGGTTCGGGGGGGCGGGAG
ACCGAGTAGGCCCGCCCATACGCGGCCCCTCCCGGCAGCC
ACGCCCCCCAGCGTCGGGTGTCACGGGGAAAGAGCAGGGG
AGAGGGGAGAGGGGGGGAGAGGGGGTATATAAACCAACGA
AAAGCGCGGGAACGGGGATACGGGGCTTGTGTGGCACGAC
GTCGTGGTTGTGTTACTGGGCAAACACTTGGGGACTGTAG
GTTTCTGTGGGTGCCGACCCTAGGCGCTATGGGGATTTTG
GGTTGGGTCGGGCTTATTGCGGTTGGGGTTTTGTGTGTGC
GGGGGGGCTTGTCTTCAACCGAATATGTTATTCGGAGTCG
GGTGGCTCGAGAGGTGGGGGATATATTAAAGGTGCCTTGT
GTGCCGCTCCCGTCTGACGATCTTGATTGGCGTTACGAGA
CCCCCTCGGCTATAAACTATGCTTTGATAGACGGTATATT
TTTGCGTTATCACTGTCCCGGATTGGACACGGTCTTGTGG
GATAGGCATGCCCAGAAGGCATATTGGGTTAACCCCTTTT
TATTTGTGGCGGGTTTTCTGGAGGACTTGAGTCACCCCGC
GTTTCCTGCCAACACCCAGGAAACAGAAACGCGCTTGGCC
CTTTATAAAGAGATACGCCAGGCGCTGGACAGTCGCAAGC
AGGCCGCCAGCCACACACCTGTGAAGGCTGGGTGTGTGAA
CTTTGACTATTCGCGCACCCGCCGCTGTGTAGGGCGACAG
GATTTGGGACCTACCAACGGAACGTCTGGACGGACCCCGG
TTCTGCCGCCGGACGATGAAGCGGGCCTGCAACCGAAGCC
CCTCACCACGCCGCCGCCCATCATCGCCACGTCGGCCCCC
ACCCCGCGACGGGACGCCGCCACAAAAAGCAGACGCCGAC
GACCCCACTCCCGGCGCCTCTAACGATGCCTCGACGGAAA
CCCGTCCGGGTTCGGGGGGCGAACCGGCCGCCTGTCGCTC
GTCAGGGCCGGCGGGCGCTCCTCGCCGCCCTAGAGGCTGT
CCCGCTGGTGTGACGTTTTCCTCGTCCGCGCCCCCCGACC
CTCCCATGGATTTAACAAACGGGGGGGTGTCGCCTGCGGC
GACCTCGGCGCCTCTGGACTGGACCACGTTTCGGCGTGTG
TTTCTGATCGACGACGCGTGGCGGCCCCTGATGGAGCCTG
AGCTGGCGAACCCCTTAACCGCCCACCTCCTGGCCGAATA
TAATCGTCGGTGCCAGACCGAAGAGGTGCTGCCGCCGCGG
GAGGATGTGTTTTCGTGGACTCGTTATTGCACCCCCGACG
AGGTGCGCGTGGTTATCATCGGCCAGGACCCATATCACCA
CCCCGGCCAGGCGCACGGACTTGCGTTTAGCGTGCGCGCG
AACGTGCCGCCTCCCCCGAGTCTTCGGAATGTCTTGGTGG
CCGTCAAGAACTGTTATCCCGAGGCACGGATGAGCGGCCA
CGGTTGCCTGGAAAAGTGGGCGCGGGACGGCGTCCTGTTA
CTAAACACGACCCTGACCGTCAAGCGCGGGGCGGCGGCGT
CCCACTCTAGAATCGGTTGGGACCGCTTCGTGGGCGGAGT
TATCCGCCGGTTGGCCGCGCGCCGCCCCGGCCTGGTGTTT
ATGCTCTGGGGCGCACACGCCCAGAATGCCATCAGGCCGG
ACCCTCGGGTCCATTGCGTCCTCAAGTTTTCGCACCCGTC
GCCCCTCTCCAAGGTTCCGTTCGGAACCTGCCAGCATTTC
CTCGTGGCGAACCGATACCTCGAGACCCGGTCGATTTCAC
CCATCGACTGGTCGGTTTGAAAGGCATCGACGTCCGGGGT
TTTTGTCGGTGGGGGCTTTTGGGTATTTCCGATGAATAAA
GACGGTTAATGGTTAAACCTCTGGTCTCATACGGGTCGGT
GATGTCGGGCGTCGGGGGAGAGGGAGTTCCCTCTGCGCTT
GCGATTCTAGCCTCGTGGGGCTGGACGTTCGACACGCCAA
ACCACGAGTCGGGGATATCGCCAGATACGACTCCCGCAGA
TTCCATTCGGGGGGCCGCTGTGGCCTCACCTAACCAACCT
TTACCGGGGGCCCGGAACGGGAGGCCCAGCGCCGTCTTTC
TCCCCAACGCGCGCGGATGACGGCCCGCCCTGTACCGACG
GGCCCTACGTGACGTTTGATACCCTGTTTATGGTGTCGTC
GATCGACGAATTAGGGCGTCGCCAGCTCACGGACACCATC
CGCAAGGACCTGCGGTTGTCGCTGGCCAAGTTTAGCATTG
CGTGCACCAAGACCTCCTCGTTTTCGGGAAACGCCCCGCG
CCACCACAGACGCGGGGCGTTCCAGCGCGGCACGCGGGCG
CCGCGCAGCAACAAAAGCCTCCAGATGTTTGTGTTGTGCA
AACGCGCCCACGCCGCTCGAGTGCGAGAGCAGCTTCGGGT
CGTTATTCAGTCCCGCAAGCCGCGCAAGTATTACACGCGA
TCTTCGGACGGGCGGCTCTGCCCCGCCGTCCCCGTGTTCG
TCCACGAGTTCGTCTCGTCCGAGCCAATGCGCCTCCACCG
AGATAACGTCATGCTGGCCTCGGGGGCCGAGTAACCGCCC
CCCCCCCATGCCACCCTCACTGCCCGTCGCGCGTGTTTGA
TGTTAATAAATAACACATAAATTTGGCTGGTTGTTTGTTG
TCTTTAATGGACCGCCCGCAAGGGGGGGGGGGCGTTTCAG
TGTCGGGTGACGAGCGCGATCCGGCCGGGATCCTAGGACC
CCAAAAGTTTGTCTGCGTATTCCAGGGTGGGGCTCAGTTG
AATCTCCCGCAGCACCTCTACCAGCAGGTCCGCGGTGGGC
TGGAGAAACTCGGCCGTCCCGGGGCAGGCGGTTGTCGGGG
GTGGAGGCGCGGCGCCCACCCCGTGTGCCGCGCCTGGCGT
CTCCTCTGGGGGCGACCCGTAAATGGTTGCAGTGATGTAA
ATGGGTCCGCGGTCCAGACCACGGTCAAAATGCCGGCCGT
GGCGCTCCGGGCGCTTTCGCCGCGCGAGGAGCTGACCCAG
GAGTCGAACGGATACGCGTACATATGGGCGTCCCACCCGC
GTTCGAGCTTCTGGTTGCTGTCCCGGCCTATAAAGCGGTA
GGCACAAAATTCGGCGCGACAGTCGATAATCACCAACAGC
CCAATGGGGGTGTGCTGGATAACAACGCCTCCGCGCGGCA
GGCGGTCCTGGCGCTCCCGGCCCCGTACCATGATCGCGCG
GGTGCCGTACTCAAAAACATGCACCACCTGCGCGGCGTCG
GGCAGTGCGCTGGTCAGCGAGGCCCTGGCGTGGCATAGGC
TATACGCGATGGTCGTCTGTGGATTGGACATCTCGCGGTG
GGTAGTGAGTCCCCCGGGCCGGGTTCGGTGGAACTGTAAG
GGGACGGCGGGTTAATAGACAATGACCACGTTCGGATCGC
GCAGAGCCGATAGTATGTGCTCACTAATGACGTCATCGCG
CTCGTGGCGCTCCCGGAGCGGATTTAAGTTCATGCGAAGG
AATTCGGAGGAGGTGGTGCGGGACATGGCCACGTACGCGC
TGTTGAGGCGCAGGTTGCCGGGCGTAAAGCAGATGGCGAC
CTTGTCCAGGCTAAGGCCCTGGGAGCGCGTGATGGTCATG
GCAAGCTTGGAGCTGATGCCGTAGTCGGCGTTTATGGCCA
TGGCCAGCTCCGTAGAGTCAATGGACTCGACAAACTCGCT
GATGTTGGTGTTGACGACGGACATGAAGCCGTGTTGGTCA
CGCAAGACCACGTAAGGCAGGGGGGCCTCTTCCAGTAACT
CGGCCACGTTGGCCGTCGCGTGCCGCCTCCGCAGCTCGTC
CGCAAAGGCAAACACCCGTGTGTACGTGTATCCCATGAGC
GTATAATTGTCCGTCTGCAGGGCGACGGACATCAGCCCCC
CGCGCGGCGAGCCGGTCAGCATCTCGCAGCCCCGGAAGAT
AACGTTGTCCACGTACGTGCTAAAGGGGGCGACTTCAAAT
GCCTCCCCGAAGAGCTCTTGGAGGATTCGGAATCTCCCGA
GGAAGGCCCGCTTCAGCAGCGCAAACTGGGTGTGAACGGC
GGCGGTGGTCTCCGGTTCCCCGGGGGTGTAGTGGCAGTAA
AACACGTCGAGCTGTTGTTCGTCCAGCCCCGCGAAAATAA
CGTCGAGGTCGTCGTCGGGAAAATCGTCCGGGCCCCCGTC
CCGCGGCCCCAGTTGCTTAAAATCAAACGCACGCTCGCCG
GGGGCGCCTGCGTCGGCCATTACCGACGCCTGCGTCGGCA
CCCCCGAAGATTTGGGGCGCAGAGACAGAATCTCCGCCGT
TAGTTCTCCCATGCGGGCGTACGCGAGGGTCCTCTGGGTC
GCATCCAGGCCCGGGCGCTGCAGAAAGTTGTAAAAGGAGA
TAAGCCCGCTAAATATGAGCCGCGACAGGAACCTGTAGGC
AAACTCCACCGAAGTCTCCCCCTGAGTCTTTACAAAGCTG
TCGTCACGCAACACTGCCTCGAAGGCCCGGAACGTCCCAC
TAAACCCAAAAACCAGTTTTCGCAGGCGCGCGGTCACCGC
GATCTGGCTGTTGAGGACGTAAGTGACGTCGTTGCGGGCC
ACGACCAGCTGCTGTTTGCTGTGCACCTCGCAGCGCATGT
GCCCCGCGTCCTGGTCCTGGCTCTGCGAGTAGTTGGTGAT
GCGGCTGGTGTTGGCCGTGAGCCACTTTTCAATAGTCAGG
CCGGGCTGGTGTGTCAGCCGTCGGTAGTGTTCAAACTCCT
TGACCGACACGAACGTAAGCACGGGGAGGTGTAGCCGTCG
GTATTCGTCAAACTCCTTTCCCTCCCCTCCCTTCCTCCCT
TTTCTTTTTCCCACTCCGCCCTCCCCCTCACGGGTCACCT
TCAGGTAGGCGTGGAGCTTGGCCATGTACGCGCTCACCTC
TTTGTGGGAGGAGAACAGCCGCGTCCAGCCGGGGAGGTTG
GCGGGGTTGGTGATGTAGTTTTCCGGGACGACGAAGCGAT
CCACGAACTGCATGTGCTCCTCGGTGATGGGCAGGCCGTA
CTCCAGCACCTTCATGAGGTTACCGAACTCGTGCTCGACG
CACCGTTTGTTGTTAATAAAAATGGCCCAGCTATACGAGA
GGCGGGCGTACTCGCGCAGCGTGCGGTTGCAGATGAGGTA
CGTGAGCACGTTCTCGCTCTGGCGGACGGAACACCGCAGT
TTCTGGTGCTCGAAGGTCGACTCCAGGGACGCCGTCTGCG
TCGGCGAGCCCCCACACACCAACACGGGCCGCAGGCGGGC
CGCGTACTGGGGGGTGTGGTACAGGGCGTTAATCATCCAC
CAGCAATACACCACGGCCGTGAGGAGGTGACGCCCAAGGA
GCCCGGCCTCGTCGATGACGATCACGTTGCTGCGGGTAAA
GGCCGGCAGCGCCCCGTGGGTGGCCGGGGCCAACCGCGTC
AGGGCGCCCTCGGCCAACCCCAGGGTCCGTTCCAGGGCGG
CCAGGGCGCGAAACTCGTTCCGCAACTCCTCGCCCCCGGA
GGCGGCCAGGGCGCGCTTCGTGAGGTCCAAAATCACCTCC
CAGTAGTACGTCAGATCTCGTCGCTGCAGGTCCTCCAGCG
AGGCGGGGTTGCTGGTCAGGGGGTACGGGTACTGTCCCAG
TTGGGCCTGGACGTGATTCCCGCGAAACCCAAATTCATGA
AAGATGGTGTTGATGGGTCGGCTGAGAAAGGCGCCCGAGA
GTTTGGCGTACATGTTTTGGGCCGCAATGCGCGTGGCGCC
CGTCACCACACAGTCCAAGACCTCGTTGATTGTCTGCACG
CACGTGCTCTTTCCGGAGCCAGCGTTGCCGGTGATAAGAT
ACACCGCGAACGGAAACTCCCTGAGGGGCAGGCCTGCGGG
GGACTCTAAGGCCGCCACGTCCCGGAACCACTGCAGACGG
GGCACTTGCGCTCCGTCGAGCTGTTGTTGCGAGAGCTCTC
GGATGCGCTTAAGGATTGGCTGCACCCCGTGCATAGACGT
AAAATTTAAAAAGGCCTCGGCCCTCCCTGGAACGGCTGGT
CGGTCCCCGGGTTGCTGAAGGTGCGGCGGGCCGGGTTTCT
GTCCGTCTAGCTGGCGCTCCCCGCCGGCCGCCGCCATGAC
CGCACCACGCTCGTGGGCCCCCACTACGCGTGCGCGGGGG
GACACGGAAGCGCTGTGCTCCCCCGAGGACGGCTGGGTAA
AGGTTCACCCCACCCCCGGTACGATGCTGTTCCGTGAGAT
TCTCCACGGGCAGCTGGGGTATACCGAGGGCCAGGGGGGG
TACAACGTCGTCCGGTCCAGCGAGGCGACCACCCGGCAGC
TGCAGGCGGCGATCTTTCACGCGCTCCTCAACGCCACCAC
TTACCGGGACCTCGAGGCGGACTGGCTCGGCCACGTGGCG
GCCCGCGGTCTGCAGCCCCAACGGCTGGTTCGCCGGTACA
GGAACGCCCGGGAGGCGGATATCGCCGGGGTGGCCGAGCG
GGTGTTCGACACGTGGCGGAACACGCTTAGGACGACGCTG
CTGGACTTTGCCCACGGGTTGGTCGCCTGCTTTGCGCCGG
GCGGCCCGAGCGGCCCGTCAAGCTTCCCCAAATATATCGA
CTGGCTGACGTGCCTGGGGCTGGTCCCCATATTACGCAAG
CGACAAGAAGGGGGTGTGACGCAGGGTCTGAGGGCGTTTC
TCAAGCAGCACCCGCTGACCCGCCAGCTGGCCACGGTCGC
GGAGGCCGCGGAGCGCGCCGGCCCCGGGTTTTTTGAGCTG
GCGCTGGCCTTCGACTCCACGCGCGTGGCGGACTACGACC
GCGTGTATATCTACTACAACCACCGCCGGGGCGACTGGCT
CGTGCGAGACCCCATCAGCGGGCAGCGCGGAGAATGTCTG
GTGCTGTGGCCCCCCTTGTGGACCGGGGACCGTCTGGTCT
TCGATTCGCCCGTCCAGCGGCTGTTTCCCGAGATCGTCGC
GTGTCACTCCCTCCGGGGACACGCGCACGTCTGCCGGCTG
CGCAATACCGCGTCCGTCAAGGTGCTGCTGGGGCGCAAGA
GCGACAGCGAGCGCGGGGTGGCCGGTGCCGCGCGGGTCGT
TAACAAGGTGTTGGGGGAGGACGACGAGACCAAGGCCGGG
TCGGCCGCCTCGCGCCTCGTGCGGCTTATCATCAACATGA
AGGGCATGCGCCACGTAGGCGACATTAACGACACCGTGCG
TGCCTACCTCGACGAGGCCGGGGGGCACCTGATAGACGCC
CCGGCCGTCGACGGTACCCTCCCTGGATTCGGCAAGGGCG
GAAACAACCGCGGGTCTGCGGGCCAGGACCAGGGGGGGCG
GGCGCCGCAGCTTCGCCAGGCCTTCCGCACGGCCGTGGTT
AACAACATCAACGGCGTGTTGGAGGGCTATATAAATAACC
TGTTTGGAACCATCGAGCGCCTGCGCGAGACCAACGCGGG
CCTGGCGACCCAATTGCAGGAGCGCGACCGCGAGCTCCGG
CGCGCAACAGCGGGGGCCCTGGAGCGCCAGCAGCGCGCGG
CCGACCTGGCGGCCGAGTCCGTGACCGGTGGATGCGGCAG
CCGCCCTGCGGGGGCGGACCTGCTCCGGGCCGACTATGAC
ATTATCGACGTCAGCAAGTCCATGGACGACGACACGTACG
TCGCCAACAGCTTTCAGCACCCGTACATCCCTTCGTACGC
CCAGGACCTGGAGCGCCTGTCGCGCCTCTGGGAGCACGAG
CTGGTGCGCTGTTTTAAAATTCTGTGTCACCGCAACAACC
AGGGCCAAGAGACGTCGATCTCGTACTCCAGCGGGGCGAT
CGCCGCATTCGTCGCCCCCTACTTTGAGTCAGTGCTTCGG
GCCCCCCGGGTAGGCGCGCCCATCACGGGCTCCGATGTCA
TCCTGGGGGAGGAGGAGTTATGGGATGCGGTGTTTAAGAA
AACCCGCCTGCAAACGTACCTGACAGACATCGCGGCCCTG
TTCGTCGCGGACGTCCAGCACGCAGCGCTGCCCCCGCCCC
CCTCCCCGGTCGGCGCCGATTTCCGGCCCGGCGCGTCCCC
GCGGGGCCGGTCCAGACGCGGTCGCCCGGAAGGAAGAACG
GCGCCAGGCGCGCCGGACCAGGGCGGGGGCATCGGGCACC
GGGATGGCCGCCGCGACGGCCGACGATGAGGGGTCGGCCG
CCACCATCCTCAAGCAGGCCATCGCCGGGGACCGCAGCCT
GGTCGAGGCGGCCGAGGCGATTAGCCAGCAGACGCTGCTC
CGCCTGGCCTGCGGGTGCGCCAGGTCGGCGCCGCCAGCCG
CGGTTTACCGCCACCAGCATCGCGCGCGTCGACGTCGCGC
CTGGGTGCCGGTTGCGGTTCGTTCTGGACGGGAGTCCCGA
GGACGCCTATGTGACGTCGGAGGATTACTTTAAGCGCTGC
TGCGGCCAGTCCAGTTATCGCGGCTTCGCGGTGGCGGTCC
TGACGGCCAACGAGGACCACGTGCACAGCCTGGCCGTGCC
CCCCCTCGTTCTGCTGCACCGGTTCTCCCTGTTCAACCCC
AGGGACCTCCTGGACTTTGAGCTTGCCTGTCTGCTGATGT
ACCTGGAGAACTGCCCCCGAAGCCACGCCACCCCGTCGAC
CTTTGCCAAGGTTCTGGCGTGGCTCGGGGTCGCGGGTCGC
CGCACGTCCCCATTCGAACGCGTTCGCTGCCTTTTCCTCC
GCAGTTGCCACTGGGTCCTAAACACACTCATGTTCATGGT
GCACGTAAAACCGTTCGACGACGAGTTCGTCCTGCCCCAC
TGGTACATGGCCCGGTACCTGCTGGCCAACAACCCGCCCC
CCGTTCTCTCGGCCCTGTTCTGTGCCACCCCGACGAGCTC
CTCATTCCGGCTGCCGGGGCCGCCCCCCCGCTCCGACTGC
GTGGCCTATAACCCCGCCGGGATCATGGGGAGCTGCTGGG
CGTCGGAGGAGGTGCGCGCGCCTCTGGTCTATTGGTGGCT
TTCGGAGACCCCAAAACGACAGACGTCGTCGCTGTTTTAT
CAGTTTTGTTGAATTTTAGGAAATAAACCCGGTTTTGTTT
CTGTGGCCTCCCGACGGATGCGCGTGTCCTTCCTCCGTCT
TGGTGGGTGGGTGTCTGTGTATCCGTCCCATCTGTGCGGA
GAGGGGGGGCATGTCGGCACGTATTCGGACAGACTCAAGC
ACACACGGGGGAGCGCTCTTGTCTCAGGGCAATGTTTTTA
TTGGTCAAACTCAGGCAAACAGAAACGACATCTTGTCGTC
AAAGGGATACACAAACTTCCCCCCCTCTCCCCATACTCCC
GCCAGCACCCCGGTAAACACCAACTCAATCTCGCGCAGGA
TTTCGCGCAGGTGATGAGCGCAGTCCACGGGGGGGAGCAC
AAGGGGCCGCGGGTGTAGATCGAGGGGACGCCGACCGACT
CACCGCCTCCGGGACAGACACGCACGACGCGCCGCCAGTA
GTGCTCTGCGTCCAGCAAGGCGCCGCCGCGGAAGGCAGTG
GGGGGCAAGGGGTCGCTAGCCTCAAGGGGGACACCCGAAC
GCTCCAGTACTCCGCGTCCAACCGTTTATTAAACGCGTCC
ACGATAAGGCGGTCGCAGGCGTCCTCCATAAGGCCCCGGG
CCGTGAGTGCGTCCTCCTCCGGCACGCCTGCCGTTGTCAG
GCCCAGGACCCGTCGCAGCGTGTCGCGTACGACCCCGGCC
GCCGTGGTGTACGCGGGCCCGCGGAGAGGAAATCCCCCAA
GATGGTCAGTGTTGTCGCGGGAGTTCCAGAACCACACTCC
CGCCTGGTTCCAGGCGACTGCGTGGGTGTAGACGCCCTCG
AGGGCCAGGCACAGTGGGTGCCGCAGCCGGAGGCCGTTGG
CCCTAAGCACGCTCCACGGCCGTCTCGATGGCCCGCCGGG
CGTCCTCGATCCCCCGGAAGCCGCATCCGCGTCTTGGGGG
TCCACGTTAAAGACACCCCAGAACGCACCCCCATCGCCCC
CGCAGACCGCGAACTTCACCGAGCTGGCCGTCTCCTCGAT
CTGCAGGCAGACGGCGGCCATTACCCCACCCAGGAGCTGC
CGCAGCGCAGGGCAGGCGTCGCACGTGTCCGGGACCAGGC
GCTCCAAGACGGCCCCGGCCCAGGGCTCTGAGGGAGCGGC
CACCACCAGCGCGTCCAGTCTTGCTAGGCCCGTCCGGCCG
TGGGGGTCCGCCAGCCCGCTCCCCCCGAGGTCGGCAAGGA
CAAAAGGAGCTGGGCGCGAAGTCCGGGGAAGCAAAACCGC
GCCGTCCAGACGGGCCCGACGGCCGCGGGCGGGTCTAACA
GTTGGATGATTTTAGTGGCGGGATGCCACCGCGCCACCGC
CTCCCGCACCGCGGGCAGGAGGCATCCGGCTGCCGCCGAG
GCCACGCCGGGCCAGGCTCGCGGGGGGAGGACGACCCTGG
CCCCCACCGCGGGCCAGGCCCCCAGGAGCGCGGCGTAAGC
GGCCGCGGCCCCGCGCACCAGGTCCCGTGCCGACTCGGCC
GTGGCCGGCACGGTGAACGTGGGCCAACCCGGAAACCCCA
GGACGGCAAAGTACGGGACGGGTCCCCCCCGGACCTCAAA
CTCGGGCCCCAGAAAGGCAAAGACGGGGGCCAGGGCCCCG
GGGGCGGCGTGGACCGTGGTATGCCACTGCCGGAAAAGGG
CGACGAGCGCCGGCGCGGAGAACTTCTCGCCGGCGCTTAC
AAAGTAGTCGTAATCGCGGGGCAGCAGCACCCGTGCCGTG
ACTCGTTGCGGGTGCCCGCGTGGCCGCAGGCCCACCTCGC
ACACCTCGACCAGGTCCCCGAACGCGCCCTCCTTCTTGAT
CGGCGGAAACGCAAGAGTCTGGTATTCGCGCGCAAATAGC
GCGGTTCCGGTGGTGATGTTAACGGTCAGCGAAGCGGCGG
ACGCGCACTGGGGGGTGTCGCGATCCGCCAGGCGCGCCCC
GCCACGCCGCGCGTCGGGATGCTCGGCAACGCGCGCCGCC
AGGGCCATAGGGTCGATGTCAATGTTGGCCTCCGCGACCA
GGAGAGCGGCGCGAGGGGCGGCGGGCGGGCCCCACGACGC
TCTCTCAACTTTCACCCCCAGTCCCGTGCGTGGGTCCGAG
CCGATACGCAGCGGGGCGAACAGGGCCACCGGCCCGGTCT
GGCGCTCCAGGGCCGCCAGGACGCACGCGTACAGCGCCCG
CCACAGAGTCGGGTTCTCCAGGGGCTCCAGCGGGGAGGCG
GCCGGCGTCGTCGCGGCGCGGGCGGCCGCCACGACGGCCT
GGACGGAGACGTCCGCGGAGCCGTAGAAATCCCGCAGCTC
CGTCGCGGTGACGGAGACCTCCGCAAAGCGCGCGCGACCC
TCCCCTGCGGCGTTGCGACATACAAAATACACCAGGGCGT
GGAAGTACTCGCGAGCGCGGGGGGGCAGCCATACCGCGTA
AAGGGTAATGGCGCTGACGCTCTCCTCCACCCACACGATA
TCTGCGGTGTCCATCGCACGGCCCCTAAGGATCACGGGCG
GTCTGTGGGTCCCATGCTGCCGTGCCTGGCCGGGCCCGGT
GGGTCGCGGAAACCGGTGACGGGGGGGGGGCGGTTTTTGG
GGTTGGGGTGGGGGTGGGAAACGGCCCGGGTCCGGGGGCC
AACTTGGCCCCTCGGTGCGTTCCGGCAACAGCGCCGCCGG
TCCGCGGACGACCACGTACCGAACGAGTGCGGTCCCGAGA
CTTATAGGGTGCTAAAGTTCACCGCCCCCTGCATCATGGG
CCAGGCCTCGGTGGGGAGCTCCGACAGCGCCGCCTCCAGG
ATGATGTCAGCGTTGGGGTTGGCGCTGGATGAGTGCGTGC
GCAAACAGCGCCCCCACGCAGGCACGCGTAGCTTGAAGCG
CGCGCCCGCAAACTCCCGCTTGTGGGCCATAAGCAGGGCG
TACAGCTGCCTGTGGGTCCGGCAGGCGCTGTGGTCGATGT
GGTGGGCGTCCAACACCCCACGATTGTCTGTTTGGTGAGG
TTTTTAACGCGCCCCGCCCCGGGAAACGTCTGCGTGCTTT
TGGCCATCTGCACGCCAAACAGTTCGCCCCAGATTATCTT
GAACAGCGCCACCGCGTGGTCCGTCTCGCTAACGGACCCG
CGCGGGGGACAGCCGCTTAGGGCGTCGGCGACGCGCTTGA
CGGCTTCCTCCGAGAGCAGAAGTCCGTCGGTTACGTTACA
GTGGCCCAGTTCGAACACCAGCTGCATGTAGCGGTCGTAG
TGGGGGGTCAGTAGGTCCAGCACGTCATCGGGGCCGAAGG
TCCTCCCAGATCCCCCGGCCGCCGAGTCCCAATGCAGGCG
CGCGGCCATGGTGCTGCACAGGCACAACAGCTCCCAGACG
GGGGTTACGTTCAGGGTGGGGGGCAGGGCCACGAGCTCCA
GCTCTCCGGTGACGTTGATCGTGGGGATGACGCCCGTGGC
GTAGTGGTCATAGACCGCCGATATGGCGCTGCTGCGGGTG
GCCATGGGAACGCGGAGACAGGCCTCCAGCAACGCCAGGT
AAATAAACCGCGTGCGTCCCATCAGGCTGTTGAGGTTGCG
CATGAGCGCGACAATTTCCGCCGGCGCGACATCGGACCGG
AGGTATTTTTCGACGAAAAGACCCACCTCCTCCGTCTCGG
CGGCCTGGGCCGGCAGCGACGCCTCGGGATCCCGGCACCG
CAGCTCCCGTAGATCGCGCTGGGCCCTGAGGGCGTCGAAA
TGTACGCCCCGCAAAAACAGACAGAAGTCCTTTGGGGTCA
GGGTATCGTCGTGTCCCCAGAAGCGCACGCGTATGCAGTT
TAGGGTCAGCAGCATGTGAAGGATGTTAAGGCTGTCCGAG
AGACACGCCAGCGTGCATCTCTCAAAGTAGTGTTTGTAAC
GGAATTTGTTGTAGATGCGCGACCCCCGCCCCAGCGACGT
GTCGCATGCCGACGCGTCACAGCGCCCCTTGAACCGGCGA
CACAGCAGGTTTGTGACCTGGGAGAACTGCGCGGGCCACT
GGCCGCAGGAACTGACCACGTGATTAAGGAGCATGGGCGT
AAAGACGGGCTCCGAGCGCGCCCCGGAGCCGTCCATGTAA
ATCAGTAGCTCCCCCTTGCGGAGGGTGCGCACCCGTCCCA
GGGACTGGTACACGGACACCATGTCCGGTCCGTAGTTCAT
GGGTTTTACGTAGGCGAACATGCCATCAAAGTGCAGGGGA
TGAAGCGGAGGCCCACGGTTACGACCGTCGTGTATATAAC
CACGCGGTATTGGCCCCACGTGGTCACGTCCCCGAGGGGG
GTGAGCGAGTGAAGCAACAGCACGCGGTCCGTAAACTGAC
GGCAGAACCGGGCCACGATCTCCGCGAAGGAGACCGTCGA
CGAAAAAATGCAGATGTTATCGCCCCCGCCAAGGCGCGCT
TCCAGCTCCCCAAAGAACGTGGCCCCCCGGGCGTCCGGAG
AGGCGTCCGGAGACGGGCCGCTCGGCGGCCCGGGCGGGCG
CAGGGCAGCCTGCAGGAGCTCGGTCCCCAGACGCGGGAGA
AACAGGCACCGGCGCGCCGAAAACCCGGGCATGGCGTACT
CGCCGACCACCACATGCACGTTTTTTTCGCCCCGGAGACC
GCACAGGAAGTCCACCAACTGCGCGTTGGCGGTTGCGTCC
ATGGCGATGATCCGAGGACAGGTGCGCAGCAGGCGTAGCA
TTAACGCATCCACGCGGCCCAGTTGCTGCATCGTTGGCGA
ATAGAGCTGGCCCAGCGTCGACATAACCTCGTCCAGAACG
AGGACGTCGTAGTTGTTCAGAAGGTTGGGGCCCACGCGAT
GAAGGCTTTCCACCTGGACGATAAGTCGGTGGAAGGGGCG
GTCGTTCATAATGTAATTGGTGGATGAGAAGTAGGTGACA
AAGTCGACCAGGCCTGACTCAGCGAACCGCGTCGCCAGGG
TCTGGGTAAAACTCCGACGACAGGAGACGACGAGCACACT
CGTGTCCGGAGAGTGGATCGCTTCCCGCAGCCAGCGGATC
AGCGCGGTAGTTTTTCCCGACCCCATTGGCGCGCGGACCA
CAGTCACGCACCTGGCCGTCGGGGCGCTCGCGTTGGGGAA
GGTGACGGGTCCGTGCTGCTGCCGCTCGATCGTTGTTTTC
GGGTGAACCCGGGGCACCCATTCGGCCAAATCCCCCCCGT
ACAACATCCGCGCTAGCGATACGCTCGACGTGTACTGTTC
GCACTCGTCGTCCCCAATGGGACGCCCGGCCCCAGAGGAT
CTCCCGACTCCGCGCCCCCCACGAAAGGCATGACCGGGGC
GCGGACGGCGTGGTGGGTCTGGTGTGTGCAGGTGGCGACG
TTTGTGGTCTCTGCGGTCTGCGTCACGGGGCTCCTCGTCC
TGGCCTCTGTGTTCCGGGCACGGTTTCCCTGCTTTTACGC
CACGGCGAGCTCTTATGCCGGGGTGAACTCCACGGCCGAG
GTGCGCGGGGGTGTAGCCGTGCCCCTCAGGTTGGACACGC
AGAGCCTTGTGGGCACTTATGTAATCACGGCCGTGTTGTT
GTTGGCCGCGGCCGTGTATGCCGTGGTCGGCGCCGTGACC
TCCCGCTACGACCGCGCCCTGGACGCGGGCCGCCGTCTGG
CTGCGGCCCGCATGGCCATGCCGCACGCCACGCTGATCGC
CGGAAACGTCTGCTCTTGGTTGCTGCAGATCACCGTCCTG
TTGCTGGCCCATCGCACCAGCCAGCTGGCCCACCTGGTTT
ACGTCCTGCACTTTGCGTGTCTGGTGTATTTTGCGGCCCA
TTTTTGCACCAGGGGGGTCCTGAGCGGGACGTATCTGCGT
CAGGTGCACGGCCTGATGGAGCCGGCCCCGACTCATCATC
GCGTCGTTGGCCCGGCTCGAGCCGTGCTGACAAACGCCTT
GCTGTTGGGCGTCTTCCTGTGCACGGCCGACGCCGCGGTA
TCCCTGAATACCATCGCCGCGTTCAACTTTAATTTTTCGG
CCCCGGGCATGCTCATATGCCTGACCGTGCTGTTCGCCCT
TCTCGTCGTATCGCTGTTGTTGGTGGTCGAGGGGGTGTTG
TGTCACTACGTGCGCGTGTTGGTGGGCCCCCACCTGGGGG
CCGTGGCCGCCACGGGCATCGTCGGCCTGGCATGCGAGCA
CTATTACACCAACGGCTACTACGTTGTGGAGACGCAGTGG
CCGGGGGCCCAGACGGGAGTCCGCGTCGCCCTCGCCCTGG
TCGCCGCCTTTGCCCTCGGCATGGCCGTGCTCCGCTGCAC
CCGCGCCTATCTGTATCACAGGCGGCACCACACCAAATTT
TTTATGCGCATGCGCGACACGCGACACCGCGCACATTCCG
CCCTCAAGCGCGTACGCAGTTCCATGCGCGGATCGCGAGA
CGGCCGCCACAGGCCCGCACCCGGCAGCCCGCCCGGGATT
CCCGAATCCTTCGAAGACCCCTACGCGATCTCATACGGCG
GCCAGCTCGACCGGTACGGAGATTCCGACGGGGAGCCGAT
TTACGACGAGGTGGCGGACGACCAAACCGACGTATTGTAC
GCCAAGATACAACACCCGCGGCACCTGCCCGACGACGAGC
CCATCTATGACACCGTTGGGGGGTACGACCCCGAGCCCGC
CGAGGACCCCGTGTACAGCACCGTCCGCCGTTGGTAGCTG
TTTGGTTCCGTTTTAATAAACCGTTTGTGTTTAACCCGAC
CGTGGTGTATGTCTGGTGTGTGGCGTCCGATCCCGTTACT
ATCACCGTTCCCCCCAAACCCCGGCGATTGTGGGTTTTTT
TAAAAACGACACGCGTGCGACCGTATACAGAACATTGTTG
TTTTTTATTCGCTATCGGACATGGGGGGTGGAAACTGGGT
GGCGGGGCAGGCGCCTCCGGGGGTTCGCCGGTGAGTGTGG
CGCGAGGGGGATCCGACGAACGCAGGCGCTGTCTCCCCGG
GGCCCGCGTAACCCCGCGCATATCCGGGGGCACGTAGAAA
TTACCTTCCTCTTCGGACTCGATATCCACGACGTCAAAGT
CGTGGGCGGTCAGCGAGACGACCTCCCCGTCGTCGGTGAT
GAGGACGTTGTTTCGGCAGCAGCAGGGCCGGGTTTCCTTT
TCCCCCGAGCCCATAGCTCGGCGAGCGTGTCGTCGAACGC
CAGGCGGCTGCTTCGCTGTATGGCCTTATAGATCTCCGGA
TCGATGCGGACGGGGGTAATGATCAGGGCGATCGGAACGG
CCTGGTTCGGGAGAATGGACGCCTTGCTGGGTCCTGCGGC
CCCGAGAGCCCCGGCGCCGTCCTCCAGGCGGAACGTTACG
CCCTCCTCCGCGCTAGTGCGGTGCCTGCCGATAAACGTCA
CCAGATGCGGGTGGGGGGGGCAGTCGGGGAAGTGGCTGTC
GAGCACGTAGCCCGCACCAAGATCTGCTTAAAGTTCGGGG
ACGGGGGGTCGCGAAGACGGGCTCGCGGCGTACCAGATCC
CCGGAGCTCCAGGACACGGGGGAGATGGTGTGGCGTCCGA
GGTCGGGGGTGCCAAACAGAAGCACCTCCGAGACAACGCC
GCTATTTAACTCCACCAAGGCCCGATCCGCGGCGGAGCAC
CGCCTTTTTTCGCCCGAGGCGTGGGCCTCTGACCAGGCCT
GGTCTTGCGTGACGAGAGCCTCCTCCGGGCCGGGGACGCG
CCCGGGCGCGAAGTATCGCACGCTGGGCTTCGGGATCGAC
CGGATAAATGCCCGGAACGCCTCCGGGGACCGGTGTGCCA
TCAAGTCCTCGTACGCGGAGGCCGTGGGGTCGCTGGGGTC
CATGGGGTCGAAAGCGTACTTGGCCCGGCATTTGACCTCG
TAAAAGGCCAGGGGGGTCTTGGGGACTGGGGCCAAGTAGC
CGTGAATGTCCCGAGGACAGACGAGAATATCCAGGGACGC
CCCGACCATCCCCGTGTGACCGTCCATGAGGACCCCACAC
GTATGCACGTTCTCTTCGGCGAGGTCGCCGGGTTCGTGGA
AGATAAAGCGCCGCGTGTCGGCGCCGGCCTCGCCGCCGTC
GTCCGCGCGGCCCACGCAGTAGCGAAACAGCAGGCTTCGG
GCCGTCGGCTCGTTCACCCGCCCGAACATCACCGCCGAAG
ACTGTACATCCGGCCGCAGGCTGGCGTTGTGCTTCAGCCA
CTGGGGCGAGAAACACGGACCCTGGGGGCCCCAGCGGAGG
TGGTATGCGGTCGTGAGGCCGCGGAGCAGGGCCCATAGCT
GGCAGTCGGCCTGGTTTTGCGTGGCCGCCTCGTAAAACCC
CATGAGGGGCCGGGGCGCCACGGCGTCCGCGGCGGCCGGG
GGCCCGCGGCGCGTCAGGCGCCATAGGTGCCGGCCGAGTC
CGCGGTCCACCATACCCGCCTCCTCGAGGACCACGGCCAG
GGAACACAGATAATCCAGGCGGGCCCCCCCCCTCTCCCCT
CTCCCCCCCTCTCCCCTGCTCTTTCCCCGCGACACCCGAC
GCTGGGGGGCGTGGCTGCCGGGAGGGGCCGCGTATGGGCG
GGCCTACTCGGTCTCCCGCCCCCCCGAACCGCCCCGCCGG
CTTTGCCCCCCTTTGATCCCCTGCTACCCCCACCCCGTGC
TCGTGGTGCGGGGTGGGGGGATGTGGGCGGGGGTGCGCGG
GAGGTGTCGGTGGTGGGGGTGGTGGTGGTGGTGGTAGTAG
GAATGGTGGTGGGGGGGAGGGCGCTGGTTGGTCAAAAAAG
GGAGGGACGGGGGCCGGCAGACCGACGGCGACAACGCTCC
CCGGCGGCCGGGTCGCGGCCTTACGGCGGCCCGCCCGCGC
CCCCCCCCCCGGGCCGTGTCCTTGCTTTCCCCCCGTCTCC
CCCCTTTTGCGTGGCCGCCTCGTAAACCCCCAGAGGGGCC
GGGGCGCCACGGCGTCCGCGGCGGCCGGGGGCCCGCGGCG
CGTCAGGCGCCATAGGTGCCGGCCGAGTCCGCGGTCCACC
ATACCCGCCTCCTCGAGGACCACGGCCAGGGAACACAGAT
AATCCAGGCGGGCCCAGAGGGGACCGATGGCCAGAGGGGC
GCGGACGCCGCGCAGCAACCCGCGCAGGTGGCGCTCGAAC
GTCTCGGCTAGTATATGGGAGGGCAGCGCGTTGGGGATCA
CCGACGCCGACCACATAGAGTCAAGGTCCGGGGAGTCGGG
ATCGGCGTCCGGGTCGCGGGCGTGGGTGCCCCCAGGAGAT
AGCGGAATGTCTGGGGTCGGAGGCCTGAGGCGTCAGAAAG
TGCCGGCGACGCGGCCCGGGGCTTTTCGTCTGCGGTGTCG
GTGGCGTGCTGATCACGTGGGGGGTTAACGGGCGAATGGG
GAGCTCGGGTCCACAGCTGACGTCGTCTGGGGTGGGGGGG
GCAGGGGACGGAAGGTGGTTGTTAGCGGAAGACTGTTAGG
GCGGGGGCGCTTGGGGGGGCTGTCGGGGCCACGAGGGGTG
TCCTCGGCCAGGGCCCAGGAACGCTTAGTCACGGTGCGTC
CCGGCGGACATGCTGGGCCTCCCGTGGACTCCATTTCCGA
GACGACGTGGGGGAGCGGTGGTTGAGCGCGCCGCCGGGTG
AACGCTGATTCTCACGACAGCGCGTGCCGCGCGCACGGGT
TGGTGTGACACAGGCGGGCCCGCCTCCTCGAGGACCACGG
CCAGGGAACACAGATAATCCAGGCGGGCCCAGAGGGGACC
GATGGCCAGAGGGGCGCGGACGCCGCGCAGCAACCCGCGC
AGGTGGCGCTCGAACGTCTCGGCTAGTATATGGGAGGGCA
GCGCGTTGGGGATCACCGACGCCGACCACATAGAGTCAAG
GTCCGGGGAGTCGGGATCGGCGTCCGGGTCGCGGGCGTGG
GTGCCCCCAGGAGATAGCGGAATGTCTGGGGTCGGAGGCC
CTGAGGCGTCAGAAAGTGCCGGCGACGCGGCCCGGGGCTT
TTCGTCTGCGGTGTCGGTGGCGTGCTGATCACGTGGGGGG
TTAACGGGCGAATGGGAGCTCGGGTCCACAGCTGACGTCG
TCTGGGGTGGGGGGGGCAGGGGACGGAAGGTGGTTGTTAG
CGGAAGACTGTTAGGGCGGGGGCGCTTGGGGGGGCTGTCG
GGGCCACGAGGGGTGTCCTCGGCCAGGGCCCAGGAACGCT
TAGTCACGGTGCGTCCCGGCGGACATGCTGGGCCTCCCGT
GGACTCCATTTCCGAGACGACGTGGGGGAGCGGTGGTTGA
GCGCGCCGCCGGGTGAACGCTGATTCTCACGACAGCGCGT
GCCGCGCGCACGGGTTGGTGTGACACAGGCGGGACACCAG
CACCAGGAGAGGCTTAAGCTCGGGAGGCAGCGCCACCGAC
GACAGTATCGCCTTGTGTGTGTGCTGGTAATTTATACACC
GATCCGTAAACGCGCGCCGAATCTTGGGATTGCGGAGGTG
GCGCCGGATGCCCTCTGGGACGTCATACGCCAGGCCGTGG
GTGTTGGTCTCGGCCGAGTTGACAAACAGGGCTGGGTGCA
GCACGTGGCGATAGGCGAGCAGGGCCAGGGCGAAGTCCAG
CGACAGCTGGTTGTTGAAATACTGGTAACCGGGAAACCGG
GTCACGGGTACGCCCAGGCTCGGGGCGACGTACACGCTAA
CCACCAACTCCAGCAGCGTCTGGCCAAGGGCGTACAGGTC
AACCGCTAACCCGACGTCGTGCTTCAGGCGGTGGTTGGTA
AATTCGGCCCGTTCGTTGTTAAGGTATTTCACCAACAGCT
CCGGGGGCTGGTTATACCCGTGACCCACCAGGGGTGAAAG
TTGGCTGTGGTTAGGGCGGTGGGCATGCCAAACATCCGGG
GGGACTTGAGGTCCGGCTCCTGGAGGCAAAACTGCCCCCG
GGCGATCGTGGAGTTGGAGTTGAGGGTGACGAGGCTAAAG
TCGGCGAGGACGGCCCGCCGGAGCGAGACGGCGTCCGACC
GCAGCATGACGAGGATGTTGGCGCGTGAATCGGGTGGCTC
CCCAGGTGGTGTTTAAAAACACAACGGCGCGGGCCAGCTC
CGTGAAGCACTGGTGGAGGGCCGTCGAGACCGAGGGGTTT
GTTGTGCGCAGGGACGCCAGTTGGCCGATATACTTACCGA
GGTCCATGTCGTACGCGGGGAACACTATCTGTCGTTGTTG
CAGCGAGAACCCGAGGGGCGCGATGAAGCCGCGGATGTTG
TGGGTGCGGCCGGCGCGTAGAGCGCACTCCCCGACCAACA
GGGTCGCGATGAGCTCAACGGCAAACCACTCCTTTTCCTT
TATGGTCTTAACGGCAAGCTTATGTTCGCGAATCAGTTGG
ACGTCGCCGTATCCCCCAGACCCCCCGAAGCTTCGGGCCC
CGGGGATCTCGAGGGTCGTGTAGTGTAGGGCGGGGTTGAT
GGCGAACACGGGGCTGCATAGCTTGCGGATGCGCGTGAGG
GTAAGGATGTGCGAGGGGGACGAGGGGGGTGCGGTTAACG
CCGCCTGGGATCTGCGCAGGGGCGGGCGGTTCAGTTGGCC
GCCGTACCGGGCGGCTCGGGGGACGCGCGGCGATGAGACG
AGCGGCTCATTCGCCATCGGGATAGTCCCGCGCGAAGCCG
CTCGCGGAGGCCGGATCGGTGGCGGGACCCGTGGGAGGAG
CGGGAGCCGGCGGCGTCCTGGAGAGAGGGGCCGCTGGGGC
GCCCGGAGGCCCCGTGTGGGTTGGGTGTATGTAGGATGCG
AGCCAATCCTTGAAGGACTGTTGGCGTGCATTGGGGGTGA
GGTGAGAGGAAAAATGACCAGCAGGTCGCTGTCTGCGGGA
CTCATCCATCCTTCGGCCAGGTCGCCGTCTTCCCACAGAG
AAGCGTTGGTCGCTGCTTCCTCGAGTTGCTCCTCCTGGTC
CGCAAGACGATCGTCCACGGCGTCCAGGCGCTCACCAAGC
GCCGGATCGAGGTACCGTCGGTGTGCGGTTAGAAAGTCAC
GACGCGCCGCTTGCTCCTCCACGCGAATTTTAACACAGGT
CGCGCGCTGTCGCATCATCTCTAAGCGCGCGCGGGACTTT
AGCCGCGCCTCCAATTCCAAGTGGGCCGCCTTTGCAGCCA
TAAAGGCGCCAACAAACCGAGGATCTTGGGTGCGACGCCC
CCCGGTGCAGCGCAGGGTCTGGTCCTTGTAAATCTCGGCT
CGGAGGTGCGTCTCGGCCAGGCGTCGGCGCAGGGCCGCGT
GGGCGGCATCTCGGTCCATTCCGCCCCCTGCGGGCGACCC
GGGGGGTGCTCTGATAGTCTCGCGTGCCCAAGGCCCGTGA
TCGGGGTACTTCGCCGCCGCGACCCGCCACCCGGTGTGCG
CGATGTTTGGTCAGCAGCTGGCGTCCGACGTCCAGCAGTA
CCTGGAGCGCCTCGAGAAACAGAGGCAACTTAAGGTGGGC
GCGGACGAGGCGTCGGCGGGCCTCACAATGGGCGGCGATG
CCCTACGAGTGCCCTTTTTAGATTTCGCGACCGCGACCCC
CAAGCGCCACCAGACCGTGGTCCCGGGCGTCGGGACGCTC
CACGACTGCTGCGAGCACTCGCCGCTCTTCCGGCCGTGGC
GCGGCGGCTGCTGTTTAATAGCCTGGTGCCGGCGCAACTA
AAGGGGCGGGAGGGCGGGGGCGACCACACGGCCAAGCTGG
AATTCCTGGCCCCCGAGTTGGTACGGGCGGTGGCGCGACT
GCGGTTTAAGGAGTGCGCGCCGGCGGACGTGGTGCCTCAG
CGTAACGCCTACTATAGCGTTCTGAACACGTTTCAGGCCC
TCCACCGCTCCGAAGCCTTTCGCCAGCTGGTGCACTTTGT
GCGGGACTTTGCCCAGCTGCTTAAAACCTCCTTCCGGGCC
TCCAGCCTCACGGAGACCACGGGCCCCCCAAAAAAACGGG
CCAAGGTGGACGTGGCCACCCACGGCCGGACGTACGGCAC
GCTGGAGCTGTTCCAAAAAATGATCCTTATGCACGCCACC
TACTTTCTGGCCGCCGTGCTCCTCGGGGACCACGCGGAGC
AGGTCAACACGTTCCTGCGTCTCGTGTTTGAGATCCCCCT
GTTTAGCGACGCGCCGTGCGCCACTTCCGCCAGCGCGCCA
CCGTGTTTCTCGTCCCCCGGCGCCACGGCAAGACCTGGTT
TCTAGTGCCCCTCATCGCGCTGTCGCTGGCCTCCTTTCGG
GGGATCAAGATCGGCTACACGGCGCACATCCGCAAGGCGA
CCGAGCCGGTGTTTGAGGAGATCGACGCCTGCCTGCGGGG
CTGGTTCGGTTCGGCCCGAGTGGACCACGTTAAAGGGGAA
ACCATCTCCTTCTCGTTTCCGGACGGGTCGCGCAGTACCA
TCGTGTTTGCCTCCAGCCACAACACAAACGTAAGTCCTCT
TTTCTTTCGCATGGCTCTCCCAAGGGGCCCCGGGTCGACC
CGACCCACACCCACCCACCCACCCACATACACACACAACC
AGACGCGGGAGGAAAGTCGGCCCCGTGGGCACTGATTTTT
ATTCGGGATCGCTTGAGGAGGCCCGGGCAACGGCCCGGGC
AACGGTGGGGCAACTCGTAGCAAATAGGCGACTGATGTAC
GAAGAGAAGACACACAGGCGCCACCCGGCGCTGGTCGGGG
GGATGTTGTCCGCGCCGCACCGTCCCCCGACGACCTCTTG
CAGACGGTCCGTGATGCAAGGACGGCGGGGGGCCTGCAGC
AGGGTGACCGTATCCACGGGATGGCCAAAGAGAAGCGGAC
ACAGGCTAGCATCCCCCTGGACCGCCAGGGTACACTGGGC
CATCTTGGCCCACAGACACGGGGCGACGCAGGGACAGGAC
TCCGTTACGACGGAGGAGAGCCACAGTGCGTTGGCGGAAT
CGATGTGGGGCGGCGGGGCGCAGGACTCGCAGCCCCCCGG
GTGGTTGGTGATCCTGGCCAGGAGCCATCCCAGATGGCGG
GCCCTGCTTCCCGGTGGACAGAGCGACCCCAGGTCGCTGT
CCATGGCCCAGCAGTAGATCTGGCCGCTGGGGAGGTGCCA
CCAGGCCCCCGGGCCCAAGGCGCAACACGCGCCCGGCTCC
GGGGGGGTCTTCGCGGGGACCAGATACGCGCCATCCAGCT
CGCCGACCACTGGCTCCTCCGCGAGCTGTTCGGTGGTTGG
GTCGGGGGTTTCCTCCGGGGGGGTGGCCGCCCGTATGCGG
GCGAACGTGAGGGTGCACAGGAGCGGGGTCAGGGGGTGCG
TCACGCTCCGGAGGTGGACGATCGAGCAGTAGCGGCGCTC
GCGGTTAAAGAAAAAGAGGGCAAAGAAGGTGTTCGGGGGC
AACCGCAGCGCCTTGGGGGCGTCAGAAAGAAAAATCTCGC
AGAAGAGGGGGCCCGGGGTCTGGGTTAGGAAGGGCCACCT
GACACAGAGGCTCGGTGAGGACCGTTAGACACCGAAAGAT
CTTGAGCCGCTCGTCCACCCGAACGACGCGCCACACAAAG
ACGGAGTTGACAATGCGCGCGATAGAGTCGACGTCCGTCC
CCAGGGCGTCGACTCTGTCGCGCGTGCCGCGAGCTCCGAC
CCGGGAATCCGGCCGGGGCAAGGTCCCCGGGGGACCAGGC
GGCGCCAGGGGCCGCCGGGGTCCCAGCTGCGCCATGCCGG
GGGCGGGGGGAGGGCAAACCCCAGAGGCGGGGGCCAACGG
CGCGGGGAGGAGTGGGTGGGCGAGGTGGCCGGGGGAAGGC
GCCCGCTAGCGAGAACGGCCGTTCCCGGACGACACCTTGC
GACAAAACCTAAGGACAGCGGCCCGCGCGACGGGGTCCGA
GAGGCTAAGGTAGGCCGCGATGTTAATGGTGAACGCAAAG
CCGCCGGGAAAGACAACTATGCCACAGAGGCGGCGATTAA
ACCCCAGGCAGAGGTAGGCGTAGCTTTCCCCGGGCAGGTA
TTGCTCGCAGACCCTGCGTGGGGCTGTGGAGGGGACGGCC
TCCATGAAGCGACATTTACTCTGCTCGCGTTTACTGACGT
CATCATCCATCGCCACGGCGATTGGACGATTGTTAAGCCG
CAGCGTGTCTCCGCTTGTGCTGTAGTAGTCAAAAACGTAA
TGGCCGTCGGAGTCGGCAAAGCGGGCCGGGAGGTCGTCGC
CGAGCGGGACGACCCGCCGCCCCCGACCGCCCCGTCCCCC
CAGGTGTGCCAGGACGGCCAGGGCATACGCGGTGTGAAAA
AAGGCGTCGGGGGCGGTCCCCTCGACGGCGCGCATCAGGT
TCTCGAGGAGAATGGGGAAGCGCCTGGTCACCTCCCCCAG
CCACGCGCGTTGGTCGGGGCCAAAGTCATAGCGCAGGCGC
TGTGAGATTCGAGGGCCGCCCTGAAGCGCGGCCCGGATGG
CCTGGCCCAGGGCCCGGAGGCACGCCAGATGTATGCGCGC
AGTAAAGGCGACCTCGGCGGCGATGTCAAAGGGCGGCAGG
ACGGGGCGCGGGTGGCGCAGGGGCACCTCGAGCGCGGGAA
AGCGGAGCAGCAGCTCCGCCTGCCCAGCGGGAGACAGCTG
GTGGGGGCGCACGACGCGTTCTGCGGCGCAGGCCTCGGGT
CGGGGCCGTGGCCAGCGCCGAGGACAGCAGCGGAGGGCGG
GCGCGTCGCCCGCCCCACGCCACGGAGTTCTCGTAGGAGA
CGACGACGAAGCGCTGCTTGGTTCCGTAGTGGTGGCGCAG
GACCACGGAGATAGAACGACGGCTCCACAGCCAGTCCGGC
CGGTCGCCGCCGGCCAGGGCTTCCCATCCGCGATCCAACC
ACTCGACCAGCGACCGCGGCTTTGCGGTACCAGGGGTCAG
GGTTAGAACGTCGTTCAGGATGTCCTCGCCCCCGGGCCCG
TGGGGCACTGGGGCCACAAAGCGGCCCCCGCCTGGGGGCT
CCAGACCCGCCAACACCGCATCTGCGTCAGCCGCCCCCAT
GGCGCCCCCGCTGACGGCCTGGTGAACCAGGGCGCCCTGG
CGGAGCCCCGATGCAACGCCACAGGCCGCACGCCCGGTCC
GAGCGCGGACCGGGTGGCGGCGGGTGACGTCCTGCACTGC
CCGCTGAACCAACGCGAGGATCTCCTCGTTCTCCTGCGCG
ATGGACACGTCCTGGGCCGCGGTCGTGTCGCCGCCGGGGG
CCGTCAGCTGCTCCTCCGGGGAGATGGGGGGGTCGGACGC
CCCGACGATGGGCGGGTCTGCGGGCGCCCCCGCGTGGGGC
CGGGCCAAGGGCTGCGGACGCGGGGACGCGCTTTCCCCCA
GACCCATGGACAGGTGGGCCGCAGCCTCCTTCGCGGCCGG
CGGGGCGGCGGCGCCAAGCAGAGCGACGTAGCGGCACAAA
TGCCGACAGACGCGCATGATGCGCGTGCTGTCGGCCGCGT
AGCGCGTGTTGGGGGGGACGAGCTCGTCGGAACTAAACAG
AATCACGCGGGCACAGCTCGCCCCCGAGCCCCACGCAAGG
CGCAGCGCCGCCACGGCGTACGGGTCATAGACGCCCTGTG
CGTCACACACCACGGGCAAGGAGACGAACAACCCCCCGGC
GCTGGACGCACGCGGAAGGAGGCCAGGGTGTGCCGGCACG
ACGGGGGCCAGAAGCTCCCCCACCGCATCCGCGGGCACGT
AGGCGGCAAACGCCGTGCACCACGGGGTACAGTCGCCGGT
GGCATGAGCCCGAGTCTGGATTTCGACCTGGAAGTTTGCG
GCCGTCCCGAGTCCGGGGCGGCCGCGCATCAGGGCGGCCA
GAGGGATTCCCGCGGCCGCCAGGCACTCGCTGGATATGAT
GACGTGAACCAAAGACGAGGGCCGACCCGGGACGTGGCCG
AGATCGTACTGGACCTCGTTGGCCAAGTGCGCGTTCATGG
TTCGGGGGTGGGTGTGGGTGTGTAGGCGATGCGGGTCCCC
CGAGTCCGCGGGAAGGGCGCGGGTTTGGCGCGCGTATGCG
TATTCGCCAACGGAGGCGTGCGTGCTTATGCGCGGCGCGT
TTCTTCTGTCTCCAGGGAATCCGAGGCCAGGACTTTAACC
TGCTCTTTGTCGACGAGGCCAACTTTATTCGCCCGGATGC
GGTCCAGACGATTATGGGCTTTCTCAACCAGGCCAACTGC
AAGATTATCTTCGTGTCGTCCACCAACACCGGGAAGGCCA
GTACGAGCTTTTTGTACAACCTCCGCGGGGCCGCCGACGA
GCTTCTCAACGTGGTGACCTATATATGCGATGATCACATG
CCGCGGGTGGTGACGCACACAAACGCCACGGCCTGTTCTT
GTTATATCCTCAACAAGCCCGTTTCAGCACGATGGACGGG
GCGGTTCGCCGGACCGCCGATTTGTTTCTGGCCGATTCCT
TCATGCAGGAGATCATCGGGGGCCAGGCCAGGGAGACCGG
CGACGACCGGCCCGTTCTGACCAAGTCTGCGGGGGAGCGG
TTTCTGTTGTACCGCCCCTCGACCACCACCAACAGCGGCC
TCATGGCCCCCGATTTGTACGTGTACGTGGATCCCGCGTT
CACGGCCAACACCCGAGCCTCCGGGACCGGCGTCGCTGTC
GTCGGGCGGTACCGCGACGATTATATCATCTTCGCCCTGG
AGCACTTTTTTCTCCGCGCGCTCACGGGCTCGGCCCCCGC
CGACATCGCCCGCTGCGTCGTCCACAGTCTGAGGTAGGGC
CAGGCCCTGCATCCCGGGGCGTTTCGCGGCGTCCGGGTGG
CGGTCGAGGGAAATAGCAGCCAGGACTCGGCCGTCGCCAT
CGCCACGCACGTGCACACAGAGATGCACCGCCTATGGCCT
CGGAGGGGGCCGACGCGGGCTCGGGCCCCGAGCTTCTCTT
CTACCACTGCGAGCCTCCCGGGAGCGCGGTGCTGTACCCC
TTGGTCCTGCTCAACAAACAGAAGACGCCCGCCTTTGAAC
ACTTTATTAAAAAGTTTAACTCCGGGGGCGTCATGGCCTC
CCAGGAGATCGTTTCCGCGACGGTGCGCCTGCAGACCGAC
CCGGTCGAGTATCTGCTCGAGCAGCTGAATAACCTCACCG
AAACCGTCTCCCCCAACACGGACGTCCGTACGTATTCCGG
AAAACGGAACGGCGCCTCGGATGACCTTATGGTCGCCGTC
ATTATGGCCATCTACCTTGCGGCCCAGGCCGGACCTCCGC
ACACATTCGCTCCCATCACACGCGTTTCGTGAGCGCCCAA
TAAACACACCCAGGTATGCTACGCACGACCACGGTGTCGC
CTGTTAAGGGGGGGGGAAGGGGGTGTTGGCGGGAAGCGTG
GGAACACGGGGGATTCTCTCACGACCGGCACCAGTACCAC
CCCCCTGTGAACACAGAAACCCCAACCCAAATCCCATAAA
CATACGACACCCGGCATATTTTGGAATTTCTTCGGTTTTT
ATTTATTTAGGTATGCTGGGGTTTCTCCCTGGATGCCCAC
CCCCACCCCCCCCGTGGGTCTAGCCGGGCCTTAGGGATAG
CGTATAACGGGGGCCATGTCTCCGGACCGCACAACGGCCG
CGCCGTCAAAGGTGCACACCCGAACCACGGGAGCCAGGGC
CAAGGTGTCTCCTAGTTGGCCCGCGTGGGTCAGCCAGGCG
ACGAGCGCCTCGTAGAGCGGCAGCCTTCGCTCTCCATCCT
GCATCAGGGCCGGGGCTTCGGGGTGAATGAGCTGGGCGGC
CTCCCGCGTGACACTCTGCATCTGCAGGAGAGCGTTCACG
TACCCGTCCTGGGCACTTAGCGCAAAGAGCCGGGGGATTA
GCGTAAGGATGATGGTGGTTCCCTCCGTGATCGAGTAAAC
CATGTTAAGGACCAGCGATCGCAGCTCGGCGTTTACGGGG
CCGAGTTGTTGGACGTCCGCCAGCAGCGAGAGGCGACTCC
CGTTGTAGTACAGCACGTTGAGGTCTGGCAGCCCTCCGGG
GTTTCTGGGGCTGGGGTTCAGGTCCCGGATGCCCCTGGCC
ACGAGCCGCGCCACGATTTCGCGCGCCAGGGGCGATGGAA
GCGGAACGGGAAACCGCAACGTGAGGTCCAGCGAATCCAG
GCGCACGTCCGTCGCTTGGCCCTCGAACACGGGCGGGACG
AGGCTGATGGGGTCCCCGTTACAGAGATCTACGGGGGAGG
TGTTGCGAAGGTTAACGGTGCCGGCGTGGGTGAGGCCCAC
GTCCAGGGGGCAGGCGACGATTCGCGTGGGAAGCACCCGG
GTGATGACCGCGGGGAAGCGCCTTCGGTACGCCAGCAACA
GCCCCAACGTGTCGGGACTGACGCCTCCGGAGACGAAGGA
TTCGTGCGCCACGTCGGCCAGCGTCAGTTGCCGGCGGATG
GTCGGAGGAATACCACCCGCCCTTCGCAGCGCTGCAGCGC
CGCCGCATCGGGGCGCGAGATGCCCGAGGGTATCGCGATG
TCAGTTTCAAAGCCGTCCGCCAGCATGGCGCCGATCCACG
CGGCAGGGAGTGCAGTGGTGGTTCGGGTGGCGGGAGGAGC
GCGGTGGGGGTCAGCGGCGTAGCAGAGACGGGCGACCAAC
CTCGCATAGGACGGGGGGTGGGTCTTAGGGGGTTGGGAGG
CGACAGGGACCCCAGAGCATGCGCGGGGAGGTCTGTCGGG
CCCAGACGCACCGAGAGCGAATCCGCCATGGGCCCGGCCT
GGGTTTTATGGGGCCCGGCCCTCGGAATCGCGGCTTGTCG
GCGGGGGCAAAGGGGGCGGGGCTAGGGGGCTTGCGGGAAC
AGAGACGGGTGGGGTAAAAGAATCGCACTACCCCAAGGAA
GGGCGGGGCGGTTTATTACAGAGCCAGTCCCTTGAGCGGG
GATGCGTCATAGACGAGATACTGCGCGAAGTGGGTCTCCC
GCGCGTGGGCTTCCCCGTTGCGGGCGCTGCGGAGGAGGGC
GGGGTCGCTGGCGCAGGTGAGCGGGTAGGCCTCCTGAAAC
AGGCCACACGGGTCCTCCACGAGTTCGCGGCACCCCGGGG
GGCGCTTAAACTGTACGTCGCTGGCGGCGGTGGCCGTGGA
CACCGCCGAACCCGTCTCCACGATCAGGCGCTCCAGGCAG
CGATGTTTGGCGGCGATGTCGGCCGACGTAAAGAACTTAA
AGCAGGGGCTGAGCACCGGCGAGGCCCCGTTGAGGTGGTA
GGCCCCGTTATAGAGCAGGTCCCCGTACGAAAATCGCTGC
GACGCCCACGGGTTGGCCGTGGCCGCGAAGGCCCGGGACG
GGTCGCTCTGGCCGTGGTCGTACATGAGGGCGGTGACACC
CCCTCCTTGCCCCCGCGTAAACGCCCCCGGGGCGCGCCCC
GGGGGGTTGCGGGGCCGGCGGAAGTAGTTGACGTCGGTCG
ACACGGGGGTGGCGATAAACTCACACACGGCGTCCTGGCC
GTGGTCCATCCCTGCGCGCCGCGGCCCCTGGGCGCACCCG
AACACGGGGACGGGCTGGGCCGGCCCCAGGCGGTTTCCCG
CCACGACCGCGTTCCGCAGGTACACGGCTGCCGCGTTGTC
CAGTAGAGGGGGAGCCCCGCGGCCCAGGTAAAAGTTTTGG
GGAAGGTTGCCCATGTCGGTGACGGGGTTGCGGACGGTTG
CCGTGGCCACGACGGCGGTGTAGCCCACGCCCAGGTCCAC
GTTCCCGCGCGGCTGGGTGAGCGTGAAGTTTACCCCCCCG
CCAGTTTCATGCCGGGCCACCTGGAGCTGGCCCAGGAAGT
ACGCCTCCGACGCGCGCTCCGAGAACAGCACGTTCTCAGT
CACAAAGCGGTCCTGTCGGACGACGGTGAACCCAAACCCG
GGATGGAGGCCCGTCTTGAGCTGATGATGCAAGGCCACGG
GACTGATCTTGAAGTACCCCGCCATGAGCGCGTAGGTCAG
CGCGTTCTCCCCGGCCGCGCTCTCGCGGACGTGCTGCACG
ACGGGCTGTCGGATCGACGAAAAGTAGTTGGCCCCCAGAG
CCGGGGGGACCAGGGGGACCTGCCGCGACAGGTCGCGCAG
GGCCGGGGGGAAATTGGGCGCGTTCGCCACGTGGTCGGCC
CCGGCGAACAGCGCGTGGACGGGGAGGGGGTAAAAATAGT
CGCCATTTTGGATGGTATGGTCCAGATGCTGGGGGGCCAT
CAGCAGGATTCCGGCGTGCAACGCCCCGTCGAATATGCGC
ATGTTGGTGGTGGACGCGGTGTTGGCGCCCGCGTCGGGCG
CCGCCGAGCAGAGCAGCGCCGTTGTGCGTTCGGCCATGTT
GTGGGCCAGCACCTGCAGCGTGAGCATGGCGGGCCCGTCC
ACTACCACGCGCCCGTTGTGAAACATGGCGTTGACCGTGT
TGGCCACCAGATTGGCCGGGTGCAGGGGGGGCGCGGGGTC
CGTCACGGGGTCGCTGGGGCAATCCTCGCCGGGGGTGATC
TCCGGGACCACCATGTTCTGCAGGGTGGCGTATACGCGGT
CGAAGCGAACCCCCGCGGTGCAGCAGCGGCCCCGCGAGAA
GGCGGGCACCATCACGTAGTAGTAAATCTTGTGGTGCACG
GTCCAGTCCGCCCCCCGGTGCGCCGGTCGTCCGCGGCGTC
CGCGGCTCGGGCCTGGGTGTTGTGCAGCAGCTGGCCGTCG
TTGCGGTTGAAGTCCGCGGTCGCCACGTTACACGCCGCTG
CGTACACGGGGTCGTGGCCCCCCGCGCTAACCCGGCAGTC
GCGATGGCGGTCCAGGGCCGCGCGCCGCATCAGGGCGTCG
CAGTCCCACACGAGGGGTGGCAGCAGCGCCGGGTCTCGCA
TTAGGTGATTCAGTTCGGCTTGCGCCTGCCCGCCCAGTTC
CGGGCCGGTCAGGGTAAAGTCATCAACCAGCTGGGCCAGG
GCCTCGACGTGCGCCACCAGGTCCCGGTACACGGCCATGC
ACTCCTCGGGAAGGTCTCCCCCGAGGTAGGTCACGACGTA
CGAGACCAGCGAGTAGTCGTTCACGAACGCCGCGCACCGC
GTGTTGTTCCAGTAGCTGGTGATGCACTGGACCACGAGCC
GGGCCAGGGCGCAGAAGACGTGCTCGCTGCCGTGTATGGC
GGCCTGCAGCAGGTAAAACCCGCCGGGTAGTTGCGGTCTT
CGAACGCCCCGCGAACGGCGGCGATGGTGGCGGGGGCCAT
GGCGTGGCGTCCCACCCCCAGCTCCAGGCCCCGGGCGTCC
CGGAACGCCGCCGGACATAGCGCCAGGGGCAAGTTGCCGT
TCACCACGCGCCAGGTGGCCTGGATCTCCCCCGGGCCGGC
CGGGGGAACGTCCCCCCCCGGCAGCTCCACGTCGGCCACC
CCCACGAAGAAGTCGAACGCGGGGTGCAGCTCAAGAGCCA
GGTTGGCGTTGTCGGGCTGCATAAACTGCTCCGGGGTCAT
CTGGCCTTCCGCGACCCATCGGACCCGCCCGTGGGCCAGG
CGCTGCCCCCAGGCGTTCAAAAACAGCTGCTGCATGTCTG
CGGCGGGGCCGGCCGGGGCCGCCACGTACGCCCCGTACGG
ATTGGCGGCTTCGACGGGGTCGCGGTAAGGCCCCCGACCG
CCGCGTCAACGTTCATCAGCGAAGGGTGGCACACGGTCCC
GATCGCGTGTTCCAGAGACAGGCGCAGCACCTGGCGGTCC
TTCCCCCAAAAAAACAGCTGGCGGGGCGGGAAGGCGCGGG
GATCCGGGTGGCCGGGGGCGGGGACTAGGTCCCCGGCGTG
CGCGGCAAACCGTTCCATGACCGGATTGAACAGGCCCAGG
GGCAGGACGAACGTCAGGTCCATGGCGCCCACCAGGGGGT
AGGGAACGTTGGTGGCGGCGTAGATGCGCTTCTCCAGGGC
CTCCAAAAAGATCAGCTTCTCGCCGATGGACACCAGATCC
GCGCGCACGCGCGTCGTCTGGGGGGCGCTCTCGAGCTCGT
CCAGCGTCTGCCGGTTCAGGTCGAGCTGCTCCTCCTGCAT
CTCCAGCAGGTGGCGGCCCACGTCGTCCAGACTTCGCACG
GCCTTGCCCATCACGAGCGCCGTGACCAGGTTGGCCCCGT
TCAGGACCATCTCGCCGTACGTCACCGGCACGTCGGCTTC
GGTGTCCTCCACTTTCAGGAAGGACTGCAGGAGGCGCTGT
TTGATCGGGGCGGTGGTGACGAGCACCCCGTCGACCGGAC
GCCCGCGCGTGTCGGCATGCGTCAGACGGGGCACGGCCAT
GGAGGGCTGCGTGGCCGTGGTGAGGTCCACGAGCCAGGCC
TCGACGGCCTCCCGGCGGTGGCCCGCCTTGCCCAGGAAAA
AGCTCGTCTCGCAGAAGCTTCGCTTTAGCTCGGCGACCAG
GGTCGCCCGGGCCACCCTGGTGGCCAGGCGGCCGTTGTCC
AGGTATCGTTGCATCGGCAACAACAAAGCCAGGGGCGGCG
CCTTTTCCAGCAGCACGTGCAGCATCTGGTCGGCCGTGCC
GCGCTCAAACGCCCCGAGGACGGCCTGGACGTTGCGAGCG
AGTTGTTGGATGGCGCGCAACTGGCGATGCGCGCTGATAC
CCGTCCCGTCCAGGGCCTCCCCCGTGGCAGGGCGATGGCC
TCGGTGGCCAGGCTGAAGGCGGCGTTCAGGGCCCGGCGGT
CGATAATCTTGGTCATGTAATTGTGTGTGGGTTGCTCGAT
GGGGTGCGGGCCGTCGCGGGCAATCAGCGGCTGGTGGACC
TCGAACTGTACGCGCCCCTCGTTCATGTAGGCCAGCTCCG
GAAACTTGGTACACACGCACGCCACCGACAACCGCGCTCC
AGAAAGCGCACGAGCGACAGGGTGTTGCAATACGACCCCA
ACAGGGCGTCGAACTCGACGTCATACAGGCTGTTTGCATC
GGAGCGCACGCGGGAAAAAAAATCGAACAGGCGTCGATGC
GACGCCACCTCGATCGTGCTAAGGAGGGACCCGGTCGGCA
CCATGGCCGCGGCATACCGGTATCCCGGAGGGTCGCGGTT
GGGAGCGGCCATGGGGTCGCGTGGAGATCGGCTGTCTCTA
GTGATATTGGCCCGGGGAGGCTAAGATCCACCCCAACGCC
CGGCCACCCGTGTACGTGCCCGACGGCCCAAGGTCCACCG
AAAGACACGACGGGCCCGGACCCAAAAAGGCGGGGGATGC
TGTGTGAGAGGCCGGGTGTCGGTCGGGGGGGAAAGGCACC
GGGAGAAGGCTGCGGCCTCGTTCCAGGAGAACCCAGTGTC
CCCAACAGACCCGGGGACGTGGGATCCCAGGCCTTATATA
CCCCCCCCGCCCCACCCCCGTTAGAACGCGACGGGTGCAT
TCAAGATGGCCCTGGTCCAAAAGCGTGCCAGGAAGAAATT
GGCAGAGGCGGCAAAGCTGTCCGCCGCCGCCACCCACATC
GAGGCCCCGGCCGCGCAGGCTATCCCCAGGGCCCGTGTGC
GCAGGGGATCGGTGGGCGGCAGCATTTGGTTGGTGGCGAT
AAAGTGGAAAAGCCCGTCCGGACTGAAGGTCTCGTGGGCG
GCGGCGAACAAGGCACACAGGGCCGTGCCTCCCAAAAACA
CGGACATCCCCCAAAACACGGGCGCCGACAACGGCAGACG
ATCCCTCTTGATGTTAACGTACAGGAGGAGCGCCCGCACC
GCCCACGTAACGTAGTAGCCGACGATGGCGGCCAGGATAC
AGGCCGGCGCCACCACCCTTCCGGTCAGCCCGTAATACAT
GCCCGCTGCCACCATCTCCAACGGCTTCAGGACCAAAAAC
GACCAAAGGAACAGAATCACGCGCTTTGAAAAGACCGGCT
GGGTATGGGGCGGAAGACGCGAGTATGCCGAACTGACAAA
AAAGTCAGAGGTGCCGTACGAGGACAATGAAAACTGTTCC
TCCAGTGGCAGTTCTCCCTCCTCCCCCCCAAAGGCGGCCT
CGTCGACCAGATCTCGATCCACCAGAGGAAGGTCATCCCG
CATGGTCATGGGGTGTGCGGTGGAGGTGGGGAGACCGAAA
CCGCAAAGGGTCGCTTACGTCAGCAGGATCCCGAGATCAA
AGACACCCGGGTTCTTGCACAAACACCACCCGGGTTGCAT
CCGCGGAGGCGAGTGTTTTGATAAGGCCGTTCCGCGCCTT
GATATAACCTTTGATGTTGACCACAAAACCCGGAATTTAC
GCCTACGCCCCAATGCCCACGCAAGATGAGGTAGGTAACC
CCCCCCCGTGGGTGTGACGTTGCGTTTAGTTCATTGGAGG
CCAAGGGGAAAATGGGGTGGGGAGGAAACGGAAAACCCAG
TAGGCCGTGTTGGGAACACGCCCGGGGTTGTCCTCAAAAG
GCAGGGTCCATACTACGGAAGCCGTCGTTGTATTCGAGAC
CTGCCTGTGCGACGCACGTCGGGGTTGCCTGTGTCCGGTT
CGGCCCCACCGCGTGCGGCACGCACGAGGACGAGTCCGCG
TGCTTTATTGGCGTTCCAAGCGTTGCCCTCCAGTTTCTGT
TGTCGGTGTTCCCCCATACCCACGCCCACATCCACCGTAG
GGGGCCTCTGGGCCGTGCACGTCGCCGCCCGCGATGGAGC
TTAGCTACGCCACCACCATGCACTACCGGGACGTTGTGTT
TTACGTCACAACGGACCGAAACCGGGCCTACTTTGTGTGC
GGGGGGTGTGTTTATTCCGTGGGGCGGCCGTGTGCCTCGC
AGCCCGGGGAGATTGCCAAGTTTGGTCTGGTCGTTCGAGG
GACAGGCCCAGACGACCGCGTGGTCGCCAACTATGTACGA
AGCGAGCTCCGACAACGCGGCCTGCAGGACGTGCGTCCCA
TTGGAGGACGAGGTGTTTCTGGACAGCGTGTGTCTTCTAA
ACCCGAACGTGAGCTCCGAGCTGGATGTGATTAACACGAA
CGACGTGGAAGTGCTGGACGAATGTCTGGCCGAGTACTGC
ACCTCGCTGCGAACCAGCCCGGGTGTGCTAATATCCGGGC
TGCGCGTGCGGGCGCAGGACAGAATCATCGAGTTGTTTGA
ACACCCAACGATAGTCAACGTTTCCTCGCACTTTGTGTAT
ACCCCGTCCCCATACGTGTTCGCCCTGGCCCAGGCGCACC
TCCCCCGGCTCCCGAGCTCGCTGGAGGCCCTGGTGAGCGG
CCTGTTTGACGGCATCCCCGCCCCACGCCAGCCACTTGAC
GCCCACAACCCGCGCACGGATGTGGTTATCACGGGCCGCC
GCGCCCCACGACCCATCGCCGGGTCGGGGGCGGGGTCGGG
GGGCGCGGGCGCCAAGCGGGCCACCGTCAGCGAGTTCGTG
CAAGTCAAACACATTGACCGCGTGGGCCCCGCTGGCGTTT
CGCCGGCGCCTCCGCCAAACAACACCGCTCAAGTTCCCGG
TGCCCGGGGCCCAGGATTCCGCCCCGCCCGGCCCCACGCT
AAGGGAGCTGTGGTGGGTGTTTTATGCCGCAGACCGGGCG
CTGGAGGAGCCCCGCGCCGACTCTGGCCTCACCCGCGAGG
AGGTACGTGCCGTACGTGGGTTCCGGGAGCAGGCGTGGAA
ACTGTTTGGCTCCGCGGGGGCCCCGCGGGCGTTTATCGGG
GCCGCGTTGGGCCTGAGCCCCCTCCAAAAGCTAGCCGTTT
ACTACTATATCATCCACCGAGAGAGGCGCCTGTCCCCCTT
CCCCGCGCTAGTCCGGCTCGTAGGCCGGTACACACAGCGC
CACGGCCTGTACGTCCCTCGGCCCGCGCTGGAGGCCCTGG
TGAGCGGCCTGTTTGACGGCATCCCCGCCCCACGCCAGCC
ACTTGACGCCCACAACCCGCGCACGGATGTGGTTATCACG
GGCCGCCGCGCCCCACGACCCATCGCCGGGTCGGGGGCGG
GGTCGGGGGGCGCGGGCGCCAAGCGGGCCACCGTCAGCGA
GTTCGTGCAAGTCAAACACATTGACCGCGTGGGCCCCGCT
GGCGTTTCGCCGGCGCCTCCGCCAAACAACACCGACTCAA
GTTCCCTGGTGCCCGGGGCCCAGGATTCCGCCCCGCCCGG
CCCCACGCTAAGGGAGCTGTGGTGGGTGTTTTATGCCGCA
GACCGGGCGCTGGAGGAGCCCCGCGCCGACTCTGGCCTCA
CCCGCGAGGAGGTACGTGCCGTACGTGGGTTCCGGGAGCA
GGCGTGGAAACTGTTTGGCTCCGCGGGGGCCCCGCGGGCG
TTTATCGGGGCCGCGTTGGGCCTGAGCCCCCTCCAAAAGC
TAGCCGTTTACTACTATATCATCCACCGAGAGAGGCGCCT
GTCCCCCTTCCCCGCGCTAGTCCGGCTCGTAGGCCGGTAC
ACACAGCGCCACGGCCTGTACGTCCCTCGGCCCGACGACC
CAGTCTTGGCCGATGCCATCAACGGGCTGTTTCGCGACGC
GCTGGCGGCCGGAACCACAGCCGAGCAGCTCCTCATGTTC
GACCTTCTCCCCCCAAAGGACGTGCCGGTGGGAAGCGACG
TGCAGGCCGACAGCACCGCTCTGCTGCGCTTTATAGAATC
GCAACGTCTCGCCGTCCCCGGGGGGGTGATCTCCCCCGAG
CACGTCGCGTACCTTGGTGCGTTCCTGAGCGTGCTGTACG
CTGGCCGCGGGCGCATGTCCGCAGCCACGCACACCGCGCG
GCTGACAGGGGTGACCTCCCTGGTGCTAGCGGTGGGTGAC
GTGGACCGTCTTTCCGCGTTTGACCGCGGAGCGGCGGGCG
CGGCCAGCCGCACGCGGGCCGCCGGGTACCTGGATGTGCT
TCTTACCGTTCGTCTCGCTCGCTCCCAACACGGACAGTCT
GTGTAACAGACCCCAATAAACGTATGTCGCTACCACACCC
TTGTGTGTCAATGGACGCCTCTCCGGGGGGGAAGGGAAAA
CAAAGAGGGGCTGGGGGAGCGGCACCACCGGGGCCTGAAC
AAACAAACCACAGACACGGTTACAGTTTATTCGGTCGGGC
GGAGAAACGGCCGAAGCCACGCCCCCTTTATTCGCGTCTC
CAAAAAAACGGGACACTTGTCCGGAGAACCTGTAGGATGC
CAGCCAGGGCGGCGGTAATCATAACCACGCCCAGCGCAGA
GGCGGCCAGAAACCCGGGCGCAATTGCGGCCACGGGCTGC
GTGTCAAAGGCTAGCAAATGAATGACGGTTCCGTTTGGAA
ATAGCAACAAGGCCGTGGACGGCACGTCGCTCGAAAACAC
GCTTGGGGCGCCCTCCGTCGGCCCGGCGGCGATTTGCTGC
TGTGTGTTGTCCGTATCCACCAGCAACACAGACATGACCT
CCCCGGCCGGGGTGTAGCGCATAAACACGGCCCCCACGAG
CCCCAGGTCGCGCTGGGTTTGGGTGCGCACCAGCCGCTTG
GACTCGATATCCCGGGTGGAGCCTTCGCATGTCGCGGGAG
GTAGGTTAGGAACAGTGGGCGTCGGACGTCGACGCCGGTG
AGCTTGTAGCCGATCCCCCGGGGGAGAGGGGAGGGGGAAG
AGAAGAGGGCGTTGTGGGTGATGGGTACCAGGATCCGTGG
CTCGACGTTGGCAGACTGCCCCCCGCACCGATGTGAGGCC
TCAGGGACGAAGGCGCGGATCAGGGCGTTGTAGTGTGCCC
AGCGCGTCAGGGTCGAGGCGAGGCCGTGGGTCTGCTGGGC
CAGGACTTCGACCGGGGTCTCGGATCGGGTGGCTTGAGCC
AGCGCGTCCAGGATAAACACGCGCTCGTCTAGATCAAAGC
GCAGGGAGGCCGCGCATGGCGAAAAGTGGTCCGGAAGCCA
AAAGAGGGTTTTCTGGTGGTCGGCCCGGGCCAGCGCGGTC
CGGAGGTCGGCGTTGGTCGCTGCGGCGACGTCGGACGTAC
ACAAGGCCGAGGCTATCAGAAGGCTCCGGCGGGCGCGTTC
CCGCTGCACCGCCGAGGGGACGCCCGCCAAGAACGGCTGC
CGGAGGACAGCCGAGGCGTAAAATAGCGCCCGGTGGACGA
CCGGGGTGGTCAGCACGCGGCCCCCTAGAAACTCGGCATA
CAGGGCGTCGATGAGATGGGCTGCGCTGGGCGCCACTGCG
TCGTACGCCGAGGGGCTATCCAGCACGAAGGCCAGCTGAT
AGCCCAGCGCGTGTAATGCCAAGCTCTGTTCGCGCTCCAG
AATCTCGGCCACCAGGTGCTGGAGCCGAGCCTCTAGCTGC
AGGCGGGCCGTGGGATCCAAGACTGACACATTAAAAAACA
CAGAATCCGCGGCACAGCCCGCGGCCCCGCGGGCGGCCAA
CCCGGCAAGCGCGCGCGAGTGGGCCAAAAAGCCTAGCAGG
TCGGAGAGGCAGACCGCGCCGTTTGCGTGGGCGGCGTTCA
CGAAAGCAAAACCCGACGTCGCGAGCAGCCCCGTTAGGCG
CCAGAAGAGAGGGGGACGCGGGCCCTGCTCGGCGCCCGCG
TCCCCCGAGAAAAACTCCGCGTATGCCCGCGACAGGAACT
GGGCGTAGTTCGTGCCCTCCTCCGGGTAGCCGCCCACGCG
GCGGAGGGCGTCCAGCGCGGAGCCGTTGTCGGCCCGCGTC
AGGGACCCTAGGACAAAGACCCGATACCGGGGGCCGCCCG
GGGGCCCGGGAAGAGCCCCCGGGGGGTTTTCGTCCGCGGG
GTCCCCGACCCGATCTAGCGTCTGGCCCGCGGGGACCACC
ATCACTTCCACCGGAGGGCTGTCGTGCATGGATATCACGA
GCCCCATGAATTCCCGCCCGTAGCGCGCGCGCACCAGCGC
GGCATCGCACCCGAGCACCAGCTCCCCCGTCGTCCAGATG
CCCACGGGCCACGTCGAGGCCGACGGGGAGAAATACACGT
ACCTACCTGGGGATCTCAACAGGCCCCGGGTGGCCAACCA
GGTCGTGGACGCGTTGTGCAGGTGCGTGATGTCCAGCTCC
GTCGTCGGGTGCCGCCGGGCCCCAACCGGCGGTCGGGGGG
GCGGCTGCAGGCGGGCCGTGGGATCCAAGACTGACACATT
AAAAAACAGAATCCGCGGCACAGCCCGCGGCCCCGCGGGC
GGCCAACCCGGCAAGCGCGCGCGAGTGGGCCAAAAAGCCT
AGCAGGTCGGAGAGGCAGACCGCGCCGTTGCGTGGGCGGC
GTTCACGAAAGCAAAACCCGACGTCGCGAGCAGCCCCGTT
AGGCGCCAGAAGAGAGGGGGACGCGGGCCCTGCTCGGCGC
CCGCGTCCCCCGAGAAAAACTCCGCGTATGCCCGCGACAG
GAACTGGGCGTAGTTCGTGCCCTCCTCCGGGTAGCCGCCC
ACGCGGCGGAGGGCGTCCAGCGCGGAGCCGTTGTCGGCCC
GCGTCAGGGACCCTAGGACAAAGACCCGATACCGGGGGCC
GCCCGGGGGCCCGGGAAGAGCCCCCGGGGGGTTTCGTCCG
CGGGGTCCCCGACCCGATCTAGCGTCTGGCCCGCGGGGAC
CACCATCACTTCCACCGGAGGGCTGTCGTGCATGGATATC
ACGAGCCCCATGAATTCCCGCCCGTAGCGCGCGCGCACCA
GCGCGGCATCGCACCCGAGCACCAGCTCCCCCGTCGTCCA
GATGCCCACGGGCCACGTCGAGGCCGACGGGGAGAAATAC
ACGTACCTACCTGGGGATCTCAACAGGCCCCGGGTGGCCA
ACCAGGTCGTGGACGCGTTGTGCAGGTGCGTGATGTCCAG
CTCCGTCGTCGGGTGCCGCCGGGCCCCAACCGGCGGCGGG
GGGGCGGTGTATCACGCGGCCCGCTCGGGTGGCTCGCCGT
CGCCACGTTGGCTCCCCGCGGGAAAAGAGGGAACGTCAGG
GCCTCGGGGTCAGGGACGGCCGAAAACGTTACCCAGGCCC
GGGAACGCAGCAACACGGAGGCGGTTGGATTGTGCAAGAG
ACCCTTAAGGGGGGCGACCGCGGGGGGAGGCTGGGCGGTC
GGCTCGACCGTGATGGGGGCGGGCAGGCTCGCGTTCGGGG
GCCGGCCGAGCAGGTAGGTCTTCGAGATGTAAAGCAGCTG
GCCGGGGTCCCGCGGAAACTCGGCCGTGGTGACCAATACA
AAACAAAAGCGCTCCTCGTACCAGCGAAGAAGGGGCAGAG
ATGCCGTAGTCAGGTTTAGTTCGTCCGGCGGCGCCAGAAA
TCCGCGCGGTGGTTTTTGGGGGTCGGGGGTGTTTGGCAGC
CACAGACGCCCGGTGTTCGTGTCGCGCCAGTACATGCGGT
CCATGCCCAGGCCATCCAAAAACCATGGGTCTGTCTGCTC
AGTCCAGTCGTGGACCTGACCCCACGCAACGCCCAAAAGA
ATAACCCCCACGAACCATAAACCATTCCCCATGGGGGACC
CCGTCCCTAACCCACGGGGCCCGTGGCTATGGCAGGGCTT
GCCGCCCCGACGTTGGCTGCGAGCCCTGGGCCTTCACCCG
AACTTGGGGGTTGGGGTGGGGAAAAGGAAGAAACGCGGGC
GTATTGGCCCCAATGGGGTCTCGGTGGGGTATCGACAGAG
TGCCAGCCCTGGGACCGAACCCCGCGTTTATGAACAAACG
ACCCAACACCCGTGCGTTTTATTCTGTCTTTTTATTTCCG
TCATAGCGCGGGTTCCTTCCGGTATTGTCTCCTTCCGTGT
TTCAGTTAGCCTCCCCCATCTCCCGGGCAAACGTGCGCGC
CAGGTCGCAGATCGTCGGTATGGAGCCTGGGGTGGTGACG
TGGGTCTGGACCATCCCGGAGGTAAGTTGCAGCAGGGCGT
CCCGGCAGCCGGCGGGCGATTGGTCGTAATCCAGGATAAA
GACGTGCATGGGACGGAGGCGTTTGGCCAAGACGTCCAAG
GCCCAGGCAAACACGTTATACAGGTCGCCGTTGGGGGCCA
GCAACTCGGGGGCCCGAAACAGGGTAAATAACGTGTCCCC
GATATGGGGTCGTGGGCCCGCGTTGCTCTGGGGCTCGGCA
CCCTGGGGCGGCACGGCCGTCCCCGAAAGCTGTCCCCAAT
CCTCCCGCCACGACCCGCCGCCCTGCAGATACCGCACCGT
ATTGGCAAGCAGCCCGTAAACGCGGCGAATCGCGGCCAAC
ATAGCCAGGTCAAGCCGCTCGCCGGGGCGCTGGCGTTTGG
CCAGGCGGTCGATGTGTCTGTCCTCCGGAAGGGCCCCCAA
CACGATGTTTGTGCCGGGCAAGGTCGGCGGGATGAGGGCC
ACGAACGCCAGCACGGCCTGGGGGGTCATGCTGCCCATAA
GGTATCGCGCGGCCGGGTAGCACAGGAGGGCGGCGATGGG
ATGGCGGTCGAAGATGAGGGTGAGGGCCGGGGGCGGGGCA
TGTGAGCTCCCAGCCTCCCCCCCGATATGAGGAGCCAGAA
CGGCGTCGGTCACGGCATAAGGCATGCCCATTGTTATCTG
GGCGCTTGTCATTACCACCGCCGCGTCCCCGGCCGATATC
TCACCCTGGTCGAGGCGGTGTTGTGTGGTGTAGATGTTCG
CGATTGTCTCGGAAGCCCCCAGCACCTGCCAGTAAGTCAT
CGGCTCGGGTACGTAGACGATATCGTCGCGCGAACCCAGG
GCCACCAGCAGTTGCGTGGTGGTGGTTTTCCCCATCCCGT
GAGGACCGTCTATATAAACCCGCAGTAGCGTGGGCATTTT
CTGCTCCAGGCGGACTTCCGTGGCTTCTTGCTGCCGGCGA
GGGCGCAACGCCGTACGTCGGTTGCTATGGCCGCGAGAAC
GCGCAGCCTGGTCGAACGCAGACGCGTGTTGATGGCAGGG
GTACGAAGCCATACGCGCTTCTACAAGGCGCTTGCCAAAG
AGGTGCGGGAGTTTCACGCCACCAAGATCTGCGGCACGCT
GTTGACGCTGTTAAGCGGGTCGCTGCAGGGTCGCTCGGTG
TTCGAGGCCACACGCGTCACCTTAATATGCGAAGTGGACC
TGGGACCGCGCCGCCCCGACTGCATCTGCGTGTTCGAATT
CGCCAATGACAAGACGCTGGGCGGGGTTTGTGTCATCATA
GAACTAAAGACATGCAAATATATTTCTTCCGGGGACACCG
CCAACAAACGCGAGCAACGGGCCACGGGGATGAAGCAGCT
GCGCCACTCCCTGAAGCTCCTGCAGTCCCTCGCGCCTCCG
GGTGACAAGATAGTGTACCTGTGCCCCGTCCTGGTGTTTG
TCGCCCAACGGACGCTCCGCGTCAGCCGCGTGACCCGGCT
CGTCCCGCAGAAGGTCTCCGGTAATATCACCGCAGTCGTG
CGGATGCTCCAGAGCCTGTCCACGTATACGGTCCCCATGG
AGCCTAGGACCCAGCGAGCCCGTCGCCGCCGCGGCGGCGC
TGCCCGGGGGTCTGCGAGCAGACCGAAAAGGTCACACTCT
GGGGCGCGCGACCCGCCCGAGCCAGCGGCCCGCCAGGTAC
CACCCGCCGACCAAACCCCCGCCTCCACGGAGGGCGGGGG
GGTGCTTAAGAGGATCGCGGCGCTCTTCTGCGTGCCCGTG
GCCACCAAGACCAAACCCCGAGCTGCCTCCGAATGAGAGT
GTTTCGTTCCTTCCCCCTCCCCCCGCGTCAGACAAACCCT
AACCACCGCTTAAGCGGCCCCCGCGAGGTCCGAAGACTCA
TTTGGATCCGGCGGGAGCCACCTGACAACAACCCCTGGGT
TTCCCCACACCAGACGCCGGTCCGCTGTGCCATCGCTCCC
CTTCATCCCACCCCCATCTTGTCCCCAAATAAAACAAGGT
CTGGTAGTTAGGACAACGACCGCAGTTCTCGTGTGTTATT
GTCGCTCTCCGCCTCTCGCAGATGGACCCGTATTGCCCAT
TTGACGCTCTGGACGTCTGGGAACACAGGCGCTTCATAGT
CGCCGATTCCCGAAACTTCATCACCCCCGAGTTCCCCCGG
GACTTTTGGCTGTCGCCCGTCTTTAACCTCCCCCGGGAGA
CGGCGGCGGAGCAGGTGGTCGTCCTGCAGGCCCAGCGCCC
AGCGGCTGCCGCTGCCCTGGAGAACGCCGCCATGCAGGCG
GCCGAGCTCCCCGTCGATATCGAGCGCCGGTTACGCCCGA
TCGAACGGAACGTGCACGAGATCGCAGGCGCCCTGGAGGC
GCTGGAGACGGCGGCGGCCGCCGCCGAAGAGGCGGATGCC
GCGCGCGGGGATGAGCCGGCGGGTGGGGGCGACGGGGGGG
CGCCCCCGGGCTGGCCGTCGCGGAGATGGAGGGCCAGATC
GTGCGCAACGACCCGCCGCTACGATACGACACCAACCTCC
CCGTGGATCTGCTACATATGGTGTACGCGGGCCGCGGGGC
GACCGGCTCGTCGGGGGTGGTGTTCGGGACCTGGTACCGC
ACTATCCAGGACCGCACCATCACGGACTTTCCCCTGACCA
CCCGCAGTGCCGACTTTCGGGACGGCCGGATGTCCAAGAC
CTTCATGACGGCGCTGGTCCTGTCCCTGCAGTCGTGCGGC
CGGCTGTATGTGGGCCAGCGCCACTATTCCGCCTTCGAGT
GCGCCGTGTTGTGTCTCTACCTGCTGTACCGAAACACGCA
CGGGGCCGCCGACGATAGCGACCGCGCTCCGGTCACGTTC
GGGGATCTGCTGGGCCGGCTGCCCCGCTACCTGGCGTGCC
TGGCCGCGGGATCGGGACCGAGGGCGGCCGGCCACAGTAC
CGCTACCGCGACGACAAGCTCCCCAAGACGCAGTTCGCGG
CCGGCGGGGGCCGCTACGAACACGGAGCGCTGGCGTCGCA
CATCGTGATCGCCACGCTGATGCACCACGGGGTGCTCCCG
GCGGCCCCGGGGGACGCCCCCGGGACGCGAGCACCCACGG
TAACCCCGACGGCGTGGCGCACCACGACGACATAAACCGC
GCCGCCGCCGCGTTCCTCAGCCGGGGCCACAACCTATTCC
TGTGGGAGGACCAGACTCTGCTGCGGGCAACCGCGAACAC
CATAACGGCCCTGGGCGTTACCCAGCGGCTCCTCGCGAAC
GGCAACGTGTACGCGGACCGCCTCAACAACCGCCTGCAGC
TGGGCATGCTGATCCCCGGAGCCGTCCCTTCGGAGGCCAT
CGCCCGTGGGGCCTCCGGGTCCGACTCGGGGGCCATCAAG
AGCGGAGACAACAATCTGGAGGCGCTATGTGCCAATTACG
TGCTTCCGCTGTACCGGGCCGACCCGGCGGTCGAGCTGAC
CCAGCTGTTTCCCGGCCTGGCCGCCCTGTGTCTTGACGCC
CAGGCGGGGCGGCCGGTCGGGTCGACGCGGCGGGTGGTGG
ATATGTCATCGGGGGCCCGCCAGGCGGCGCTGGTGCGCCT
CACCGCCCTGGAACTCATCAACCGCACCCGCACAAACCCC
ACCCCCGTGGGGGAGGTTATCCACGCCCACGACGCCCTGG
CGATCCAATACGAACAGGGGCTTGGCCTGCTGGCGCAGCA
GGCACGCATTGGCTTGGGCTCCAACACCAAGCGTTTCTCC
GCGTTCAACGTTAGCAGCGACTACGACATGTTGTACTTTT
TATGTCTGGGGTTCATTCCACAGTACCTGTCGGCGGTTTA
GTGGGTGGTGGGCGAGGGGGGAGGGGGCATTAGGGAGAAA
GAACAAGAGCCTCCGTTGGGTTTTCTTTGTGCCTGTCTCA
AAAGGTCATCCCCGTAAACGGCGGGCTCCAGTCCCGGCCC
GGCGGTTGGCGTGAACGCAACGGCGGGGCTGGGTTAGCGT
TTAGTTTAGCATTCGCTCTCGCCTTTCCGCCCGCCCCCGA
CCGTTGAGCCTTTTTTTTTTTTTTTTTTTTTTTTTTTTTT
CGTCCACCAAAGTCTCTGTGGGTGCGCGCATGGCAGCCGA
TGCCCCGGGAGACCGGATGGAGGAGCCCCTGCCAGACAGG
GCCGTGCCCATTTACGTGGCTGGGTTTTTGGCCCTGTATG
ACAGCGGGGACTCGGGCGAGTTGGCATTGGATCCGGATAC
GGTGCGGGCGGCCCTGCCTCCGGATAACCCACTCCCGATT
AACGTGGACCACCGCGCTGGCTGCGAGGTGGGGCGGGTGC
TGGCCGTGGTCGACGACCCCCGCGGGCCGTTTTTTGTGGG
ACTGATCGCCTGCGTGCAACTGGAGCGCGTCCTCGAGACG
GCCGCCAGCGCTGCGATTTTCGAGCGCCGCGGGCCGCCGC
TCTCCCGGGAGGAGCGCCTGTTGTACCTGATCACCAACTA
CCTGCCCTCGGTCTCCCTGGCCACAAAACGCCTGGGGGGC
GAGGCGCACCCCGATCGCACGCTGTTCGCGCACGTAGCGC
TGTGCGCGATCGGGCGGCGCCTTGGCACTATCGTCACCTA
CGACACCGGTCTCGACGCCGCCATCGCGCCCTTTCGCCCC
TGTCGCCGGCGTCTCGCGAGGGGGCGCGGCGACTGGCCGC
CGAGGCCGAGCTCGCGCTATCCGGACGCCCCTGGGCGCCC
GGCGTGGAGGCGCTGCCCCACACGCTGCTTTCCACCGCCG
TTAACAACATGATGCTGCGGGACCGCTGGAGCCTGGTGGC
CGAGCGGCGGCGGCAGGCCGGGATCGCCGGACACACCTAC
CTCCAGGCGAGCGAAAAATTCAAAATGTGGGGGGCGGAGC
CTGTTTCCGCGCCGGCGCGCGGGTATAAGAACGGGGCCCC
GGAGTCCACGGACAACCGCCCGGCTCGATCGCTGCCGCGC
CGCAGGGTGACCGGTGCCCAATCGTCCGTCAGCGCGGGGT
CGCCTCGCCCCCGGTACTGCCCCCCATGAACCCCGTTCCA
ACATCGGGCACCCCGGCCCCCGCGCCGCCCGGCGACGGGA
GCTACCTGTGGATCCCGGCCTCCCATACAACCAGCTCGTC
GCCGGCCACGCCGCGCCCCAACCCCAGCCGCATTCCGCGT
TTGGTTCCCGGCTGCGGCGGGGGCCGTGGCCTATGGGCCT
CACGGCGCGGGTCTTTCCCAGCATTACCCTCCCCACGTCG
CCCATCAGTATCCCGGGGTGCTGTTCTCGGGACCCAGCCC
ACTCGAGGCGCAGATAGCCGCGTTGGTGGGGGCCATAGCC
GCGGACCGCCAAGCGGGCGGTCAGCCGGCCGCGGGAGACC
CTGGGGTCCGGGGGTCGGGAACTCCCTTCCTCTTCCTTTG
CCTCGGAGTCCTACTGCCCCACCGACGAACCGGACGCGGA
CTACCCGTACTACCCCGGGGAGGCTCGAGGCGGGCCGCGC
GGGGGCGACTCTCGGCGCGCGGCCCGCCAGTCTCCCGGGA
CCAACGAGACCATCACGGCGCTGATGGGGGCGGGACGTCT
CTGCAGCAGGAACTGGCGCACATGCGGGCTCGGACCAGCG
CCCCCTATGGAATGTACACGCCGGTGGCGCACTATCGCCC
TCAGGTGGGGGAGCCGGAACCAACAACGACCCACCCGGCC
CTTTGTCCCCCGGAGGCCGTGTATCGCCCCCCCCCACACA
GCGCCCCCTACGGTCCTCCCCAGGGTCCGGCGTCCCATGC
CCCCACTCCCCCGTATGCCCCAGCTGCCTGCCCGCCAGGC
CCGCCACCGCCCCCATGTCCTTCCACCCAGACGCGCGCCC
CTCTACCGACGGAGCCCGCGTTCCCCCCCGCCGCCACCGG
ATCCCAACCGGAGGCATCCAACGCGGAGGCCGGGGCCCTT
GTCAACGCCAGCAGCGCAGCACACGTGGACGTTGACACGG
CCCGCGCCGCCGATTTGTTCGTCTCTCAGATGATGGGGGC
CCGCTGATTCGCCCCGGTCTTTGGTACCATGGGATGTCTT
ACTGTATATCTTTTTAAATAAACCAGGTAATACCAAATAA
GACCCATTGGTGTATGTTCTTTTTTTTTTATTGGGAGGGG
CGGGTAGGCGGGTAGCTTTACAATGCAAAAGCCTTTGACG
TGGAGGAAGGCGTGGGGGGGAGGAAATCGGCACTGACCAA
GGGGGTCCGTTTTGTCACGGGAAAGGAAAGAGGAAACAGG
CCGCGGACACCCGGGGGAGTTTATGTGTTCCTTTTTCTTT
CTTCCCACACACACACAAAAGGCGTACCAAACAAAAAAAC
CAAAAGATGCGCATGCGGTTTAACACCCGTGGTTTTTATT
TACAACAAACCCCCCGTCACAGGTCGTCCTCGTCGGCGTC
ACCGTCTTTGTTGGGAACTTGGGTGTAGTTGGTGTTGCGG
CGCTTGCGCATGACCATGTCGGTGACCTTGGCGCTGAGCA
GCGCGCTCGTGCCCTTCTTCTTGGCCTTGTGTTCCGTGCG
CTCCATGGCCGACACCAGGGCCATGTACCGTATCATCTCC
CTGGCCTCGGCTAGCTTGGCCTCGTCAAAGTCGCCGCCCT
CCTCGCCCTCCCCGGACGCGTCCGGGTTGGTGGGGTTCTT
GAGCTCCTTGGTGGTTAGAGGGTACAGGGCCTTCATGGGG
TTGCTCTGCAGCCGCATGACGTAACGAAAGGCGAAGAAGG
CCGCCGCCAGGCCGGCCAGGCCAACAGCCCACGGCCAGCG
CCCCAAAGGGGTTGGACATGAAGGAGGACACGCCCGACAC
GGCCGATACCACGCCGCCCACGATGCCCATCACCACCTTG
CCGACCGCGCGCCCCAGGTCGCCCATCCCCTCGAAGAACG
CGCCCAGGCCCGCGAACATGGCGGCGTTGGCGTCGGCGTG
GATGACCGTGTCGATGTCGGCGAAGCGCAGGTCGTGCAGC
TGGTTGCGGCGCTGGACCTCCGTGTAGTCCAGCAGGCCGC
TGTCCTTGATCTCGTGGCGGGTGTACACCTCCAGGGGGAC
AAACTCGTGATCCTCCAGCATGGGGATGTTGAGGTCGATG
AAGGGCTGACGGTGGTGATGTCGGCGCGGCTCAGCTGGTG
GGAGTACGCGTACTCCTCGAAGTACACGTAGCCCCCACCG
AAGGTGAAGTAGCGCCGGTGTCCCACGGTGCACGGCTCGA
TCGCATCGCGCGTCAGCCGCAGCTCGTTGTTCTCCCCCAG
CTGCCCCTCGACCAACGGGCCCTGGTCTTCGTACCGAAAG
CTGACCAGGGGGCGGCTGTAGCAGGCCCCGGGCCGCGAGC
TGATGCGCATCGAGTTTTGGACGATCACGTTGTCCGCGGC
GACCGGCCCGCACGTGGAGACGGCCATCACGTCGCCGAGC
ATCCGCGCGCTCACCCGCCGGCCCACGGTGACCGAGGCGA
TGGCGTTGGGGTTCAGCTTGCGGGCCTCGTTCCACAGGGT
CAGCTCGTGATTCTGTAGCTCGCACCACGCGATGGCAACG
CGGCCCAACATATCGTTGACATGGCGCTGTATGTGGTTGT
ACGTAAACTGCAGCCGGGCGAACTCGATGGAGGAGGTGGT
CTTGATGCGCTCCACGGACGCGTTGGCGCTGGCCCCGGGC
GGCGGGGGCGTGGGGTTTGGGGGCTTGCGGCTCTGCTCTC
GGAGGTGTCCCGCACGTACAGCTCCGCGAGCGTGTTGCTG
AGAAGGGGCTGGTACGCGATCAGAAAGCCCCCATTGGCCA
GGTAGTACTGCGGCTGGCCCACCTTGATGTGCGTCGCGTT
GTACCTGCGGGCGAAGATGCGGTCCATGGCGTCGCGGGCG
TCCTTGCCGATGCAGTCCCCCAGGTCCACGCGCGAGAGCG
GGTACTCGGTCAGGTTGGTGGTGAAGGTGGTGGATATGGC
GTCGGAGGAGAATCGGAAGGAGCCGCCGTACTCGGAGCGC
AGCATCTCGTCCACTTCCTGCCACTTGGTCATGGTGCAGA
CCGACGGGCGCTTTGGCACCCAGTCCCAGGCCACGGTGAA
CTTGGGGGTCGTGAGCAGGTTCCGGGTGGTCGGCGCCGGG
CCCGGGCCTTGGTGGTGAGGTCGCGCGCGTAGAAGCCGTC
GACCTGCTTGAAGCGGTCGGCGGCGTAGCTGGTGTGTTCG
GTGTGCGACCCCTCCCGGTAGCCGTAAAACGGGGACATGT
ACACAAAGTCGCCAGTCGCCAGCACAAACTCGTCGTACGG
GTACACCGAGCGCGCGTCCACCTCCTCGACGATGCAGTTT
ACCGTCGTCCCGTACCGGTGGAACGCCTCCACCCGCGAGG
GGTTGTACTTGAGGTCGGTGGTGTGCCAGCCCCGGCTCGT
GCGGGTCGCGGCGTTGGCCGGTTTCAGCTCCATGTCGGTC
TCGTGGTCGTCCCGGTGAAACGCGGTGGTCTCCAGGTTGT
TGCGCACGTACTTGGCCGTGGACCGACAGACCCCCTTGGC
GTGATCTTGTCGATCACCTCCTCGAAGGGGACGGGGGCGC
GGTCCTCAAAGATCCCCATAAACTGGGAGTAGCGGTGGCC
GAACCACACCTGCGAAACGGTGACGTCTTTGTAGTACATG
GTGGCCTTGAACTTGTACGGGGCGATGTTCTCCTTGAAGA
CCACCGCGATGCCCTCCGTGTAGTTCTGACCCTCGGGCCG
GGTCGGGCAGCGGCGCGGCTGCTCGAACTGCACCACCGTG
GCGCCCGTGGGGGGTGGGCACACGTAAAAGTTTGCATCGG
TGTTCTCCGCCTTGATGTCCCGCAGGTGCTCGCGCAGGGT
GGCGTGGCCCGCGGCGACGGTCGCGTTGTCGCCGGCGGGG
CGTGGGGGCGTTGGGTTTTTCGGTTTTTTGTTCTTCTTCG
GTTTCGGGTCCCCCGTTGGGGCGGCGCCAAGGGCGGGCGG
CGCCGGAGTGGCAGGGCCCCCGTTCGCCGCCTGGGTCGCG
GCCGCGACCCCAGGCGTGCCGGGGGAACTCGGAGCCGCCG
ACGCCACCAGGACCCCCAGCGTCAACCCCAAGAGCGCCCA
TACGACGAACCACCGGCGCCCCCACGAGGGGGCGCCCTGG
TGCATGGCGGGACTACGGGGGCCCGTCGTGCCCCCCGTCA
GGTAGCCTGGGGGCGAGGTGCTGGAGGACCGAGTAGAGGA
TCGAGAAAACGTCGCGGTCGTAGACCACGACGACCGGGGG
CCGATACAGCCGTCGGGGGCGCTCTCGACGATGGCCACCA
GCGGACAGTCGGAGTCGTACGTGAGATATACGCCGGGCGG
GTAACGGTAACGACCTTCGGAGGTCGGGCGGCTGCAGTCC
GGGCGGGCAACTCGAGCTCCCCGCACCGGTAGACCGAGGC
AAAGAGTGTGGTGGCGATAATCAGCTCGCGAATATATCGC
CAGGCGGCGCGCTGAGTGGGCGTTATTCCGGAAATGCCGT
CAAAACAGTAAAACCTCTGAAATTCGCTGACGGCCCAATC
AGCACCCGAGCCCCCCGCCCCCATGATGAACCGGGCGAGC
TCCTCCTTCAGGTGCGGCAGGAGCCCCACGTTCTCGACGC
TGTAATACAGCGCGGTGTTGGGGGGCTGGGCGAAGCTGTG
GGTGGAGTGATCAAAGAGGGGCCCGTTGACGAGCTCGAAG
AAGCGATGGGTGATGCTGGGGAGCAGGGCCGGGTCCACCT
GGTGTCGCAGGAGAGACGCTCGCATGAACCGGTGCGCGTC
GAACACGCCCGGCGCCGAGGGTTGTCGATGACCGTGCCCG
CGCCCGCCGTCAGGGCGCAGAAGCGCGCGCGCGCCGCAAA
GCCGTTGGCGACCGCGGCGAACGTCGCGGGCAGCACCTCG
CCGTGGACGCTGACCCGCAGCATCTTCTCGAGCTCCCCGC
GCTGCTCGCGGACGCAGCGCCCCAGGCTGGCCAACGACCG
CTTCGTCAGGCGGTCCGCGTACAGCCGCCGTCGCTCCCGT
ACGTCCGCGGCCGCTTGCGTGGCGATGTCCCCCCACGTCT
CGGGCCCCTGCCCCCCGGGCCCGCGGCGACGGTCTTCGTC
CTCGCCCCCGCCCCCAGGAGCTCCCAACCCCCGTGCCCCT
TCCTCTACGGCGACACGGTCCCCGTCGTCGTCGGGGCCCG
CGCCGCCCTTGGGCGCGTCCGCCGCGCCCCCCGCCCCCAT
GCGCGCCAGCACGCGACGCAGCGCCTCCTCGTCGCACTGT
TCGGGGCTGACGAGGCGCCGCAAGAGCGGCGTCGTCAGGT
GGTGGTCGTAGCACGCGCGGATGAGCGCCTCGATCTGATC
GTCGGGTGACGTGGCCTGACCGCCGATTATTAGGGCGTCC
ACCATATCCAGCGCCGCCAGGTGGCTCCCGAACGCGCGAT
CGAAATGCTCCGCCCGCCGCCCGAACAGCGCCAGTTCCAC
GGCCACCGCGGCGGTCTCCTGCTGCAACTCGCGCCGCGCC
AGCGCGGTCAGGTTGCTGGCAAACGCGTCCATGGTGGTCT
GGCCGGCGCGGTCGCCGGACGCGAGCCAGAATCGCAATTC
GCTGATGGCGTACAGGCCGGGCGTGGTGGCCTGAAACACG
TCGTGCGCCTCCAGCAGGGCGTCGGCCTCCTTGCGGACCG
AGTCGTTCTCGGGCGACGGGTGGGGCTGCCCGTCGCCCCC
CGCGGTCCGGGCCAGCGCATGGTCCAACACGGAGAGCGCC
CGCGCGCGGTCGGCGTCCGACAGCCCGGCGGCGTGGGGCA
GGTACCGCCGCAGCTCGTTGGCGTCCAGCCGCACCTGCGC
CTGCTGGGTGACGTGGTTACAGATACGGTCCGCCAGGCGG
CGGGCGATCGTCGCCCCCTGGTTCGCCGTCACACACAGTT
CCTCGAAACAGACCGCGCAGGGGTGGGACGGGTCGCTAAG
CTCCGGGGGGACGATAAGGCCCGACCCCACCGCCCCCACC
ATAAACTCCCGAACGCGCTCCAGCGCGGCGGGGGGCAGGT
ACCGCCGCAGCTCGTTGGCGTCCAGCCGCACCTGCGCCTG
CTGGGTGACGTGGTTACAGATACGGTCCGCCAGGCGGCGG
GCGATCGTCGCCCCCTGGTTCGCCGTCACACACAGTTCCT
CGAAACAGACCGCGCAGGGGTGGGACGGGTCGCTAAGCTC
CGGGGGGACGATAAGGCCCGACCCCACCGCCCCCACCATA
AACTCCCGAACGCGCTCCAGCGCGGCGGTGGCGCCGCGCG
AGGGGGTGATGAGGGGCAGTAGTTTAGCTGCTTTAGAAAG
TTCTCGACGTCGTGCAGGAAACACAGCTCCATATGGACGG
TCCCGCCATACGTATCCAGCCTGACCCGTTGGTGATACGG
ACAGGGTCGGGCCAGGCCCATGGTCTCGGTGAAAAACGCC
GCGACGTCTCCCGCGTACGCGAACGTCTCCAGGTTGCCCA
GGAGCCGCTCGCCCTCGCGCCACGCGTACTCTAGCAGCAA
CTCCAGGGTGACCGACAGCGGGGTGAGAAAGGCCCCGGCC
TGGGCCTCCAGGCCCGGCCTCAGACGACGCCGCAGCGCCC
GCACCTGAAGCGCGTTCAGCTTCAGTTGGGGGAGCTTCCC
CCGTCCGATGTGGGGGTCGCACCGCCGGAGCAGCTCTATC
TGAAACACATAGGTCTGCACCTGCCCGAGCAGGGCTAACA
ACTTTTGACGGGCCACGGTGGGCTCGGACACCGGGGCGGC
CATCTCGCGGCGCCGATCTGTACCGCGGCCGGAGTATGCG
GTGGACCGAGGCGGTCCGTACGCTACCCGGTGTCTGGCTG
AGCCCCGGGGTCCCCCTCTTCGGGGCGGCCTCCCGCGGGC
CCGCCGACCGGCAAGCCGGGAGTCGGCGGCGCGTGCGTTT
CTGTTCTATTCCCAGACACCGCGGAGAGGAATCACGGCCC
GCCCAGAGATATAGACACGGAACACAAACAAGCACGGATG
TCGTAGCAATAATTTATTTTACACACATTCCCCGCCCCGC
CCTAGGTTCCCCCACCCCCCAACCCCTCACAGCATATCCA
ACGTCAGGTTTCCCTTTTTGTCGGGGGGCCCCTCCCCAAA
CGGGTCATCCCCGTGGAACGCCCGTTTGCGGCCGGCAAAT
GCCGGTCCCGGGGCCCCCGGGCCGCCGAACGGCGTCGCGT
TGTCGTCCTCGCAGCCAAAATCCCCAAAGTTAAACCCCTC
CCCGGCGTTGCCGAGTTGGCTGACTAGGGCCTCGGCCTCG
TGCGCCACCTCCAGGGCCGCGTCCGTCGACCACTCGCCGT
TGCCGCGCTCCAGGGCACGCGCGGTCAGCTCCATCATCTC
CTCGCTTAGGTACTCGTCCTCCAGGAGCGCCAGCCAGTCC
TCGATCTGCAGCTGCTGGGTGCGGGGCCCCAGGCTTTTCA
CGGTCGCCACGAACACGCTACTGGCGACGGCCGCCCCGCC
CTCGGAGATAATGCCCCGGAGCTGCTCGCACAGCGAGCTT
TCGTGCGCTCCGCCGCCGAGGTTCGAGGCCGCGCACACAA
ACCCGGCCCGGGGACAGGCCAGGACGAACTTGCGGGTGCG
GTCAAAAATAAGGAGAGGGAGGTTTTTGCCGCCCATCAGG
CTGGCCCAGTTCCCGGCCTGAAACACACGGTCGTTGCCGG
CCATGCCGTAGTATTTGCTGTTGCACAACCCCAACACGAC
ACTGGGGCGCGCCGCCATGACGGGCCGCAGCAGGTTGCAG
CTGGCGAACATGGACGTCCACGCGCCCGGATGCGCGTCCA
CGGCGTCCATCAGCGCGCGGGCCCCGGCCTCCAGGCCCGC
CCCGCCCTGCGCGGACCACGCGGCCGCCGCCTGCACGCTG
GGGGGACGGCGGGACCCCGCGATGATGGCCGTGAGGGTGT
TGATGAAGTATGTCGAGTGATCGCAGTACCGCAGAATCTG
GTTTGCCATGTAGTACATCGCCAGCTCGCTCACGTTGTTG
GGGGCCAGGTAATAAAGTTTATCGCGCCGTAGTCCAGGGA
AAACTTTTTAATGAACGCGATGGTCTCGATGTCCTCGCGC
GACAGGAGCCGGGCGGGAAGCTGGTTGCGTTGGAGGGCCG
TCCAGAACCACTGCGGGTTCGGCTGGTTGGACCCCGGGGG
CTTGCCGTTGGGGAAGATGGCCGCGTGGAACTGCTTCAGC
AGAAAGCCCAGCGGTCCGAGGAGGATGTCCACGCGCTTGT
CGGGCTGCTGGGGGGGGGTGGGGAGGCTGGCGACCCGCGC
CTTGGCGGCCTCGGACGCGTTGGCGCTCGCGCCCGCGAAC
AACACGCGGCTCTTGACGCGCAGCTCCTTGGGAAACCCCA
GGGTCACGCGGGCAACGTCGCCCTCGAAGCTGCTCTCGGC
GGGGGCCGTCTGGCCGGCCGTTAGGCTGGGGGCGCAGATA
GCCGCCCCCTCCGAGAGCGCGACCGTCAGCGTTTTGGCCG
ACAGAAACCCGTTGTTAAACATGTCCATCACGCGCCGCCG
CAGCACCGGTTGGAATTGATTGCGAAAGTTGCGCCCCTCG
ACCGACTGCCCGGCGAACACCCCGTGGCACTGGCTCAGGG
CCAGGTCCTGATACACGGCGAGGTTGGATCGCCGCCCGAG
AAGCTGAAGCAGGGGGCATGGCCCGCACGCGTACGGGTCC
AGCGTCAGGGACATGGCGTGGTTGGCCTCGCCCAGACCGT
CGCGAAACTTGAAGTTCCTCCCCTCCCCAGGTTGCGCATC
AGCTGCTCCACCTCGCGGTCCACGACCTGCCTGACGTTGT
TCACCACCGTATGCAGGGCCTCGCGGTTGGTGATGATGGT
CTCCAGCCGCCCCATGGCCGTGGGGACCGCCTGGTCCACG
TACTGCAGGGTCTCGAGTTCGGCCATGACGCGCTCGGTCG
CCGCGCGGTACGTCTCCTGCATGATGGTCCGGGCGGTCTC
GGATCCGTCCGCGCGCTTCAGGGCCGAGAAGGCGGCGTAG
TTTCCCAGCACGTCGCAGTCGCTGTACATGCTGTTCATGG
TCCCGAAGACGCCGATGGCTCCGCGGGCGGCGCTGGCGAA
CTTGGGATGGCGCGCCCGGAGGCGCATGAGCGTCGTGTGT
ACGCAGGCGTGGCGCGTGTCGAAGGTGCACAGGTTGCAGG
GCACGTCGGTCTGGTTGGAGTCCGCGACGTATCGAAACAC
GTCCATCTCCTGGCGCCCGACGATCACGCCGCCGTCGCAG
CGCTCCAGGTAAAACAGCATCTTGGCCAGCAGCGCCGGGG
AAAACCCACACAGCATGGCCAGGTGCTCGCCGGCAAATTC
CTGGGTTCCGCCGACGAGGGGCGCGGTGGGCCGACCCTCG
AACCCGGGCACCACGTGTCCCTCGCGGTCCACCTGTGGGT
TGGCCGCCACGTGGGTCCCGGGCACGAGGAAGAAGCGGTA
AAAGGAGGGTTTGCTGTGGTCCTTTGGGTCCGCCGGACCG
GCGTCGTCCACCTCGGTGAGATGGAGGGCCGAGTTGGTGC
TAAATACCATGGCCCCCACGAGTCCCGCGGCGCGCGCCAG
GTACGCCCCGACGGCGTTGGCGCGGGCCGCGGCCGTGTCC
TGGCCCTCGCACAGCGGCCACGCGGAGATGTCGGTGGGCG
GCTCGTCGAAGACGGCCATCGACACGATAGACTCGAGGGC
CAGGGCGGCGTCTCCGGCCATGACGGAGGCCAGGCGCTGT
TCGAACCCGCCCGCCGGGCCCTTGCCGCCGCCGTCGCGCC
CACCCCGCGGGGTCTTACCCTGGCTGGCTTCGAAGGCCGT
GAACGTAATGTCGGCGGGGAGGGCGGCGCCCTCGTGGTTT
TCGTCAAACGCCAGGTGGGCGGCCGCGCGGGCCACGGCGT
CCACGTTTCGGCATCGCAGTGCCACGGCGGCGGGTCCCAC
GACCGCCTCGAACAGGAGGCGGTTGAGGGGGCGGTTAAAA
AACGGAAGCGGGTAGGTAAAATTCTCCCCGATCGATCGGT
GGTTGGCGTTGAACGGCTCGGCGATGACCCGGCTAAAATC
CGGCATGAACAGCTGCAACGGATACACGGGTATGCGGTGC
ACCTCCGCCCCGCCTATGGTTACCTTGTCCGAGCCTCCCA
GGTGCAGAAAGGTGTTGTTGATGCACACGGCCTCCTTGAA
GCCCTCGGTAACGACCAGATACAGGAGGGCGCGGTCCGGG
TCCAGGCCGAGGCGCTCACACAGCGCCTCCCCCGTCGTCT
CGTGTTTGAGGTCGCCGGGCCGGGGGGTGTAGTCCGAAAA
GCCAAAATGGCGGCGTGCCCGCTCGCAGAGTCGCGTCAGG
TTTGGGGCCTGGGTGCTGGGGTCCAGGTGCCGGCCGCCGT
GAAAGACGTACACGGACGAGCTGTAGTGCGATGGCGTCAG
TTTCAGGGACACCGCGGTACCCCCGAGCCCCGTCGTGCGA
GAACCCACGACCACGGCTACGTTGGCCTCAAAGCCGCTCT
CCACGGTCAGGCCCACGACCAGGGGCGCCACGGCGACGTC
GGCATCGCCGCTGCGCGCCGACAGTAACGCCAGAAGCTCG
ATGCCTTCGGACGGACACGCGCGAGCGTACACGTATCCCA
GGGGCCCGGGGGGGACCTTGATGGTGGTTGCCGTCTTGGG
CTTTGTCTCCATGTCCTCCTGGCAATCGGTCCGCAAACGG
AGGTAATCCCGGCACGACGACGGACGCCCGACGAGGTATG
TCTCCCGAGCGTCAAAATCCGGGGGGGGGGCGGCGACGGT
CAAGGGGAGGGTGGGAGACCGGGGTTGGGGAATGAATCCC
TACCCTTCACAGACAACCCCCGGGTAACCACGGGGTGCCG
ATGAACCCCGGCGGCTGGCAACGCGGGGTCCCTGCGAGAG
GCACAGATGCTTACGGTCAGGTGCTCCGGGCCGGGTGCGT
CTGATATGCGGTTGGTATATGTACACTTTACCTGGGGGCG
TGCCGGACCGCCCCAGCCCCTCCCACACCCCGCGCGTCAT
CAGCCGGTGGGCGTGGCCGCTATTATAAAAAAAGTGAGAA
CGCGAAGCGTTCGCACTTTGTCCTAATAATATATATATTA
TTAGGACAAAGTGCGAACGCTTCGCGTTCTCACTTTTTTT
ATAATAGCGGCCACGCCCACCGGCTACGTCACGCTCCTGT
CGGCCGCCGGCGGTCCATAAGCCCGGCCGGCCGGGCCGAC
GCGAATAAACCGGGCCGCCGGCCGGGGCGCCGCGCAGCAG
CTCGCCGCCCGGATCCGCCAGACAAACAAGGCCCTTGCAC
ATGCCGGCCCGGGCGAGCCTGGGGGTCCGGTAATTTTGCC
ACCCCCCCAGCGGCTTTTGGGGTTTTTCCTCTTCCCCCCT
CCCCACATCCCCCCCCTTTAGGGGTTCGGGTGGGACAACC
GCGATGTTTTCCGGTGGCGGCGGCCCGCTGTCCCCCGGAG
GAAAGTCGGCGGCCAGGGCGGCGTCCGGGTTTTTTGCGCC
CGCCGGCCCTCGCGGAGCCGGCCGGGGACCCCCGCCTTGT
TTGAGGCAAAACTTTTACAACCCCTACCTCGCCCCAGTCG
GGACGCAACAGAAGCCGACCGGGCCAACCCAGCGCCATAC
GTACTATAGCGAATGCGATGAATTTCGATTCATCGCCCCG
CGGGTGCTGGACGAGGATGCCCCCCCGGAGAAGCGCGCCG
GGGTGCACGACGGTCACCTCAAGCGCGCCCCCAAGGTGTA
CTGCGGGGGGGACGAGCGCGACGTCCTCCGCGTCGGGTCG
GGCGGCTTCTGGCCGCGGCGCTCGCGCCTGTGGGGCGGCG
TGGACCACGCCCCGGCGGGGTTCAACCCCACCGTCACCGT
CTTTCACGTGTACGACATCCTGGAGAACGTGGAGCACGCG
TACGGCATGCGCGCGGCCCAGTTCCACGCGCGGTTTATGG
ACGCCATCACACCGACGGGGACCGTCATCACGCCCCTGGG
CCTGACTCCGGAAGGCCACCGGGTGGCCGTTCACGTTTAC
GGCACGCGGCAGTACTTTTACATGAACAAGGAGGAGGTTG
ACAGGCACCTACAATGCCGCGCCCCACGAGATCTCTGCGA
GCGCATGGCCGCGGCCCTGCGCGAGTCCCCGGGCGCGTCG
TTCCGCGGCATCTCCGCGGACCACTTCGAGGCGGAGGTGG
TGGAGCGCACCGACGTGTACTACTACGAGACGCGCCCCGC
TCTGTTTTACCGCGTCTACGTCCGAAGCGGGCGCGTGCTG
TCGTACCTGTGCGACAACTTCTGCCCGGCCATCAAGAAGT
ACGAGGGTGGGGTCGACGCCACCACCCGGTTCATCCTGGA
CAACCCCGGGTTCGTCACCTTCGGCTGGTACCGTCTCAAA
CCGGGCCGGAACAACACGCTAGCCCAGCCGCGGGCCCCGA
TGGCCTTCGGGACATCCAGCGACGTCGAGTTTAACTGTAC
GGCGGACAACCTGGCCATCGAGGGGGGCATGAGCGACCTA
CCGGCATACAAGCTCATGTGCTTCGATATCGAATGCAAGG
CGGGGGGGGAGGACGAGCTGGCCTTTCCGGTGGCCGGGCA
CCCGGATGACCTGGTTATTCAGATATCCTGTCTGCTCTAC
GACCTGTCCACCACCGCCCTGGAGCACGTCCTCCTGTTTT
CGCTCGGTTCCTGCGACCTCCCCGAATCCCACCTGAACGA
GCTGGCGGCCAGGGGCCTGCCCACGCCCGTGGTTCTGGAA
TTCGACAGCGAATTCGAGATGCTGTTGGCCTTCATGACCC
TTGTGAAACAGTACGGCCCCGAGTTCGTGACCGGGTACAA
CATCATCAACTTCGACTGGCCCTTCTTGCTGGCCAAGCTG
ACGGACATTTACAAGGTCCCCCTGGACGGGTACGGCCGCA
TGAACGGCCGGGGCGTGTTTCGCGTGTGGGACATAGGCCA
GAGCCACTTCCAGAAGCGCAGCAAGATAAAGGTGAACGGC
ATGGTGAACATCGACATGTACGGGATCATAACCGACAAGA
TCAAGCTCTCGAGCTACAAGCTCAACGCCGTGGCCGAAGC
CGTCCTGAAGGACAAGAAGAAGGACCTGAGCTATCGCGAC
ATCCCCGCCTACTACGCCACCGGGCCCGCGCAACGCGGGG
TGATCGGCGAGTACTGCATACAGGATTCCCTGCTGGTGGG
CCAGCTGTTTTTTAAGTTTTTGCCCCATCTGGAGCTCTCG
GCCGTCGCGCGCTTGGCGGGTATTAACATCACCCGCACCA
TCTACGACGGCCAGCAGATCCGCGTCTTTACGTGCCTGCT
GCGCCTGGCCGACCAGAAGGGCTTTATTCTGCCGGACACC
AGGGGCGATTTAGGGGCGCCGGGGGGGAGGCGCCCAAGCG
TCCGGCCGCAGCCCGGGAGGACGAGGAGCGGCCAGAGGAG
GAGGGGGAGGACGAGGACGAACGCGAGGAGGGCGGGGGCG
AGCGGGAGCCGGAGGGCGCGCGGGAGACCGCCGGCCGGCA
CGTGGGGTACCAGGGGGCCAGGGTCCTTGACCCCACTTCC
GGGTTTCACGTGAACCCCGTGGTGGTGTTCGACTTTGCCA
GCCTGTACCCCAGCATCATCCAGGCCCACAACCTGTGCTT
CAGCACGCTCTCCCTGAGGGCCGACGCAGTGGCGCACCTG
GAGGCGGGCAAGGACTACCTGGAGATCGAGGTGGGGGGGC
GACGGCTGTTCTTCGTCAAGGCTCACGTGCGAGAGAGCCT
CCTCAGCATCCTCCTGCGGGACTGGCTCGCCATGCGAAAG
CAGATCCGCTCGCGGATTCCCCAGAGCAGCCCCGAGGAGG
CCGTGCTCCTGGACAAGCAGCAGGCCGCCATCAAGGTCGT
GTGTAACTCGGTGTACGGGTTCACGGGAGTGCAGCACGGA
CTCCTGCCGTGCCTGCACGTTGCCGCGACGGGACGACCAT
CGGCCTGGAGATCGAGGTGGGGGGGCGACGGCTGTTCTTC
GTCAAGGCTCACGTGCGAGAGAGCCTCCTCAGCATCCTCC
TGCGGGACTGGCTCGCCATGCGAAAGCAGATCCGCTCGCG
GATTCCCCAGAGCAGCCCCGAGGAGGCCGTGCTCCTGGAC
AAGCAGCAGGCCGCCATCAAGGTCGTGTGTAACTCGGTGT
ACGGGTTCACGGGAGTGCAGCACGGACTCCTGCCGTGCCT
GCACGTTGCCGCGACGGTGACGACCATCGGCCGCGAGATG
CTGCTCGCGACCCGCGAGTACGTCCACGCGCGCTGGGCGG
CCTTCGAACAGCTCCTGGCCGATTTCCCGGAGGCGGCCGA
CATGCGCGCCCCCGGGCCCTATTCCATGCGCATCATCTAC
GGGGACACGGACTCCATATTTGTGCTGTGCCGCGGCCTCA
CGGCCGCCGGGCTGACGGCCATGGGCGACAAGATGGCGAG
CCACATCTCGCGCGCGCTGTTTCTGCCCCCCATCAAACTC
GAGTGCGAAAAGACGTTCACCAAGCTGCTGCTGATCGCCA
AGAAAAAGTACATCGGCGTCATCTACGGGGGTAAGATGCT
CATCAAGGGCGTGGATCTGGTGCGCAAAAACAACTGCGCG
TTTATCAACCGCACCTCCAGGGCCCTGGTCGACCTGCTGT
TTTACGACGATACCGTTCCGGAGCGGCCGCCGCGTTAGCC
GAGCGCCCCGCAGAGGAGTGGCTGGCGCGCCCCTGCCCGA
GGGACTGCAGGCGTTCGGGGCCGTCCTCGTAGACGCCCAT
CGGCGCATCACCGACCCGGAGAGGGACATCCAGGACTTTG
TCCTCACCGCCGAACTGAGCAGACACCCGCGCGCGTACAC
CAACAAGCGCCTGGCCCACCTGACGGTGTATCAGCTCATG
GCCCGCCGCGCGCAGGTCCCGTCCATCAAGGACCGGATCC
CGTCGTGTCGGGCCCGCCCGCGAGGAGGGAGACGGTCGCG
CGGCTGGCCGCCCTCCGCGAGCTAGACGCCGCCGCCCCAG
GGGACGAGCCCGCCCCCCCCGCGGCCCTGCCCTCCCCGGC
CAAGCGCCCCCGGGAGACGCCGTCGCATGCCGACCCCCCG
GGAGGCGCGTCCAAGCCCCGCAAGCTGCTGGTGTCCGAGC
TGGCCGAGGCATCCCGCATACGCCATTGCCCACGGCGTCG
CCCTGAACACGGACTATTACTTCTCCCACCTGTTGGGGGC
GGCGTGCGTGACATTCAAGGCCCTGTTTGGGAATAACGCC
AAGATCACCGAGAGTCTGTTAAAAAGGTTTATTCCCGAAG
TGTGGCACCCCCCGGACGACGTGGCCGCGCGGCTCCGGGC
CGCAGGGTTCGGGGCGGTGGGTGCCGGCGCTACGGCGGAG
GAAACTCGTCGAATGTTGCATAGAGCCTTTGATACTCTAG
CATGAGCCCCCCGTCGAAGCTGATGTCCCTCATTTTACAA
TAAATGTCTGCGGCCGACACGGTCGGAATCTCCGCGTCCG
TGGGTTTCTCTGCGTTGCGCCGGACCACGAGCACAAACGT
GCTCTGCCACACGTGGGCGACGAACCGGTACCCCGGGCAC
GCGGTGAGCATCCGGTCTATGAGCCGGTAGTGCAGGTGGG
CGGACGTGCCGGGAAAGATGACGTACAGCATGTGGCCCCC
GTAAGTGGGGTCCGGGAAAACAACAGCCGCGGGTCGCACG
CCCCGCCTCCGCGCAGGATCGTGTGGACGAAAAAAAGTCG
GGTGGCAAGAATCCCGGCCAAGAGGTCCTGGAGGGGGGCG
TTGTGGCGGTCGGCCAACACGACCAAGGAGGCCAGGAAGG
CGCGATGCTCGAATATCGTGTTGATCTGCTGCACGAAGGC
CAGGATTAGGGCCTCGCGGCTGGTGGCGGCGAACCGCCCG
TCTCCCGCGTTGCACGCGGGACAGCAACCCCCGATGCCTA
GGTAGTAGCCCATCCCGGAGAGGGTCAGGCAGTTGTCGGC
CACGGTCTGGTCCAGACAGAAGGGCAGCGACACGGGAGTG
GTCTTCACCAGGGGCACCGAGAACGAGCGCACGATGGCGA
TCTCCTCGGAGGGCGTCTGGGCGAGGGCGGCGAAAAGGCC
CCGATAGCGCTGGCGCTCGTGTAAACACAGCTCCTGTTTG
CGGGCGTGAGGCGGCAGGCTCTTCCGGGAGGCCCGACGCC
CACGCCCAGAGTCCCGCCGGCCGCAGAGGAGCACGACCGC
CGGCGCTCCTTGCCGTGATAGGGCCCGGGCCGGGAGCCGC
GGCGATGGGGGTCGGTATCATACATAGGTACACAGGGTGT
GCTCCAGGGACAGGAGCGAGATCGAGTGGCGTCTAAGCAG
CGCGCCCGCCTCACGGACAAATGTGGCGAGCGCGGTGGGC
TTTGGTACAAATACCTGATACGTCTTGAAGGTGTAGATGA
GGGCACGCAACCGCTATGCAGACACGCCCCTCGAACTCGT
TCCCGCAGGCCAGCTTGGCCTTGTGGAGCAGCAGCTCGTC
GGGATGGGTGGCGGGGGGATGGCCGAACAGAACCCAGGGG
TCAACCTCCATCTCCGTGATGGCGCACATGGGGTCACAGA
ACATGTGCTTAAAGATGGCCTCGGGCCCCGCGGCCCGCAG
CAGGCTCACAAACCGCCCGTCCCCGGGCTGCGTCTCGGGG
TCCGCCTCGAGCTGGTCGACGACGGGTACGATACAGTCGA
AGAGGCTCGTGTTGTTTTCCGAGTAGCGGACCACGGAGGC
CCGGAGTCTGCGCAGGGCCAGCCAGTAAGCCCGCACCAGT
AACAGGTTACACAGCAGGCATTCTCCGCCGGTGCGCCCGC
GCCCCCGGCCGTGTTTCAGCACGGTGGCCATCAGAGGGCC
CAGGTCGAGGTCGGGCTGGGCATCGTGTTCGGTAAACTGC
GCAAAGCGCGGAGCCACGTCGCGCGTGCGTGCCCCGCGAT
GCGCTTCCCAGGACTGGCGGACCGTGGCGCGACGGGCCTC
CGCGGCAGCGCGCAGCTGGGGCCCCGACTCCCAGACGGCG
GGGGTGCCGGCGAGGGCAGCAGGCCAGATCCGCGTACGCC
CACGTATCCGGCGACTCCTCCGGCTCGCGGTCCCCGGCGA
CCGTCTCGAATTCCCCGTTGCGAGCGGCGGCGCGCGTACA
GCAGCTGTCCCCGCCCCCGCGCCGACCCTCCGTGCAGTCC
AGGAGACGGGCGCAATCCTTCCAGTTCATCAGCGCGGTGG
TGAGCGACGGCTGCGTGCCGGATCCCGCCGCCGACCCCGC
CCCCTCCTCGCCCCCGGAGGCCAAGGTTCCGATGAGGGCC
CGGGTGGCAGACTGCGCCAGGAACGAGTAGTTGGAGTACT
GCACCTTGGCGGCTCCCGGGGAGGGCGAGGGCTTGGGTTG
CTTCTGGGCATGCCGCCCGGGCACCCCGCCGTCGGTACGG
AAGCAGCAGTGGAGAAAAAAGTGCCGGTGGATGTCGTTTA
TGGTGAGGGCAAAGCGTGCGAAGGAGCCGACCAGGGTCGC
CTTCTTGGTGCGCAGAAAGTGGCGGTCCATGACGTACACA
AACTCGAACGCGGCCACGAAGATGCTAGCGGCGCAGTGGG
GCGCCCCCAGGCATTTGGCACAGAGAAACGCGTAATCGGC
CACCCACTGGGGCGAGAGGCGGTAGGTTTGCTTGTACAGC
TCGATGGTGCGGCAGACCAGACAGGGCCGGTCCAGCGCGA
AGGTGTCGATGGCCGCCGCGGAAAAGGGCCCGGGGTCCAA
AAGCCCCTCCCCACAGGGATCCGGGGGCGGGTTGCGGGGT
CCTCCGCGCCCGCCCGAACCCCCTCCGTCGCCCGCCCCCC
CGCGGGCCCTTGAGGGGGCGGTGACCACGTCGGCGGCGAC
GTCCTCGTCGAGCGTACCGACGGGCGGCACACCTATCACG
TGACTGGCCGCCAGGAGCTCGGCGCAGAGAGCCTCGTTAA
GAGCCAGGAGGCTGGGATCGAAGGCCACATACGCGCGCTC
GAACGCCCCCGCCTTCCAGCTGCTGCCGGGGGACTCTTCG
CACACCGCGACGCTCGCCAGGACCCCGGGGGGCGAAGTTG
CCATGGCTGGGCGGGAGGGGCGCACGCGCCAGCGAACTTT
ACGGGACACAATCCCCGACTGCGCGCTGCGGTCCCAGACC
CTGGAGAGTCTAGACGCGCGCTACGTCCGCGAGACGGCGC
GCATGACGCGGCCGTCTGGTTCGAGGATATGACCCCCGCC
GAGCTGGAGGTTGTCTTCCCGACTACGGACGCCAAGCTGA
ACTACCTGTCGCGGACGCAGCGGCTGGCCTCCCTCCTGAC
GTACGCCGGGCCTATAAAAGCGCCCGACGACGCCGCCGCC
CCGCAGACCCCGGACACCGCGTGTGTGCACGGCGAGCTGC
TCGCCCGCAAGCGGGAAAGATTCGCGGCGGTCATTAACCG
GTTCCTGGACCTGCACCAGATTCTGCGGGGCTGACGCGCG
CGCTGTTGGGTGGGACGGTTCGCGAACCCTTTGGTGGGTT
TACGCGGGCACGCACGCTCCCATCGCGGGCGCCATGGCGG
GACTGGGCAAGCCCTACCCCGGCCACCCAGGTGACGCCTT
CGAGGGTCTCGTTCAGCGAATTCGGCTTATCGTCCCATCT
ACGTTGCGGGGCGGGGACGGGGAGGCGGGCCCCTACTCTC
CCTCCAGCCTCCCCTCCAGGTGCGCCTTTCAGTTTCATGG
CCATGACGGGTCCGACGAGTCGTTTCCCATCGAGTATGTA
CTGCGGCTTATGAACGACTGGGCCGAGGTCCCGTGCAACC
CTTACCTGCGCATACAGAACACCGGCGTGTCGGTGCTGTT
TCAGGGGTTTTTTCATCGCCCACACAACGCCCCCGGGGGC
GCGATTACGCCAGAGCGGACCAATGTGATCCTGGGCTCCA
CCGAGACGACGGGGCTGTCCCTCGGCGACCTGGACACCAT
CAAGGGGCGGCTCGGCCTGGATGCCCGGCCGATGATGGCC
AGCATGTGGATCAGCTGCTTTGTGCGCATGCCCCGCGTGC
AGCTCGCGTTTCGGTTCATGGGCCCCGAAGATGCCGGACG
GACGAGACGGATCCTGTGCCGCGCCGCCGAGCAGGCTATT
ACCCGTCGCCGCCGAACCCGGCGGTCCCGGGAGGCGTACG
GGGCCGAGGCCGGGCTGGGGGTGGCTGGAACGGGTTTCCG
GGCCAGGGGGGACGGTTTTGGCCCGCTCCCCTTGTTAACC
CAAGGGCCCTCCCGCCCGTGGCCCAGGCCCTGCGGGGTCT
TAAGCCCTACGGATTGGCCCCCCCGCGCTCGTTTTGGCGG
CGGGACTCGTCCTGGGGGCCGCTATTTGGTGGGTGGTTGG
TGCTGGCGCGCGCCTATAAAAAAGGACGCACCGCCGCCCT
AATCGCCAGTGCGTTCCGGACGCCTTCGCCCCACACAGCC
CTCCCGTCCGACACCCCCATATCGCTTCCCGACCTCCGGT
CCCGATGGCCGTCCCGCAATTTCACCGCCCCAGCACCGTT
ACCACCGATAGCGTCCGGGCGCTTGGCATGCGCGGGCTCG
TCTTGGCCACCAATAACTCTCAGTTTATCATGGATAACAA
CCACCCGCACCCCCAGGGCACCCAAGGGGCCGTGCGGGAG
TTTCTCCGCGGTCAGGCGGCGGCGCTGACGGACCTTGGTC
TGGCCCACGCAAACAACACGTTTACCCCGCAGCCTATGTT
CGCGGGCGACGCCCCGGCCGCCTGGTTGCGGCCCGCGTTT
GGCCTGCGGCGCACCTATTCACCGTTTGTCGTTCGAGAAC
CTTCGACGCCCGGGACCCCGTGAGGCCCGGGGAGTTCCTT
CTGGGGAAAACACCCCACAGCAAAAAAATCAATAAAAGAC
CACACCAACGCACGAGCCTTGCGTTTAATGTCGAGGGGTT
TATTCAAGGGAGTGGGATAGGGTTCGACGGTTCGAAACTT
AACACACAAAATAATCGAGCGCGTCTAGCCCAGTAACATG
TGCACGTGATGTAGGCTGGTCAGCACGGCGTCGCTGTGAT
GAAGCAGCGCCCGGCGGGTCCGCTGTAACTGCTGTTGTAG
GCGGTAACAGGCGCGGATCAGCACCGCCAGGGCGCTACGA
CCGGTGCGTTGCACGGAGCGTCGCGACAGAACTGCGTTTG
CCGATACGGGCGGGGGGCCGAATTGTAAGCGCGTCACCTC
TTGGGAGTCATCGGCGGATAACGCACTGAATGGTTCGTTG
GTTATGGGGGAGTGTGGTTCCCGAGGGAGTGGGTCGAGCG
CCTCGGCCTCGGAATCCGAGAGGAACAACGAGGTGGTGTC
GGAGTCTTCGTCGTCAGAGACATACAGGGTCTGAAGCAGC
GACACGGGCGGGGGGGTAGCGTCAATGTGTAGCGCGAGGG
AGGATGCCCACGAAGACACCCCAGACAAGGAGCTGCCCGT
GCGTGGATTTGTGGACGACGCGGAAGCCGGGACGGATGGG
CGGTTTTGCGGTGCCCGGAACCGAACCGCCGGATACTCCC
CGGGTGCTACATGCCCGTTTTGGGGCTGGGGTTGGGGCTG
GGGTGGGGCTGGGGTTGACGGGTTGGGGCTGGGGCTGGGG
CTGGGGTTGGGGCTGGGGTTGGGGTTGGGGCTGGGGTTGG
GGTTGGGGCTGGGGTTGGGGCTGGGGGGCTGGGGCGCGGA
CAGGCGGTTGACGGGCAAATGCCCCCGGGGGCGCGCAGAT
GTGGGGGCGTGGCCACCGGCTGCCGGGTAGTGGGGCGGCG
GGAAACCGGGCCTCCGGGCGTAACACCGCCCTCCAGCGTC
AAGTATGTGGGGGGCGGGCCTGACGTCGGGGGCGGGGTGA
CGGGTTGGACCGCGGGAGGCGGGGGAGAGGGACCTGCGGG
AGAGGATGAGGTCGGCTCGGCCGGGTTGCGGCCTAAAACA
GGGGCCGTGGGGTCGGCGGGGTCCCAGGGTGAAGGGAGGG
ATTCCCGCGATTCGGACAGCGACGCGACAGCGGGGCGCGT
AAGGCGCCGCTGCGGCCCGCCTACGGGAACCCTGGGGGGG
GTTGGCGCGGGACCCGAGGTTAGCGGGGGGCGGCGGTTTT
CGCCCCCGGGCAAAACCGTGCCGGTTGCGACCGGGGGCGG
AACGGGATCGATAGGGAGAGCGGGAGAAGCCTGGCCGGCG
AACTGGGGACCGAGCGGGAGGGGCACACCAGACACCAAAG
CGTGGAGCGCTGGCTCTGGGGGTTTGGGAGGGGCCGGGGG
GCGCGCGAAATCGGTAACCGGGGCGACCGTGTCGGGGAGG
GCAGGCGGCCGCCAACCCTGGGTGGTCGCGGAAGCCTGGG
TGGCGCGCGCCAGGGAGCGTGCCCGGCGGTGTCGGCGCGC
GCGCGACCCGGACGAAGAAGCGGCAGAAGCGCGGGAGGAG
GCGGGGGGGCGGGGGGCGGGGCGGGGGGACGGCAAGCGCC
GGAAGTCGTCGCGGGGGCCCACGGGCGCCGGCCGCGGCTT
TCGGCCGGGACGCCCGGTCGTGCTTCGCGAGCCGGGACTG
CCGGCCCAGGGGGCCGCGGTGCACACTGGGACGTGGGGAA
GGGGGCCGGGGCAAGGAGGGGCGCGGGGCCGCCGGAGTCG
TCAGACGCGAGCTCCTCCAGGCCGTGAATCCATGCCCACA
TGCGAGGGGGGACGGGCTCGCCGGGGGTGGCGTCGGTGAA
TAGCGTGGGGGCCAGGCTTCCGGGCCCCAACGAGCCCTCC
GTCCCAACAAGGTCCGCCGGGCCGGGGGTCGGGTTCGGGA
CCGAGGGGCTCTGGTCGTCGGGGGCGCGCTGGTACACCGG
ATGCCCCGGGATAGCTCCCCCGACAGGAGGGAGGCGTCGA
ACGGCCGCCCGAGGATAGCTCGCGCGAGGAAGGGGTCCTC
GCGGTGGCGCTGGCGGCGAGGACGTCCTCGCCGCCCGCCA
CAAACGGGAGCTCCTCGGTGGCCTCGCTGCCAACAAACCG
CACGTCGGGGGGGCCGGGGGGGTCCGGGTTTTCCCACAAC
ACCGCGACCGGGGTCATGGAGATGTCCACGAGCACCAGAC
ACGGCGGGCCCCGGGCGGGGGGGGTCCGGGTTTTCCCACA
ACACCGCGACCGGGGTCATGGAGATGTCCACGAGCACCAG
ACACGGCGGGCCCCGGGCGAGGGGCCGCTCGGCGATGAGC
GCGGACAGGCGCGGGAGCTGTGCCGCCAGACACGCGTTTT
CAATCGGGTTCAGGTCGGCGTGCAGGAGGCGGACGGCCCA
CGTCTCGATGTCGGACGACACGGCATCGCGCAAGGCGGCG
TCCGGCCCGCGAGCGCGTGAGTCAAACAGCGTGAGACACA
GCTCCAGCTCCGACTCGCGGGAAAAGGCCGTGGTGTTGCG
GAGCGCCACGACGACGGGCGCGCCCAGGAGCACTGCCGCC
AGCACCAGGTCCATGGCCGTAACGCGCGCCGCGGGGGTGC
GGTGGGTGGCGGCGGCCGGCACGGCGACGTGCTGGCCCGT
GGGCCGGTAGAGGGCGTTGGGGGGAGCGGGGGGTGACGCC
TCGCGCCCCCCCGAGGGGCTCAGCGTCTGCCCAGATTCCA
GACGCGCGGTCAGAAGGGCGTCGAAACTGTCATACGGTAG
TCGGCGCGCCCCCCGAGGGGCTCAGCGTCTGCCCAGATTC
CAGACGCCTCCGGCGTCGAAACTGTCATACTCTGTGTAGT
CGTCCGGAAACATGCAGGTCCAAAGAGCGGCCAGGGCGGT
GCTTGGGAGACACATGCGCCCGAGGACGCTCACCGCCGCC
AGCGCCTGGGCGGGACTCAGCTTTCCCAGCGCGGCGCCGC
GCTCGGTTCCCAGCTCGGGGACCGAGCGCCAGGGCGCCAG
GGGGTCGGTTTCGGACAACTTGCCGCGGCGCCAGTCTGCC
AGCCGCGTGCCGAACATGAGGCCCCGGGTCGGAGGGCCTC
CGGTCTATAATCTGGCAGCCGCGGATGCGGGCGTCTGGAT
GCGGGGTCAGGCGCTGCACGAATAGCATGGAATCTGCTGC
GTTCTGAAACGCACGGGGGAGGGTGAGATGCATGTACTCG
TGTTGGCGGACCAGATCCAGGCGCCAAAAGGTGTAAATGT
GTTCCGGGGAGCTGGCCACCAGCGCCACCAGCACGTCGTT
CTCGTTAAAGGAAACGCGGTGCCTAGTGGAGCTGTGGGGC
CCGAGCGGCGGTCCCGGGGCCGCCGCGTCACCCCCCCATT
CCAGCTGGGCCCAGCGACACCCAAACTCGCGCGTGAGAGT
GGTCGCGACGAGGGCGACGTAGAGCTCGGCCGCCGCATCC
ATCGAGGCCCCCCATCTCGCCTGGCGGTGGCGCACAAAGC
GTCCGAAGAGCTGAAAGTTGGCGGCCTGGGCGTCGCTGAG
GGCCAGCTGAAGCCGGTTGATGACGGTGATGACGTACATG
GCCGTGACGGTCGAGGCCGACTCCAGGGTGTCCGTCGGAA
GCGGGGGGCGAATGCATGCCGCCTCGGGACACATCAGCAG
CGCGCCGAGCTTGTCGGTCACGGCCGGGAAGCAGAGCGCG
TACTGCAGTGGCGTTCCATCCGGGACCAAAAAGCTGGGGG
CGAACGGCCGATCCAGCGTACTGGTGGCCTCGCGCAGCAC
CAGGGGCCCCGGGCCTCCGCTCACTCGCAGGTACGCCTCG
CCCCGGCGGCGCAGCATCTGCGGGTCGGCCTCTTGGCCGG
GTGGGGCGGACGCCCGGGCGCGTGCGTCTCGGGCGCGAAG
ATCCACGAGCAGGGGCGCGGGCGCGGCGGCCGCGCCCGCG
CCCGTCTGGCCTGTGGCCTTGGCGTACGCGCTATATAAGC
CCATGCGGCGTTGGATGAGCTCCCGCGCGCCCCGGAACTC
CTCCACCGCCCATGGGGCCAGGTCCCCGGCCACCGCGTCG
AATTCCGCCAACAGGCCCCCCAGGGTGTCAAAGTTCATCT
CCCAGGCCACCCTTGGCACCACCTCGTCCCGCAGCCGGGC
GCTCAGGTCGGCGTGTTGGGCCACGCGCCCCCCGAGCTCC
TCCACGGCCCCGGCCCGCTCGGCGCTCTTGGCGCCCAGGG
CGCCCTGGTACTTGGCGGGAAGGCGCTCGTAGTCCCGCTG
GGCTCGCAGCCCCGACACAGTGTTGGTGGTGTCCTGCAGG
GCGCGAAGCTGCTCGCATGCCGCGCGAAATCCCTCGGGCG
ATTTCCAGGCCCCCCCGCGAACGCGGCCGAAGCGACCCCA
TACCTCGTCCCACTCCGCCTCGGCCTCCTCGAGAGACCTC
CGCAGGGCCTCGACGCGGCGACGGGTGTCGAAGAGCGCCT
GCAGGCGCGCGCCCTGTCGCGTCAGGAGGCCCGGGCCGTC
GCCGCTGGCCGCGTTTAGCGGGTGCGTCTCAAAGGTACGC
TGGGCATGTTCCAACCAGGCGACCGCCTGCACGTCGAGCT
CGCGCGCCTTCTCCGTCTGGTCCAACAGAATTTCGACCTG
ATCCGCGATCTCCTCCGCCGAGCGCGCCTGGTCCAGCGTC
TTGGCCACGGTCGCCGGGACGGCGACCACCTTCAGCAGGG
TCTTCAGATTGGCCAGACCCTCGGCCTCGAGCTGGGCCCG
GCGCTCGCGCGCGGCCAGCACCTCCCGCAGCCCCGCCGTG
ACCCGCTCGGTGGCTTCGGCGCGCGCTGTTTGGCGCGCAC
CACGCGTCCTTGGTATCGGCCAGGCCCTGTCGGGTCACGA
ATGCGACGTAGTCGGCGTACGCCGTGTCCTTCACGGGGCT
CTGGTCCACGCGCTCCAGCGCCGCCACGCACGCCACCAGC
GCGTCCTCGCTCGGGCAGGGCAGGGTGACCCCTGCCCGGA
CAAGCTCGGCGGCCGCCGCCGGGTCGTTGCGCACCGCGGA
TATCTCCTCCGCGGCGGCGGCCAGGTCCAGCGCCACGCTT
CCGATCGCGCGCCGCGCGTCGGCCCGGAGGGCGTCCAGGC
GATCGCGGATATCCACGTACTCGGCGTAGCCCTTTTGAAA
AAACGGCACGTACTGGCGCAGGGCCGGCACGCCCCCCAAG
TCTTCCGACAGGTGTAGGACGGCCTCGTGGTAGTCGATAA
ACCCGTCGTTCGCCTGGGCCCGCTCCAGCAGCCCCCCCGC
CAGCCGCAGAAGCCGCGCCAGGGGCTCGGTGTCCACCCGA
AACATGTCGGCGTACGTGTCGGCCGCGGCCCCGAAGGCCG
CGCTCCAGTCGATGCGGTGAATGGCTGCGAGCGGGGGGAG
CATGGGGTGGCGCTGGTTCTCGGGGGGGTATGGGTTAAAC
GCAAGGGCCGTCTCCAGGGCAAGGGTCACCGCCTTGGCGT
TGGTTCCCAGCGCCTGTTCGGCCCGCTTTCGGAAGTCCCG
GGGGTTGTAGCCGTGCGTGCCCGCCAGCGCCTGCAGGCGA
CGGAGCTCGACCACGTCAAACTCGGCACCGCTTTCCACGC
GGTCCAGCACGGCCTCCACGTCGGCGGCCCAGCGCTCGTG
GCTACTGCGGGCGCGCTGGGCCGCCATCTTCTCTCTCAGG
TCGGCGATGGCGGCCTCAAGTTCGTCGGCGCGGCGTCGCG
TGGCGCCGATGACCTTTCCCAGCTCCTGCAGGGCGCGCCC
GCTGGGGGAGTGGTCCCCGGCCGTCCCTTCGGCGTGCAAC
GGCCCCCGAACCTGCCCTCGTGGCCCGCGAGGCTTTCCCG
CGCGCCGGTGGTCGCGCGCGTCGCGGCCTGGATCAGGGAG
GCATGCTCTCCCTCCGGTTGGTTGGCGGCCCGGCGCACCT
GGACGACAAGGTCGGCTGCCGCCGACCCTAAGGTCGTGAG
CTGGGCGATGGCCCCCCGCGCGTCCAGGGCCAACCGAGTC
GCCTTGACGTATCCCGCGGCGCTGCGGCCATGGCCGCTAG
GAAGGCCAGGGGGGAGGCCGGGTCGCTGGCGGCCGCGCCC
AGGGCCGTCACCGCGTCGACCAGGACGCGGTGCGCCCGCA
CGGCCGCATCCACCGTCGACGCGGGGTCTGCCGTCGCGAC
GGCGGCGCTGCCGGCGTTGATGGCGTTCGAGACGGCGTGG
GCTATGATCGGGGCGTGATCGGCGAAGAACGCAAGGAAAC
GGAGTCTCTGGGGCGTCGGCGACAGGTTCTTCAGCACCAC
CACGAAGCTGGGATGCAAGCCAGACAGAGCCGCGCCGTGC
CCGGGACGGGTGCTCCAGGGCATCTCGGTACTGCCCCAGC
AGCCCCCACATGTCCGCCCGCAGCGCCGCCGTAACCTCAG
GGGGCGCCCCCCGAACGGCCTCGGGGAGGTCCGACCAGCC
CGCCGGCAGGGAGGCCCGCAGGGTCGCCAGGACGGCCGGA
CAGGCCTTTAGCCCCACAAAGTCAGGGAGGGGGCGCAGGA
CCCCCTGGAGTTTGTGCAAGAACTTCTCCCGGGCGTCGCG
GGCCACCTTCGCCCGCTCCCGCGCTCCCTCGAGCATTGCC
TCCAGGGAGCGCGCGCGCTCCCGCAAACGGGCACGCGCAT
CGGGGGCGAGCTCTGCCGTCAGCTTGGCGGCATCCATGGC
CCGCGCCTGCCGCAGCGCTTCCCGGCCATGCGCGTGGCCT
CTGGCGACAGCCCGCCGTCGTCGGGGTAGGGCGACGCGCC
GGGCGCAGGAACAAAGGCCGCGTCGCTGTCCAGCTGCTGG
CCCAGGGCCGCATCTAGGGCGTCGAAGCGCCGCAGCTCGG
CCAGACCCGAGCTGCGGCGCGCCTGCTGGTCGTTAATGTC
GCGGATGCTGTGCGCCAGCTCGTCCAGCGGCTTGCGTTCT
ATCAGCCCTTGGTTGGCGGCGTCCGTCAGGACGGAGAGCC
AGGCCGCCAGGTCCTCGGGGGCGTCCAGCGTCTGGCCCCG
CTGGATCAGATCCCGCAACAGGATGGCCGTGGGGCTGGTC
GCGATCGGGGGCGGGGCGGGCGCGCCGGGCGCAGGAACAA
AGGCCGCGTCGCTGGCCAGCTGCTGGCCCAGGGCCGCATC
TAGGGCGCGAAGCGCCGCGGCGCGGCCGGCCCCGGGCGGG
GGCGCGCCTGCTGGTCGTTAATGTCGCGGATGCTGTGCGC
CAGCTCGTCCAGCGGCTTGCGTTCTATCAGCCCTTGGTTG
GCGGCGTCCGTCAGGACGGAGAGCCGGCCGCCGGTCCTCG
GGGGCGTCCAGCGTCTGGCCCCGCTGGATCAGATCCCGCA
ACAGGATGGCCGTGGGGCTGGTCGCGATCGGGGGCGGGGC
GGGAATGGCGGCGCGCTGCGCGATGTCCCGCGGTGCTGGT
CGAAGACAGGCAGGGACTCGAGCAGCTGGACCACGGGCAC
GACGGCGGCCGAAGCCACGTGAAACCGGCGGTCGTTGTTG
TCGCTGGCCTGTAGAGCCTTGGCGCTGTATACGGCCCCCC
GGTAAAAGTACTCCTTAACCGCGCCCTCGATCGCCCGACG
GGCCTGGGTCCGCACCTCCTCCAGCCGAACCTGAACGGCC
TCGGGGCCCAGGGGGGGTGGGCGCGGAGCCCCCTGCGGGG
CCGCCCCGCCGGGGGAAGTAAGAAGAGGGGCCCGGCGTGC
TGTGAGACCGCGTCGACCCCGCGAGCGAGGGCGTCGAGGG
CCTCGCGCATCTGGCGATCCTCCGCCTCCACCCTAATCTC
TTCGCCACGGGCAAATTTGGCCAGAGCCTGGACTCTATAC
AGAAGCGGTTCTGGGTGCTTCGGGGTGGCGGGGGCAAAAA
GGGTGTCCGGGTGGGCCTGCGAGCGCTCCAGAAGCCACTC
GCCGAGGCGTGTATACAGATTGGCCGGCGGGGCCGCGCGA
AGCTGCAGCTCCAGGGCCGCGAGTTCCCCGTAAAAGGCGT
CCGTCTCCCGAATGACATCCCTAGCCACAAGGATCAGCTT
CGCCAGCGCCAGGCGACCGATCAGAGAGTTTTCGTCCAGC
ACGTGCTGGACGAGGGGCAGATGGGCGGCCACGTCGGCCA
GGCTCAGGCGCGTGGAGGCCAGAAAGTCCCCCACGGCCGT
TTTCCGGGGCAGCATGCTCAGGGTAAACTCCAGCAGGGCG
GCGGCCGGGCCGGCCACCCCGGCCTGGGGGTGCGTCCGGG
CCCCGTTCTCGATGAGAAAGGCGAGGACGCGTTCAAAGAA
AAAAATAACACAGAGCTCCAGCAGCCCCGGAGAAGCCGGA
TACGGCGACCGTAAGGCGCTGATGGTGAGCCGCGAACACG
CGGCGCCCTCGCGGGCCAGGGTGGCGGAGCACGCGGTGAA
CTTAACCGCCGTGGCGGCCACGTTTGGGTGGGCCTCGAAC
AGCTGGGCGAGGTCTGCGCCCGGGGGCTCGGGTGAGCGGC
GAGTCTTCAGCGCCTCGAGGGCCTGTGAGGACGCCGGAAC
CATGGGCCCGTCGTCCTCGCCCGCCTCGGCGACCGGCGGC
CCGGCCGGGTCGGGGGGTGCCGAGGCGAGGACAGGCTCCG
GAACGGAGGCGGGGACCGCGGCCCCGACGGGGGTTTTGCC
TTTGGGGGTGGATTTCTTCTTGGTTTTGGCAGGGGGGGCC
GAGCGTTTCGTTTTCTCCCCCGAAGTCAGGTCTTCGACGC
TGGAAGGCGGAGTCCAGGTGGGTCGGCGGCGCTTGGGAAG
GCCGGCCGAGTAGCGTGCCCGGTGCCGACCAACCGGGACG
ACGCCCATCTCCAGGACCCGCATGTCGTCGTCATCTTCTT
CGGCCGCCTCTGCGGCGGGGGTCTTGGGGGCGGAGGGAGG
CGGTGGTGGGATCGCGGAGGGTGGGTCGGCGGAGGGGGGA
TCCGTGGGTGGGGTACCCTTTAGGGCCACCGCCCATACAT
CGTCGGGCGCCCGATTCGGGCGCTTGGCCTCTGGTTTTGC
CGACGGACCGGCCGTCCCCCGGGATGTCTCGGAGGCCCTG
TCGTCGCGACGGGCCCGGGTCGGTGGCGGCGACTGGGCGG
CTGTGGGCGGGTGTGGCCCCGGCCCCCCTCCCCCCTCCCG
GGGGCCCACGCCGACGCAGGGCTCCCCCAGGCCCGCGATC
TCGCCCCGCAGGGGGGGCGTGATGGCCACGCGCCGTTCGC
TGAACGCTTCGTCCTGCATGTAAGTCTCGCTGGCCCCGTA
AAGATGCAGAGCCGCGGCCGTCAAGTCCGCAGGAGCCGCG
GGTTCCGGGCCCGACGGCACGAAAAACACCATGGCTCCCG
CCCACCGTACGTCCGGGCGATCGCGGGTGTAATACGTCAG
GTATGGATACATGTCCCCCGCCCGCACTTTGGCGATGAAC
GCGGGGGTGCCCTCCGGAAGGCCATGCGGGTCAAAAGGTA
GGCGGTGTCGCCGTCCCTGAACAGCCCCATCCCTAGGGGG
CCAATGGTTAGGAGCGTGTACGACAGGGGGCGCAGGGCCC
ACGGGCCGGCGAAGAACGTGTGTGCGGGGCATTGTGTCTC
CAGCAGGCCTGCCGCGGGCTCCCCGAAGAAGCCCACCTCG
CCGTATACGCGCGAGAAGACACAGCGCAGTCCGCCGCGCG
CCCCTGGGTACTCGAGGAAGTTGGGGAGCTCGACGATCGA
ACACATGCGCGGCGGCCCAGGGCCCGCAGTCGCGCGCGTC
CACTCGCCCCCCTCGACCAAACATCCCTCGATGGCCTCCG
CGGACAGAACGTCGCGAGGGCCCACATCAAATATGAGGCT
GAGAAAGGCAGCGACGAGCGCATGCACGATACCGACCCCC
CCGGCTCCAGGTCGGGCGCGAACTGGTTCCGAGCACCGGT
GACCACGATGTCGCGATCCCCCCCGCGTTCCATCGTGGAG
TGCGGTGGGGTGCCCGCGATCATATTGCCCTGCGGGCCAG
AGACCCGGCCTGTTTATGGACCGGACCCCCGGGGTTAGTG
TTGTTTCCGCCACCCACGCCCCCGTACCATGGCCCCGGTT
CCCCTGATTAGGCTACGAGTCGCGGTGATCGCTTCCCAAA
AACCGAGCTGCGTTTGTCTGTCTTGGTCTTCCCCCCCCCC
AGCCCGCACACCATAACACCGAGAACAACACACGGGGGTG
GGCGGAACATAATAAAGCTTTATTGGTAACTAGTTAACGG
CAAGTCCGTGGGTGGCGCGACGGTGTCCTCCGGGATCATC
TCGTCGTCCTCGACGGGGGTGTTGGAATGAGGCGCCTCCT
CGCGGTCCACCTGGCGTGGGCCGTGCCCATAGGCCTCCGG
CTTCTGTGCGTCCATGGGCGTAGGCGCGGGGAGACTGTTT
CCGGCGTCGCGGACCTCCAGGTCCCTGGGAGCCTCCGGTC
CGGCTAACGGACGAAACGCGGAAGCGCGAAACACGCCGTC
GGTGACCCGCAGGAGCTCGTTCATCAGTAACCAATCCATA
CTCAGCGTAACGGCCAGCCCCTGGCGAGACAGATCCACGG
AGTCCGGAACCGCGGTCGTCTGGCCCAGGGGGCCGAGGCT
GTAGTCCCCCCAGGCCCCTAGGTCGCGACGGCTCGTAAGC
ACGACGCGGTCGGCCGCGGGGCTTTGCGGGGGGGCGTCCT
CGGGCGCATGCGCCATTACCTCTCGGATGGCCGCGGCGCG
CTGGTCGGCCGAGCTGACCAAGGGCGCCACGACCACGGCG
CGCTCCGTCTGCAGGCCCTTCCACGTGTCGTGGAGTTCCT
GGACAAACTCGGCCACGGGCTCGGGTCCCGCGGCCGCGCG
CGCGGCTTGATAGCAGGCCGACAGACGCCGCCAGCGCGCT
AGAAACTGACCCATGAAACAACCCCCGTGTACCTGGTCTC
CCGACAGCAGCTTCGACGCCCGGGCGTGAATGCCGGCCAC
GACGGACAGAAACCCGTGAATTTCGCGCCGGACCACGGCC
AGCACGTTGTCCTCGTGCGACACCTGGGCCGCCAGCTCGT
CGCACACCCCCAGGTGCGCCGTGGTTTCGGTGATGACGGA
ACGCAGGCTCGCGAGGGACGCGACCAGCGCGCGCTTGGCG
TCGTGATACATGCTGCAGTACTGACTCACCGCGTCCCCCA
TGGCCTCGGGGGGCCAGGGCCCCAGGCGGTCGGGCGTGTC
CCCGACCACCGCATACAGGCGGCGCCCGTCGCTCTCGAAC
CGACACTCGAAAAAGGCGGAGAGCGTGCGCATGTGCAGCC
GCAGCAGCACGATGGCGTCCTCCAGTTGGCGAATCAGGGG
GTCGGCGCGCTCGGCGAGGTCCTGCAGCACCCCCCGGGCA
GCCAGGGCGTACATGCTAATCAACAGGAGGCTGGTGCCCA
CCTCGGGGGGCGGGGGGGGCTGCAGTTGGACCAGGGGCCG
CAGCTGCTCGACGGCACCCCTGGAGATCACGTACAGCTCC
CGGAGCAGCTGCTCTATGTTGTCGGCCATCTGCATAGTGG
GGCCGAGGCCGCCCCGGGCGGCCGGTTCGAGGAGAGTGAT
CAGCGCGCCCAGTTTGGTGCGATGGCCCTCGACCGTGGGG
AGATAGCCCAGCCCAAAGTCCCGGGCCCAGGCCAACACAC
GCAGGGCGAACTCGACCGGGCGGGGAAGGTAGGCCGCGCT
ACACGTGGCCCTCAGCGCGTCCCCAACCACCAGGGCCAGA
ACGTAGGGGACGAAGCCCGGGTCGGCGAGGACGTTGGGGT
GAATGCCCTCGAGGGCGGGGAAGCGGATCTGGGTCGCCGC
GGCCAGGTGGACAGAGGGGGCATGGCTGGGCTGCCCGACG
GGGAGAAGCGCGGACAGCGGCGTGGCCGGGGTGGTGGGGG
TGATGTCCCAGTGGGTCTGACCATACACGTCGATCCAGAT
GAGCGCCGTCTCGCGGAGAAGGCTGGGTTGACCGGAACTA
AAGCGGCGCTCGGCCGTCTCAAACTCCCCCACGAGCGCCC
GCCGCAGGCTCGCCAGATGTTCCGTCGGCACGGCCGGCCC
CATGATACGCGCCGCGTCTGGCTCAGAACGCCCCCCGACA
GGCCGCCGCCTCACAGCGCCGCCCGTGCGTGTGCTCGCTG
GCGCCCTGGCCCGCCTGAAAGTTTTTACGTAGTTGGCATA
GTACCCGTATCCCGCGCCAGACCAAACACGTTCGCCCCCG
CGAGGGCAATGCCCCAAAGAGCTGCTGGACTTCGCCGAGT
CCGTGGCCGGCGGGCGTCCGCGCGGGGACGCCCGCCGCCA
GAAACCCCTCCAGGGCCGAAAGGGAGTGCGTGCAGTGCGA
GGGCGTGAACCCAGCGTCGATCAGGGTGTTGATCACCACG
GAGGGCGAATTGGATTCTGGATCAACGTCCACGTCTGCTG
CAGCAGAGCCAGCAGCCGCTGCTGGGCGCCGGCGGAGGGC
TGCTCCCCGAGCTGCAGCAGGCTGGAGACGGCAGGCTGGA
AGACTGCCAGTGCCGACGAACTCAGGAACGGCACGTCGGG
ATCAAACACGGCCACGTCCGTCCGCACGCGCGCCATTAGC
GTCCCCGGGGGCGCACAGGCCGAGCGCGGGCTGACGCGGC
TGAGGGCCGTCGACACGCGCACCTCCTCGCGGCTGCGAAC
CATCTTGTTGGCCTCCAGTGGCGGAATCATTATGGCCGGG
TCGATCTCCCGCACGGTGTGCTGAAACTGCGCCAACAGGG
GCGGCGGGACCACAGCCCCCCGCTCGGGGGTCGTCAGGTA
CTCGTCCACCAGGGCCAACGTAAAGAGGGCCCGTGTGAGG
GGAGTGAGGGTCGCGTCGTCTATGCGCTGGAGGTGCGCCG
AGAACAGCGTCACCCGATTACTCCCCCCAAGAACCGGAGG
CCCTCTTGCACGAACGGGGCGGGGAAGAGCAGGCTGTACG
CCGGGGTGGTAAGGTTCGCGCTGGGCTGCCCCAACGGGAC
CGGCGCCAGCTTGAGCGACGTCTCCCCAAGGGCCTCGATG
GAGGTCCGCGGGCTCATGGCCAAGCAGCTCTTGGTGACGG
TTTGCCAGCGGTCTATCCACTCCACGGCGCACTGCGGCGC
GGACCGGCCCCAGGGCCGCCGCGGTGCGCAGGCCGGCGGA
CTCCAGCGCATGGGACGTGTCGGAGCCGGTGACCGCGAGG
ATGGTGTCCTTGATGACCTCCATCTCCCGGAAGGCCTGGT
CGGGGGCCTCGGGGAGAGCCACCACCAAGCGGTGTACGAG
CAACCCGGGGAGGTTCTCGGCCAAGAGCGCCGTCTCCGGA
AGCCCGTGGGCCCGGTGGAGCGCGCACAGGTGTTCCAGCA
GCGGCCGCCAGCATGCCCGCGCGTCTGCCGGGGCGATGGC
CGTTCCCGACAACAGAAACGCCGCCATGGCGGCGCGCAGC
TTGGCCGTGGCCAGAAACGCCGGGTCGTCCGCCCCGTTTG
CCGTCTCGGCCGTGGGGGTTGGCGGTTGGCGAAGGCCGGC
TAGGCTCGCCAATAGGCGCTGCATAGGTCCGTCCGAGGGC
GGACCGGCGGGTGAGGTCGTGACGACGGGGGCCTCGGACG
GGAGACCGCGGTCTGCCATGACGCCCGGCTCGCGTGGGGG
GGGGACAGCGTAGACCAACGACGAGACCGGGCGGGAATGA
CTGTCGTGCGCTGTAGGGAGCGGCGAATTATCGATCCCCC
GCGGCCCTCCAGGAACCCCGCAGGCGTTGCGAGTACCCCG
CGTCTTCGCGGGGTGTTATACGGCCACTTAAGTCCCGGCA
TCCCGTTCGCGGACCCAGGCCCGGGGGATTGTCCGGATGT
GCGGGCAGCCCGGACGGCGTGGGTTGCGGACTTTCGGCGG
GGCGGCCCAAATGGCCCTTTAAACGTGTGTATACGGACGC
GCCGGGCCAGTCGGCCAACACAACCCACCGGAGGCGGTAG
CCGCGTTTGGCTGTGGGGTGGGTGGTTCCGCCTTGCGTGA
GTGTCCTTTCGACCCCCCCCCTCCCCCGGGTCTTGCTAGG
TCGCGATCTGTGGTCGCAATGAAGACCAATCCGCTACCCG
CAACCCCTTCCGTGTGGGGCGGGAGTACCGTGGAACTCCC
CCCCACCACACGCGATACCGCGGGGCAGGGCCTGCTTCGG
CGCGTCCTGCGCCCCCCGATCTCTCGCCGCGACGGCCCAG
TGCTCCCCAGGGGGTCGGGACCCCGGAGGGCGGCCAGCAC
GCTGTGGTTGCTTGGCCTGGACGGCACAGACGCGCCCCCT
GGGGCGCTGACCCCCAACGCGATACCGAACAGGCCCTGGA
CAAGATCCTGCGGGGCACCATGCGCGGGGGGGCGGCCCTG
ATCGGCTCCCCGCGCCATCATCTAACCCGCCAAGTGATCC
TGACGGATCTGTGCCAACCCAACGCGGATCGTGCCGGGAC
GCTGCTTCTGGCGCTGCGGCACCCCGCCGACCTGCCTCAC
CTGGCCCACCAGCGCGCCCCGCCAGGCCGGCAGACCGAGC
GGCTGGGCGAGGCCTGGGGCCAGCTGATGGAGGCGACCGC
CCTGGGGTCGGGGCGAGCCGAGAGCGGGTGCACGCGCGCG
GGCCTCGTGTCGTTTAACTTCCTGGTGGCGGCGTGTGCCG
CCTCGTACGACGCGCGCGACGCCGCCGATGCGGTACGGGC
CCACGTCACGGCCAACTACCGCGGGACGCGGGTGGGGGCG
CGCCTGGATCGTTTTTCCGAGTGTCTGCGCGCCATGGTTC
ACACGCACGTCTTCCCCCACGAGGTCATGCGGTTTTTCGG
GGGGCTGGTGTCGTGGGTCACCCAGGACGAGCTAGCGAGC
GTCACCGCCGTGTGCGCCGGGCCCCAGGAGGCGGCGCACA
CCGGCCACCCGGGCCGGCCCCGCTCGGCCGTGATCCTCCC
GGCGTGTGCGTTCGTGGACCTGGACGCCGAGCTGGGGCTG
GGGGGCCCGGGCGCGGCGTTTCTGTACCTGGTTCACTTAC
CGCCAGCGGGACCAGGAGCTGTGTTGTGTGTACGTGATCA
AGAGCCAGCTCCCCCCGCGCGGGTTGGAGCCGGCCCTGGA
GCGGCTGTTTGGGCGCCTCCGGATCCCAACACGATTCACG
GCACCGAGGACATGACGCCCCCGGCCCCAAACCGAAACCC
CGACTTCCCCCTCGCGGGCCTGGCCGCCAATCCCCAAACC
CCGCGTTGCTCTGCTGGCCAGGTCACGAACCCCCAGTTCG
CCGACAGGCTGTACCGCTGGCAGCCGGACCTGCGGGGGCG
CCCCACCGCACGCACCTGTACGTACGCCGCCTTCGCAGAG
CTCGGCATGATGCCCGAGGATAGTCCCCGCTGCCTGCACC
GCACCGAGCGCTTTGGGGCGGTCAGCGTCCCCGTTGTCAT
CCTGGAAGGCGTGGTGTGGCGCCCCGGCGAGTGGCGGGCC
TGCGCGTGAGCGTAGCAAACGCCCCGCCCACACAACGCTC
CGCCCCCAACCCCTTCCCCGCTGTCACTCGTTGTTCGTTG
ACCCGGACGTCCGCCAAATAAAGCCACTGAAACCCGAAAC
GCGAGTGTTGTAACGTCCTTTGGGCGGGAGGAAGCCACAA
AATGCAAATGGGATACATGGAAGGAACACACCCCCGTGAC
TCAGGACATCGGCGTGTCCTTTTGGGTTTCACTGAAACTG
GCCCGCGCCCCACCCCTGCGCGATGTGGATAAAAAGCCAG
CGCGGGTGGTTTAGGGTACCACAGGTGGGTGCTTTGGAAA
CTTGTCGGTCGCCGTGCTCCTGTGAGCTTGCGTCCCTCCC
CGGTTTCCTTTGCGCTCCCGCCTTCCGGACCTGCTCTCGC
CTATCTTCTTTGGCTCTCGGTGCGATTCGTCAGGCAGTGG
CCTTGTCGAATCTCGACCCCACCACTCGCCGGACCCGCCG
ACGTCCCCTCTCGAGCCCGCCGAAACCCGCCGCGTCTGTT
GAAATGGCCAGCCGCCCCGCCGCATCCTCTCCCGTCGAAG
CGCGGGCCCCGGTTGGGGGACAGGAGGCCGGCGGCCCCAG
CGCAGCCACCCAGGGGGAGGCCGCCGGGGCCCCTCTCGCC
CGCGGCCACCACGTGTACTGCCAGCGAGTCAATGGCGTGA
TGGTGCTTTCCGACAAGACGCCCGGGTCCGCGTCCTACCG
CATCAGCGATAGCAACTTTGTCCAATGTGGTTCCAACTGC
ACCATGATCATAGACGGAGACGTGGTGCGCGGGCGCCCCC
AGGACCCGGGGGCCGCGGCATCCCCCGCTCCCTTCGTTGC
GGTGACAAACATCGGAGCCGGCAGCGACGGCGGGACCGCC
GTCGTGGCATTCGGGGGAACCCCACGTCGCTCGGCGGGGA
CGTCTACCGGTACCCAGACGACCGACGTCCCCACCGAGGC
CCTTGGGGGCCCCCCTCCTCCTCCCCGCTTCACCCTGGGG
GGCGGCTGTTGTTCCTGTCGCGACACACGGCGCCGCTCTG
CGGGATTCGGGGGGGAGGGGGATCCCGTCGGCCCCGCGTT
GTCGTCTCGGACGACCGTTGCTCCGATTCCGACTCGGATG
ACTCGGAGGACACCGACTCGGAGACGCTGTCACACGCCTC
CTCGGACGTGTCCGGCGGGGCCACGTACGACGACGCCCTT
GACTCCGATTCGTCATCGGATGACTCCCTGCAGATAGATG
GCCCCGTGTGTCGCCCGTGGAGCAATGACACCGCGCCCCT
GGATGTTTGCCCCGGGACCCCCGGCCCGGGCGCCGACGCC
GGTGGTCCCTCAGCGGTAGACCCACACGCACCGACGCCAG
GGGCCGGCGCTGGTCTTGCGGCCGATCCCGCCGTGGCCCG
GGACGACGCGGAGGGGCTTTCGGACCCCCGGCCACGTCTG
GGAACGGGCACGGCCTACCCCGTCCCCCTGGAACTCACGC
CCGAGAACGCGGAGGCCGTGGCGCGCTTTCTGGGAGATGC
CGTGAACCGCGAACCCGCGCTCATGCTGGAGTACTTTTGC
CGGTGCGCCCGCGAGGAAACCAAGCGTGTCCCCCCCAGGA
CATTCCGCCCGGGTCCGCGTCCTACCGCATCAGCGATAGC
AACTTTGTCCAATGTGGTTCCAACTGCACCATGATCATAG
ACGGAGACGTGGTGCGCGGGCGCCCCCAGGACCCGGGGGC
CGCGGCATCCCCCGCTCCCTTCGTTGCGGTGACAAACATC
GGAGCCGGCAGCGACGGCGGGACCGCCGTCGTGGCATTCG
GGGGAACCCCACGTCGCTCGGCGGGGACGTCTACCGGTAC
CCAGACGACCGACGTCCCCACCGAGGCCCTTGGGGGCCCC
CCTCCTCCTCCCCGCTTCACCCTGGGTGGCGGCTGTTGTT
CCTGTCGCGACACACGGCGCCGCTCTGCGGTATTCGGGGG
GGAGGGGGATCCCGTCGGCCCCGCGGAGTTCGTCTCGGAC
GACCGGTCGTCCGATTCCGACTCGGATGACTCGGAGGACA
CCGACTCGGAGACGCTGTCACACGCCTCCTCGGACGTGTC
CGGCGGGGCCACGTACGACGACGCCCTTGACTCCGATTCG
TCATCGGATGACTCCCTGCAGATAGATGGCCCCGTGTGTC
GCCCGTGGAGCAATGACACCGCGCCCCTGGATGTTTGCCC
CGGGACCCCCGGCCCGGGCGCCGACGCCGGTGGTCCCTCA
GCGGTAGACCCACACGCCCGACGCCAGGGGCCGGCGCTGG
TCTTGCGGCCGATCCCGCCGTGGCCCGGGACGACGCGGAG
GGGCTTTCGGACCCCCGGCCACGTCTGGGAACGGGCACGG
CCTACCCCGTCCCCCTGGAACTCACGCCCGAGAACGCGGA
GGCCGTGGCGCGCTTTCTGGGAGATGCCGTGAACCGCGAA
CCCGCGCTCATGCTGGAGTACTTTTGCCGGTGCGCCCGCG
AGGAAACCAAGCGTGTCCCCCCCAGGACATTCTGCAGCCC
CCCTCGCCTCACGGAGGACGACTTTGGGCTTCTCAACTAC
GCGCTCGTGGAGATGCAGCGCCTGTGTCTGGACGTTCCTC
CGGTCCCGCCGAACGCATACATGCCCTATTATCTCAGGGA
GTATGTGACGCGGCTGGTCAACGGGTTCAAGCCGCTGGTG
AGCCGGTCCGCTCGCCTTTACCGCATCCTGGGGGTTCTGG
TGCACCTGCGGATCCGGACCCGGGAGGCCTCCTTTGAGGA
GTGGCTGCGATCCAAGGAAGTGGCCCTGGACTTTGGCCTG
ACGGAAAGGCTTCGCGAGCACGAAGCCCAGCTGGTGATCC
TGGCCCAGGCTCTGGACCATTACGACTGTCTGATCCACAG
CACACCGCACACGCTGGTCGAGCGGGGGCTGCAATCGGCC
CTGAAGTATGAGGAGTTTTACCTAAAGCGCTTTGGCGGGC
ACTACATGGAGTCCGTCTTCCAGATGTACACCCGCATCGC
CGGCTTTTTGGCCTGCCGGGCCACGCGCGGCATGCGCCAC
ATCGCCCTGGGGCGAGAGGGGTCGTGGTGGGAAATGTTCA
AGTTCTTTTTCCACCGCCTCTACGACCACCAGATCGTACC
GTCGACCCCCGCCATGCTGAACCTGGGGACCCGCAACTAC
TACACCTCCAGCTGCTACCTGGTAAACCCCCAGGCCACCA
CAAACAAGGCGACCCTGCGGGCCATCACCAGCAACATCAG
CGCCATCCTCGCCCGCAACGGGGGCATCGGGCTATGCGTG
CAGGCGTTTAACGACTCCGGCCCCGGGACCGCTAGCGTCA
TACCCGCCCTCAAGGTCCTCGACTCGCTGGTGGCGGCGCA
CAACAAAGGAGCGCGCGTCCAACCGGCGCGTGCGTGTACC
TGGAGCCGTGGCACACCGACGTGCGGGCCGTGCTCCGGAT
GAAGGGGGTCCTCGCCGGCGAAGAGGCCCAGCGCTGCGAC
AATATCTTCAGCGCCCTCTGGATGCCAGACCTGTTTTTCA
AGCGCCTGATTCGCCACCTGGACGGCGAGAAGAACGTCAC
ATGGACCCTGTTCGACCGGGACACCAGCATGTCGCTCGCC
GACTTTCACGGGGAGGAGTTCGAGAAGCTCTACCAGCACC
TCGAGGTCATGGGGTTCGGCGAGCAGATACCCATCCAGGA
GCTGGCCTATGGCATTGTGCGCAGTGCGGCCACGACCGGG
AGCCCCTTCGTCATGTTCAAAGACGCGGTGAACCGCCACT
ACATCTACGACACCCAGGGGGCGGCCATCGCCGGCTCCAA
CCTCTGCACCGAGATCGTCCATCCGGCCTCCAAGCGATCC
AGTGGGGTCTGCAATCTGGGAAGCGTGAATCTGGCCCGAT
GCGTCTCCAGGCAGACGTTTGACTTTGGGCGGCTCCGCGA
CGCCGTGCAGGCGTGCGTGCTGATGGTGAACATCATGATC
GACAGCACGCTACAACCCACGCCCCAGTGCACCCGCGGCA
ACGACAACCTGCGGTCCATGGGAATCGGCATGCAGGGCCT
GCACACGGCCTGCCTGAAGCTGGGGCTGGATCTGGAGTCT
GTCGAATTTCAGGACCTGAACAAACACATCGCCGAGGGAT
GCTGCTGTCGGCGATGAAGACCAGCAACGCGCTGTGCGTT
CGCGGGGCCCGTCCCTTCAACCACTTTAAGCGCAGCATGT
ATCGCGCCGGCCGCTTTCACTGGGAGCGCTTTCCGGACGC
CCGGCCGCGGTACGAGGGCGAGTGGGAGATGCTACGCCAG
AGCTGGATGAAACACGGCCTGCGCAACAGCCAGTTTGTCG
CGCTGATGCCCACCGCCGCCTCGGCGCAGATCTCGGACGT
CAGCGAGGGCTTTGCCCCCCTGTTCACCAACCTGTTCAGC
AAGGTGACCCGGGACGGCGAGACGCTGCGCCCCAACACGC
TCCTGCTAAAGGAACTGGAACGCACGTTTAGCGGGAAGCG
CCTCCTGGAGGTGATGGACAGTCTCGACGCCAAGCAGTGG
TCCGTGGCGCAGGCGCTCCCGTGCCTGGAGCCCACCCACC
CCCTCCGGCGATTCAAGACCGCGTTTGACTACGACCAGAA
GTTGCTGATCGACCTGGTGCGGACCGCGCCCCCTACGTCG
ACCATAGCCAATCCATGACCCTGTATGTCACGGAGAAGGC
GGACGGGACCCTCCCAGCCTCCACCCTGGTCCGCCTTCTG
GTCCACGCATATAAGCGCGGACTAAAAACAGGGATGTACT
ACTGCAAGGTTCGCAAGGCGACCAACAGCGGGGTCTTTGG
CGGCGACGACAACATTGTCTGCACGAGCTGCGCGCTGTGA
CCGACAAACCCCCTCCGCGCCAGGCCCGCCGCCACTGTCG
TCGCCGTCCCACGCGCTCCCCCGCTGCCATGGATTCCGCG
GCCCCAGCCCTCTCCCCCGCTCTGACGGCCCATACGGGCC
AGAGCGCGCCGGCGGACCTGGCGATCCAGATTCCAAAGTG
CCCCGACCCCGAGAGGTACTTCTACACCTCCCAGTGTCCC
GACATTAACCACCTGCGCTCCCTCAACATCCTTAACCGCT
GGCTGGAAACCGAGCTTGTTTTCGTGGGGGACGAGGAGGA
CGTCTCCAAGCTTTCCGAGGGCGAGCTCAGCTTTTACCGC
TTCCTCTTCGCTTTCCTGTCGGCCGCCGACGACCTGGTTA
CGGAAAACCTGGGCGGCCTCTCCGGCCTGTTTGAGCAGAA
GGACATTCTCCACTACTACGTGGAGCAGGAATGCATCGAA
GTCGTACACTCGCGCGTGTACAACATCATCCAGCTGGTGC
TTTTTCACAACAACGACCAGGCGCGCCGCGAGTACGTGGC
CGGCACCATCAACCACCCGGCCATCCGCGCCAAGGTGGAC
TGGCTGGAAGCGCGGGTGCGGGAATGCGCCTCCGTTCCGG
AAAAGTTCATCCTCATGATCCTCATCGAGGGCATCTTTTT
TGCCGCCTCGTTTGCCGCCATCGCCTACCTTCGCACCAAC
AACCTTCTGCGGGTCACCTGCCAGTCAAACGACCTCATCA
GCCGGGACGAGGCCGTGCACACGACGGCCTCGTGTTACAT
CTACAACAACTACCTCGGCGGGCACGCCAAGCCCCCGCCC
GACCGCGTGTACGGGCTGTTCCGCCAGGCGGTCGAGATCG
AGATCGGATTTATCCGATCCCAGGCGCCGACGGACAGCCA
TATCCTGAGCCCGGCGGCGCTGGCGGCCATCGAAAACTAC
GTGCGATTCAGCGCGGATCGCCTGTTGGGCCTTATCCACA
TGAAGCCACTGTTTTCCGCCCCACCCCCCGACGCCGTATG
TCCCGGAGAAGGCGGACGGGACCCTCCCAGCCTCCCCCTG
GTCCGCCTTCTGGTCCACGCATATAAGCGCGGACTAAAAA
CAGGGATGTACTACTGCAAGGTTCGCAAGGCGACCAACAG
CGGGGTCTTTGGCGGCGACGACAACATTGTCTGCACGAGC
TGCGCGCTGTGACCGACAAACCCCCCCGCGCCAGGCCCGC
CGCCACTGTCGTCGCCGTCCCACGCGCTCCCCCGCTGCCA
TGGATTCCGCGGCCCCAGCCCCCCCCCGCTCGACGGCCCA
TACGGGCCAGAGCGCGCCGGCGGACCTGGCGATCCAGATT
CCAAAGTGCCCCGACCCCGAGAGGTACTTCTACACCTCCC
AGTGTCCCGACATTAACCACCTGCGCTCCCTCAACATCCT
TAACCGCTGGCTGGAAACCGAGCTTGTTTTCGTGGGGGAC
GAGGAGGACGTCTCCAAGCTTTCCGAGGGCGAGCTCAGCT
TTTACCGCTTCCTCTTCGCTTTCCTGTCGGCCGCCGACGA
CCTGGTTACGGAAAACCTGGGCGGCCTCTCCGGCCTGTTT
GAGCAGAAGGACATTCTCCACTACTACGTGGAGCAGGAAT
GCATCGAAGTCGTACACTCGCGCGTGTACAACATCATCCA
GCTGGTGCTTTTTCACAACAACGCCAGGCGCGCCGCGAGT
ACGTGGCCGGCACCATCAACCACCCGGCCATCCGCGCCAA
GGTGGACTGGCTGGAAGCGCGGGTGCGGGAATGCGCCTCC
GTTCCGGAAAAGTTCATCCTCATGATCCTCATCGAGGGCA
TCTTTTTTGCCGCCTCGTTTGCCGCCATCGCCTACCTTCG
CACCAACAACCTTCTGCGGGTCACCTGCCAGTCAAACGAC
CTCATCAGCCGGGACGAGGCCGTGCACACGACGGCCTCGT
GTTACATCTACAACAACTACCTCGGCGGGCACAACCTTCT
GCGGGTCACCTGCCAGTCAAACGACCTCATCAGCCGGGAC
GAGGCCGTGCACACGCGGCCTCGTGTTACATCTACAACAA
CTACCTCGGCGGGCACGCCAAGCCCCCGCCCGACCGCGTG
TACGGGCTGTTCCGCCAGGCGGTCGAGATCGAGATCGGAT
TTATCCGATCCCAGGCGCCGACGGACAGCCATATCCTGAG
CCCGGCGGCGCTGGCGGCCATCGAAAACTACGTGCGATTC
AGCGCGGATCGCCTGTTGGGCCTTATCCACATGAAGCCAC
TGTTTTCCGCCCCACCCCCCGACGCCAGCTTTCCGCTGAG
CCTCATGTCCACCGACAAACACACCAATTTTTTCGAGTGT
CGCAGCACCTCCTACGCCGGGGCGGTCGTCAACGATCTGT
GAGGGTCGCGGCGCGCTTCTACCCGTGTTTGCCCATAATA
AACCTCTGAACCAAACTTTGGGTCTCATTGTGATTCTTGT
CAGGGACGCGGGGGTGGGAGAGGATAAAAGGCGGCGCAAA
AAGCAGTAACCAGGTCCGTCCAGATTCTGAGGGCATAGGA
TACCATAATTTTATTGGTGGGTCGTTTGTTCGGGGACAAG
CGCGCTCGTCTGACGTTTGGGCTACTCGTCCCAGAATTTG
GCCAGGACGTCCTTGTAGAACGCGGGTGGGGGGGCCTGGG
TCCGCAGCTGCTCCAGAAACCTGTCGGCGATATCAGGGGC
CGTGATATGCCGGGTCACAATAGATCGCGCCAGGTTTTCG
TCGCGGATGTCCTGGTAGATAGGCAGGCGTTTCAGAAGAG
TCCACGGCCCCCGCTCCTTGGGGCCGATAAGCGATATGAC
GTACTTAATGTAGCGGTGTTCCACCAGCTCGGTGATGGTC
ATGGGATCGGGGAGCCAGTCCAGGGACTCTGGGGCGTCGT
GGATGACGTGGCGTCGCCGGCTGGCCACATAACTGCGGTG
CTCTTCCAGCAGCTGCGCGTTCGGGACCTGGACGAGCTCG
GGCGGGGTGAGTATCTCCGAGGAGGACGACCTGGGGCCGG
GGTGGCCCCCGGTAACGTCCCGGGGATCCAGGGGGAGGTC
CTCGTCGTCTTCGTATCCGCCGGCGATCTGTTGGGTTAGA
ATTTCGGTCCACGAGACGCGCATCTCGGTGCCGCCGGCGG
CCGGCGGCAAAGGGGGCCTGGTTTCCGTGGAGCGCGAGCT
GGTGTGTTCCCGGCGGATGGCCCGCCGGGTCTGAGAGCGA
CTCGGGGGGGTCCAGTGACATTCGCGCAGCACATCCTCCA
CGGAGGCGTAGGTGTTATTGGGATGGAGGTCGGTGTGGCA
GCGGACAAAGAGGGCCAGGAACTGGGGGTAGCTCATCTTA
AAGTACTTTAGTATATCGCGACTTGATCGTGGGAATGTAG
CAGGCGCTAATATCCAACACAATATCACAGCCCATCAACA
GGAGGTCAGTGTCTGTGGTGTACACGTACGCGACCGTGTT
GGTGTGATAGAGGTTGGCGCAGGCATCGTCCGCCTCCAGC
TGACCCGAGCTAATGTAGGGACCCCAGGGCCCGGAGAACG
CGAATACAGAACAGATGCGCCAGACGCAGGGCCGGCTTCG
AGGGCGCGGCGGACGGCAGCGCGGCTCCGGCCCGGCCGTC
CCCCGGGTCCCCGAGGCCAGAGAGGTGCCGCGCCGGCGCA
TGTTGGAAAAGGCAGAGCTGGGTCTGGAGTCGGTGATGGG
GGAAGGCGGTGGAGAGGCGTCCACGTCACTGGCCTCCTCG
TCCGTCCGGCATTGGGCCGTCGTGCGGGCCAGGATGGCCT
TGGCTCCAAACACAACCGGCTCCATACAATTGACCCCGCG
ATCGGTAACGAAGATGGGGAAAAGGGACTTTTGGGTAAAC
ACCTTTAATAAGCGACAGAGGCAGTGTAGCGTAATGGCCT
CGCGGTCGTAACTGGGGTAGCGGCGCTGATATTTGACCAC
CAACGTGTACATGACGTTCCACAGGTCCACGGCGATGGGG
GTGAAGTACCCGGCCGGGGCCCCAAGGCCCTGGCGCTTGA
CCAGATGGTGTGTGTGGGCAAACTTCATCATCCCGAACAA
ACCCATGTCAGGTCGATTGTAACTGCGGATCGGCCTAACT
AAGGCGTGGTTGGTGCGACGGTCCGGGACACCCGAGCCTG
TCTCTCTGTGTATGGTGACCCAGACAACAACACCGACACA
AGAGGACAATAATCCGTTAGGGGACGCTCTTTATAATTTC
GATGGCCCAACTCCACGCGGATTGGTGCAGCACCCTGCAT
GCGCCGGTGTGGGCCAAACTTCCCCCCGCTCATTGCCTCT
TCCAAAAGGGTGTGGCCTAACGAGCTGGGGGCGTATTTAA
TCAGGCTAGCGCGGCGGGCCTGCCGTAGTTTCTGGCTCGG
TGAGCGACGGTCCGGTTGCTTGGGTCCCCTGGCTGCCAGC
AAAACCCCACCCTCGCAGCGGCATACGCCCCCTCCGCGTC
CCGCACCCGAGACCCCGGCCCGGCTGCCCTCACCACCGAA
GCCCACCTCGTCACTGTGGGGTGTTCCCAGCCCGCATTGG
GATGACGGATTCCCCTGGCGGTGTGGCCCCCGCCTCCCCC
GTGGAGGACGCGTCGGACGCGTCCCTCGGGCAGCCGGAGG
AGGGGGCGCCCTGCCAGGTGGTCCTGCAGGGCGCCGAACT
TAATGGAATCCTACAGGCGTTTGCCCCGCTGCGCACGAGC
CTTCTGGACTCGCTTCTGGTTATGGGCGACCGGGGCATCC
TTATCCATAACACGATCTTTGGGGAGCAGGTGTTCCTGCC
CCTGGAACACTCGCAATTCAGTCGGTATCGCTGGCGCGGA
CCCACGGCGGCGTTCCTGTCTCTCGTGGACCAGAAGCGCT
CCCTCCTGAGCGTGTTTCGCGCCAACCAGTACCCGGACCT
ACGTCGGGTGGAGTTGGCGATCACGGGCCAGGCCCCGTTT
CGCACGCTGGTTCAGCGCATATGGACGACGACGTCCGACG
GCGAGGCCGTTGAGCTAGCCAGCGAGACGCTGATGAAGCG
CGAACTGACGAGCTTTGTGGTGCTGGTTCCCCAGGGAACC
CCCGACGTTCAGTTGCGCCTGACGAGGCCGCAGCTCACCA
AGGTCCTTAACGCGACCGGGGCCGATAGTGCCACGCCCAC
CACGTTCGAGCTCGGGGTTAACGGCAAATTTTCCGTGTTC
ACCACGAGTACCTGCGTCACATTTGCTGCCCGCGAGGAGG
GCGTGTCGTCCAGCACCAGCACCCAGGTCCAGATCCTGTC
CAACGCGCTCACCAAGGCGGGCCAGGCGGCCGCCAACGCC
AAGACGGTGTACGGGGAAAATACCCATCGCACCTTCTCTG
TGGCGTCGACGATTGCAGCAGCGGGCGGTGCTCCGGCGAC
TGCAGGTCGCCGGGGGCACCCTCAAGTTCTTCCTCACGAC
CCCCGTCCCCAGTCTGTGCGTCACCGCCACCGGTCCCAAC
GCGGTATCGGCGGTATTTCTCCTGAAACCCCAGAAGATTT
GCCTGGACTGGCTGGGTCATAGCCAGGGGTCTCCTTCAGC
CGGGAGCTCGGCCTCCCGGGCCTCTGGGAGCGAGCCAACA
GACAGCCAGGACTCCGCGTCGGACGCGGTCAGCCACGGCG
ATCCGGAAGACCTCGATGGCGCTGCCCGGGCGGGAGAGGC
GGGGGCCTCGCACGCCTGTCCGATGCCGTCGTCGACCACG
CGGGTCACTCCCACGACCAAGCGGGGGCGCTCGGGGGGCG
AGGATGCGCGCGCGGACACGGCCCTAAAGAAACCTAAGAC
GGGGTCGCCCACCGCACCCCCGCCCACAGATCCAGTCCCC
CTGGACACGGAGGACGACTCCGATGCGGCGGACGGGACGG
CGGCCCGTCCCGCCGCTCCAGACGCCCGGAGCGGAAGCCG
TTACGCGTGTTACTTTCGCGACCTCCCGACCGGAGAAGCA
AGCCCCGGCGCCTTCTCCGCCTTCCGGGGGGGCCCCCAAA
CCCCGTATGGTTTTGGATTCCCCTGACGGGGCGGGGCCTT
GGCGGCCGCCCAACTCTCGCACCATCCCGGGGTAATGTAA
ATAAACTTGGTATTGCCCAACACTCTCCCGCGTGTCGCGT
GTGGTTCATGTGTGTGCCTGGCGTCCCCCACCCTCGGGGT
CGTGTATTTCCTTTCCCTGTCCTTATAAAAGCCGTATGTG
GGGCGCTGACGGAACCACCCCGCGTGCCATCACGGCCAAG
GCGCGGGATGCTCCGCAACGACAGCCACCGGGCCGCGTCC
CCGGAGGACGGCCAGGGACGGGTCGACGACGGACGGCCAC
ACCTCGCGTGCGTGGGGGCCCTGGCGCGGGGGTTCATGCA
TATCTGGCTTCAGGCCGCCACGCTGGGTTTTGCGGGATCG
GTCGTTATGTCGCGCGGGCCGTACGCGATGCCGCGTCTGG
GGCGTTCGCCGTCGGGGCGCCGTGCTGGGCTTTATGCGCG
CACCCCCCCCCTCGCGCGGCCCACCGCGCGGATATACGCC
TGGCTCAAACTGGCGGCCGGTGGAGCGGCCCTTGTTCTGT
GGAGTCTCGGGGAGCCCGGAACGCAGCCGGGGGCCCCGGG
CCCGGCCACCCAGTGCCTGGCGCTGGGCGCCGCCTATGCG
GCGCTCCTGGTGCTCGCCGATGACGTCTATCCGCTCTTTC
TCCTCGCCCCGGGGCCCCTGTTCGTCGGCACCCTGGGGAT
GGTCGTCGGCGGGCTGACGATCGGAGGCAGCGCGCGCTAC
TGGTGGATCGGTGGGCCCGCCGCGGCCGCCTTGGCCGCGG
CGGTGTTGGCGGGCCCGGGGGCGACCACCGCCAGGGCTGC
TTCTCCAGGGCGTGCCCCGACCACCGCCGCGTCTGCGTCA
TCGTCGCAGGCGAGTCTGTTTCCCGCCGCCCCCCGGAGGA
CCCAGAGCGACCCGGGGACCCCGGGCCACCGTCCCCCCCG
ACACCCCAACGATCCCAGGGGCCGCCGGCCGATGAGGTCG
CACCGGCCGGGGTAGCGCGGCCCGAAAACGTCTGGGTGCC
CGTGGTCACCTTTCTGGGGGCGGGCGCGCTCGCCGTCAAG
ACGGTGCGAGAACATGCCCGGGAAACGCCGGGCCCGGGCC
TGCCGCTGTGGCCCCAGGTGTTTCTCGGAGGCCATGTGGC
GGTGGCCCTGACGGAGCTGTGTCAGGCGCTTATGCCCTGG
GACCTTACGGACCCGCTGCTGTTTGTTCACGCCGGACTGC
AGGTCATCAACCTCGGGTTGGTGTTTCGGTTTTCCGAGGT
TGTCGTGTATGCGGCGCTAGGGGGTGCCGTGTGGATTTCG
TTGGCGCAGGTGCTGGGGCTCCGGCGTCGCCTGCACAGGA
AGGACCCCGGGGACGGGGCCCGGTTGGCGGCGACGCTTCG
GGGCCTCTTCTTCTCCGTGTACGCGCTGGGGTTTGGGGTG
GGGGCGCTGCTGTGCCCTCCGGGGTCAACGGGCGGGTGGT
CGGGCGATTGATATATTTTTCAATAAAAGGCATTAGTCCG
AAACCGCCGGTGTGTGATGATTTCGCCATAACACCCAAAC
CCCGGATGGGGCCCGGGAAAATTCCGGAAGGGGACACGGG
CTACCCTCACTACCGAGGGCGCTTGGTCGGGAGGCCGCAT
CGAACGCACACCCCCATCCGGTGGTCCGTGTGGAGGTCGT
TTTTCAGTGCCCGGTCTCGCTTTGCCGGGAACGCTAGCCG
ATCCCTCGCGAGGGGGAGGCGTCGGGCATGGCCCCGGGGC
GGGTGGGCCTTGCCGTGGTCCTGTGGAGCCTGTTGTGGCT
CGGGGCGGGGGTGGCCGGGGGCTCGGAAACTGCCTCCACC
GGGCCCACGATCACCGCGGGAGCGGTGACAAACGCGAGCG
AGGCCCCCACATCGGGGTCCCCCGGGTCAGCCGCCAGCCC
GGAAGTCACCCCCACATCGACCCCAAACCCCAACAATGTC
ACACAAAACAAAACCACCCCCACCGAGCCGGCCAGCCCCC
CAACAACCCCCAAGCCCACCTCCACGCCCAAAAGCCCCCC
CACGCCCCCCCCCGACCCCAAACCCAAGAACAACACCCCC
CCCGCCAAGTCGGGCCGCCCCACTAAACCCCCCGGGCCCG
TGTGGTGCGACCGCCGCGACCCATTGGCCCGGTACGGCTC
GCGGGTGCAGATCCGATGCCGGTTTCGGAATTCCACCCGC
ATGGAGTTCCGCCTCCAGATATGGCGTTACTCCATGGGTC
CGTCCCCCCCAATCGCTCCGGCTCCCGACCAGAGGAGGTC
CTGACGAACATCCCGCCCCACCCGGGGGACTCCTGGTGTA
CGACAGCGCCCCCAACCTGACGGACCCCCACGTGTCTGGG
CGGAGGGGGCCGGCCCGGGCGCCGACCCTCCGTTGTATTC
GTCCCGGGCCGCTGCCGACCCAGCGGCTGATTATCGGCGA
GGTGACGCCCGCGACCCAGGGAATGTATTACTTGGCCTGG
GGCCGGATGGACAGCCCGCACGAGTACGGGACGTGGGTGC
GCGTCCGCATGTTCCGCCCCCCGTCTCTGACCCTCCAGCC
CCACGCGGTGATGGAGGGTCAGCCGTTCAAGGCGACGTGC
ACGGCCGCCGCCTACTACCCGCGTAACCCCGTGGAGTTTG
TCTGGTTCGAGGACGACCGCCAGGTGTTTAACCCGGGCCA
GATCGACACGCAGACGCACGAGCACCCCGACGGGTTCACC
ACAGTCTCTACCGTGACCTCCGAGGCTGTCGGCGGCCAGG
TCCCCCCGCGGACCTTCACCTGCCAGATGACGTGGCACCG
CGACTCCGTGATGTTCTCGCGACGCAATGCCACCGGGCTG
GCCCTGGTGCTGCCGCGGCCAACCATCACCATGGAATTTG
GGGTCCGGCATGTGGTCTGCACGGCCGGCTGCGTCCCCGA
GGGCAAAAGAGGGAGTGACGTTGCCTGGTTCCTGGGGGAC
GACCCCTCACCGGCGGCTAAGTCGGCCGTTACGGCCCAGG
AGTCGTGCGACCACCCCGGGCTGGCTACGGTCCGGTCCAC
CCTGCCCATTTCGTACGACTACAGCGAGTACATCTGTCGG
TTGACCGGATATCCGGCCGGGATTCCCGTCTAGAGCACCA
CGGCAGTCACCAGCCCCCACCCAGGGACCCCACCGAGCGG
CAGGTGATCGAGGCGATCGAGTGGGTGGGGATTGGAATCG
GGGTTCTCGCGGCGGGGGTCCCGGTCGTAACGGCAATCGT
GTACGTCGTCCGCACATCACAGTCGCGGCAGCGTCATCGG
CGGTAACGCGAGACCCCCCCGTTACCTTTTTAATATCTAT
ATAGTTTGGTCCCCCTCTATCCCGCCCACCGCTGGGCGCT
ATAAAGCCGCCACCCTCTCTTCCCTCAGGTCATCCTTGGT
CGATCCCGAACGACACACGGCGTGGAGCAAAACGCCTCCC
CCTGAGCCGCTTTCCTACCAACACAACGGCATGCCTCTGC
GGGCATCGGAACACGCCTACCGGCCCCTGGGCCCCGGGAC
ACCCCCCATGCGGGCTCGGCTCCCCGCCGCGGCCTGGGTT
GGCGTCGGGACCATCATCGGGGGAGTTGTGATCATTGCCG
CGTTGGTCCTCGTGCCCTCGCGGGCCTCGTGGGCACTTTC
CCCATGCGACAGCGGATGGCACGAGTTCAACCTCGGGTGC
ATATCCTGGGATCCGACCCCCATGGAGCACGAGCAGGCGG
TCGGCGGCTGTAGCGCCCCGGCGACCCTGATCCCCCGCGC
GGCTGCCAAACAGCTGGCCGCCGTCGCACGCGTCCAGTCG
GCAAGATCCTCGGGCTACTGGTGGGTGAGCGGAGACGGCA
TTCGGGCCTGCCTGCGGCTCGTCGACGGCGTCGGCGGTAT
TGACCAGTTTTGCGAGGAGCCCGCCCTTCGCATATGCTAC
TATCCCCGCAGTCCCGGGGGCTTTGTTCAGTTTGTAACTT
CGACCCGCAACGCGCTGGGGCTGCCGTGAGGCGCGTGTAC
TGCGGTCTGTCTCGTCTCCTCTTCTCCCCTTCCCTCCCCC
TCCGCATCCCAGGATCACACCGGCCAACGAGGGTTGGGGG
GGGGTCCGGCACGGACCCAAAATAATAAACACACAATCAC
GTGCGATAAAAAGAACACGCGGTCCCCTGTGGTGTTTTTG
GTTATTTTTATTAAATCTCGTCGACAAACAGGGGGAAAGG
GGCGTGGTCTAGCGACGGCAGCACGGGCGGAGGCGTTCAC
CGGCTCCGGCGTCCTTCGCGTTTAAGCTTGGTCAGGAGGG
CGCTCAGGGCGGCGACGTTGGTCGGGCCGTCGTTGGTCAG
GGCGTTGGCTCGATGGCGGGCGAGGACGGGCGAGGGGCTC
AACGGCGGGGGCGGGGGTCCGGTGCGGCCCGGGGGGGAAA
ATAGGGCGGATCCCCCCCAGTCGTACAGGGGGTTTTCCGC
CTCAATGTACGGGGAGGCCGGCGCTGCATTCGCCGTGTTC
ACGCAGACGTTTTCGTAGACCCGCATCCATGGTATTTCCT
CGTAGACACGCCCCCCGTCCTCGCTCCCCGCCGTATATTG
ACTCGTCGTCCTCGTAGGGGGCGTGCCGTTCGCGGGCCGA
GGCGGCGTGGGTGGCTTTGCGGCGGGCGTCGTCGTCGTCG
TCGTCGGCCGTCAGATACGTGGCTTCCATCTGGTCGGGTT
CTCCCTCCGGGGCGGGTCCCCACACCCGTGGCCGATCGAG
GCTCCCCAGAGACGCGCGCCGGACAAGAAGGGGGCACGTC
GCCGCCGGCGGTCGCCTGTCGGGTCCCGCGACGTTACGGG
CCGGGAGGCGCGGGGGCACCCCCCCCATGTGCGTGTAATA
CGTGGCCGGCTGTGCGGCCGCAGCGGGGGGCTCGGCGACC
GGGTCGTCCGCATCCGGAAGCGGGGGCCCCGCGCCGTCCG
CACGGCGCCTCCGGAACCGCCGGGTGGACGGCGCGGGGGT
CGAGTGTAGGCGAGGTCGGGGGAGGGGCGGGGGCTCGTTG
TCGCGCCGCGCCCGCTGAATCTTTTCCCGACAGGTCCCAC
CCCCCGCGCGATGCCCCCCCGGGCCGCGGGCCATGTCGTC
CGGGGGAGGCCCCGCGGACCACGTCGTCCGGCGAGACGCC
ACGAGCCGCAGGATGGACTCGTAGTGGAGCGACGGCGCCC
CGCTGCGGAGCAGATCCGCGGCCAGGGCGGCCCCGAACCA
AGCCTTGATGCTCACTCCATCCGGGCCCAGCTGGGGGCGG
TCATCGTGGGGAACAGGGGGGCGGTGGTCCGACAGAAACG
CTCCTGGCTGTCCACCGCGGCCCGCAGATACTCGTTGTTC
AGGCTGTCGGTGGCCCAGACGCCGTACCCGGTGAGGGTCG
CGTTGATGATATACTGGGCGTGGTGATGGACGATCGACAG
AACCTCCACCGTGGATACCACGGTATCCACGGTCCCGTAC
GTACCGCCGCTCCGCTTGCCGGTCTGCCACAGGTTGGCTA
GGCACGTCAGGTGGCCCAGGACGTCGCTGACCGCCGCCCT
GAGCGCCATGCACTGCATGGAGCCGGTCGTGCCGCTGGGA
CCCCGGTCCAGATGGCGCGCGAACGTTTCCGCGGGCGCCT
CCGGGCTGCCGCCGAGCGGGAGGAACCGGCGATTGGAGGG
ACTCAGCCGGGACATACGTGCTTGTCCGTCGTCCACAGCA
TCCAGGACGCCCACCGGTACAGCACGGGGACGTAGGCCAG
GAGCTCGTTGAGCCGCAGTGCGGTGTCGGTGCTGGGGCGG
CTTGGGTCCGCCGGGCGCAAGAACATGTCGCTGATCCGAT
GGAGGGCGTCGCGCAGGCCGGCCACGGTGGCGGCGTACTT
GGCCGCCGCGGCCCCGCTCTTGACGGGGTGCGCGCCAGCA
GCTTTGGCGCCAGGGTGGGCCGCAGCAGCACGTGAAGGCT
GGGGTCGCAGTCGCCCACGGGGTCCTCGGGGACGTCCAGG
CCGCTGGGCACCACCGTCTGCAGGTACTTCCAGTACTGCG
TGAGGATGGAGAGGAGAAAAGGGCCGCCGGGCAGCTCCAC
CTCGCCCAGCGCCTGGGTGGCGGCCGAAGCGTAGTGCCGG
ATGTACCGTAGTGCGGGTCGCTGGCGAGCCCGTCCACGAT
CAAACTCTCGGGAACCGTGTTGTGTTGCCGCGCGGCCAAC
CGGACGCTGCGATCGGTGCAGGTCAGAAACGCCGGCTGCG
CGTCGTCGGAGCGCTGCCGCAAGGCGCCCACGGCCGCGCT
AAGGAGCCCCTCCGGGGTGGGGAGCAGACACCCGCCGAAG
ATGCGCCGCTCGGGAACGCCCGCGTTGTCGCCGCGGATCA
GGTTGGCAGGCGTCAGGCACCGCGCCAGCCGCAGGGAGCT
CGCGCCGCGCGTCCGGCGCTGCATGGTGACGCCCGTTCGG
TCGGGACCCGCCGGTCGGAGTTATGCCGCGTCCAGGGCCA
TCGGGGCGCTTTTTATCGGGAGGAGCTTATGGGCGTGGCG
GGCCTCCCAGCCCGGTCGCGCGCCTCCCCGACACGTGCGC
CCGCAGGGCGGCGGCCCCCTCGTCTCCCATCAGCAGTTTC
CTAAACTGGGACATGATGTCCACCACGCGGACCCGCGGGC
CCAACACGGACCCGCCGCTTACGGGGGCGGGGGGGAAGGG
CTCCAGGTCCTTGAGCAGAAAGGCGGGGTCTGCCGTCCCG
GACACGGGGGCCCGGGGCGCGGAGGAGGCGGGGCGCAGAT
CCACGTGCTCCGCGGCCGCGCGGACGTCCGCCCAGAACTT
GGCGGGGGTGGTGCGCGCGTACAGGGGCTGGGTCGCTCGG
AGGACACACGCGTAGCGCAGGGGGGTGTACGTGCCCACCT
CGGGGGCCGTGAATCCCCCGTCAAACGCGGCCAGTGTCAC
GCACGCCACCACGGTGTCGGCAAAGCCCAGCAGCCGCTGC
AGGACGAGCCCGGCGGCCAGAATGGCGCGCGTGGTCGCAG
CGTCGTCCCGGCGCCGGTGCGCGTCCCCGCACGCCCGGGC
GTACTTTAAGGTCACTGTCGCCAGGGCCGTGTGCAGCGCG
TACACCGCAGCGCCCAGCACGGCGTTGAGCCCGCTGTTGG
CGAGCAGCCGGCGCGCTGCGGTGTCGCCCAGCGCCTCGTG
CTCGGCCCCCACGACCGCGGGGCTTCCCAGGGGCAGGGCG
CGAAACAGCTCCTCCCGCGCCACGTCCGCAAAGGCGGGGT
GGTGCACGTGCGGGTGCAGGCGCGCCCCCACGACCACCGA
GAGCCACTGGACCGTCTGCTCCGCCATCACCGCCAACACA
TCCAGCACGCGCCCCAGGAAGGCGGCCTCCCGCGTCAAAA
CGCACCGGACGGCGTCGGGATTGAAGCGGGCGAGCAGGGC
CCCGGTGGCCAGGTACGTCATGCGGCCGGCATAGCGGGCG
GCCACGCGACAGTCGCGGTCCAGCAGCGCGCGCACCCCGG
GCCAGTACAGCAGGGACCCCAGCGAGCTGCGAAACACCGC
GGCGTCGGGGCCGGATTGGGGGGACACTAACCCCCCCGCG
CTCAGTAACGGCACGGCCGCGGCCCCGACGGGACGCCCGC
CTCTCGCGAACTGCCGCCTCAGCTCGGCAGCCCTGTCGTC
CAGGTCCGACCCGCGCGCCTCTGCGTGAAGGCGCGTCCCG
CACACCCACCCGTTGATGGCCAGCCGCACGACGGCATCCG
CCAAAAAGCTCATCGCCTGGGCGGGGCTGGTTTTTGTTCG
ACGATCCGTCAGGTCAAGAATCCCATCGCCCGTGATATAC
CAGGCCAACGCCTCGCCCTGCTGCAGGGTTTGGCGGAAAA
ACACCGCGGGGTTGTCGGGGGAGGCGAAGTGCATGACCCC
CACGCGCGATAACCCGAACGCGCTATCCGGACACGGGTAA
AACCCGGCCGGATGCCCCAGGGCTAGGGCGGAGCGCACGG
ACCGTCACACACGGCAACCTGAGGGGCCAGTCGATCCAAC
GGGAATGCCGCCCGGAGCTCCGGGCCCGGCCCGCGTCCCT
CCAGACCCTCCCCTTGGGCGGGGAACGGGCCCCGCCGCCG
TCCTCCGGCCCGACGTCTTCCGGGTAGTCGTCCTCCTCGT
ACTGCAGTTCCTCTAGGAACAGCGGCGACGGCGCCCCCCG
CGAACCGCCGACCCGCCCCAAAATAGCCCGCGCGTCGACG
GGACCCAGGTATCCCCCCTGCCGGGCCTGCGGAGGACCGC
GGGGAACCTCATCATCATCGTCCAGGCGACCGCGCACCGA
CTGGCTACGGGCCGCATCGGGCCCGGGGCGCTGCCGGGAC
GCTCGGCGATGGGATGAGGGCGGGGCTTCCGACGCGCGCC
GTCGTCGGGCTCGCGGGCCTTCCCGTCGACGGCGCACGGG
CGGCTCGTCGCCCGCCATCTCCTCCAGAGCCTCTAGCTCG
CTGTCGTCATCCCCGCGGAACACCGCACGCAGGTACCCCA
TGAACCCCCCCCATCGCCCGCTGGCTCGTCCGCCACGGGC
GAGGCGCGGGGGCGGGTGGATGCGCGCCTCCTGCGCCCCG
CGGGTTCGCGAGCCGACATGGTGGCGATAGACGCGGGTAT
CGGATGTCCGCTACCCCCCAAAAAAGAAAAAGACCCCACA
GCGCGGATGGAGGTCGGGGTAGGTGCCGCCGGACCCCCTC
GCGATGGGAATGGACGGGAGCGACGGGGCCGGCGCAAAAA
ACGCAGTATCTCCCGCGAAGGCTACCCGCCGCCCCAGCCC
CCGGCCAAATGCGGAAACGGTCCCGCGCTCTCGCCTTTAT
ACGCGGGCCGCCCTGCGACACAATCACCCGTCCGTGGTTT
CGAATCTACACGACAGGCCCGCAGACGCGGCTAACACACA
CGCCGGCAACCCAGACCCCAGTGGGTTGGTTGCGCGGTCC
CGTCTCCTGGCTAGTTCTTTCCCCCACCACCAAATAATCA
GACGACAACCGCAGGTTTTTGTAATGTATGTGCTCGTGTT
TATTGTGGATACGAACCGGGGACGGGAGGGGAAAACCCAG
ACGGGGGATGCGGGTCCGGTCGCGCCCCCTACCCACCGTA
CTCGTCAATTCCAAGGGCATCGGTAAACATCTGCTCAAAC
TCGAAGTCGGCCATATCCAGAGCGCCGTAGGGGGCGGAGT
CGTGGGGGGTAAATCCCGGACCCGGGGAATCCCCGTCCCC
CAACATGTCCAGATCGAAATCGTCTAGCGCGTCGGCATGC
GCCATCGCCACGTCCTCGCCGTCTAAGTGGAGCTCGTCCC
CCAGGCTGACATCGGTCGGGGGGGCCGTCGACAGTCTGCG
CGTGTGTCCCGCGGGGAGAAAGGACAGGCGCGGAGCCGCC
AGCCCCGCCTCTTCGGGGGCGTCGTCGTCCGGGAGATCGA
GCAGGCCCTCGATGGAGACCCGTAATTGTTTTTCGTACGC
GCGCGGCTGTACGCGTGTTCCCGCATGACCGCCTCGGAGG
GCGAGGTCGTGAAGCTGGAATACGAGTCCAACTTCGCCCG
AATCAACACCATAAAGTACCCAGAGGCGCGGGCCTGGTTG
CCATGCAGGGTGGGAGGGGTCGTCAACGGCGCCCCTGGCT
CCTCCGTAGCCGCGCTGCGCACCAGCGGGAGGTTAAGGTG
CTCGCGAATGTGGTTTAGCTCCCGCAGCCGGCGGGCCTCG
ATTGGCACTCCCCGGACGGTGAGCGCTCCGTTGACGAACA
TGAAGGGCTGGAACAGACCCGCCAACTGACGCCAGCTCTC
CAGGTCGCAACAGAGGCAGTCAAACAGGTCGGGCCGCATC
ATCTGCTCGGCGTACGCGGCCCATAGGATCTCGCGGGTCA
AAAATAGATACAAATGCAAAAACAGAACACGCGCCAGACG
AGCGGTCTCTCGGTAGTACCTGTCCGCGATCGTGGCGCGC
AGCATTTCTCCCAGGTCGCGATCGCGTCCGCGCATGTGCG
CCTGGCGGTGCAGCTGCCGGACGCTGGCGCGCAGGTACCG
GTACAGGGCCGAGCAGAAGTTGGCCAACACGGTTCGATAG
CTCTCCTCCCGCGCCCGTAGCTCGGCGTGGAAGAAACGAG
AGAGCGCTTCGTAGTAGAGCCCGAGGCCGTCGCGGGTGGC
CGGAAGCGTCGGGAAGGCCACGTCGCCGTGGGCGCGAATG
TCGATTTGGGCGCGTTCGGGGACGTACGCGTCCCCCCATT
CCACCACATCGCTGGGCAGCGTTGATAGGAATTTACACTC
CCGGTACAGGTCGGCGTTGGTCGGTAACGCCGAAAACAAA
TCCTCGTTCCAGGTATCGAGCATGGTACATAGCGCGGGGC
CCGCGCTAAAGCCCAAGTCGTCGAGGAGACGGTTAAAGAG
GGCGGCGGGGGGGACGGGCATGGGCGGGGAGGGCATGAGC
TGGGCCTGGCTCAGGCGCCCCGTTGCGTACAGCGGAGGGG
CCGCCGGGGTGTTTTTGGGACCCCCGGCCGGGCGGGGGGG
TGGTGGCGAAGCGCCGTCCGCGTCCATGTCGGCAAACAGC
TCGTCGACCAAGAGGTCCATTGGGTGGGGTTGATACGGGA
AAGACGATATCGGGCTTTTGATGCGATCGTCCCCGCCCGC
CCCGCGAGTGTGGGACGCCCGACGGCGCGGGAAGAGAAAA
ACCCCCAAACGCGTTAGAGGACCGGACGGACCTTATGGGG
GGAAGTGGGCAGCGGGAACCCCGTCCGTTCCCGAGGAATG
ACAGCCCGTGGTCGCCACCCCGCATTTAAGCAACCCGCAC
GGGCCGCCCCGTACCTCGTGACTTCCCCCCACATTGGCTC
CTGTCACGTGAAGGCAAACCGAGGGCGGCTGTCCAACCCA
CCCCCCGCCACCCAGTCACGGTCCCCGTCGGATTGGGAAA
CAAAGGCACGCAACGCCAACACCGAATGAACCCCTGTTGG
TGCTTTATTGTCTGGGTACGGAAGTTTTTCACTCGACGGG
CCGTCTGGGGCGAGAAGCGGAGCGGGCTGGGGCTCGAGGT
CGCTCGGTGGGGCGCGACGCCGCAGAACGCCCTCGAGTCG
CCGTGGCCGCGTCGACGTCCTGCACCACGTCTGGATTCAC
CAACTCGTTGGCGCGCTGAATCAGGTTTTTGCCCTCGCAG
ACCGTCACGCGGATGGTGGTGATGCCAAGGAGTTCGTTGA
GGTCTTCGTCTGTGCGCGGACGCGACATGTCCCAGAGCTG
GACCGCCGCCATCCGGGCATGCATGGCCGCCAGGCGCCCA
ACCGCGGCGCAGAAGACGCGCTTGTTAAAGCCGGCCACCC
GGGGGGTCCATGGCGCGTCGGGGTTTGGGGGGGCGGTGCT
AAAGTGCAGCTTTCTGGCCAGCCCCTGCGCGGGTGTCTTG
GATCGGGTTGGCGCCGTCGACGCGGGGGCGTCTGGGAGTG
CGGCGGATTCTGGCTGGGCCGATTTCCTGCCGCGGGTGGT
CTCCGCCGCCGGGGCCGCGGGGGCCTTAGTCGCCACCCGC
TGGGTTCGGGGGGCCCGGGGGGCGGTGGTGGGTGGCGTCC
GGCCCCTCCGGACCCAGCGGGCGGCGGGGGCGCCCGCGCA
GGCCCCGGGGCGGACAAAACCGCCCCGGAAACGGGACGCC
GCGTCCGGGGGACCTCCGGGTGTTCGTCGTCTTCGGATGA
CGAGCCCCCGTAGAGGGCATAATCCGACTCGTCGTACTGG
ACGAAACGGACCTCGCCCCTTGGGCGCGCGCGTGTCTGTA
GGGCGCCACGGCGGGAGGTGTCAGGCGGACTATCGGGACT
CGCCATACATGAAGACGGGGGTAGTACAGATCCTCGTACT
CATCGCGCGGAACCTCCCGCGGACCCGACTTCACGGAGCG
GCGAGAGGTCATGGTTCCACGAACACGCTAGGGTCGGATG
CGCGGACAATTAGGCCTGGGTTCGGACGGCGGGGGTGGTG
CAGGTGTGGAGAGGTCGAGCGATAGGGGCGGCCCGGGAGA
GAAGAGAGGGTCCGCAAAACCCACTGGGGATGCGTGAGTG
GCCCTCTGTGGGCGGTGGGGGAGAGTCTTATAGGAAGTGC
ATATAACCACAACCCATGGGTCTAACCAATCCCCAGGGGC
CAAGAAACAGACACGCCCCAAACGGTCTCGGTTTCCGCGA
GGAAGGGGAAGTCCTGGGACACCCTCCACCCCCACCCCTC
ACCCCACACAGGGCGGGTTCAGGCGTGCCCGGCAGCCAGT
AGCCTCTGGCAGATCTGACAGACGTGTGCGATAATACACA
CGCCCATCGAGGCCATGCCTACATAAAAGGGCACCAGGGC
CCCCGGGGCAGACATTTGGCCAGCGTTTTGGGTCTCGCAC
CGCGCGCCCCCGATCCCATCGCGCCCGCCCTCCTCGCCGG
GCGGCTCCCCGTGCGGGCCCGCGTCTCCCGCCGCTAAGGC
GACGAGCAAGACAAACAACAGGCCCGCCCGACAGACCCTT
CTGGGGGGGCCCATCGTCCCTAACAGGAAGATGAGTCAGT
GGGGATCCGGGGCGATCCTTGTCCAGCCGGACAGCTTGGG
TCGGGGGTACGATGGCGACTGGCACACGGCCGTCGCTACT
CGCGGGGGCGGAGTCGTGCAACTGAACCTGGTCAACAGGC
GCGCGGTGGCTTTTATGCCGAAGGTCAGCGGGGACTCCGG
ATGGGCCGTCGGGCGCGTCTCTCTGGACCTGCGAATGGCT
ATGCCGGCTGACTTTTGTGCGATTATTCACGCCCCCGCGC
TATCCAGCCCAGGGCACCACGTAATACTGGGTCTTATCGA
CTCGGGGTACCGCGGAACCGTTATGGCCGTGGTCGTAGCG
CCTAAAAGGACGCGGGAATTTGCCCCCGGGACCCTGCGGG
TCGACGTGACGTTCCTGGACATCCTGGCGACCCCCCCGGC
CCTCACCGAGCCGATTTCCCTGCGGCAGTTCCCGCAACTG
GCGCCCCCCCTCAACGGGGCCGGGATACGCGCAGATCCTT
GGTTGGAGGGGGCGCTCGGGGACCCAAGCGTGACTCCTGC
CCTACCGGCGCGACGCGAGGGCGGTCCCGCGCCCATGCCG
GCGAGCTGACGCCGGTTCAGACGGAACACGGGGACGGCGT
ACGAGAAGCCATCGCCTTCCTTCCAAAACGCGAGGAGGAT
GCCGGTTTCGACATTGTCGTCCGTCGCCCGGTCACCGTCC
CGGCAAACGGCACCACGGTCGTGCAGCCATCCCTCCGCAT
GCTCCACGCGGACGCCGGGCCCGCGGCCTGCTATGTGCTG
GGGCGGTCGTCGCTCAACGCCCGCGGCCTCCTGGTCGTTC
CTACGCGCTGGCTCCCCGGGCACGTATGTGCGTTTGTTGT
TTACAACCTTACGGGGGTTCCTGTGACCCTCGAGGCCGGC
GCCAAGGTCGCCCAGCTCCTGGTTGCGGGGGCGGACGCTC
TTCCTTGGATCCCCCCGGACAACTTTCACGGGACCAAAGC
GCTTCGAAACTACCCCAGGGGTGTTCCGGACTCAACCGCC
GAACCCAGGAACCCGCCGCTCCTGGTGTTTACGAACGAGT
TTGACGCGGAGGCCCCCCCGAGCGAGCGCGGGACCGGGGG
TTTTGGCTCTACCGGTATTTAGCCCATAGCTTGGGGTTCG
TTCCGGGCAATAAAAAACGTTTGTATCTCATCTTTCCTGT
GTGTAGTTGTTTCTGTTGGATGCCTGTGGGTCTATCACAC
CCGCCCCTCCATCCCACAAACACAGAACACACGGGTTGGA
TGAAAACACGCATTTATTGACCCAAAACACACGGAGCTGC
TCGAGATGGGCCAGGGCGAGGTGCGGTTGGGGAGGCTGTA
GGTCTGGGAACGGACACGCGGGGACACGATTCCGGTTTGG
GGTCCGGGAGGGCGTCGCCGTTTCGGGCGGCAGGCGCCAG
CGTAACCCGGGGGCGGCGTGTGGGGGTGCCCCAAGGAGGG
CGCCTCGGTCACCCCAAGCCCCCCCGAGCGGGTCCCCCGG
CAACCCCGAAGGCGGAGAGGCCAAGGGCCCGGGCGGCGAT
GGCCACATCCTCCATGACCACGTCGCTCTCGGCCATGCTC
CGAATAGCCTGGGAGACGAGCACATCCGCGGACTTGTCAG
CCGCCCCCACGGACATGTACATCTGCAGGATGGTGGCCAT
ACACGTGTCCGCCAGGCGCCGCATCTTGTCCTGATGGGCC
GCCACGGCCCCGTCGATCGTGGGGGCCTCGAGCCCGGGGG
GTGGCGCGCCAGTCGTTCTAGGTTCACCATGCAGGCGTGG
TACGTGCGGGCCAAGGCGCGGGCCTTCACGAGGCGTCGGG
TGTCGTCCAGGGACCCCAGGGCGTCATCGAGCGTGATGGG
GGCGGGAGTAGCCCGCCCCTCCATCCCACAAACACAGAAC
ACACGGGTTGGATGAAAACACGCATTTATTGACCCAAAAC
ACACGGAGCTGCTCGAGATGGGCCAGGGCGAGGTGCGGTT
GGGGAGGCTGTAGGTCTGGGAACGGACACGCGGGGACACG
ATTCCGGTTTGGGGTCCGGGAGGGCGTCGCCGTTTCGGGC
GGCAGGCGCCAGCGTAACCTCCGGGGGCGGCGTGTGGGGG
TGCCCCAAGGAGGGCGCCTCGGTCACCCCAAGCCCCCCCG
AGCGGGTTCCCCCGGCAACCCCGAAGGCGGAGAGGCCAAG
GGCCCGTTCGGCGATGGCCACATCCTCCATGACCACGTCG
CTCTCGGCCATGCTCCGAATAGCCTGGGAGACGAGCACAT
CCGCGGACTTGTCAGCCGCCCCCACGGACATGTACATCTG
CAGGATGGTGGCCATACACGTGTCCGCCAGGCGCCGCATC
TTGTCCTGATGGGCCGCCACGGCCCCGTCGATCGTGGGGG
CCTCGAGCCCGGGGTGGTGGCGCGCCAGTCGTTCTAGGTT
CACCATGCAGGCGTGGTACGTGCGGGCCAAGGCGCGGGCC
TTCACGAGGCGTCGGGTGTCGTCCAGGGACCCCAGGGCGT
CATCGAGCGTGATGGGGGCGGGAAGTAGCGCGTTAACGAC
CACCAGGGCCTCCTGCAGCCGCGGCTCCGCCTCCGAGGGC
GGACCGGCCGCGCGGATCATCTCATATTGTTCCTCGGGGC
GCGCTCCCCAGCCACATATAGCCCCGAGAAGAAGCATCGC
GGGCGGGTACGGCTTGGGCGCGCGGACGCAATGGGGCAGG
AAGACGGGAACCGCGGGGAGAGGCGGGCGGCCGGGACTCC
CGTGGAGGTGACCGCGCTTTATGCGACCGACGGGGGCGTT
ATTACCTCTTCGATCGCCCTCCTCACAAACTCTCTACTGG
GGGCCGAGCCGGTTTATATATTCAGCTACGACGCATACAC
GCACGATGGCCGTGCCGACGGGCCCACGGAGCAAGACAGG
TTCGAAGAGAGTAGGGCGCTCTACCAAGCGTCGGGCGGGC
TAAATGGCGACTCCTTCCGAGTAACCTTTTGTTTATTGGG
GACGGAAGTGGGTGGGACCCACCAGGCCCGCGGGCGAACC
CGACCCATGTTCGTCTGTCGCTTCGAGCGAGCGGACGACG
TCGCCGCGCTACAGGACGCCCTGGCGCACGGGACCCCGCT
ACAACCGGACCACATCGCCGCCACCCTGGACGCGGAGGCC
ACGTTCGCGCTGCATGCGAACATGACCTGGCTCTCACCGT
GGCCGTCAACAACGCCAGCCCCCGCACCGGACGCGACGCC
GCCGCGGCGCAGTATGATCAGGGCGCGTCCCTACGCTCGC
TCGTGGGGCGCACGTCCCTGGGACAACGCGGCCTTACCAC
GCTATACGTCCACCACGAGGCGCGCGTGCTGGCCGCGTAC
CGCAGGGCGTATTATGGAAGCGCGCAGAGTCCCTTCTGGT
TTCTTAGCAAATTCGGGCCTGACGAAAAAAGCCTGGTGCT
CACCACTCGGTACTACCTGCTTCAGGCCCAGCGTCTGGGG
GGCGCGGGGGCCACGTACGACCTGCAGGCCATCAAGGACA
TCTGCGCCACCTACGCGATTCCCCACGCCCCCCGCCCCGA
CACCGTCAGCGCCGCGTCCCTGACCTCGTTTGCCGCCATC
CGCGGTTCTGTTGCACGAGCCAGTACGCCCGCGGGGCCGC
GGCGGCCGGGTTTCCGCTTTACGTGGAGCGCCGTATTGCG
GCCGACGTCCGCGAGACCAGTGCGCTGGAGAAGTTCATAA
CCCACGATCGCAGTTGCCTGCGCGTGTCCGACCGTGAATT
CATTACGTACTTTCCCTGGCCCATTTTGAGTGTTTCAGCC
CCCCGCGCCTAGCCACGCATCTTCGGGCCGTGACGACCCA
GGACCCCAACCCCGCGGCCAACACGGAGCAGCCCTCGCCC
CTGGGCAGGGAGGCCGTGGAACAATTTTTTTGCCACGTGC
GCGCCCAACTGAATATCGGGGAGTACGTCAAACACAACGT
GACCCCCCGGGAGACCGTCCTGGATGGCGATACGGCCAAG
GCCTACCTGCGCGCTCGCACGTACGCGCCCGGGGCCCTGA
CGCCCGCCCCCGCGTATTGCGGGGCCGTGGACTCCGCCAC
CAAAATGAGGGGCGTTTGGCGGACGCCGAAAAGCTCCTGG
TCCCCCGCGGGTGGCCCGCGTTTGCGCCCGCCAGTCCCGG
GGAGGATACGGCGGAGGATACGGCGGGCGGCACGCCGCCC
CCACAGCCTGCGGAATCGCAAGCGCCTCCTGAGACTGGCC
GCCACGGAACAACAGGACACCACGCCCCCGGCGATCGCGG
CGCTTATCCGTAATGCGGCGGTGCAGACTCCCCTGCCCGT
CTACCGGATATCCATGGTCCCCACGGGACAGGCATTTGCC
GCGCTGGCCTGGGACGACTGGGCCCGCATAACGCGGGACG
CTCGCCTGGCCGAAGCGGTCGTGTCCGCCGAAGCGGCGGC
GCACCCCGACCACGGCGCGCTGGGCAGGCGGCTCACGGAT
CGCATCCGCGCCCAGGGCCCCGTGATGCCCCCTGGCGGCC
TGGATGCCGGGGGGCAGATGTACGTGAATCGCAACGAGAT
ATTTAACGGCGCGCTGGCAATCACAAACATCATCCTGGAT
CTCGACATCGCCCTGAAGGAGCCCGTCCCCTTTCGCCGGC
TCCACGAGGCCCTGGGCCACTTTAGGCGCGGGGCTCTGGC
GGCGGTTCAGCTCCTGTTTCCCGCGGCCCGCGTGGCCCCG
ACGCATATCCCTGTTATTTTTTCAAAAGCGCATGTCGGCC
CGGCCCGGCGTCCGTGGGTTCCGGCAGCGGACTCGGCAAC
GACGACGACGGGGACTGGTTTCCCTGCTACGACGCCGCCG
GTGATGAGGAGTGGGCGGAGGACCCGGGCGCCATGGACAC
ATCCCACGATCCCCCGGACGACGAGGTTGCCTACTTTGAC
CTGTGCCACGAAGTCGGCCCCACGGCGGAACCTCGCGAAA
CGGATTCGCCCGTGTGTTCCTGCACCGACAAGATCGGACT
GCGGGTGTGCATGCCCGTCCCCGCCCCGTACGTCGTCCAC
GGTTCTCTACGATGCGGGGGGTGGCACGGGTCATCCAGCA
GGCGGTGCTGTTGGACCGAGATTTTGTGGAGGCCATCGGG
AGCTACGTAAAAACTTCCTGTTGATCGATACGGGGGTGTA
CGCCCACGGCCACAGCCTGCGCTTGCCGTATTTTGCCAAA
ATCGCCCCCGACGGGCCTGCGTGCGGAAGGCTGCTGCCAG
TGTTTGTGATCCCCCCCGCCTGCAAAGACGTTCCGGCGTT
TGTCGCCGCGCACGCCGACCCGCGGCGCTTCCATTTTCAC
GCCCCGCCCACCTATCTCGCTTCCCCCCGGGAGATCCGTG
TCCTGCACAGCCTGGGTGGGGCTATGTGAGCTTCTTTGAA
AGGAAGGCGTCCCACAACGCGCTGGAACACTTTGGGCGAC
GCGAGACCCTGACGGAGGTCCTGGGTCGGTACAACGTACA
GCCGGATGCGGGGGGGACCGTCGAGGGGTTCGCATCGGAA
CTGCTGGGGCGGATAGTCGCGTGCATCGAAACCCACTTTC
CCGAACACGCCGGCGAATATCAGGCCGTATCCGTCCGGCG
GGCCGTCAGTAAGGACGACTGGGTCCTCCTACAGCTAGTC
CCCGTTCGCGGTACCCTGCAGCAAAGCCTGTCGTGTCTGC
GCTTTAAGCACGGCCGGGCGAGTCGCGCCACGGCGCGGAC
ATTCGTCGCGCTGAGCGTCGGGGCCAACAACCGCCTGTGC
GTGTCCTTGTGTCAGCAGTGCTTTGCCGCCAAATGCGACA
GCAACCGCCTGCACACGCTGTTTACCATTGACGCCGGCAC
GCCATGCTCGCCGTCCGTTCCCTGCAGCACCTCTCAACCG
TCGTCTTGATAACGGCGTACGGCCTCGTGCTCGTGTGGTA
CACCGTCTTCGGTGCCAGTCCGCTGCACCGATGTATTTCG
TGGTACGCCCCACCGGCACCAACAACGACACCGCCCTCGT
GTGGATGAAAATGAACCAGACCCTATTGTTTCTGGGGGCC
CCGACGCACCCCCCCAACGGGGGCTGGCGCAACCACGCCC
ATATCTGCTACGCCAATCTTATCGCGGGTAGGGTCGTGCC
CTTCCAGGTCCCACCCGACGCCACGAATCGTCGGATCATG
AACGTCCACGAGGCAGTTAACTGTCTGGAGACCCTATGGT
ACACACGGGTGCGTCTGGTGGTCGTAGGGTGGTTCCTGTA
TCTGGCGTTCGTCGCCCTCCACCAACGCCGATGTATGTTT
GGTGTCGTGAGTCCCGCCCACAAGATGGTGGCCCCGGCCA
CCTACCTCTTGAACTACGCAGGCCGCATCGTATCGAGCGT
GTTCCTGCAGTCCCCCTACACGAAAATTACCCGCCTGCTC
TGCGAGCTGTCGGTCCAGCGGCAAAACCTGGTTCAGTTGT
TTGAGACGGACCCGGTCACCTTCTTGTACCACCGCCCCGC
CATCGGGGTCATCGTAGGCTGCGAGTTGATGCTACGCTTT
GTGGCCGTGGGTCTCATCGTCGGCACCGCTTTCATATCCC
GGGGGGCATGTGCGATCACATACCCCCTGTTTCTGACCAT
CACCACCTGGTGTTTTGTCTCCACCATCGGCCTGACAGAG
CTGTATTGTATTCTGCGGCGGGGCCCGGCCCCCAAGAACG
CAGACAAGGCCGCCGCCCCGGGGCGATCCAAGGGGCTGTC
GGGCGTCTGCGGGCGCTGTTGTTCCATCATCCTGTCGGGC
ATCGCAATGCGATTGTGTTATATCGCCGTGGTGGCCGGGG
TGGTGCTCGTGGCGCTTCACTACGAGCAGGAGATCCAGAG
GCGCCTGTTTGATGTATGACGTCACATCCAGGCCGGCGGA
AACCGGAACGGCATATGCAAACTGGAAACTGTCCTGTCTT
GGGGCCCACCCACCCGACGCGTCATATGTAAATGAAAATC
GTTCCCCCGAGGCCATGTGTAGCCTGGATCCCAACGACCC
CGCCCATGGGTCCCAATTGGCCGTCCCGTTACCAAGACCA
ACCCAGCCAGCGTATCCACCCCCGCCCGGGTCCCCGCGGA
AGCGGAACGGTGTATGTGATATGCTAATTAAATACATGCC
ACGTACTTATGGTGTCTGATTGGTCCTTGTCTGTGCCGGA
GGTGGGGCGGGGGCCCCGCCCGGGGGGCGGAACTAGGAGG
GGTTTGGGAGAGCCGGCCCCGGCACCACGGGTATAAGGAC
ATCCACCACCCGGCCGCCCCGCCCATGGGTCCCAATTGGC
CGTCCCGTTACCAAGACCAACCCAGCCAGCGTATCCACCC
CCGCCCGGGTCCCCGCGGAAGCGGAACGGTGTATGTGATA
TGCTAATTAAATACATGCCACGTACTTATGGTGTCTGATT
GGTCCTTGTCTGTGCCGGAGGTGGGGCGGGGGCCCCGCCC
GGGGGGCGGAACTAGGAGGGGTTTGGGAGAGCCGGCCCCG
GCACCACGGGTATAAGGACATCCACCACCCGGCCGCTCCC
TATCAGTGATAGAGATCTCCCTATCATGATAGAGATCGCT
GCACTGAGGTGCAGGTACATCCAGCTGACGAGTCCCAAAT
AGGACGAAACGCGCTTCGGTGTGTCCTGGATTCCACTGCT
ATCCACCGGTGCGCCACCACCAGAGGCCATATCCGACACC
CCAGCCCCGACGGCAGCCGACAGCCCGGTCATGGCGACTG
ACATTGATATGCTAATTGACCTCGGCCTGGACCTCTCCGA
CAGCGATCTGGACGAGGACCCCCCCGAGCCGGCGGAGAGC
CGCCGCGACGACCTGGAATCGGACAGCAACGGGGAGTGTT
CCTCGTCGGACGAGGACATGGAAGACCCCCACGGAGAGGA
CGGACCGGAGCCGATACTCGACGCCGCTCGCCCGGCGGTC
CGCCCGTCTCGTCCAGAAGACCCCGGCGTACCCAGCACCC
AGACGCCTCGTCCGACGGAGCGGCAGGGCCCCAACGATCC
TCAACCAGCGCCCCACAGTGTGTGGTCGCGCCTCGGGGCC
CGGCGACCGTCTTGCTCCCCCGAGCGGCACGGGGGCAAGG
TGGCCCGCCTCCAACCCCCACCGACCAAAGCCCAGCCTGC
CCGCGGCGGACGCCGTGGGCGTCGCAGGGGTCGGGGTCGC
GGTGGTCCCGGGGCCGCCGATGGTTTGTCGGACCCCCGCC
GGCGTGCCCCCAGAACCAATCGCAACCCGGGGGGACCCCG
CCCCGGGGCGGGGTGGACGGACGGCCCCGGCGCCCCCCAT
GGCGAGGCGTGGCGCGGAAGTGAGCAGCCCGACCCACCCG
GAGGCCCGCGGACACGGAGCGTGCGCCAAGCACCCCCCCC
GCTAATGACGCTGGCGATTGCCCCCCCGCCCGCGGACCCC
CGCGCCCCGGCCCCGGAGCGAAAGGCGCCCGCCGCCGACA
CCATCGACGCCACCACGCGGTTGGTCCTGCGCTCCATCTC
CGAGCGCGCGGCGGTCGACCGCATCAGCGAGAGCTTCGGC
CGCAGCGCACAGGTCATGCACGACCCCTTTGGGGGGCAGC
CGTTTCCCGCCGCGAATAGCCCCTGGGCCCCGGTGCTGGC
GGGCCAAGGAGGGCCCTTTGACGCCGAGACCAGACGGGTC
TCCTGGGAAACCTTGGTCGCCCACGGCCCGAGCCTCTATC
GCACTTTTGCCGGCAATCCTCGGGCCGCATCGACCGCCAA
GGCCATGCGCGACTGCGTGCTGCGCCAAGAAAATTTCATC
GAGGCGCTGGCCTCCGCCGACGAGACGCTGGCGTGGTGCA
AGATGTGCATCCACCACAACCTGCCGCTGCGCCCCCAGGA
CCCCATTATCGGGACGGCCGCGGCGGTGCTGGATAACCTC
GCCACCCGCCTGCGGCCCTTTCTCCAGTGCTACCTGAAGG
CGCGAGGCCTGTGCGGCCTGGACGAACTGTGTTCGCGGCG
GCGTCTGGCGGGCATTAAGGACATTGCATCCTTCGTGTTT
GTCATTCTGGCCAGGCTCGCCAACCGCGTCGAGCGTGGCG
TCGCGGAGATCGACTACGCGACCCTTGGTGTCGGGGTCGG
AGAGAAGATGCATTTCTACCTCCCCGGGGCCTGCATGGCG
GGCCTGATCGAAATCCTAGACACGCACCGCCAGGAGTGTT
CGAGTCGTGTCTGCGAGTTGACGGCCAGTCACATCGTCGC
CCCCCCGTACGTGCACGGCAAATATTTTTATTGCAACTCC
CTGTTTTAGGTACAATAAAAACAAAACATTTCAAACAAAT
CGCCCCACGTGTTGTCCTTCTTTGCTCATGGCCGGCGGGG
CGTGGGTCACGGCAGATGGCGGGGGTGGGCCCGGCGTACG
GCCTGGGTGGGCGGAGGGAACTAACCCAACGTATAAATCC
GTCCCCGCTCCAAGGCCGGTGTCATAGTGCCCTTAGGAGC
TTCCCGCCCGGGCGCATCCCCCCTTTTGCACTATGACAGC
GACCCCCCTCCCCAACCTGTTCTTACGGGCCCCGGACATA
ACCCACGTGGCCCCCCCTTACTGCCTCAACGCCACCTGGC
AGGCCGAAACGGCCATGCACACCAGCAAAACGGACTCCGC
TTGCGTGGCCGTGCGGAGTTACCTGGTCCGCGCCTCCTGT
GAGACCAGCGGCACAATCCACTGCTTTTTCTTTGCGGTAT
ACAAGGACCCCCACCATCCCCCTCCGCTGATTACCGAGCT
CCGCAACTTTGCGGACCTGGTTAACCACCCGCCGGTCCTA
CGCGAACTGGAGGATAAGCGCGGGGTGCGGCTGCGGTGTG
CGCGGCTGCGGTGTGCGCGGCCGTTTAGCGTCGGGACGAT
TAAGGACGTCTCTGGGTCCGGCGCGTCCTCGGCGGGAGAG
TACACGATAAACGGGATCGTGTACCACTGCCACTGTCGGT
ATCCGTTCTCAAAAACATGCTGGATGGGGGCCTCCGCGGC
CCTACAGCACCTGCGCTCCATCAGCTCCAGCGGCATGGCC
GCCCGCGCGGCAGAGCATCGACGCGTCAAGATTAAAATTA
AGGCGTGATCTCCAACCCCCCATGAATGTGTGTAACCCCC
CCCCCCCAAAAAAATAAAGAGCCGTAACCCAACCAAACCA
GGCGTGGTGTGAGTTTGTGGACCCAAAGCCCTCAGAGACA
ATGCGACAGGCCAGTATGGACCGTGATACTTTTATTTATT
AACTCACAGGGGCGCTTACCGCCACAGGAATACCAGAATA
ATGACCACCACAATCGCGACCAGCCCTGTCGCCGGATGGG
GCATGATCAGACGAGCCGCGCGCCGCGCGTTGGGCCCTGT
ACAGCTCGCGCGAATTGACCCTAGGAGGCCGCCACGCGCC
CGAGTTTTGCGTTCGTCGCTGGTCGTCGGGCGCCAAAGCC
CCGGACGGCTGTTCGGTCGAACGAACGGCCACGACAGTGG
CATAGGTTGGGGGGTGGTCCGACATAGCCTCGGCGTACGT
CGGGAGGCCCGACAAGAGGTCCCTTGTGATGTCGGGTGGG
GCCACAAGCCTGGTTTCCGGAAGAAACAGGGGGGTTGCCA
ATAACCCGCCAGGGCCAAAACTCCGGCGCTGCGCACGTCG
TTCGGCGCGGCGCCGGGCGCGCCGAGCGGCTCGCTGGGCG
GCTTGGCGTGAGCGGCCCCGCTCCGACGCCTCGCCCTCTC
CGGAGGAGGTTGGCGGAATTGGCACGGACGACAGGGGCCC
AGCAGAGTACGGTGGAGGTGGGTCCGTGGGGGTGTCCAGA
TCAATAACGACAAACGGCCCCTCGTTCCTACCAGACAAGC
TATCGTAGGGGGGCGGGGGATCAGCAAACGCGTTCCCCGC
GCTCCATAGACCCGCGTCGGGTTGCGCCGCCTCCGAAGCC
ATGGATGCGCCCCAAAGCCACGACTCCCGCGCGCTAGGTC
CTTGGGGTAAGGGAAAAGGCCCTACTCCCCATCCAAGCCA
GCCAAGTTAACGGGCTACGCCTTCGGGGATGGGACTGGCA
CCCCGGCGGATTTTGTTGGGCTGGTACGCGTCGCCCAACC
GGGCACGGACGACAGGGGCCCAGCAGAGTACGGTGGAGGT
GGGTCCGTGGGGGGGGCCAGGTCAATAACGACAAACGGCC
CCTCGTTCCTACCGACAAGCTATCGTAGGGGGGCGGGGGA
TCAGCAAACGCGTTCCCCGCGCTCCATAGACCCGCGTCGG
GTTGCGCCGCCTCCGAAGCCATGGATGCGCCCCAAAGCCA
CGACTCCCGCGCGCTAGGTCCTTGGGGTAAGGGAAAAGGC
CCTACTCCCCATCCAAGCCAGCCAAGTTAACGGGCTACGC
CTTCGGGGATGGGACTGGCACCCCGGCGGATTTTGTTGGG
CTGGTACGCGTCGCCCAACCGAGGGCCGCGTCCACGGGAC
GCGCCTTTTATAACCCCGGGGTCATTCCCAACGATCACAT
GCAATCTAACTGGCTCCCCTCTCCCCTCTCCCCCCCTCTC
CCCGCTGGGGCTGGGGAGGGCTGGGGCTGGGGAGGGGCGG
TGGTGTGTAGCAGGAGCGGTGTGTTGCGCCGGGGTACGTC
TGGAGGAGCGGGAGGTGCGCGGTGACGTGTGGATGAGGAA
CAGGAGTTGTTGCGCGGTGAGTTGTCGCTGTGAGTTGTGT
TGTTGGGCAGGTGTGGTGGATGACGTGACGTGTGACGTGC
GGATTGCGCCGTGCTTTGTTGGTGTTGTTTTACCTGTGGC
AGCCCGGGCCCCCCGCGGGCGCGCGCGCGCGCAAAAAAGG
CGGGCGGCGGTCCGGGCGGCGTGCGCGCGCGCGGCGGGCG
TTGGGGGCGGGGCCGCGGGAGCGGGGGAGGAGCGGGGGAG
GAGCGGGGGAGGAGCGGGGGAGGAGCGGGGGGGGGAGCGG
GGGGGGGGGGGGGGGGGGGGGGGGGGGGGGGGGGGGGGGG
GGGGGGGGGGGGGGGGGGGGGGGGGGGAGGGGGGGGGGGA
GGAGCGGGGGAGGAGCGGGGCCGCGCGCGGCCCCCGGGGG
GTGTGTTTTGGGGGGGCCCGTTTCCGGGGTCTGGCCGCTC
CTCCCCCGCTCCTCCCCCCGCTCCTCCCCCCGCTCCTCCC
CCCCCCCCCCCCCCCCCCCCCCCCCCCCCCCCCCCCCCCC
CCCCCCCCCCCACCCCCCCCCCCCCCCCCCCCCCCCCCCC
CCCCCCGCCCCCCCCCCGCTCCCCCCCCGCTCCTCCCCCG
CTCCCGCGGCCCCGCCCCCAACGCCCGCCGCGCGCGCGCA
CGCCGCCCGGACCGCCGCCCGCCTTTTTTGCGCGCGCGCG
CGCCCGCGGGGGGCCCGGGCTGCCACAGGTAAAACAACAC
CAACAAAGCACGGCGCAATCCGCACGTCACACGTCACGTC
ATCCACCACACCTGCCCAACAACACAACTCACAGCGACAA
CTCACCGCGCAACAACTCCTGTTCCTCATCCACACGTCCC
CGCGCCCCTCCCGCTCCTCCAGACGTACCCCGGCGCAACA
CACCGCTCCTGCTACACACCACCGCCCCTCCCCAGCCCCA
GCCCTCCCCACCCCACCCCCCCCCCCCCCCCCCCCCCCCC
GCCCCCACCCCCCCCCCCCCCCCCACCCCCCCCCCCCCCC
CCCCCCACCCCAGCCCCCCCCAGCCCCAGCCCTCCCCAGC
CCCAGCCCTCCCCAGCCGCGTCCCGCGCTCCCTCGGGGGG
GTTCGGGCATCTCTACCTCAGTGCCGCCAATCTCAGGTCA
GAGATCCAAACCCTCCGGGGGCGCCCGCGCACCCCCCCCC
CCCCCCCCCCCCCCCCCCCCCCCCCCCCCCCCCCCCCCCC
CCCCCCCCCCCCCACCCCCCGGGGGGGGGGGGGGGAGGGG
GGGGGGGGGGGGGGGGGGGGGGGGGGGGGGGGGGGGGGGG
GGGGGGGGGGGGGGGGGGGGTGGGGGTGCGCGGGCGCCCC
CGGAGGGTTTGGATCTCTGACCTGAGATTGGCGGCACTGA
GGTAGAGATGCCCGAACCCCCCCGAGGGAGCGCGGGACGC
GGCTGGGGAGGGCTGGGGCTGGGGAGGGCTGGGGCTGGGG
AGGGCTGGGGCTGGGGGGGGGGGGGGGGGGGGAGGGCGGG
GGGGGGGGGGGGCCCCCCCCCCGCCCCCCTCCCCCCCCCC
CCCCCCCCCCCCCCCCCCCCGCCCCTCCCCCCCTCCCGCC
CCTCGAATAAACAACGCTACTGCAAAACTTAATCAGGTCG
TTGCCGTTTATTGCGTCTTCGGGTTTCACAAGCGCCCCGC
CCCGTCCCGGCCCGTTACAGCACCCCGTCCCCCTCGAACG
CGCCGCCGTCGTCTTCGTCCCAGGCGCCTTCCCAGTCCAC
AACGTCCCGTCGCGGGGGCGTGGCCAAGCCCGCCTCCGCC
CCCAGCACCTCCACGGCCCCCGCCGCCGCCAGCACGGTGC
CGCTGCGGCCCGTGGCCGAGGCCCAGCGAATCCCGGGCGG
CGCCGGCGGCAGGGCCCCCGGGCGGTCGTCGGCGCGCCGC
GCAGCACCAGCGGGGGGGCGTCGTCGTCGGGCTCCAGCAG
GGCGCGGGCGCAAAAGTCCCTCCGCGGCCCGCGCCACCGG
GCCGGGCCGGCGCGCACCGCCTCGCGCCCCAGCGCCACGT
ACACGGGCCGCAGCGGCGCGCCCAGGCCCCAGCGCGCGCA
GGCGCGGTGCGAGTGGGCCTCCTCCTCGCAGAAGTCCGGC
GCGCCGGGCGCCATGGCGTCGGTGGTCCCCGAGGCCGCCG
CCCGGCCGTCCAGCGCCGGCAGCACGGCCCGGCGGTACTC
GCGCGGGGACATGGGCACCGGCGTGTCCGGGCCGAAGCGC
GTGCGCACGCGGTAGCGCACGTTGCCGCCGCGGCACAGGC
GCAGCGGCGGCGCGTCGGGGTACAGGCGCGCGTGCGCGGC
CTCCACGCGCGCGAAGACCCCCGGGCCGAACACGCGGCCC
GAGGCCAGCACCGTGCGGCGCAGGTCCCGCGCCGCCGGCC
AGCGCACGGCGCACTGCACGGCGGGCAGCAGGTCGCACGC
CAGGTAGGCGTGCTGCCGCGACACCGCGGGCCCGTCGGCG
GGCCAGTCGCAGGCGCGCACGGTGTTGACCACGATGAGCC
GCCGGTCGCCGGCGCTGGCGAGCAGCCCCAGAAACTCCAC
GGCCCCGGCGAAGGCCAGGTCCCGCGTGGACAGCAGCAGC
ACGCCCTGCGCGCCCAGCGCCGACACGTCGGGGGCGCCGG
TCCAGTTGCCCGCCCAGGCGGCCGTGTCCGGCCCGCACAG
CCGGTTGGCCAGGGCCGCCAGCAGGCAGGACAGCCCGCCG
CGCTCGGCGGACCACTCCGGCGGCCCCCCCGAGGCCCCGC
CGCCGGCCAGGTCCTCGCCCGGCAGCGGCGAGTACAGCAC
CACCACGCGCACGTCCTCGGGGTCGGGGATCTGGCGCATC
CAGGCCGCCATGCGGCGCAGCGGGCCCGAGGCGCGCAGGG
GGCCAAAGAGGCGGCCCCCGGCGGCCCCGTGGGGGTGGGG
GTTCTCGTCGTCGTCGCCGCCGCACGCGGCCTGGGCGGCG
GGGGCGGGCCCGGCGCACCGCGCGGCGATCGAGGCCAGGG
CCCGCGGGTCAAACATGAGGGCCGGTCGCCAGGGGACGGG
GAACAGCGGGTGGTCCGTGAGCTCGGCCACGGCGCGCGGG
GAGCAGTAGGCCTCCAGGGCGGCGGCCGCGGGCGCCGCCG
TGGGCTGGGCCCCCGGGGCTGCCGCCGCCAGCCGCCCAGG
GGGTCGGGGCCCTCGGCGGGCGGGCGCGACAGCGCCACGG
GGCGCGGGCGGGCCTGCGCCGCGGCGCCCCGGGCCGCCGC
GGGCTGGGCGGGTGTGTGCTCGGGCCCAGGCCGCGTGCGG
CGGCGACGACGACGAGAACCCCCACCCCCACGGGGCCGCC
GGGGCCGCCTCTTTGGCCCCCTGCGCGCCTCGGGCCCGCT
GCGCCGCATGGCGGCCTGGATGCGCCAGATCCCCGACCCC
GAGGACGTGCGCGTGGTGGTGCTGTCTCGCCGCTGCCGGG
CGAGGACCTGGCCGGCGGCGGGGCCTCGGGGGGGCCGCCG
GGGGTCCGCCGAGCGCGGCGGGCTGTCCTGCCTGCTGGCG
GCCCTGGCCAACCGGCTGTGCGGGCCGGACACGGCCGCCT
GGGCGGGCAACTGGACCGGCGCCCCCGACGTGTCGGCGCT
GGGCGCGCAGGGCGTGCTGCTGCTGTCCACGCGGGACCTG
GCCTTCGCCGGGGCCGTGGAGTTTCTGGGGCTGCTCGCCA
GCGCCGGCGACCGGCGGCTCATCGTGGTCAACACCGTGCG
CGCCTGCGACTGGCCCGCCGACGGGCCCGCGGTGTCGCGG
CAGCACGCCTACCTGGCGTGCGACCTGCTGCCCGCCGTGC
AGTGCGCCGTGCGCTGGCCGGCGGCGCGGGACCTGCGCCG
CACGGTGCTGGCCTCGGGCCGCGTGTTCGGCCCGGGGGTC
TTCGCGCGCGTGGAGGCCGCGCACGCGCGCCTGTACCCCG
ACGCGCCGCCGCTGCGCCTGTGCCGCGGCGGCAACGTGCG
CTACCGCGTGCGCACGCGCTTCGGCCCGGACACGCCGGTG
CCCATGTCCCCGCGCGAGTACCGCCGGGCCGTGCTGCCGG
CGCTGGACGGCCGGGCGGCGGCCTCGGGGACCACCGACGC
CATGGCGCCCGGCGCGCCGGACTTCTGCGAGGAGGAGGCC
CACTCGCACCGCGCCTGCGCGCGCTGGGGCCTGGGCGCGC
CGCTGCGGCCCGTGTACGTGGCGCTGGGGCGCGAGGCGGT
GCGCGCCGGCCCGGCCCGGTGGCGCGGGCCGCGGAGGGAC
TTTTGCGCCCGCGCCCTGCTGGAGCCCGACGACGACGCCC
CCCCGCTGGTGCTGCGCGGCGACGACGACGACGGCCCGGG
GGCCCGCCGCCGGCGCCGCCCGGGATTCGCTGGGCCTCGG
CCACGGGCCGCAGCGGCACCGTGCTGGCGGCGGCGGGGGC
CGTGGGGTGCTGGGGGCGGAGGCGGGCTTGGCCACGCCCC
CGCGACGGGACGTTGTGGACTGGGAAGGCGCCTGGGACGA
AGACGACGGCGGCGCGTTCGAGGGGGACGGGGTGCTGTAA
CGGGCCGGGACGGGGCGGGGCGCTTGTGAAACCCGAAGAC
GCAATAAACGGCAACGACCTGATTAAGGTTTGCAGGAGCG
TTGTTTATTCGAGGGGCGGGAGGGGGCGGGGGGGGGGGGG
GGGGGGGGGGGGGGGGGGGGGGGGGGGGGGGGGGGGGGGG
GGGGCGGCGGCGCCCGCGGCCGCCGCCCTGGAGGCCTACT
GCTCCCCGCGCGCCGTGGCCGAGCTCACGGACCCCCCGCT
GTTCCCCGTCCCCTGGCGACCGGCCCTCATGTTTGCCCCG
CGGGCCCTGGCCTCGATCGCCGCGCGGTGCGCCGGGCCCG
CCCCCGCCGCCCTGGGTGGGTGGGGAGTGGGTGGGTGGGG
AGTGGGTGGGTGGGGAGTGGCAAGGAAGAAACAAGCCCGA
CCACCAGACAGAAAATGTAACCATACCCAAACCGACTCTG
GGGGCTGTTTGTGGGGTCGGAACCATAGGATGAACAAACC
ACCCCGTACCTCCCGCACCCAAGGGTGCGGGTGGCTCATC
GGCATCTGTCCGGTATGGGTTGTTCCCCACCCACTCGCGT
TCGGACGTCTTAGAATCATGGCGGTTTTCTATGCCGACAT
CGGTTTTCTCCCCCGCAATAAGACACGATGCGATAAAATC
TGTTTGTAAAATTTATTAAGGGTACAAAATGCCCTAGCAC
AGGGGTGGGGGTAGGGCCGGGGCCCCACACCCAAACGCAC
CAAACAGATGCAGGCAGTGGGTCGAGTACAGCCCCGCGTA
CGAACACGTCGATGCGTGTGTCAGACAGCACCAGAAAGCA
CAGGCCATCAACAGGTCGTGCATGTGTCGGTGGGTTTGGA
CGCGGGGGGCCATGGTGGTGATAAAGTTAATGGCCGCCGT
CCGCCAGGGCCACAGGGGCGCCGTCTCTTGGTTGGCCCGG
AGCCACTGGGTGTGGACCAGCCGCGCGTGGCGGCCCAACA
TGGCCCCTGTAGCCGGGGGCGGGGGATCGCGCACGTTTGC
AGCGCACATGCGAGACACCTCGACCACGGTTCGAAAGAAG
GCCCGGTGGTCCGCGGGCAACATCACCAGGTGCGCAAGCG
CCCGGGCGTCCAGAGGGTAGAGCCCTGAGTCATCCGAGGT
TGGCTCATCGCCCGGGTCTTGCCGCAAGTGCGTGTGGGTT
GGGCTTCCGGTGGGCGGGACGCGAACCGCGGTGTGGATCC
CGACGCGGGCCCGAGCGTATGCTCCATCTTGTGGGGAGAA
GGGGTCTGGGCTCGCCAGGGGGGCATACTTGCCCGGGCTA
TACAGACCCGCGAGCCGTACGTGGTTCGCGGGGGGTGCGT
GGGGTCCGGGGTCCCTGGGAGACCGGGGTTGTCGTGGATC
CCTGGGGTCACGCGGTACCCTGGGGTCTCTGGGAGCTCGC
GGTACTCTGGGTCCCTAGGTTCTCGGGGTGGTCGCGGACC
CGGGGCTCCCGGGGAACACGCGGTGTCCTGGGGATTGTTG
GCGGTCGGACGGCTTCAGATGGCTTCGAGATCGTAGTGTC
CGCACCGACTCGTAGTAGACCCGAATCTCCACATTGCCCC
GCCGCTTGATCATTATCACCCCGTTGCGGGGGTCCGGAGA
TCATGCGCGGGTGTCCTCGAGGTGCGTGAACACCTCTGGG
GTGCATGCCGGCGGACGGCACGCCTTTTAAGTAAACATCT
GGGTCGCCCGGCCCAACTGGGGCCGGGGGTTGGGTCTGGC
TCATCTCGAGAGACACGGGGGGGAACCACCCTCCGCCCAG
AGACTCGGGTGATGGTCGTACCCGGGACTCAACGGGTTAC
CGGATTACGGGGACTGTCGGTCACGGTCCCGCCGGTTCTT
CGATGTGCCACACCCAAGGATGCGTTGGGGGCGATTTCGG
GCAGCAGCCCGGGAGAGCGCAGCAGGGGACGCTCCGGGTC
GTGCACGGCGGTTCTGGCCGCCTCCCGGTCCTCACGCCCC
CTTTTATTGATCTCATCGCGTACGTCGGCGTACGTCCTGG
GCCCAACCCGCATGTTGTCCAGGAAGGTGTCCGCCATTTC
CAGGGCCCACGACATGCTTTTCCCGACGAGCAGGAAGCGG
TCCACGCAACGGTCGCCGCCGGTCGCCTCGACGAGGGCGT
TCCTCCTGCGGGAAGGCACGAACGCGGGTGAGCCCCCTCC
TCCGCCCCCGCGTCCCCCCTCCCCCGCCCCCGCGTCCCCC
CCCTCCGCCCCCGCGTCCCCCCCTCCTCCGCCCCCGCGTC
CCCCCCCCTCCGCCCCCGCGTCCCCCCTCCTCCCCCCCCA
AGGTGCTTACCCGTGCAAAAAGGCGGACCGGTGGGTTTCT
GTCGTCGGAGGCCCCCGGGGTGCGTCCCCTGTGTTTCGTG
GGTGGGGTGGGCGGGTCTTTCCCCCCCGCGTCCGCGTGTC
CCTTTCCGATGCGATCCCGATCCCGAGCCGGGGCGTCGCG
ATGCCGACGCCGTCCGCTCCGACGGCCCTCTGCGACTCCC
GCTCCCGGTCCGCGTGCTCCGCAGCCGCTCCCGTCGTTCG
TGGCCGGCGCCGTCTGCGGGCGTCGGTCGCGCCGGGCCTT
TATGTGCGCCGGAGAGACCCGCCCCCCGCCGCCCGGGTCC
GCCCCCGGGGCCGGCGCGGAGTCGGGCACGGCGCCAGTGC
TCGCACTTCGCCCTAATAATATATATATATTGGGACGAAG
TGCGAACGCTTCGCGTTCTCACTTCTTTTCCCCGGCGGCC
CCGCCCCCTTGGGGCGGTCCCGCCCGCCGGCCAATGGGGG
GGCGGCAAGGCGGGCGGCCCTTGGGCCGCCCGCCGTCCCG
TTGGTCCCGGCGTCCGGCGGGCGGGACCGGGGGCCCGGGG
ACGGCCAACGGGCGCGCGGGGCTCGATCTCATTACCGCCG
AACCGGGAAGTCGGGGCCCGGGCCCCGCCCCCTGCCCGTT
CCTCGTTAGCATGCGGAACGGAAGCGGAAACCGCCGGATC
GGGCGGTAATGAGATGCCATGCGGGGCGGGGCGCGGACCC
ACCCGCCCTCGCGCCCCGTCCATGGCAGATGGCGCGGATG
GGCGGGGCCGGGGGTTCGACCAACGGGCCGCGGCCACGGG
CCCCCGGCGTGCCGGCGTCGGGGCGGGGTCGTGCATAATG
GAATTCCGTTCGGGGTGGGCCCGCCGGGGGGCGGGGGGGC
GGCGGCCTCCGCTGCTCCTCCTTCCCGCCGGCCCCTGGGA
CTATATGAGCCCGAGGACGCCCCGATCGTCCACACGGAGC
GCGGCTGCCGACACGGATCCACGACCCGACGCGGGACCGC
CAGAGACAGACCGTCAGACGCTCGCCGCGCCGGGACGCCG
ATACGCGGACGAAGCGCGGGAGGGGGATCGGCCGTCCCTG
TCCTTTTTCCCCACCCAAGCATCGACCGGTCCGCGCTAGT
TCCGCGTCGACGGCGGGGGTCGTCGGGGTCCGTGGGTCTC
GCCCCCTCCCCCCTCGAGAGTCCGTAGGTGACCTACCGTG
CTACGTCCGCCGTCGCAGCCGTATCCCCGGAGGATCGCCC
CGCATCGGCGATGGCGTCGGAGAACAAGCAGCGCCCCGGC
TCCCCGGGCCCCACCGACGGGCCGCCGCCCACCCCGAGCC
CAGACCGCGACGAGCGGGGGGCCCTCGGGTGGGGCGCGGA
GACGGAGGAGGGCGGGGACGACCCCGACCACGCCCCGACC
ACCCCCACGACCTCGACGACGCCCGGCGGGACGGGAGGGC
CCCCGCGGCGGGCACCGACGCCGGCGAGGACGCCGGGGAC
GCCGCTCGCCGCGACAGCTGGCTCTGCGGCCTCCCTGGTA
GAGGAGGCCGGCCGGACGATCCCGACGCCCGACCCCGCGG
CCTCGCCGCCCCGGACCCCCGCCTTTCTAGCCGACGACGA
TGACGGGGACGAGTACGACGACGCAGCCGACGCCGCCGGC
GACCGGGCCCCGGCCCGGGGCCGCGAACGGGAGGCCCCGC
TACGCGGCGCGTATCCGGACCCCACGGACCGCCTGTCCCG
CGCCCGCCGGCCCAGCCGCCGCGGAGACGTCGTCACGGCC
GGCGGCGGCCATCGGCGCATCGACCTCGGCGGACTCCGGG
TCCTCGTCCTCGTCGTCCGCATCCTCTTCGTCCTCGTCGT
CCGACGAGGACGAGGACGACGACGGCAACGACGCGGCCGA
CCACGCACGCGAGGCGCGGGCCGTCGGGCGGGGTCCGTCG
AGCGCGGCGCCGGAAGCCCCCGGGCGGACGCCGCCCCCGC
CCGGGCCACCCCCCCTCTCCGAGGCCGCGCCCAAGCCCCG
GGCGGCGGCGAGGACCCCCGCGGCCTCCGCGGGCCGCATC
GAGCGCCGCCGGGCCCGCGCGGCGGTGGCCGGCCGCGACG
CCACGGGCCGCTTCACGGCCGGGCAGCCCCGGCGGGTCGG
GCTGGACGCCGACGCGGCCTCCGGCGCCTTCTACGCGCGC
TATCGCGACGGGTACGTCAGCGGGGAGCCGTGGCCCGGCG
CCGGGCCCCCGCCCCCGGGGCGGGTGCTGTACGGCGGCCT
GGGCGACAGCCGCCCGGGCCTCTGGGGGGCGCCCGAGGCG
GAGGAGGCGCGACGCCGGTTCGAGGCCTCGGGCGCCCCGG
GGCCGTGTGGGGCCCGAGCGGGGAGACGCCGCGCAGCAGA
CGCCCTGATCACGCGGCTGCTGTACACCCCGGACGCGGAG
GCCAGTCTGGGGCTCCAGACCCGCGTGGTCCCCGGGGACG
TGGCGCTGGACCAGGCCTGCTTCCGGATCTCGGGCGCCGC
GCGCAACAGCAGCTCCTTCATCCCGGCAGCGTGGCGCGGG
CCGTGCCCCACCTGGGCTACGCCATGGCGGCCGGCCGCTT
CGGCTGGGGCCTGGCGCACGCGGCGGCCGCCGTGGCCATG
AGCCGCCGATACGACCGCGCGCAGAAGGGCTTCCTGCTGA
CCAGCCTGCGCCGCGCCTACGCGCCCCTGTTGGCGCGCGA
GAACGCGGCGCTGACGGGGGCCGCGGGGAGCCCCGGCGCC
GGCGCAGATGACGAGGGGGTCGCCGCCGTCGCCGCCGCCG
CACCGGGCGGCGCGCGGGCCCGCCGGGTACGGCGCCGCGG
GGATCCTCGCCGCCCTGGGGCGGCTGTCCGCCGCGCCCGC
CTCCCCCGTGGGGGGCGACGACCCCGACGCCGCCCGCCAC
GCCGACGCCGACCCGGGCGCCGCGCCCAGGCCGGCCGCGT
GGCCGTCGAGTGCCTGGCCGCCTGCCGCGGGTCCTGGCGG
CGCTGGCCGAGGGCTTCGACGGCGACCTGGCGGCCGTCCC
GGGGCTGGCCGGGGCCCGGCCCGCCAGCCCCCCGCGCCGG
AGGGACCCGCGGACCCCGCTTCCCCGCCGCCGCCGCACGC
CGACGCGCCCCGCCTGCGCGCGGGCTGCGCGGCGCGGTCG
TGCGCGCCGCGCTGGTGCTCATGCCCCTGCGCGGGGACCT
GCGCGTGGCCGGCGGCAGCGGGCCGCCGTGGCCGCCGTGC
GCGCCGGAGCCTGGTCGCCGGGGCCCTGGGCCCCGCGCTG
CCGCGGGACCCGCGCCTGCCGAGCTCCGCGGCCGCCGCCG
CCGCGGACCGCTGTTTGAGAACCAGAGCCTCCGCCCCCTG
CTGGCGGCGGCGGCCAGCGCACCGGACGCCGCCGACGCGC
TGGCGGCCGCCGCCGCCTCCGCCGCCCGCGGGAGGGGCGC
AAGCGCAAGAGTCCCGGCCCGGCCCGGCCGCCCGGAGGCG
GCGGCCCGCGACCCCCGAAGACGAAGAAGAGCGGCGCGGA
CGCCCCCCCCGCGCGCCCCCCGCCCCCCCCCCCCCGCCCC
CCCCCCCCCCCCCCCGGGGCCCGAGCCCCCCCCCGCCCAG
CCCGCGGCGGCCCGGGGCGCCGCGGCGCAGGCCCGCCCGC
GCCCCGTGGCGCTGTCGCGCCGGCCCGCCGAGGGCCCCGA
CCCCCTGGGCGGCTGGCGGCGGCAGCCCCGGGGGCCCAAC
CCCACAGCGGCGGGGGGGGGGGGGGGGGGGGGGGGGGGGG
GGGCGGGCGGCCGGGCCGGGGGCGTCCGCGCCGCTCTTCT
TCGTCTTCGGGGGTCGCGGGCCGCCGCCTCCGGGCGGCCG
GGCCGGGCCGGGACTCTTGCGCTTGCGCCCCTCCCGCGGC
GCGGCGGGGGCGGCGGCGGCCGCCAGCGCGTCGGCGGCGT
CCGGTGCGCTGGCCGCCGCCGCCAGCAGGGGGCGGAGGCT
CTGGTCTCAAACAGCAGGTCCGCGGCGGCGGCGGCCGCGG
AGCTCGGCAGGCCGGTCCCGCGGCAGCGCGGGACAAGGGC
CCCGGCGACCAGGCTCACGGCGCGCACGGCGGCCACGGCG
GCCTCGCTGCCGCCGGCCACGCGCAGGTCCCCGCGCAGGC
GCATGAGCACCAGCGCGTCGCGCACGAACCGCAGCTCGCG
CAGCCACGCGCGCAGGCGGGGCGCGTCGGCGTGCGGCGGC
GGCGGGGAAGCGGGGTCCGCGGGTCCCTCCGGCCGCGGGG
GGCTGGCGGGCCGGGCCCCGGCCAGCCCCGGGACGGCCGC
CAGGTCGCCGTCGAAGCCCTCGGCCAGCGCCTCCAGGATC
CCGCGGCAGGCGGCCAGGCACTCGACGGCCACGCGGCCGG
CCTGGGCGCGGCGCCCGGCGTCGGCGTCGGCGTGGCGGGC
GGCGTCGGGGTCGTCGCCCCCCACGGGGGAGGCGGGCGCG
GCGGACAGCCGCCCCAGGGCGGCGAGGATCCCCGCGGCGC
CGTACCCGGCGGGCACCGCGCGCTCGCCCGGTGCGGCGGC
GGCGACGGCGGCGACCCCCTCGTCATCTGCGCCGGCGCCG
GGGCTCCCCGCGGCCCCCGTCAGCGGGTGGGAACAGGGGC
GCGTAGGCGCGGCGCAGGCTGGTCAGCAGGAAGCCCTTCT
GCGCCTCGTATCGGCGCTCATGGCCACGGCGGCCGCCGCG
TGCGCCAGGCCCCAGCCGAAGCGGCCGGCCGCCATGGCGT
AGCCCAGGTGGGGCACGGCCCGCGCCACGCTGCCGGTGAT
GAAGGAGCTGCTGTTGCGCGCGGCGCCCGAGATCCGGAAG
CAGGCCTGGTCCAGCGCCACGTCCCCGGGGACCACGCGCG
GGTTCTGGAGCCACCCCTGGCCTCCGCGTCCGGGGTGTAC
AGCAGCCGCGTGATCAGGGCGTACTGCTGCGCGGCGTCGC
CCAGCTCGGGCGCCCACACGGCCGCCGGGGCGCCCGAGGC
CTCGAACCGGCGTCGCGCCTCCTCCGCCTCGGGCGCCCCC
CAGAGGCCCGGGCGGCTGTCGCCCAGGCCGCCGTACAGCA
CCCGCCCCGGGGGCGGGGGCCCGGCGCCGGGCCACGGCTC
CCCGCTGACGTACCCGTCGCGATAGCGCGCGTAGAAGGCG
CCGGAGGCCGCGTCGGCGTCCAGCTCGACCCGCCGGGGCT
GCCCGGCCGTGAAGCGGCCCGTGGCGTCGCGGCCGGCCAC
CGCCGCGCGGGCCCGGCGGCGCTCGATGCGGCCCGCGGAG
GCCGCGGGGGTCCGCGCCGCCGCCCGGGGCTTGGGCGCGG
CCTCGGAGAGGGGGGGTGGCCCGGGCGGGGGCGGCGTCCG
CCCGGGGGCTTCCGGCGCCGCGCTCGACGGACCCCGCCCG
ACGGCCCGCGCCCGCGGCGTGGTCGGCCGCGTCGTTGCCG
TCGTCGTCCCGTCCTCGTCGGACGACGGGACGAAGCGGAT
GCGGACGGCGAGGACGAGGACCCGGAGTCCGGCGAGGCCG
AGACGCCGATGGCCGCCGCCGGCCGTGACGACGTCTCCGC
GGCGGCTGGGCCGGCGGGCGCGGCGACAGGCGGTCCGTGG
GGTCCGGATACGCGCCGCGTAGCGGGGCCTCCCGTTCGCG
GCCCCGGGCCGGGGCCCGGTCGCCGGCGGCGTCGGCTGCG
TCGTCGTACTCGTCCCCGTCATCGTCGTCGGCTAGAAAGG
CGGGGGCCGGGGCGGCGAGGCCGCGGGGTCGGGCGTCGGG
ATCGTCCGGACGGCCTCCTCTACCATGGAGGCCAGCGAGC
CAGCTGTCGCGGCGAGACGGCGTCCCCGGCGTCCTCGCCG
GCGTCGGTGCCCGCCGCGGGGGCCCTCCCGTCCCGCCGGG
CGTCGTCGAGGTCGTGGGGGTGGTCGGGGTCGTGGTCGGG
GTCGTCCCCGCCCTCCTCCGTCTCCGCGCCCCACCCGAGG
GCCCCCCGCTCGTCGCGGTCTGGGCTCGGGGGGGCGGCGG
CCCGTCGGTGGGGCCCGGGGGCCGGGGCGCTGCTTGTTCT
CCGACGCCATCGCCGATGCGGGGCGATCCTCCGGGGATAC
GGCTGCGACGGCGGACGTAGCACGGTAGGTCACCTACGGA
CTCTCGATGGGGGGAGGGGGCGAGACCCACGGACCCCGAC
GACCCCCGCCGTCGACGCGGAACTAGCGCGGACCGGTCGA
TGCTTGGGTGGGGAAAAAGGACAGGGACGGCCGATCCCCC
TCCCGCGCTTCGTCCGCGTATCGGCGTCCCGGCGCGGCGA
GCGTCTGACGGTCTGTCTCTGGCGGTCCCGCGTCGGGTCG
TGGATCCGTGTCGGCAGCCGCGCTCCGTGTGGACGATCGG
GGCGTCCTCGGGCTCATATAGCCCAGGGGCCGGCGGGAAG
GAGGAGCAGCGGAGGCCGCCGGCCCCCCGCCCCCCGGCGG
GCCCACCCCGAACGGAATTCCATTATGCACGACCCCGCCC
CGACGCCGGCACGCCGGGGGCCCGTGGCCGCGGCCCGTTG
GTCGAACCCCCGGCCCCGCCCATCCGCGCCATCTGCCATG
GACGGGGCGCGAGGGCGGGTGGGTCCGCGCCCCGCCCCGC
ATGGCATCTCATTACCGCCCGATCCGGCGGTTTCCGCTTC
CGTTCCGCATGCTAACGAGGAACGGGCAGGGGGCGGGGCC
CGGGCCCCGACTTCCCGGTTCGGCGGTAATGAGATACGAG
CCCCGCGCGCCCGTTGGCCGTCCCCGGGCCCCCGGTCCCG
CCCGCCGGACGCCGGGACCAACGGGACGGCGGGCGGCCCA
AGGGCCGCCCGCCTTGCCGCCCCCCCATTGGCCGGCGGGC
GGGACCGCCCCAAGGGGGCGGGGCCGCCGGGTAAAAGAAG
TGAGAACGCGAAGCGTTCGCACTTCGTCCCAATATATATA
TATTATTAGGGCGAAGTGCGAGCACTGGCGCCGTGCCCGA
CTCCGCGCCGGCCCCGGGGGAGGGAGGAGGGGGGCGGGTC
TCTCCGGCGCACATAAAGGCCCGGCGCGACCGACGCCCGC
AGACGGCGCCGGCCACGAACGACGGGAGCGGCTGCGGAGC
ACGCGGACCGGGAGCGGGAGTCGCAGAGGGCCGTCGGAGC
GGACGGCGTCGGCATCGCGACGCCCCGGCTCGGGATCGGG
ATCGCATCGGAAAGGGACACGCGGACGCGGGGGGGAAAGA
CCCGCCCACCCCACCCACGAAACACAGGGGACGCACCCCG
GGGGCCTCCGACGACAGAAACCCACCGGTCCGCCTTTTTT
GCACGGGTAAGCACCTTGGGGGGGGGAGGAGGGGGGACGC
GGGGGCGGAGGAGGGGGGACGCGGGGGCGGAGGAGGGGGG
ACGCGGGGGCGGGGGGGGGGGCGCGGGGGCGGAGGAGGGG
GGGACGCGGGGGGGGGGGGGGGGGGCGCGGGGGCGGAGGA
GGGGGCTCACCCGCGTTCGTGCCTTCCCGCAGGAGGAACG
CCCTCGTCGAGGCGACCGGCGGCGACCGTTGCGTGGACCG
CTTCCTGCTCGTCGGGGGGGGGGGGAGCCACTGTGGTCCT
CCGGGACGTTTTCTGGATGGCCGACATTTCCCCAGGCGCT
TTTGTGCCTTGTGTAAAAGCGCGGCGTCCCGCTCTCCGAT
CCCCGCCCCTGGGCACGCGCAAGCGCAAGCGCCCTGCCCG
CCCCCTCTCATCGGAGTCTGAGGTCGAATCCGAGACAGCC
TTGGAGTCTGAGGTCGATCCGAGACAGCATCGGATTCGAC
CGAGTCTGGGGACCAGGAGGAAGCCCCGCATCGGTGGCCG
TAGGGCCCCCCGGAGGCTTGGGGGGCGGTTTTTTCTGGAC
ATGTCGGCGGAATCCACCACGGGGACGGAAACGGATGCGT
CGGTGTCGGACGACCCCGACGACACGTCCGACTGGTCTTG
TGACGACATTCCCCCACGACCCAAGCGGGCCCGGGTAAAC
CTGCGGCTCACTAGCTCTCCCGATCGGCGGGATGGGGTTA
TTTTTCCTAAGATGGGGCGGGTCCGGTCTACCCGGGAAAC
GCAGCCCCGGGCCCCCACCCCGTCGGCCCCAAGCCCAAAT
GCAATGCTCCGGCGCTCGGTGCGCCAGGCCCAGAGGCGGA
GCAGCGCACGATGGACCCCCGACCTGGGCTACATGCGCCA
GTGTATCAATCAGCTGTTTCGGGTCCTGCGGGTCGCCCGG
GACCCCCACGGCAGTGCCAACCGCCTGCGCCACCTGATAC
GCGACTGTTACCTGATGGGATACTGCCGAGCCCGTCTGGC
CCCGCGCACGTGGTGCCGCTTGCTGCAGGTGTCCGGCGGA
ACCTGGGGCATGCACCTGCGCAACACCATACGGGAGGTGG
AGGCTCGATTCGACGCCACCGCAGAACCCGTGTGCAAGCT
TCCTTGTTTGGAGGCCAGACGGTACGGCCCGGAGTGTGAT
CTTAGTAATCTCGAGATTCATCTCAGCGCGACAAGCGATG
ATGAAATCTCCGATGCCACCGATCTGGAGGCCGCCGGTTC
GGACCACACGCTCGCGTCCCAGTCCGACACGGAGGATGCC
CCCTCCCCCGTTACGCTGGAAACCCCAGAACCCCGCGGGT
CCCTCGCTGTGCGTCTGGAGGATGAGTTTGGGGAGTTTGA
CTGGACCCCCCAGGAGGGCTCCCAGCCCTGGCTGTCTGCG
GTCGTGGCCGATACCAGCTCCGTGGAACGCCCGGGCCCAT
CCGATTCTGGGGCGGGTCGCGCAGCAGAAGACCGCAAGTG
TCTGGACGGCTGCCGGAAAATGCGCTTCTCCACCGCCTGC
CCCTATCCGTGCAGCGACACGTTTCTCCGGCCGTGAGTCC
GGTCGCCCCGACCCCCTTGTATGTCCCCAAAATAAAAGAC
CAAAATCAAAGCGTTTGTCCCAGCGTCTTAATGGCGGGAA
GGGCGGAGAGAAACAGACCACGCGTACATGGGGGGTGTTT
GGGGGTTTATTGACATCGGGGCTACAGGGTGGTAACCGGA
TAGCAGATGTGAGGAAGTCTGGGCCGTTCGCCGCGAACGG
CGATCAGAGGGTCCGTTTCTTGCGGACCACGGCCCGGTGA
TGTGGGTTGCTCGTCTAAAATCTCGGGCATACCCATACAC
GCACAACACGGACGCCGCACCGAATGGGACGTCGTAAGGG
GGTGGGAGGTAGCTGGGTGGGGTTTGTGCAGAGCAATCAG
GGACCGCAGCCAGCGCATACAATCGCGCTCCCGTCCGTTG
GTCCCGGGCAGGACCACGCCGTACTGGTATTCGTACCGGC
TGAGCAGGGTCTCCAGGGGGTGGTTGGGTGCCGCGGGGAA
CGGGGTCCACGCCACGGTCCACTCGGGCAAAAACCGAGTC
GGCACGGCCCACGGTTCTCCCACCCACGCGTCTGGGGTCT
TGATGGCGATAAATCTTACCCCGAGCCGGATTTTTTGGGC
GTATTCGAGAAACGGCCCACACAGGTCCGCCGCGCCTACC
ACCCACAAGTGGTAGAGGCGAGGGGGGCTGGGTTGGTCTC
GGTGCAACAGTCGGAAGCACGCCACGGCGTCCACGACCTC
GGTGCTCTCCAAGGGGCTGTCCTCCGCAAACAGGCCCGTG
GTGGTGTTTGGGGGGCAGCGACAGGACCTAGTGCGCACGA
TCGGGCGGGTGGGTTTGGGTAAGTCCATCAGCGGCTCGGC
CAACCGTCGAAGGTTGGCCGGGCGAACGACGACCGGGGTA
CCCAGGGGTTCTGATGCCAAAATGCGGCACTGCCTAAGCA
GGAAGCTCCACAGGGCCGGGCTTGCGTCGACGGAAGTCCG
GGGCAGGGCGTTGTTCTGGTCAAGGGGGGGCATTACGTTG
ACGACAACAACGCCCCTGTTGGGATATTACAGGCCCGTGT
CCGGTTTGGGGCACTTGCAGATTTGTAAGGCCACGCACGG
CGGGGAGACAGGCCGACGCGGGGGCTGCTCTAAAAATTTA
AGGGCCCTACGGTCCACAGACCCGCCTTCCCGGGGGGGCC
CTTGGAGCGACCGGCAGCGGAGGCGTCCGGGGGAGGGGAG
GGTTATTTACGGGGGGGTAGGTCAGGGGGTGGGTCGTCAA
ACTGCCGCTCCTTAAAACCCCGGGGCCCGTCGTTCGGGGT
GCTCGTTGGTTGGCACTCACGGGGCGGCGAATGGCCTGTC
GTAAGTTTTGTCGCGTTTACGGGGGACAGGGCAGGAGGAA
GGAGGAGGCCGTCCCGCCGGAGACAAAGCCGTCCCGGGTG
TTTCCTCATGGCCCCTTTTATACCCCAGCCGAGGACGCGT
GCCTGGACTCCCCGCCCCCGGAGACCCCCAAACCTTCCCA
CACCACACCACCCGGCGATGCCGAGCGCCGGCATCTGCAG
GAGAGGCAGATGGACGGAAACCAGGACTACCCCATAGAGG
ACGACCCCAGCGCGGATGCCGCGGACGATGTCGACGAGGA
CGCCCCGGACGACGTGGCCTATCCGGAGGAATACGCAGAG
GAGCTTTTTCTGCCCGGGGACGCGACCGGTCCCCTTATCG
GGGCCAACGACCACATCCCTCCCCCGCGTGGCGCATCTCC
CCCCGGTATACGACGACGCAGCCGGGATGAGATTGGGGCC
ACGGGATTTACCGCAGAAGAGCTGGACGCCATGGACAGGC
AGGCGGCTCGAGCCATCAGCCGCGGCGGCAAGCCCCCCTC
GACCAATGGCCAAGCTGGTGACTGGCATGGGCTTTACGAT
CCACGGAGCGCTCACCCCAGGATCGGAGGGGTGTGTCTTT
GACAGCAGCCACCCAGATTACCCCCAACGGGTAATCGTGA
AGGCGGGGTGGTACACGAGCACGAGCCACGAGGCGCGACT
GCTGAGGCGACTGGACCACCCGGCGATCCTGCCCCTCCTG
GACCTGCATGTCGTCTCCGGGGTCACGTGTCTGGTCCTCC
CCAAGTACCAGGCCGACCTGTATACCTATCTGAGTAGGCG
CCTGAACCCACTGGGACGCCCGCAGATCGCAGCGGTCTCC
CGGCAGCTCCTAAGCGCCGTTGACTACATTCACCGCCAGG
GCATTATCCACCGCGACATTAAGACCGAAAATTTTTTATT
AACACCCCCGAGGACATTTGCCTGGGGGACTTTGGTGCCG
CGTGCTTCGTGCAGGGTTCCCGATCAAGCCCCTTCCCCTA
CGGAATCGCCGGAACCATCGACACCAACGCCCCCGAGGTC
CTGGCCGGGGATCCGTATACCACGACCGTCGACATTTGGA
GCGCCGGTCTGGTGATCTTCGAGACTGCCGTCCACAACGC
GTCCTTGTTCTCGGCCCCCCGCGGCCCCAAAAGGGGCCCG
TGCGACAGTCAGATCACCCGCATCATCCGACAGGCCCAGG
TCCACGTTGACGAGTTTTCCCCGCATCCAGAATCGCGCCT
CCCTCGCGCTACCGCTCCCGCGCGGCCGGGAACAATCGCC
CGCCTTACACCCGACCGGCCTGGACCCGCTACTACAAGAT
GGACATAGACGTCGAATATCTGGTTTGCAAAGCCCTCACC
TTCGACGGCGCGCTTCGCCCCAGCGCCGCAGAGCTGCTTT
GTTTGCCGCTGTTTCAACAGAAATGACCGCCCCCGGGGGG
CGGTGCTGTTTGCGGGTTGGCACAATAAGACCCCGACCCG
CGTCTGTGGTGTTTTTGGCATCATGTCGCCGGGCGCCATG
CGTGCCGTTGTTCCCATTATCCCATTCCTTTTGGTTCTTG
TCGGTGTATCGGGGGTTCCCACCAACGTCTCCTCCACCAC
CCAACCCCAACTCCAGACCACCGGTCGTCCCTCGCATGAA
GCCCCCAACATGACCCAGACCGGCACCACCGACTCTCCCA
CCGCCATCAGCCTTACCACGCCCGACCACACACCCCCCAT
GCCAAGTATCGGACTGGAGGAGGAGGAGGAAGAGGAGGAG
GGGGCCGGGGATGGCGAACATCTTAAGGGGGGAGATGGGA
CCCGTGACACCCTACCCCAGTCCCCGGGTCCAGCCGTCCC
GTTGGCCGGGGATGACGAGAAGGACAAACCCAACCGTCCC
GTAGTCCCACCCCCCGGTCCCAACAACTCCCCCGCGCGCC
CCGAGACCAGTCGACCGAAGACACCCCCCACCAGTATCGG
GCCGCTGGCAACTCGACCCACGACCCAACTCCCCTCAAAG
GGGCGACCCTTGGTTCCGACGCCTCAACATACCCCGCTGT
TCTCGTTCCTCACTGCCTCCCCCGCCCTGGACACCCTCTT
CGTCGTCAGCACCGTCATCCACACCTTATCGTTTGTGTGT
ATTGTTGCTATGGCGACACACCTGTGTGGTGGTTGGTCCA
GACGCGGGCGACGCACACACCCTAGCGTGCGTTACGTGTG
CCTGCCGCCCGAACGCGGGTAGGGTATGGGGCGGGGATGG
GGAGAGCCCACACGCGGAAAGCAAGAACAATAAAGGCGGC
GGGATCTAGTTGATATGCGTCTCTGGGTGTTTTTGGGGTG
TGGTGGGCGCGGGGCGGTCATTGGACGGGGGTGCAGTTAA
ATACATGCCCGGGACCCATGAAGCATGCGCGACTTCCGGG
CCTCGGAACCCACCCGAAACGGCCAACGGACGTCTGAGCC
AGGCCTGGCTATCCGGAGAAACAGCACACGACTTGGCGTT
CTGTGTGTCGCGATGTCTCTGCGCGCAGTCTGGCATCTGG
GGCTTTTGGGAAGCCTCGTGGGGGCTGTTCTTGCCGCCAC
CCATCTGGGACCTGCGGCCAACACAACGGACCCCTTAACG
CACGCCCCAGTGTCCCCTCACCCCAGCCCCCTGGGGGGCT
TTGCCGTCCCCCTCGTAGTCGGTGGGCTGTGTGCCGTAGT
CCTGGGGGCGGCGTGTCTGCTTGAGCTCCTGCGTCGTACG
TGCCGCGGGTGGGGGCGTTACCATCCCTACATGGACCCAG
TTGTCGTATAATTTTTTCCCCCCCCCCCTTCTCCGCATGG
GTGATGTCGGGTCCAAACTCCCGACACCACCAGCTGGCAT
GGTATAAATCACCGGTGCGCCCCCCAAACCATGTCCGGCA
GGGGGATGGGGGGCGAATGCGGAGGGCACCCAACAACACC
GGGCTAACCAGGAAATCCGTGGCCCCGGCCCCCAACAAAG
ATCGCGGTAGCCCGGCCGTGTGACATTATCGTCCATACCG
ACCACACCGACGAATCCCCTAAGGGGGAGGGGCCATTTTA
CGAGGAGGAGGGGTATAACAAAGTCTGTCTTTAAAAAGCA
GGGGTTAGGGAGTTGTTCGGTCATAAGCTTCAGTGCGAAC
GACCAACTACCCCGATCATCAGTTATCCTTAAGGTCTCTT
TTGTGTGGTGCGTTCCGGTATGGGGGGGGCTGCCGCCAGG
TTGGGGGCCGTGATTTTGTTTGTCGTCATAGTGGGCCTCC
ATGGGGTCCGCGGCAAATATGCCTTGGCGGATGCCTCTCT
CAAGATGGCCGACCCCAATCGCTTTCGCGGCAAAGACCTT
CCGGTCCTGGACCAGCTGACCGACCCTCCGGGGGTCCGGC
GCGTGTACCACATCCAGGCGGGCCTACCGGACCCGTTCCA
GCCCCCCAGCCTCCCGATCACGGTTTACTACGCCGTGTTG
GAGCGCGCCTGCCGCAGCGTGCTCCTAAACGCACCGTCGG
AGGCCCCCCAGATTGTCCGCGGGGCCTCCGAAGACGTCCG
GAAACAACCCTACAACCTGACCATCGCTTGGTTTCGGATG
GGAGGCAACTGTGCTATCCCCATCACGGTCATGGAGTACA
CCGAATGCTCCTACAACAAGTCTCTGGGGGCCTGTCCCAT
CCGAACGCAGCCCCGCTGGAACTACTATGACAGCTTCAGC
GCCGTCAGCGAGGATAACCTGGGGTTCCTGATGCACGCCC
CCGCGTTTGAGACCGCCGGCACGTACCTGCGGCTCGTGAA
GATAAACGACTGGACGGAGATTACACAGTTTATCCTGGAG
CACCGAGCCAAGGGCTCCTGTAAGTACGCCCTCCCGCTGC
GCATCCCCCCGTCAGCCTGCCTCTCCCCCCAGGCCTACCA
GCAGGGGGTGACGGTGGACAGCATCGGGATGCTGCCCCGC
TCATCCCCGAGACCAGCGCACCGTCGCCGTATACAGCTTG
AAGATCGCCGGGTGGCACGGGCCCAAGGCCCCATACACGA
GCACCCTGCTGCCCCCGGAGCTGTCCGAGACCCCCAACGC
CACGCAGCCAGAACTCGCCCCGGAAGACCCCGAGGATTCG
GCCCTCTTGGAGGACCCCGTGGGGACGGTGGCGCCGCAAA
TCCCACCAAACTGGCACATCCCGTCGATCCAGGACGCCGC
GACGCCTTCCATCCCCCGGCCACCCCGAACAACATGGGCC
TGATCGCCGGCGCGGTGGGCGGCAGTCTCCTGGCAGCCCT
GGTCATTTGCGGAATTGTGTACTGGATGCACCGCCGCACT
CGGAAAGCCCCAAAGCGCATACGCCTCCCCCACATCCGGG
AAGACGACCAGCCGTCCTCGCACCAGCCCTTGTTTTACTA
GATACCCCCCCCTTAATGGGTGCGGGGGGGGTCAGGTCTG
CGGGGTTGGGATGGGACCTTAACTCCATATAAAGCGAGTC
TGGAAGGGGGGAAAGGCGGACAGTCGATAAGTCGGTAGCG
GGGGACGCGCACCTGTTCCGCCTGTCGCACCCACAGCTTT
TTCGCGAACCGTCCCGTTTCGGGATGCCGTGCCGCCCGTT
GCAGGGCCTGGTGCTCGTGGGCCTCTGGGTCTGTGCCACC
AGCCTGGTTGTCCGTGGCCCCACGGTCAGTCTGGTATCAA
ACTCATTTGTGGACGCCGGGGCCTTGGGGCCCGACGGCGT
AGTGGAGGAAGACCTGCTTATTCTCGGGGAGCTTCGCTTT
GTGGGGGACCAGGTCCCCCACACCACCTACTACGATGGGG
TCGTAGAGCTGTGGCACTACCCCATGGGACACAAATGCCC
ACGGGTCGTGCATGTCGTCACGGTGACCGCGTGCCCACGT
CGCCCCGCCGTGGCATTTGCCCTGTGTCGCGCGACCGACA
GCACTCACAGCCCCGCGGTGCGGGGGGGGTCAGGTCTGCG
GGGTTGGGATGGGACCTTAACTCCATATAAGCGAGTCGGA
GGGGGGAAAGGCGGACAGTCGATAAGTCGGTAGCGGGGGA
CGCGCACCTGTTCCGCCTGTCGCACCCACAGCTTTTTCGC
GAACCGTCCCGTTTCGGGATGCCGTGCCGCCCGTTGCAGG
GCCTGGTGCTCGTGGGCCTCTGGGTCTGTGCCACCAGCCT
GGTTGTCCGTGGCCCCACGGTCAGTCGGTATCAAACTCAT
TTGTGGACGCCGGGGCCTTGGGGCCCGACGGCGTAGTGGA
GGAAGACCTGCTTATTCTCGGGGAGCTTCGCTTTGTGGGG
GACCAGGTCCCCCACACCACCTACTACGATGGGGTCGTAG
AGCTGTGGCACTACCCCATGGGACACAAATGCCCACGGGT
CGTGCATGTCGTCACGGTGACCGCGTGCCCACGTCGCCCC
GCCGTGGCATTTGCCCTGTGTCGCGCGACCGACAGCACTC
ACAGCCCCGCATATCCCACCCTGGAGCTGAATCTGGCCCA
ACAGCCGCTTTTGCGGGTCCGGAGGGCGACGCGTGACTAT
GCCGGGGTGTACGTGTTACGCGTATGGGTCGGGGACGCAC
CAAACGCCAGCCTGTTTGTCCTGGGGATGGCCATAGCCGC
CGAAGGTACTCTGGCGTACAACGGCTCGGCCCATGGCTCC
TGCGACCCGAAACTGCTTCCGTCTTCGGCCCCGCGTCTGG
CCCCGGCGAGCGTATACCAACCCGCCCCTAACCCGGCCTC
CACCCCCTCCACCACCACCTCCCCCCCTCGACCACCACCT
CCACCCCCTCGACCACCATCCCCGCTCCCCAAGCATCGAC
CACACCCTTCCCCACGGGAGACCCAAAACCCCAACCTCAC
GGGGTCAACCACGAACCCCCATCGAATGCCACGCGAGCGA
CCCGCGACTCGCGATATGCGCTAACGGTGACCCAGATAAT
CCAGATAGCCATCCCCGCGTCCATTATAGCCCTGGTGTTT
CTGGGGAGCTGTATTTGCTTTATACACAGATGTCAACGCC
GCTACCGACGCTCCCGCCGCCCGATTTACAGCCCCCAGAT
ACCCACGGGCATCTCATGCGCGGTGAACGAAGCGGCCATG
GCCCGCCTCGGAGCCGAGCTCAAATCGCATCCGAGCACCC
CCCCCAAATCCCGGCGCCGGTCGTCACGCACGCCAATGCC
CTCCCTGACGGCCATCGCCGAAGAGTCGGAGCCCGCGGGG
GCGGCTGGGCTTCCGACGCCCCCCGTGGACCCCACGACAT
CCCCCCAACGCCTCCCCTGTTGGTATAGGTCCACGGCCAC
TGGCCGGGGGCACCACATAACCGACCGCAGTCACTGAGTT
GGGAATAAACCGGTATTATTTTCCTATATCCGTGTATGTC
CATTTCTTTCTTCCCCCCCCCCCCCGGAAACCAAAGAAGG
AAGCAAAGAATGGATGGGAGGAGTTCAGGAAGCCGGGGAG
AGGGCCCGCGGCGCATTTAAGGCGTTGTTGTGTTGACTTT
GGCTCTTCTGGCGGGTTGGTGCGGTGCTGTTTGTTGGGCT
CCCATTTTACCCGAAGATCGGCTGCTATCCCCGGGCATGG
ATCGCGGGGCGGTGGGGGGGCTTCTTCTCGGTGTTTGTGT
TGTATCGTGCTTGGCGGGAACGCCCAAAACGTCCTGGAGA
CGGGTGAGTGTCGGCGAGGACGTTTCGTTGCTTCCACTCG
GGGCCTACGGGGCGCGGCCCGACCCAGAAACTACTATGGG
CCGTGGAACCCCTGGATGGGTGCGGCCCCTTACACCCGTC
GTGGGTCTCGCTGATGCCCCCCAAGCAGGTGCCCGAGACG
GTCGTGGATGCGGCGTGCATGCGCGCTCCGGTCCCGCTGG
CGATGGCGTACGCCCCCCCGGCCCCATCTGCGACCGGGGG
TCTACGAACGGACTTCGTGTGGCAGGAGCGCGCGGCCGTG
GTTAACCGGAGTCTGGTTATTCACGGGGTCCGAGAGACGG
ACAGCGGCCTGTATACCCTGTCCGTGGGCGACATAAAGGA
CCCGGCTCGCCAAGTGGCCTCGGTGGTCCTGGTGGTGCAA
CCGGCCCCAGTTCCGACCCCACCCCCGACCCCAGCCGATT
ACGACGAGGATGACAATGACGAGGGCGAGGACGAAAGTCT
CGCCGGCACTCCCGCCAGCGGGACCCCCCGGCTCCCGCCT
CCCCCCCCCCCCCGAGGTCTTGGCCCAGCGCCCCCGAAGT
CTCACATGTGCGTGGGGTGACCGTGCGTATGGAGACTCCG
GAAGCTATCCTGTTTTCCCCCGGGGAGACGTTCAGCACGA
ACGTCTCCATCCATGCCATCGCCCACGACGACCAGACCTA
CTCCATGGACGTCGTCTGGTTGAGGTTCGACGTGCCGACC
TCGTGTGCCGAGATGCGAATATACGAATCGTGTCTGTATC
ACCCGCAGCTCCCAGAATGTCTGTCCCCGGCCGACGCGCC
GTGCGCCGCGAGTACGTGGACGTCTCGCCTGGCCGTCCGC
AGCTACGCGGGGTGTTCCAGAACAAACCCCCCACCGCGCT
GTTCGGCCGAGGCTCACATGGAGCCCGTCCCGGGGCTGGC
GTGGCAGGCGGCCTCCGTCAATCTGGAGTTCCGGGACGCG
TCCCCACAACACTCCGGCCTGTATCTGTGTGTGGTGTACG
TCAACGACCATATTCACGCCTGGGGCCACATTACCATCAG
CACCGCGGCGCAGTACCGGAACGCGGTGGTGGAACAGCCC
CTCCCACAGCGCGGCGCGGATTTGGCCGAGCCCACCCACC
CGCACGTCGGGGCCCCTCCCCACGCGCCCCCAACCCACGG
CGCCCTGCGGTTAGGGGCGGTGATGGGGGCCGCCCTGCTG
CTGTCTGCGCTGGGGTTGTCGGTGTGGGCGTGTATGACCT
GTTGGCGCAGGCGTGCCTGGCGGGCGGTTAAAAGCAGGGC
CTCGGGTAAGGGGCCCACGTACATTCGCGTGGCCGACAGC
GAGCTGTACGCGGACTGGAGCTCGGACAGCGAGGGAGAAC
GCGACCAGGTCCCGTGGCTGGCCCCCCCGGAGAGACCCGA
CTCTCCCTCCACCAATGGATCCGGCTTTGAGATCTTATCA
CCAACGGCTCCGTCTGTATCCCCCGTAGCGACGGGCATCA
ATCTCGCCGCCAGCTCACAACCTTTGGATCCGGAAGGCCC
GATCGCCGTTACTCCCAGGCCTCCGATTCGTCCGTCTTCT
GGTAAGGCGCCCCATCCCGAGGCCCCACGTCGGTCGCCGA
ACTGGGCGACCGCCGGCGAGGTGGACGTCGGAGACGAGCT
AATCGCGATTTCCGACGAACGCGGACCCCCCCGACATGAC
CGCCCGCCCCTCGCCACGTCGACCGCGCCCTCGCCACACC
CGCGACCCCCGGGCTACACGGCCGTTGTCTCCCCGATGGC
CCTCCGGCTGTCGACGCCCCCTCCCTGTTTGTCGCCTGGC
TGGCCGCTCGGTGGCTCCGGGGGGCTTCCGGCCTGGGGGC
CGTCCTGTGTGGGATTGCGTGGTATGTGACGTCAATTGCC
CGAGGCGCACAAAGGGCCGGTGGTCCGCCTAGCCGCAGCA
AATTAAAAATCGTGAGTCACAGCGACCGCAACTTCCCACC
CGGAGCTTTCTTCCGGCCTCGATGACGTCCCGGCTCTCCG
ATCCCAACTCCTCAGCGCGATCCGACATGTCCGTGCCGCT
TTATCCCACGGCCTCGCCAGTTTCGGTCGAAGCCTACTAC
TCGGAAAGCGAAGACGAGGCGGCCAACGACTTCCTCGTAC
GCATGGGCCGCCAACAGTCGGTATTAAGGCGTTGACGCAG
ACGCACCCGCTGCGTCGGCATGGTGATCGCCTGTCTCCTC
GTGGCCGTTCTGTCGGGCGGATTTGGGGCGCTCCTGATGT
GGCTGCTCCGCTAAAAGACCGCATCGACACGCGCGTCCTT
CTTGTCGTCTCTCTTCCCCCCCATCACCCCGCAATTTGCA
CCCAGCCTTTAACTACATTAAATTGGGTTCGATTGGCAAT
GTTGTCTCCCGGTTGATTTTTGGGTGGGTGGGGAGTGGGT
GGGTGGGGAGTGGGTGGGGGAATGGGTGGG (SEQ ID NO: 1)
SEQ ID NO: 9 is a nucleotide sequence
that encodes pSH-tetR.
tcgcgcgtttcggtgatgacggtgaaaacctctgacacat
gcagctcccggagacggtcacagcttgtctgtaagcggat
gccgggagcagacaagcccgtcagggcgcgtcagcgggtg
ttggcgggtgtcggggctggcttaactatgcggcatcaga
gcagattgtactgagagtgcaccatatgcggtgtgaaata
ccgcacagatgcgtaaggagaaaataccgcatcaggcgcc
attcgccattcaggctgcgcaactgttgggaagggcgatc
ggtgcgggcctcttcgctattacgccagctggcgaaaggg
ggatgtgctgcaaggcgattaagttgggtaacgccagggt
tttcccagtcacgacgttgtaaaacgacggccagtgccaa
gcttggctgcaggtcaacaccagagcctgcccaacatggc
acccccactcccacgcacccccactcccacgcacccccac
tcccacgcacccccactcccacgcacccccactcccacgc
acccccactcccacgcacccccactcccacgcacccccac
tcccacgcacccccactcccacgcatccccgcgatacatc
caacacagacagggaaaagatacaaaagtaaacctttatt
tcccaacagacagcaaaaatcccctgagtttttttttatt
agggccaacacaaaagacccgctggtgtgtggtgcccgtg
tctttcacttttcccctccccgacacggattggctggtgt
agtgggcgcggccagagaccacccagcgcccgaccccccc
ctccccacaaacacggggggcgtcccttattgttttccct
cgtcccgggtcgaccagacatgataagatacattgatgag
tttggacaaaccacaactagaatgcagtgaaaaaaatgct
ttatttgtgaaatttgtgatgctattgctttatttgtaac
cattataagctgcaataaacaagttctgctttaataagat
ctgaattcccgggatccgctgtacgcggacccactttcac
atttaagttgtttttctaatccgcatatgatcaattcaag
gccgaataagaaggctggctctgcaccttggtgatcaaat
aattcgatagcttgtcgtaataatggcggcatactatcag
tagtaggtgtttccctttcttctttagcgacttgatgctc
ttgatcttccaatacgcaacctaaagtaaaatgccccaca
gcgctgagtgcatataatgcattctctagtgaaaaacctt
gttggcataaaaaggctaattgattttcgagagtttcata
ctgtttttctgtaggccgtgtacctaaatgtacttttgct
ccatcgcgatgacttagtaaagcacatctaaaacttttag
cgttattacgtaaaaaatcttgccagctttccccttctaa
agggcaaaagtgagtatggtgcctatctaacatctcaatg
gctaaggcgtcgagcaaagcccgcttattttttacatgcc
aatacaatgtaggctgctctacacctagcttctgggcgag
tttacgggttgttaaaccttcgattccgacctcattaagc
agctctaatgcgctgttaatcactttacttttatctaatc
tagacatatcaattcgccctatagtgagtcgtattacaat
tctttgccaaaatgatgagacagcacaataaccagcacgt
tgcccaggagctgtaggaaaaagaagaaggcatgaacatg
gttagcagaggggcccggtttggactcagagtattttatc
ctcatctcaaacagtgtatatcattgtaaccataaagaga
aaggcaggatgatgaccaggatgtagttgtttctaccaat
aagaatatttccacgccagccagaatttatatgcagaaat
attctaccttatcatttaattataacaattgttctctaaa
actgtgctgaagtacaatataatataccctgattgccttg
aaaaaaaagtgattagagaaagtacttacaatctgacaaa
taaacaaaagtgaatttaaaaattcgttacaaatgcaagc
taaagtttaacgaaaaagttacagaaaatgaaaagaaaat
aagaggagacaatggttgtcaacagagtagaaagtgaaag
aaacaaaattatcatgagggtccatggtgatacaagggac
atcttcccattctaaacaacaccctgaaaactttgccccc
tccatataacatgaattttacaatagcgaaaaagaaagaa
caatcaagggtccccaaactcaccctgaagttctcaggat
cgatccggagctttttgcaaaagcctaggcctccaaaaaa
gcctcttcactacttctggaatagctcagaggccctagag
gatccccggcggggtcgtatgcggctggagggtcgcggac
ggagggtccctgggggtcgcaacgtaggcggggcttctgt
ggtgatgcggagagggggcggcccgagtctgcctggctgc
tgcgtctcgctccgagtgccgaggtgcaaatgcgaccaga
ctgtcgggccagggctaacttataccccacgcctttcccc
tccccaaaggggcggcagtgacgattcccccaatggccgc
gcgtcccaggggaggcaggcccaccgcggggcggccccgt
ccccggggaccaacccggcgcccccaaagaatatcattag
catgcacggcccggcccccgatttgggggcccaacccggt
gtcccccaaagaaccccattagcatgcccctcccgccgac
gcaacaggggcttggcctgcgtcggtgccccggggcttcc
cgccttcccgaagaaactcattaccatacccggaacccca
ggggaccaatgcgggttcattgagcgacccgcgggccaat
gcgcgaggggccgtgtgttccgccaaaaaagcaattagca
taacccggaaccccaggggagtggttacgcgcggcgcggg
aggcggggaataccggggttgcccattaagggccgcggga
attgccggaagcgggaagggcggccggggccgcccattaa
tgagtttctaattaccataccgggaagcggaacaaggcct
cttgcaagtttttaattaccataccgggaagtgggcggcc
cggcccattgggcggtaactcccgcccaatgggccgggcc
ccgaagactcggcggacgctggttggccgggccccgccgc
gctggcggccgccgattggccagtcccgcccccgaggcgg
cccgccctgtgagggcgggctggctccaagcgtatatatg
cgcggctcctgccatcgtctctccggagagcggcttggtg
cggagctcgaattcggtaatcatggtcatagctgtttcct
gtgtgaaattgttatccgctcacaattccacacaacatac
gagccggaagcataaagtgtaaagcctggggtgcctaatg
agtgagctaactcacattaattgcgttgcgctcactgccc
gctttccagtcgggaaacctgtcgtgccagctgcattaat
gaatcggccaacgcgcggggagaggcggtttgcgtattgg
gcgctcttccgcttcctcgctcactgactcgctgcgctcg
gtcgttcggctgcggcgagcggtatcagctcactcaaagg
cggtaatacggttatccacagaatcaggggataacgcagg
aaagaacatgtgagcaaaaggccagcaaaaggccaggaac
cgtaaaaaggccgcgttgctggcgtttttccataggctcc
gcccccctgacgagcatcacaaaaatcgacgctcaagtca
gaggtggcgaaacccgacaggactataaagataccaggcg
tttccccctggaagctccctcgtgcgctctcctgttccga
ccctgccgcttaccggatacctgtccgcctttctcccttc
gggaagcgtggcgctttctcaatgctcacgctgtaggtat
ctcagttcggtgtaggtcgttcgctccaagctgggctgtg
tgcacgaaccccccgttcagcccgaccgctgcgccttatc
cggtaactatcgtcttgagtccaacccggtaagacacgac
ttatcgccactggcagcagccactggtaacaggattagca
gagcgaggtatgtaggcggtgctacagagttcttgaagtg
gtggcctaactacggctacactagaaggacagtatttggt
atctgcgctctgctgaagccagttaccttcggaaaaagag
ttggtagctcttgatccggcaaacaaaccaccgctggtag
cggtggtttttttgtttgcaagcagcagattacgcgcaga
aaaaaaggatctcaagaagatcctttgatcttttctacgg
ggtctgacgctcagtggaacgaaaactcacgttaagggat
tttggtcatgagattatcaaaaaggatcttcacctagatc
cttttaaattaaaaatgaagttttaaatcaatctaaagta
tatatgagtaaacttggtctgacagttaccaatgcttaat
cagtgaggcacctatctcagcgatctgtctatttcgttca
tccatagttgcctgactccccgtcgtgtagataactacga
tacgggagggcttaccatctggccccagtgctgcaatgat
accgcgagacccacgctcaccggctccagatttatcagca
ataaaccagccagccggaagggccgagcgcagaagtggtc
ctgcaactttatccgcctccatccagtctattaattgttg
ccgggaagctagagtaagtagttcgccagttaatagtttg
cgcaacgttgttgccattgctacaggcatcgtggtgtcac
gctcgtcgtttggtatggcttcattcagctccggttccca
acgatcaaggcgagttacatgatcccccatgttgtgcaaa
aaagcggttagctccttcggtcctccgatcgttgtcagaa
gtaagttggccgcagtgttatcactcatggttatggcagc
actgcataattctcttactgtcatgccatccgtaagatgc
ttttctgtgactggtgagtactcaaccaagtcattctgag
aatagtgtatgcggcgaccgagttgctcttgcccggcgtc
aatacgggataataccgcgccacatagcagaactttaaaa
gtgctcatcattggaaaacgttcttcggggcgaaaactct
caaggatcttaccgctgttgagatccagttcgatgtaacc
cactcgtgcacccaactgatcttcagcatcttttactttc
accagcgtttctgggtgagcaaaaacaggaaggcaaaatg
ccgcaaaaaagggaataagggcgacacggaaatgttgaat
actcatactcttcctttttcaatattattgaagcatttat
cagggttattgtctcatgagcggatacatatttgaatgta
tttagaaaaataaacaaataggggttccgcgcacatttcc
ccgaaaagtgccacctgacgtctaagaaaccattattatc
atgacattaacctataaaaataggcgtatcacgaggccct
ttcgtc (SEQ ID NO: 9)
SEQ ID NO: 10 is a nucleotide sequence
that encodes the open reading frame
of UL24 (strain KOS).
atg gccgcgagaa cgcgcagcct ggtcgaacgc
agacgcgtgt tgatggcagg ggtacgaagc
catacgcgct tctacaaggc gcttgccaaa
gaggtgcggg agtttcacgc caccaagatc
tgcggcacgc tgttgacgct gttaagcggg
tcgctgcagg gtcgctcggt gttcgaggcc
acacgcgtca ccttaatatg cgaagtggac
ctgggaccgc gccgccccga ctgcatctgc
gtgttcgaat tcgccaatga caagacgctg
ggcggggttt gtgtcatcat agaactaaag
acatgcaaat atatttcttc cggggacacc
gccagcaaac gcgagcaacg ggccacgggg
atgaagcagc tgcgccactc cctgaagctc
ctgcagtccc tcgcgcctcc gggtgacaag
atagtgtacc tgtgccccgt cctggtgttt
gtcgcccaac ggacgctccg cgtcagccgc
gtgacccggc tcgtcccgca gaaggtctcc
ggtaatatca ccgcagtcgt gcggatgctc
cagagcctgt ccacgtatac ggtccccatg
gagcctagga cccagcgagc ccgtcgccgc
cgcggcggcg ctgcccgggg gtctgcgagc
agaccgaaaa ggtcacactc tggggcgcgc
gacccgcccg agccagcggc ccgccaggta
ccacccgccg accaaacccc cgcctccacg
gagggcgggg gggtgcttaa gaggatcgcg
gcgctcttct gcgtgcccgt ggccaccaag
accaaacccc gagctgcctc cgaatga (SEQ ID NO: 10)
SEQ ID NO: 11 is a nucleotide sequence
that encodes the open reading frame
of gK (strain KOS).
atgctcgccg tccgttccct gcagcacctc
tcaaccgtcg tcttgataac ggcgtacggc
ctcgtgctcg tgtggtacac cgtcttcggt
gccagtccgc tgcaccgatg tatttacgcg
gtacgcccca ccggcaccaa caacgacacc
gccctcgtgt ggatgaaaat gaaccagacc
ctattgtttc tgggggcccc gacgcacccc
cccaacgggg gctggcgcaa ccacgcccat
atctgctacg ccaatcttat cgcgggtagg
gtcgtgccct tccaggtccc acccgacgcc
acgaatcgtc ggatcatgaa cgtccacgag
gcagttaact gtctggagac cctatggtac
acacgggtgc gtctggtggt cgtagggtgg
ttcctgtatc tggcgttcgt cgccctccac
caacgccgat gtatgtttgg tgtcgtgagt
cccgcccaca agatggtggc cccggccacc
tacctcttga actacgcagg ccgcatcgta
tcgagcgtgt tcctgcagta cccctacacg
aaaattaccc gcctgctctg cgagctgtcg
gtccagcggc aaaacctggt tcagttgttt
gagacggacc cggtcacctt cttgtaccac
cgccccgcca tcggggtcat cgtaggctgc
gagttgatgc tacgctttgt ggccgtgggt
ctcatcgtcg gcaccgcttt catatcccgg
ggggcatgtg cgatcacata ccccctgttt
ctgaccatca ccacctggtg ttttgtctcc
accatcggcc tgacagagct gtattgtatt
ctgcggcggg gcccggcccc caagaacgca
gacaaggccg ccgccccggg gcgatccaag
gggctgtcgg gcgtctgcgg gcgctgttgt
tccatcatcc tgtcgggcat cgcaatgcga
ttgtgttata tcgccgtggt ggccggggtg
gtgctcgtgg cgcttcacta cgagcaggag
at ccagaggc gcctgtttga tgtatga (SEQ ID NO: 11)

1.-26. (canceled)

27. An oncolytic Herpes Simplex Virus (HSV) comprising recombinant DNA, wherein the recombinant DNA comprises:
a) a gene comprising a 5′ untranslated region and an HSV-1, or HSV-2, ICP27 gene that is operably linked to an ICP27 promoter comprising a TATA element;
b) a tetracycline operator sequence positioned between 6 and 24 nucleotides 3′ to said TATA element, wherein the ICP27 gene lies 3′ to said tetracycline operator sequence;
c) a ribozyme sequence located in said 5′ untranslated region of said gene;
d) a gene sequence encoding tetracycline repressor operably linked to an HSV immediate-early promoter, wherein the gene sequence is located at the ICP0 locus; and
e) at least two genes that are variant relative to wild typeHSV-1 or wild-type HSV-2, wherein the at least two variant genes are (i) a glycoprotein K (gK) gene variant and (ii) a UL24 gene variant,
wherein said oncolytic HSV does not encode functional ICP0 and functional ICP34.5 protein.

28. The oncolytic HSV of claim 27, wherein one of the at least two variant genes is a gK variant gene that encodes an amino acid substitution selected from the group consisting of: an Ala to Val amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.

29. The oncolytic HSV of claim 27, wherein one of the at least two variant genes is a UL24 variant gene that encodes a Ser to Asn amino acid substitution corresponding to amino acid 113 of SEQ ID NO: 3.

30. The oncolytic HSV of claim 27, wherein the tetracycline operator sequence comprises two Op2 repressor binding sites.

31. The oncolytic HSV of claim 27, wherein the ICP27 promoter is an HSV-1 or HSV-2 ICP27 promoter.

32. The oncolytic HSV of claim 27, wherein the immediate-early promoter is an HSV-1 or HSV-2 immediate-early promoter.

33. The oncolytic HSV of claim 32, wherein the HSV immediate-early promoter is selected from the group consisting of: ICP0 promoter and ICP4 promoter.

34. The oncolytic HSV of claim 27, wherein the recombinant DNA is part of the HSV-1 genome or HSV-2 genome.

35. The oncolytic HSV of claim 27, further encoding at least one polypeptide that can increase the efficacy of the oncolytic HSV to induce an anti-tumor-specific immunity, wherein the at least one polypeptide encodes a product selected from the group consisting of: interleukin 2 (IL-2), interleukin 12 (IL-12), interleukin 15 (IL-15), an anti-PD-1 antibody or antibody reagent, an anti-PD-L1 antibody or antibody reagent, an anti-OX40 antibody or antibody reagent, CTLA-4 antibody or antibody reagent, TIM-3 antibody or antibody reagent, and TIGIT antibody or antibody reagent.

36. A composition comprising an oncolytic HSV of claim 27.

37. The composition of claim 36, further comprising a pharmaceutically acceptable carrier.

38. A method for treating cancer, the method comprising administering the oncolytic HSV of claim 27 to a subject having cancer, wherein the cancer is breast cancer.

39. The method of claim 38, further comprising administering an agent that regulates a tetracycline operator-containing promoter.

40. The method of claim 39, wherein the agent is doxycycline or tetracycline.

41. The method of claim 39, wherein the agent is administered locally or systemically.


SEQUENCE LISTING (11 sequences)

SEQ ID NO: 1 - DNA, 147630 bp, synthetic construct
  misc_feature 1..147630: note = Description of Artificial Sequence: Synthetic polynucleotide
  source 1..147630: mol_type = other DNA
ccctagaggatctgcggctggagggtcgctgacggagggtccctgggggtcgcaacgtaggcttttcttctttttttcttcttccctcccccgcccgagggggcgcccgagtctgcctggctgctgcgtctcgctccgagtgccgaggtgcaaatgcgaccagaccgtcgggccagggctaacttataccccacgcctttcccctccccaaaggggcggcagtgacgattcccccaatggccgcgcgtcccaggggaggcaggcccaccgcggagcggccccgtccccggggaccaacccggcgcccccaaagaatatcattagcatgcacggcccggcccccgatttgggggaccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccactgcgcgaggggccgtgtgttccgccaaaaaagcaattaacataacccggaaccccaggggagtggttacgcgcggcgcgggaggcggggaataccggggttgcccattaagggccgcgggaattgccggaagcgggaagggcggccggggccgcccattaatgagtttctaattaccatcccgggaagcggaacaaggcctctgcaagtttttaattaccataccgggaagtgggcggcccggcccactgggcgggagttaccgcccagtgggccgggccccgacgactcggcggacgctggttggccgggccccgccgcgctggcggccgccgattggccagtcccgccccccgagggcgggcccgcctcgggggcgggccggccccaagcgaatatgcgcggctcctgccttcgtctctccggagagcggcttggtggcggggcccggccaccagcgtccgccgagtcgtcggggcccggcccactgggcggtaactcccgcccagtgggccgggccgcccacttcccggtatggtaattaaaaacttgcagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgttaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttggtcccccaaatcgggggccgggccgtgcatgctaatgatattctttgggggcgccgggttggtccccggggacggggccgctccgcggtgggcctgcctcccctgggacgcgcggccattgggggaatcgtcactgccgcccctttggggaggggaaaggcgtggggtataagttagccctggcccgacggtctggtcgcatttgcacctcggcactcggagcgagacgcagcagccaggcagactcgggccgccccctctccgcatcaccacagaagccccgcctacgttgcgacccccagggaccctccgtcagcgaccctccagccgcatacgaccccccggggatcctctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaacgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgacccgggacgagggaaaacaataagggacgcccccgtgtttgtggggaggggggggtcgggcgctgggtggtctctggccgcgcccactacaccagccaatccgtgtcggggaggtggaaagtgaaagacacgggcaccacacaccagcgggtcttttgtgttggccctaataaaaaaaactcaggggatttttgctgtctgttgggaaataaaggtttacttttgtatcttttccctgtctgtgttggatgtatcgcgggggtgcgtgggagtgggggcccccactcccacgcacccccactcccacgcacccccactcccacgcacccccgcgatacatccaacacagacagggaaaagatacaaaagtaaacctttatttcccaacagacagcaaaaatcccctgagttttttttattagggccaacacaaaagacccgctggtgtgtggtgcccgtgtctttcactttccacctccccgacacggattggctggtgtagtgggcgcggccagagaccacccagcgcccgccccccccccccccacaaccccgggggcgtcccttattgtttccctcgtcccgggtcgacgtcgacccgggacgagggaaaacaataagggacgcccccgtgtttgtggggaggggggggtcgggcgctgggtggtctctggccgcgcccactacaccagccaatccgtgtcggggaggtggaaagtgaaagacacgggcaccacacaccagcgggtcttttgtgttggccctaataaaaaaaactcaggggatttttgctgtctgttgggaaataaaggtttacttttgtatcttttccctgtctgtgttggatgtatcgcgggggtgcgtgggagtgggggtgcgtgggagtgggggtgcgtgggagtgggggtgggggggggggtgcgtgggggagggggggcgtgggagtgggggtgcgtgggggtgggggtgcgtgggagtggcccggagagccgcggcccccggacgcgcccggaaagtctttcgcccaccggcgatcggcacggccgcacccccgcttttataaaggctcagatgacgcagcaaaaacaggccacagcaccacatgggtaggggatgtaattttattttcctcgtctgcggcctaatggatttccgggcgcggtgcccctgtctgcagagcacttaacggattgatatctcgcgggcacgcgcgcccttaaggggccggggggggcggggggccggatacccacacgggcgggggggggtgtcgcgggccgtctgctggcccgcggccacataaacaatgactcggggcctttctgcctctgccgcttgtgtgtgcgcgcgccggctctgcggtgtcggcggcggcggcggcggtggccgccgtgttcggtctcggtagccggccggcgggggactcgcggggggccggagggtggaaggcaggggggtgtaggatgggtatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgtgggcgggggtgctcgtcgatcgaccgggctcagtgggggcgtggggtgggtgggagaaggcgaggagactggggtgggggcgcccccactgagcccggtcgatcgacgagcacccccgcccccccccgcccctgccccggcgacccccggcccgcacgatcccgacaacaataacaaccccaacggaaagcggcggggtgttgggggaggcgaggaacaaccgaggggaacgggggatggaaggacgggaagtggaagtcctgatacccatcctacccccccctgccttcccccctccggccccccgcgagtccacccgccggccggctaccgagaccgaacacggcggccaccgccgccgccgccgccgacaccgcagagccggcgcgcgcacacacaagcggcagaggcagaaaggccccgagtcattgtttatgtggccgcgggccagcagacggcccgcgacacccccccccgcccgtgtgggtatccggccccccgccccgcgccggccccttaagggcgcgcgtgcccgcgagatatcaatccgttaagtgctctgcagacaggggcaccgcgcccggaaatccattaggccgcagacgaggaaaataaaattacatcacctacccatgtgggctgtggcctgttttgctgcgtcatctgagcctttataaaagcgggggcgcggtcgttccgatcgccggtggtgcgaaagactttccgggcgctggggtgggggtgtcggtgggtggttgtttttttttttgtggttgttttttgtgtctgtttccgtcccccgtcacccccctccctccgtcccctccgtccccccgtcgcgggtgtttgtgtttgtttattccgacatcggtttatttaaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtatcccgaggtgggtgggtgggcggcggtggccgggccgggccgggccgggccgggccagcgccccgccggcccccccccccgccgctaaaccccatcccgcccccgggaccccacatataagcccccagccacacgcaagaacagacacgcagaacggctgtgtttattttaaataaaccgatgtcggaataaacaaacacaaacacccgcgacggggggacggaggggacggagggaggggggtgacgggggacggaaacagacacaaaaaacaaccacaaaaaaaaaaacaaccacccaccgcacccccccccttctcctcctcctcctcgttttccaaccccgcccacccggcccggcccggcccggcccggccccgccgcccacccacccacctcgggatacccagccccggtcccccgttccccgggggccgttatctccagcgggggtttggaaaaacgaggaggaggaggagaaggcggggggggagacggggggaaagcaaggacacggcccggggggtgggagcgcgggccgggccgctcgtaagagccgcgacccggccgccggggagcgttgtcgccgtcggtctgccggcccccgtccctcccttttttgaccaaccagcgccctcccccccaccaccattcctactaccaccaccaccaccacccccaccaccgacacctcccgcgcacccccgcccacatccccccaccccgcaccacgagcacggggtgggggtagcaggggatcaaaggggggcaaagccggcggggcggttcgggggggcgggagaccgagtaggcccgcccatacgcggcccctcccggcagccacgccccccagcgtcgggtgtcacggggaaagagcaggggagaggggagagggggggagagggggtatataaaccaacgaaaagcgcgggaacggggatacggggcttgtgtggcacgacgtcgtggttgtgttactgggcaaacacttggggactgtaggtttctgtgggtgccgaccctaggcgctatggggattttgggttgggtcgggcttattgcggttggggttttgtgtgtgcgggggggcttgtcttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttctggaggacttgagtcaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcaaccgaagcccctcaccacgccgccgcccatcatcgccacgtcggcccccaccccgcgacgggacgccgccacaaaaagcagacgccgacgaccccactcccggcgcctctaacgatgcctcgacggaaacccgtccgggttcggggggcgaaccggccgcctgtcgctcgtcagggccggcgggcgctcctcgccgccctagaggctgtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgatggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggtggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgcttcgtgggcggagttatccgccggttggccgcgcgccgccccggcctggtgtttatgctctggggcgcacacgcccagaatgccatcaggccggaccctcgggtccattgcgtcctcaagttttcgcacccgtcgcccctctccaaggttccgttcggaacctgccagcatttcctcgtggcgaaccgatacctcgagacccggtcgatttcacccatcgactggtcggtttgaaaggcatcgacgtccggggtttttgtcggtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcggggatatcgccagatacgactcccgcagattccattcggggggccgctgtggcctcacctaaccaacctttaccgggggcccggaacgggaggcccagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccatgccaccctcactgcccgtcgcgcgtgtttgatgttaataaataacacataaatttggctggttgtttgttgtctttaatggaccgcccgcaagggggggggggcgtttcagtgtcgggtgacgagcgcgatccggccgggatcctaggaccccaaaagtttgtctgcgtattccagggtggggctcagttgaatctcccgcagcacctctaccagcaggtccgcggtgggctggagaaactcggccgtcccggggcaggcggttgtcgggggtggaggcgcggcgcccaccccgtgtgccgcgcctggcgtctcctctgggggcgacccgtaaatggttgcagtgatgtaaatgggtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcacgcaagaccacgtaaggcaggggggcctcttccagtaactcggccacgttggccgtcgcgtgccgcctccgcagctcgtccgcaaaggcaaacacccgtgtgtacgtgtatcccatgagcgtataattgtccgtctgcagggcgacggacatcagccccccgcgcggcgagccggtcagcatctcgcagccccggaagataacgttgtccacgtacgtgctaaagggggcgacttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggccattaccgacgcctgcgtcggcacccccgaagatttggggcgcagagacagaatctccgccgttagttctcccatgcgggcgtacgcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggtgttggccgtgagccacttttcaatagtcaggccgggctggtgtgtcagccgtcggtagtgttcaaactccttgaccgacacgaacgtaagcacggggaggtgtagccgtcggtattcgtcaaactcctttccctcccctcccttcctcccttttctttttcccactccgccctccccctcacgggtcaccttcaggtaggcgtggagcttggccatgtacgcgctcacctctttgtgggaggagaacagccgcgtccagccggggaggttggcggggttggtgatgtagttttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcggttgcagatgaggtacgtgagcacgttctcgctctggcggacggaacaccgcagtttctggtgctcgaaggtcgactccagggacgccgtctgcgtcggcgagcccccacacaccaacacgggccgcaggcgggccgcgtactggggggtgtggtacagggcgttaatcatccaccagcaatacaccacggccgtgaggaggtgacgcccaaggagcccggcctcgtcgatgacgatcacgttgctgcgggtaaaggccggcagcgccccgtgggtggccggggccaaccgcgtcagggcgccctcggccaaccccagggtccgttccagggcggccagggcgcgaaactcgttccgcaactcctcgcccccggaggcggccagggcgcgcttcgtgaggtccaaaatcacctcccagtagtacgtcagatctcgtcgctgcaggtcctccagcgaggcggggttgctggtcagggggtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagacggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtttctgtccgtctagctggcgctccccgccggccgccgccatgaccgcaccacgctcgtgggcccccactacgcgtgcgcggggggacacggaagcgctgtgctcccccgaggacggctgggtaaaggttcaccccacccccggtacgatgctgttccgtgagattctccacgggcagctggggtataccgagggccagggggggtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggccccccttgtggaccggggaccgtctggtcttcgattcgcccgtccagcggctgtttcccgagatcgtcgcgtgtcactccctccggggacacgcgcacgtctgccggctgcgcaataccgcgtccgtcaaggtgctgctggggcgcaagagcgacagcgagcgcggggtggccggtgccgcgcgggtcgttaacaaggtgttgggggaggacgacgagaccaaggccgggtcggccgcctcgcgcctcgtgcggcttatcatcaacatgaagggcatgcgccacgtaggcgacattaacgacaccgtgcgtgcctacctcgacgaggccggggggcacctgatagacgccccggccgtcgacggtaccctccctggattcggcaagggcggaaacaaccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacacgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgagtcagtgcttcgggccccccgggtaggcgcgcccatcacgggctccgatgtcatcctgggggaggaggagttatgggatgcggtgtttaagaaaacccgcctgcaaacgtacctgacagacatcgcggccctgttcgtcgcggacgtccagcacgcagcgctgcccccgcccccctccccggtcggcgccgatttccggcccggcgcgtccccgcggggccggtccagacgcggtcgcccggaaggaagaacggcgccaggcgcgccggaccagggcgggggcatcgggcaccgggatggccgccgcgacggccgacgatgaggggtcggccgccaccatcctcaagcaggccatcgccggggaccgcagcctggtcgaggcggccgaggcgattagccagcagacgctgctccgcctggcctgcgggtgcgccaggtcggcgccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcctccgcagttgccactgggtcctaaacacactcatgttcatggtgcacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttaggaaataaacccggttttgtttctgtggcctcccgacggatgcgcgtgtccttcctccgtcttggtgggtgggtgtctgtgtatccgtcccatctgtgcggagagggggggcatgtcggcacgtattcggacagactcaagcacacacgggggagcgctcttgtctcagggcaatgtttttattggtcaaactcaggcaaacagaaacgacatcttgtcgtcaaagggatacacaaacttccccccctctccccatactcccgccagcaccccggtaaacaccaactcaatctcgcgcaggatttcgcgcaggtgatgagcgcagtccacgggggggagcacaaggggccgcgggtgtagatcgaggggacgccgaccgactcaccgcctccgggacagacacgcacgacgcgccgccagtagtgctctgcgtccagcaaggcgccgccgcggaaggcagtggggggcaaggggtcgctagcctcaagggggacacccgaacgctccagtactccgcgtccaaccgtttattaaacgcgtccacgataaggcggtcgcaggcgtcctccataaggccccgggccgtgagtgcgtcctcctccggcacgcctgccgttgtcaggcccaggacccgtcgcagcgtgtcgcgtacgaccccggccgccgtggtgtacgcgggcccgcggagaggaaatcccccaagatggtcagtgttgtcgcgggagttccagaaccacactcccgcctggttccaggcgactgcgtgggtgtagacgccctcgagggccaggcacagtgggtgccgcagccggaggccgttggccctaagcacgctccacggccgtctcgatggcccgccgggcgtcctcgatcccccggaagccgcatccgcgtcttgggggtccacgttaaagacaccccagaacgcacccccatcgcccccgcagaccgcgaacttcaccgagctggccgtctcctcgatctgcaggcagacggcggccattaccccacccaggagctgccgcagcgcagggcaggcgtcgcacgtgtccgggaccaggcgctccaagacggccccggcccagggctctgagggagcggccaccaccagcgcgtccagtcttgctaggcccgtccggccgtgggggtccgccagcccgctccccccgaggtcggcaaggacaaaaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgcgccctccttcttgatcggcggaaacgcaagagtctggtattcgcgcgcaaatagcgcggttccggtggtgatgttaacggtcagcgaagcggcggacgcgcactggggggtgtcgcgatccgccaggcgcgccccgccacgccgcgcgtcgggatgctcggcaacgcgcgccgccagggccatagggtcgatgtcaatgttggcctccgcgaccaggagagcggcgcgaggggcggcgggcgggccccacgacgctctctcaactttcacccccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacggggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcaggcacgcgtagcttgaagcgcgcgcccgcaaactcccgcttgtgggccataagcagggcgtacagctgcctgtgggtccggcaggcgctgtggtcgatgtggtgggcgtccaacaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagaccgccgatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtgattaaggagcatgggcgtaaagacgggctccgagcgcgccccggagccgtccatgtaaatcagtagctcccccttgcggagggtgcgcacccgtcccagggactggtacacggacaccatgtccggtccgtagttcatgggttttacgtaggcgaacatgccatcaaagtgcaggggatgaagcggaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgacgaaaaaatgcagatgttatcgcccccgccaaggcgcgcttccagctccccaaagaacgtggccccccgggcgtccggagaggcgtccggagacgggccgctcggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacaggtgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgccagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggccccagaggatctcccgactccgcgccccccacgaaaggcatgaccggggcgcggacggcgtggtgggtctggtgtgtgcaggtggcgacgtttgtggtctctgcggtctgcgtcacggggctcctcgtcctggcctctgtgttccgggcacggtttccctgcttttacgccacggcgagctcttatgccggggtgaactccacggccgaggtgcgcgggggtgtagccgtgcccctcaggttggacacgcagagccttgtgggcacttatgtaatcacggccgtgttgttgttggccgcggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcaccagccagctggcccacctggtttacgtcctgcactttgcgtgtctggtgtattttgcggcccatttttgcaccaggggggtcctgagcgggacgtatctgcgtcaggtgcacggcctgatggagccggccccgactcatcatcgcgtcgttggcccggctcgagccgtgctgacaaacgccttgctgttgggcgtcttcctgtgcacggccgacgccgcggtatccctgaataccatcgccgcgttcaactttaatttttcggccccgggcatgctcatatgcctgaccgtgctgttcgcccttctcgtcgtatcgctgttgttggtggtcgagggggtgttgtgtcactacgtgcgcgtgttggtgggcccccacctgggggccgtggccgccacgggcatcgtcggcctggcatgcgagcactattacaccaacggctactacgttgtggagacgcagtggccgggggcccagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaagcgcgtacgcagttccatgcgcggatcgcgagacggccgccacaggcccgcacccggcagcccgcccgggattcccgaatccttcgaagacccctacgcgatctcatacggcggccagctcgaccggtacggagattccgacggggagccgatttacgacgaggtggcggacgaccaaaccgacgtattgtacgccaagatacaacacccgcggcacctgcccgacgacgagcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgttccccccaaaccccggcgattgtgggtttttttaaaaacgacacgcgtgcgaccgtatacagaacattgttgttttttattcgctatcggacatggggggtggaaactgggtggcggggcaggcgcctccgggggttcgccggtgagtgtggcgcgagggggatccgacgaacgcaggcgctgtctccccggggcccgcgtaaccccgcgcatatccgggggcacgtagaaattaccttcctcttcggactcgatatccacgacgtcaaagtcgtgggcggtcagcgagacgacctccccgtcgtcggtgatgaggacgttgtttcggcagcagcagggccgggtttccttttcccccgagcccatagctcggcgagcgtgtcgtcgaacgccaggcggctgcttcgctgtatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctagtgcggtgcctgccgataaacgtcaccagatgcgggtggggggggcagtcggggaagtggctgtcgagcacgtagcccgcaccaagatctgcttaaagttcggggacggggggtcgcgaagacgggctcgcggcgtaccagatccccggagctccaggacacgggggagatggtgtggcgtccgaggtcgggggtgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgccatcaagtcctcgtacgcggaggccgtggggtcgctggggtccatggggtcgaaagcgtacttggcccggcatttgacctcgtaaaaggccaggggggtcttggggactggggccaagtagccgtgaatgtcccgaggacagacgagaatatccagggacgccccgaccatccccgtgtgaccgtccatgaggaccccacacgtatgcacgttctcttcggcgaggtcgccgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggaggtggtatgcggtcgtgaggccgcggagcagggcccatagctggcagtcggcctggttttgcgtggccgcctcgtaaaaccccatgaggggccggggcgccacggcgtccgcggcggccgggggcccgcggcgcgtcaggcgccataggtgccggccgagtccgcggtccaccatacccgcctcctcgaggaccacggccagggaacacagataatccaggcgggccccccccctctcccctctccccccctctcccctgctctttccccgcgacacccgacgctggggggcgtggctgccgggaggggccgcgtatgggcgggcctactcggtctcccgcccccccgaaccgccccgccggctttgcccccctttgatcccctgctacccccaccccgtgctcgtggtgcggggtggggggatgtgggcgggggtgcgcgggaggtgtcggtggtgggggtggtggtggtggtggtagtaggaatggtggtgggggggagggcgctggttggtcaaaaaagggagggacgggggccggcagaccgacggcgacaacgctccccggcggccgggtcgcggccttacggcggcccgcccgcgcccccccccccgggccgtgtccttgctttccccccgtctccccccttttgcgtggccgcctcgtaaacccccagaggggccggggcgccacggcgtccgcggcggccgggggcccgcggcgcgtcaggcgccataggtgccggccgagtccgcggtccaccatacccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggcctgaggcgtcagaaagtgccggcgacgcggcccggggcttttcgtctgcggtgtcggtggcgtgctgatcacgtggggggttaacgggcgaatggggagctcgggtccacagctgacgtcgtctggggtggggggggcaggggacggaaggtggttgttagcggaagactgttagggcgggggcgcttgggggggctgtcggggccacgaggggtgtcctcggccagggcccaggaacgcttagtcacggtgcgtcccggcggacatgctgggcctcccgtggactccatttccgagacgacgtgggggagcggtggttgagcgcgccgccgggtgaacgctgattctcacgacagcgcgtgccgcgcgcacgggttggtgtgacacaggcgggcccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggccctgaggcgtcagaaagtgccggcgacgcggcccggggcttttcgtctgcggtgtcggtggcgtgctgatcacgtggggggttaacgggcgaatgggagctcgggtccacagctgacgtcgtctggggtggggggggcaggggacggaaggtggttgttagcggaagactgttagggcgggggcgcttgggggggctgtcggggccacgaggggtgtcctcggccagggcccaggaacgcttagtcacggtgcgtcccggcggacatgctgggcctcccgtggactccatttccgagacgacgtgggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctgggacgtcatacgccaggccgtgggtgttggtctcggccgagttgacaaacagggctgggtgcagcacgtggcgataggcgagcagggccagggcgaagtccagcgacagctggttgttgaaatactggtaaccgggaaaccgggtcacgggtacgcccaggctcggggcgacgtacacgctaaccaccaactccagcagcgtctggccaagggcgtacaggtcaaccgctaacccgacgtcgtgcttcaggcggtggttggtaaattcggcccgttcgttgttaaggtatttcaccaacagctccgggggctggttatacccgtgacccaccaggggtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcgtgaatcgggtggctccccaggtggtgtttaaaaacacaacggcgcgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagagcgcactccccgaccaacagggtcgcgatgagctcaacggcaaaccactccttttcctttatggtcttaacggcaagcttatgttcgcgaatcagttggacgtcgccgtatcccccagaccccccgaagcttcgggccccggggatctcgagggtcgtgtagtgtagggcggggttgatggcgaacacggggctgcatagcttgcggatgcgcgtgagggtaaggatgtgcgagggggacgaggggggtgcggttaacgccgcctgggatctgcgcaggggcgggcggttcagttggccgccgtaccgggcggctcgggggacgcgcggcgatgagacgagcggctcattcgccatcgggatagtcccgcgcgaagccgctcgcggaggccggatcggtggcgggacccgtgggaggagcgggagccggcggcgtcctggagagaggggccgctggggcgcccggaggccccgtgtgggttgggtgtatgtaggatgcgagccaatccttgaaggactgttggcgtgcattgggggtgaggtgagaggaaaaatgaccagcaggtcgctgtctgcgggactcatccatccttcggccaggtcgccgtcttcccacagagaagcgttggtcgctgcttcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgcgacgccccccggtgcagcgcagggtctggtccttgtaaatctcggctcggaggtgcgtctcggccaggcgtcggcgcagggccgcgtgggcggcatctcggtccattccgccccctgcgggcgacccggggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcacaatgggcggcgatgccctacgagtgccctttttagatttcgcgaccgcgacccccaagcgccaccagaccgtggtcccgggcgtcgggacgctccacgactgctgcgagcactcgccgctcttccggccgtggcgcggcggctgctgtttaatagcctggtgccggcgcaactaaaggggcgggagggcgggggcgaccacacggccaagctggaattcctggcccccgagttggtacgggcggtggcgcgactgcggtttaaggagtgcgcgccggcggacgtggtgcctcagcgtaacgcctactatagcgttctgaacacgtttcaggccctccaccgctccgaagcctttcgccagctggtgcactttgtgcgggactttgcccagctgcttaaaacctccttccgggcctccagcctcacggagaccacgggccccccaaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcgccgtgcgccacttccgccagcgcgccaccgtgtttctcgtcccccggcgccacggcaagacctggtttctagtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacccacatacacacacaaccagacgcgggaggaaagtcggccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttggtgatcctggccaggagccatcccagatggcgggccctgcttcccggtggacagagcgaccccaggtcgctgtccatggcccagcagtagatctggccgctggggaggtgccaccaggcccccgggcccaaggcgcaacacgcgcccggctccgggggggtcttcgcggggaccagatacgcgccatccagctcgccgaccactggctcctccgcgagctgttcggtggttgggtcgggggtttcctccgggggggtggccgcccgtatgcgggcgaacgtgagggtgcacaggagcggggtcagggggtgcgtcacgctccggaggtggacgatcgagcagtagcggcgctcgcggttaaagaaaaagagggcaaagaaggtgttcgggggcaaccgcagcgccttgggggcgtcagaaagaaaaatctcgcagaagagggggcccggggtctgggttaggaagggccacctgacacagaggctcggtgaggaccgttagacaccgaaagatcttgagccgctcgtccacccgaacgacgcgccacacaaagacggagttgacaatgcgcgcgatagagtcgacgtccgtccccagggcgtcgactctgtcgcgcgtgccgcgagctccgacccgggaatccggccggggcaaggtccccgggggaccaggcggcgccaggggccgccggggtcccagctgcgccatgccgggggcggggggagggcaaaccccagaggcgggggccaacggcgcggggaggagtgggtgggcgaggtggccgggggaaggcgcccgctagcgagaacggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcatcatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggcgtcgggggcggtcccctcgacggcgcgcatcaggttctcgaggagaatggggaagcgcctggtcacctcccccagccacgcgcgttggtcggggccaaagtcatagcgcaggcgctgtgagattcgagggccgccctgaagcgcggcccggatggcctggcccagggcccggaggcacgccagatgtatgcgcgcagtaaaggcgacctcggcggcgatgtcaaagggcggcaggacggggcgcgggtggcgcaggggcacctcgagcgcgggaaagcggagcagcagctccgcctgcccagcgggagacagctggtgggggcgcacgacgcgttctgcggcgcaggcctcgggtcggggccgtggccagcgccgaggacagcagcggagggcgggcgcgtcgcccgccccacgccacggagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgcggtaccaggggtcagggttagaacgtcgttcaggatgtcctcgcccccgggcccgtggggcactggggccacaaagcggcccccgcctgggggctccagacccgccaacaccgcatctgcgtcagccgcccccatggcgcccccgctgacggcctggtgaaccagggcgccctggcggagccccgatgcaacgccacaggccgcacgcccggtccgagcgcggaccgggtggcggcgggtgacgtcctgcactgcccgctgaaccaacgcgaggatctcctcgttctcctgcgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcagcctccttcgcggccggcggggcggcggcgccaagcagagcgacgtagcggcacaaatgccgacagacgcgcatgatgcgcgtgctgtcggccgcgtagcgcgtgttgggggggacgagctcgtcggaactaaacagaatcacgcgggcacagctcgcccccgagccccacgcaaggcgcagcgccgccacggcgtacgggtcatagacgccctgtgcgtcacacaccacgggcaaggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggcggccgcgcatcagggcggccagagggattcccgcggccgccaggcactcgctggatatgatgacgtgaaccaaagacgagggccgacccgggacgtggccgagatcgtactggacctcgttggccaagtgcgcgttcatggttcgggggtgggtgtgggtgtgtaggcgatgcgggtcccccgagtccgcgggaagggcgcgggtttggcgcgcgtatgcgtattcgccaacggaggcgtgcgtgcttatgcgcggcgcgtttcttctgtctccagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgccgacgagcttctcaacgtggtgacctatatatgcgatgatcacatgccgcgggtggtgacgcacacaaacgccacggcctgttcttgttatatcctcaacaagcccgtttcagcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgaggtagggccaggccctgcatcccggggcgtttcgcggcgtccgggtggcggtcgagggaaatagcagccaggactcggccgtcgccatcgccacgcacgtgcacacagagatgcaccgcctatggcctcggagggggccgacgcgggctcgggccccgagcttctcttctaccactgcgagcctcccgggagcgcggtgctgtaccccttggtcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctgaataacctcaccgaaaccgtctcccccaacacggacgtccgtacgtattccggaaaacggaacggcgcctcggatgaccttatggtcgccgtcattatggccatctaccttgcggcccaggccggacctccgcacacattcgctcccatcacacgcgtttcgtgagcgcccaataaacacacccaggtatgctacgcacgaccacggtgtcgcctgttaagggggggggaagggggtgttggcgggaagcgtgggaacacgggggattctctcacgaccggcaccagtaccacccccctgtgaacacagaaaccccaacccaaatcccataaacatacgacacccggcatattttggaatttcttcggtttttatttatttaggtatgctggggtttctccctggatgcccacccccaccccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtagagcggcagccttcgctctccatcctgcatcagggccggggcttcggggtgaatgagctgggcggcctcccgcgtgacactctgcatctgcaggagagcgttcacgtacccgtcctgggcacttagcgcaaagagccgggggattagcgtaaggatgatggtggttccctccgtgatcgagtaaaccatgttaaggaccagcgatcgcagctcggcgtttacggggccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccagggggcaggcgacgattcgcgtgggaagcacccgggtgatgaccgcggggaagcgccttcggtacgccagcaacagccccaacgtgtcgggactgacgcctccggagacgaaggattcgtgcgccacgtcggccagcgtcagttgccggcggatggtcggaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtggttcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgccatgggcccggcctgggttttatggggcccggccctcggaatcgcggcttgtcggcgggggcaaagggggcggggctagggggcttgcgggaacagagacgggtggggtaaaagaatcgcactaccccaaggaagggcggggcggtttattacagagccagtcccttgagcggggatgcgtcatagacgagatactgcgcgaagtgggtctcccgcgcgtgggcttccccgttgcgggcgctgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcgaaggcccgggacgggtcgctctggccgtggtcgtacatgagggcggtgacaccccctccttgcccccgcgtaaacgcccccggggcgcgccccggggggttgcggggccggcggaagtagttgacgtcggtcgacacgggggtggcgataaactcacacacggcgtcctggccgtggtccatccctgcgcgccgcggcccctgggcgcacccgaacacggggacgggctgggccggccccaggcggtttcccgccacgaccgcgttccgcaggtacacggctgccgcgttgtccagtagagggggagccccgcggcccaggtaaaagttttggggaaggttgcccatgtcggtgacggggttgcggacggttgccgtggccacgacggcggtgtagcccacgcccaggtccacgttcccgcgcggctgggtgagcgtgaagtttacccccccgccagtttcatgccgggccacctggagctggcccaggaagtacgcctccgacgcgcgctccgagaacagcacgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgtggacggggagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccgggtgcagggggggcgcggggtccgtcacggggtcgctggggcaatcctcgccgggggtgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcgccggtcgtccgcggcgtccgcggctcgggcctgggtgttgtgcagcagctggccgtcgttgcggttgaagtccgcggtcgccacgttacacgccgctgcgtacacggggtcgtggccccccgcgctaacccggcagtcgcgatggcggtccagggccgcgcgccgcatcagggcgtcgcagtcccacacgaggggtggcagcagcgccgggtctcgcattaggtgattcagttcggcttgcgcctgcccgcccagttccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggaccacgagccgggccagggcgcagaagacgtgctcgctgccgtgtatggcggcctgcagcaggtaaaacccgccgggtagttgcggtcttcgaacgccccgcgaacggcggcgatggtggcgggggccatggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacgaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggtaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggactaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccaaaaagatcagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccactttcaggaaggactgcaggaggcgctgtttgatcggggcggtggtgacgagcaccccgtcgaccggacgcccgcgcgtgtcggcatgcgtcagacggggcacggccatggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagttgttggatggcgcgcaactggcgatgcgcgctgatacccgtcccgtccagggcctcccccgtggcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaaccgcgctccagaaagcgcacgagcgacagggtgttgcaatacgaccccaacagggcgtcgaactcgacgtcatacaggctgtttgcatcggagcgcacgcgggaaaaaaaatcgaacaggcgtcgatgcgacgccacctcgatcgtgctaaggagggacccggtcggcaccatggccgcggcataccggtatcccggagggtcgcggttgggagcggccatggggtcgcgtggagatcggctgtctctagtgatattggcccggggaggctaagatccaccccaacgcccggccacccgtgtacgtgcccgacggcccaaggtccaccgaaagacacgacgggcccggacccaaaaaggcgggggatgctgtgtgagaggccgggtgtcggtcgggggggaaaggcaccgggagaaggctgcggcctcgttccaggagaacccagtgtccccaacagacccggggacgtgggatcccaggccttatataccccccccgccccacccccgttagaacgcgacgggtgcattcaagatggccctggtccaaaagcgtgccaggaagaaattggcagaggcggcaaagctgtccgccgccgccacccacatcgaggccccggccgcgcaggctatccccagggcccgtgtgcgcaggggatcggtgggcggcagcatttggttggtggcgataaagtggaaaagcccgtccggactgaaggtctcgtgggcggcggcgaacaaggcacacagggccgtgcctcccaaaaacacggacatcccccaaaacacgggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaagtcagaggtgccgtacgaggacaatgaaaactgttcctccagtggcagttctccctcctcccccccaaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaacccccccccgtgggtgtgacgttgcgtttagttcattggaggccaaggggaaaatggggtggggaggaaacggaaaacccagtaggccgtgttgggaacacgcccggggttgtcctcaaaaggcagggtccatactacggaagccgtcgttgtattcgagacctgcctgtgcgacgcacgtcggggttgcctgtgtccggttcggccccaccgcgtgcggcacgcacgaggacgagtccgcgtgctttattggcgttccaagcgttgccctccagtttctgttgtcggtgttcccccatacccacgcccacatccaccgtagggggcctctgggccgtgcacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgctcaagttcccggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcaagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaacagaccccaataaacgtatgtcgctaccacacccttgtgtgtcaatggacgcctctccgggggggaagggaaaacaaagaggggctgggggagcggcaccaccggggcctgaacaaacaaaccacagacacggttacagtttattcggtcgggcggagaaacggccgaagccacgccccctttattcgcgtctccaaaaaaacgggacacttgtccggagaacctgtaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgcttggggcgccctccgtcggcccggcggcgatttgctgctgtgtgttgtccgtatccaccagcaacacagacatgacctccccggccggggtgtagcgcataaacacggcccccacgagccccaggtcgcgctgggtttgggtgcgcaccagccgcttggactcgatatcccgggtggagccttcgcatgtcgcgggaggtaggttaggaacagtgggcgtcggacgtcgacgccggtgagcttgtagccgatcccccgggggagaggggagggggaagagaagagggcgttgtgggtgatgggtaccaggatccgtggctcgacgttggcagactgccccccgcaccgatgtgaggcctcagggacgaaggcgcggatcagggcgttgtagtgtgcccagcgcgtcagggtcgaggcgaggccgtgggtctgctgggccaggacttcgaccggggtctcggatcgggtggcttgagccagcgcgtccaggataaacacgcgctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacaaggccgaggctatcagaaggctccggcgggcgcgttcccgctgcaccgccgaggggacgcccgccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagagggggacgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggctgcaggcgggccgtgggatccaagactgacacattaaaaaacagaatccgcggcacagcccgcggccccgcgggcggccaacccggcaagcgcgcgcgagtgggccaaaaagcctagcaggtcggagaggcagaccgcgccgttgcgtgggcggcgttcacgaaagcaaaacccgacgtcgcgagcagccccgttaggcgccagaagagagggggacgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggtttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggcgggggggcggtgtatcacgcggcccgctcgggtggctcgccgtcgccacgttggctccccgcgggaaaagagggaacgtcagggcctcggggtcagggacggccgaaaacgttacccaggcccgggaacgcagcaacacggaggcggttggattgtgcaagagacccttaaggggggcgaccgcggggggaggctgggcggtcggctcgaccgtgatgggggcgggcaggctcgcgttcgggggccggccgagcaggtaggtcttcgagatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaagaataacccccacgaaccataaaccattccccatgggggaccccgtccctaacccacggggcccgtggctatggcagggcttgccgccccgacgttggctgcgagccctgggccttcacccgaacttgggggttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttatttccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacgtgcatgggacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccaatcctcccgccacgacccgccgccctgcagataccgcaccgtattggcaagcagcccgtaaacgcggcgaatcgcggccaacatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacctgccagtaagtcatcggctcgggtacgtagacgatatcgtcgcgcgaacccagggccaccagcagttgcgtggtggtggttttccccatcccgtgaggaccgtctatataaacccgcagtagcgtgggcattttctgctccaggcggacttccgtggcttcttgctgccggcgagggcgcaacgccgtacgtcggttgctatggccgcgagaacgcgcagcctggtcgaacgcagacgcgtgttgatggcaggggtacgaagccatacgcgcttctacaaggcgcttgccaaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccaacaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccatggagcctaggacccagcgagcccgtcgccgccgcggcggcgctgcccgggggtctgcgagcagaccgaaaaggtcacactctggggcgcgcgacccgcccgagccagcggcccgccaggtaccacccgccgaccaaacccccgcctccacggagggcgggggggtgcttaagaggatcgcggcgctcttctgcgtgcccgtggccaccaagaccaaaccccgagctgcctccgaatgagagtgtttcgttccttccccctccccccgcgtcagacaaaccctaaccaccgcttaagcggcccccgcgaggtccgaagactcatttggatccggcgggagccacctgacaacaacccctgggtttccccacaccagacgccggtccgctgtgccatcgctccccttcatcccacccccatcttgtccccaaataaaacaaggtctggtagttaggacaacgaccgcagttctcgtgtgttattgtcgctctccgcctctcgcagatggacccgtattgcccatttgacgctctggacgtctgggaacacaggcgcttcatagtcgccgattcccgaaacttcatcacccccgagttcccccgggacttttggctgtcgcccgtctttaacctcccccgggagacggcggcggagcaggtggtcgtcctgcaggcccagcgcccagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggctggccgtcgcggagatggagggccagatcgtgcgcaacgacccgccgctacgatacgacaccaacctccccgtggatctgctacatatggtgtacgcgggccgcggggcgaccggctcgtcgggggtggtgttcgggacctggtaccgcactatccaggaccgcaccatcacggactttcccctgaccacccgcagtgccgactttcgggacggccggatgtccaagaccttcatgacggcgctggtcctgtccctgcagtcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcgggatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgcccccgggacgcgagcacccacggtaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttacccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccccgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaagaacaagagcctccgttgggttttctttgtgcctgtctcaaaaggtcatccccgtaaacggcgggctccagtcccggcccggcggttggcgtgaacgcaacggcggggctgggttagcgtttagtttagcattcgctctcgcctttccgcccgcccccgaccgttgagccttttttttttttttttttttttttttttttcgtccaccaaagtctctgtgggtgcgcgcatggcagccgatgccccgggagaccggatggaggagcccctgccagacagggccgtgcccatttacgtggctgggtttttggccctgtatgacagcggggactcgggcgagttggcattggatccggatacggtgcgggcggccctgcctccggataacccactcccgattaacgtggaccaccgcgctggctgcgaggtggggcgggtgctggccgtggtcgacgacccccgcgggccgttttttgtgggactgatcgcctgcgtgcaactggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtagcgctgtgcgcgatcgggcggcgccttggcactatcgtcacctacgacaccggtctcgacgccgccatcgcgccctttcgcccctgtcgccggcgtctcgcgagggggcgcggcgactggccgccgaggccgagctcgcgctatccggacgcccctgggcgcccggcgtggaggcgctgccccacacgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacaaccgcccggctcgatcgctgccgcgccgcagggtgaccggtgcccaatcgtccgtcagcgcggggtcgcctcgcccccggtactgccccccatgaaccccgttccaacatcgggcaccccggcccccgcgccgcccggcgacgggagctacctgtggatcccggcctcccatacaaccagctcgtcgccggccacgccgcgccccaaccccagccgcattccgcgtttggttcccggctgcggcgggggccgtggcctatgggcctcacggcgcgggtctttcccagcattaccctccccacgtcgcccatcagtatcccggggtgctgttctcgggacccagcccactcgaggcgcagatagccgcgttggtgggggccatagccgcggaccgccaagcgggcggtcagccggccgcgggagaccctggggtccgggggtcgggaactcccttcctcttcctttgcctcggagtcctactgccccaccgacgaaccggacgcggactacccgtactaccccggggaggctcgaggcgggccgcgcgggggcgactctcggcgcgcggcccgccagtctcccgggaccaacgagaccatcacggcgctgatgggggcgggacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgcccccccccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttcttttttttttattgggaggggcgggtaggcgggtagctttacaatgcaaaagcctttgacgtggaggaaggcgtgggggggaggaaatcggcactgaccaagggggtccgttttgtcacgggaaaggaaagaggaaacaggccgcggacacccgggggagtttatgtgttcctttttctttcttcccacacacacacaaaaggcgtaccaaacaaaaaaaccaaaagatgcgcatgcggtttaacacccgtggtttttatttacaacaaaccccccgtcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggccgacaccagggccatgtaccgtatcatctccctggcctcggctagcttggcctcgtcaaagtcgccgccctcctcgccctccccggacgcgtccgggttggtggggttcttgagctccttggtggttagagggtacagggccttcatggggttgctctgcagccgcatgacgtaacgaaaggcgaagaaggccgccgccaggccggccaggccaacagcccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcgaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggggatgttgaggtcgatgaagggctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcccgcacgtggagacggccatcacgtcgccgagcatccgcgcgctcacccgccggcccacggtgaccgaggcgatggcgttggggttcagcttgcgggcctcgttccacagggtcagctcgtgattctgtagctcgcaccacgcgatggcaacgcggcccaacatatcgttgacatggcgctgtatgtggttgtacgtaaactgcagccgggcgaactcgatggaggaggtggtcttgatgcgctccacggacgcgttggcgctggccccgggcggcgggggcgtggggtttgggggcttgcggctctgctctcggaggtgtcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaggagaatcggaaggagccgccgtactcggagcgcagcatctcgtccacttcctgccacttggtcatggtgcagaccgacgggcgctttggcacccagtcccaggccacggtgaacttgggggtcgtgagcaggttccgggtggtcggcgccgggcccgggccttggtggtgaggtcgcgcgcgtagaagccgtcgacctgcttgaagcggtcggcggcgtagctggtgtgttcggtgtgcgacccctcccggtagccgtaaaacggggacatgtacacaaagtcgccagtcgccagcacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgtgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgtgggggcgttgggtttttcggttttttgttcttcttcggtttcgggtcccccgttggggcggcgccaagggcgggcggcgccggagtggcagggcccccgttcgccgcctgggtcgcggccgcgaccccaggcgtgccgggggaactcggagccgccgacgccaccaggacccccagcgtcaaccccaagagcgcccatacgacgaaccaccggcgcccccacgagggggcgccctggtgcatggcgggactacgggggcccgtcgtgccccccgtcaggtagcctgggggcgaggtgctggaggaccgagtagaggatcgagaaaacgtcgcggtcgtagaccacgacgaccgggggccgatacagccgtcgggggcgctctcgacgatggccaccagcggacagtcggagtcgtacgtgagatatacgccgggcgggtaacggtaacgaccttcggaggtcgggcggctgcagtccgggcgggcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgtacgtccgcggccgcttgcgtggcgatgtccccccacgtctcgggcccctgccccccgggcccgcggcgacggtcttcgtcctcgcccccgcccccaggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcgttctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggggcagtagtttagctgctttagaaagttctcgacgtcgtgcaggaaacacagctccatatggacggtcccgccatacgtatccagcctgacccgttggtgatacggacagggtcgggccaggcccatggtctcggtgaaaaacgccgcgacgtctcccgcgtacgcgaacgtctccaggttgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgcccgagcagggctaacaacttttgacgggccacggtgggctcggacaccggggcggccatctcgcggcgccgatctgtaccgcggccggagtatgcggtggaccgaggcggtccgtacgctacccggtgtctggctgagccccggggtccccctcttcggggcggcctcccgcgggcccgccgaccggcaagccgggagtcggcggcgcgtgcgtttctgttctattcccagacaccgcggagaggaatcacggcccgcccagagatatagacacggaacacaaacaagcacggatgtcgtagcaataatttattttacacacattccccgccccgccctaggttcccccaccccccaacccctcacagcatatccaacgtcaggtttccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacccctccccggcgttgccgagttggctgactagggcctcggcctcgtgcgccacctccagggccgcgtccgtcgaccactcgccgttgccgcgctccagggcacgcgcggtcagctccatcatctcctcgcttaggtactcgtcctccaggagcgccagccagtcctcgatctgcagctgctgggtgcggggccccaggcttttcacggtcgccacgaacacgctactggcgacggccgccccgccctcggagataatgccccggagctgctcgcacagcgagctttcgtgcgctccgccgccgaggttcgaggccgcgcacacaaacccggcccggggacaggccaggacgaacttgcgggtgcggtcaaaaataaggagagggaggtttttgccgcccatcaggctggcccagttcccggcctgaaacacacggtcgttgccggccatgccgtagtatttgctgttgcacaaccccaacacgacactggggcgcgccgccatgacgggccgcagcaggttgcagctggcgaacatggacgtccacgcgcccggatgcgcgtccacggcgtccatcagcgcgcgggccccggcctccaggcccgccccgccctgcgcggaccacgcggccgccgcctgcacgctggggggacggcgggaccccgcgatgatggccgtgagggtgttgatgaagtatgtcgagtgatcgcagtaccgcagaatctggtttgccatgtagtacatcgccagctcgctcacgttgttgggggccaggtaataaagtttatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggctgctgggggggggtggggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgttaggctgggggcgcagatagccgccccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactggctcagggccaggtcctgatacacggcgaggttggatcgccgcccgagaagctgaagcagggggcatggcccgcacgcgtacgggtccagcgtcagggacatggcgtggttggcctcgcccagaccgtcgcgaaacttgaagttcctcccctccccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaacttgggatggcgcgcccggaggcgcatgagcgtcgtgtgtacgcaggcgtggcgcgtgtcgaaggtgcacaggttgcagggcacgtcggtctggttggagtccgcgacgtatcgaaacacgtccatctcctggcgcccgacgatcacgccgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccggaccggcgtcgtccacctcggtgagatggagggccgagttggtgctaaataccatggcccccacgagtcccgcggcgcgcgccaggtacgccccgacggcgttggcgcgggccgcggccgtgtcctggccctcgcacagcggccacgcggagatgtcggtgggcggctcgtcgaagacggccatcgacacgatagactcgagggccagggcggcgtctccggccatgacggaggccaggcgctgttcgaacccgcccgccgggcccttgccgccgccgtcgcgcccaccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggttgagggggcggttaaaaaacggaagcgggtaggtaaaattctccccgatcgatcggtggttggcgttgaacggctcggcgatgacccggctaaaatccggcatgaacagctgcaacggatacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcagagtcgcgtcaggtttggggcctgggtgctggggtccaggtgccggccgccgtgaaagacgtacacggacgagctgtagtgcgatggcgtcagtttcagggacaccgcggtacccccgagccccgtcgtgcgagaacccacgaccacggctacgttggcctcaaagccgctctccacggtcaggcccacgaccaggggcgccacggcgacgtcggcatcgccgctgcgcgccgacagtaacgccagaagctcgatgccttcggacggacacgcgcgagcgtacacgtatcccaggggcccgggggggaccttgatggtggttgccgtcttgggctttgtctccatgtcctcctggcaatcggtccgcaaacggaggtaatcccggcacgacgacggacgcccgacgaggtatgtctcccgagcgtcaaaatccggggggggggcggcgacggtcaaggggagggtgggagaccggggttggggaatgaatccctacccttcacagacaacccccgggtaaccacggggtgccgatgaaccccggcggctggcaacgcggggtccctgcgagaggcacagatgcttacggtcaggtgctccgggccgggtgcgtctgatatgcggttggtatatgtacactttacctgggggcgtgccggaccgccccagcccctcccacaccccgcgcgtcatcagccggtgggcgtggccgctattataaaaaaagtgagaacgcgaagcgttcgcactttgtcctaataatatatatattattaggacaaagtgcgaacgcttcgcgttctcactttttttataatagcggccacgcccaccggctacgtcacgctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccacccccccagcggcttttggggtttttcctcttcccccctccccacatcccccccctttaggggttcgggtgggacaaccgcgatgttttccggtggcggcggcccgctgtcccccggaggaaagtcggcggccagggcggcgtccgggttttttgcgcccgccggccctcgcggagccggccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgcccctgggcctgactccggaaggccaccgggtggccgttcacgtttacggcacgcggcagtacttttacatgaacaaggaggaggttgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgcgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccgcgggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggatgacctggttattcagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggatcataaccgacaagatcaagctctcgagctacaagctcaacgccgtggccgaagccgtcctgaaggacaagaagaaggacctgagctatcgcgacatccccgcctactacgccaccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacaccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggccggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacgggacgaccatcggcctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatatttgtgctgtgccgcggcctcacggccgccgggctgacggccatgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgttccggagcggccgccgcgttagccgagcgccccgcagaggagtggctggcgcgcccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtatcagctcatggcccgccgcgcgcaggtcccgtccatcaaggaccggatcccgtcgtgtcgggcccgcccgcgaggagggagacggtcgcgcggctggccgccctccgcgagctagacgccgccgccccaggggacgagcccgccccccccgcggccctgccctccccggccaagcgcccccgggagacgccgtcgcatgccgaccccccgggaggcgcgtccaagccccgcaagctgctggtgtccgagctggccgaggcatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccgggccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaaccggtaccccgggcacgcggtgagcatccggtctatgagccggtagtgcaggtgggcggacgtgccgggaaagatgacgtacagcatgtggcccccgtaagtggggtccgggaaaacaacagccgcgggtcgcacgccccgcctccgcgcaggatcgtgtggacgaaaaaaagtcgggtggcaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgacacgggagtggtcttcaccaggggcaccgagaacgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcccacgcccagagtcccgccggccgcagaggagcacgaccgccggcgctccttgccgtgatagggcccgggccgggagccgcggcgatgggggtcggtatcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaaccgctatgcagacacgcccctcgaactcgttcccgcaggccagcttggccttgtggagcagcagctcgtcgggatgggtggcggggggatggccgaacagaacccaggggtcaacctccatctccgtgatggcgcacatggggtcacagaacatgtgcttaaagatggcctcgggccccgcggcccgcagcaggctcacaaaccgcccgtccccgggctgcgtctcggggtccgcctcgagctggtcgacgacgggtacgatacagtcgaagaggctcgtgttgttttccgagtagcggaccacggaggcccggagtctgcgcagggccagccagtaagcccgcaccagtaacaggttacacagcaggcattctccgccggtgcgcccgcgcccccggccgtgtttcagcacggtggccatcagagggcccaggtcgaggtcgggctgggcatcgtgttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgagggcagcaggccagatccgcgtacgcccacgtatccggcgactcctccggctcgcggtccccggcgaccgtctcgaattccccgttgcgagcggcggcgcgcgtacagcagctgtccccgcccccgcgccgaccctccgtgcagtccaggagacgggcgcaatccttccagttcatcagcgcggtggtgagcgacggctgcgtgccggatcccgccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactggggcgagaggcggtaggtttgcttgtacagctcgatggtgcggcagaccagacagggccggtccagcgcgaaggtgtcgatggccgccgcggaaaagggcccggggtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgccaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtccgcgagacggcgcgcatgacgcggccgtctggttcgaggatatgacccccgccgagctggaggttgtcttcccgactacggacgccaagctgaactacctgtcgcggacgcagcggctggcctccctcctgacgtacgccgggcctataaaagcgcccgacgacgccgccgccccgcagaccccggacaccgcgtgtgtgcacggcgagctgctcgcccgcaagcgggaaagattcgcggcggtcattaaccggttcctggacctgcaccagattctgcggggctgacgcgcgcgctgttgggtgggacggttcgcgaaccctttggtgggtttacgcgggcacgcacgctcccatcgcgggcgccatggcgggactgggcaagccctaccccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctgggctccaccgagacgacggggctgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggctggaacgggtttccgggccaggggggacggttttggcccgctccccttgttaacccaagggccctcccgcccgtggcccaggccctgcggggtcttaagccctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgtccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccgcacccccagggcacccaaggggccgtgcgggagtttctccgcggtcaggcggcggcgctgacggaccttggtctggcccacgcaaacaacacgtttaccccgcagcctatgttcgcgggcgacgccccggccgcctggttgcggcccgcgtttggcctgcggcgcacctattcaccgtttgtcgttcgagaaccttcgacgcccgggaccccgtgaggcccggggagttccttctggggaaaacaccccacagcaaaaaaatcaataaaagaccacaccaacgcacgagccttgcgtttaatgtcgaggggtttattcaagggagtgggatagggttcgacggttcgaaacttaacacacaaaataatcgagcgcgtctagcccagtaacatgtgcacgtgatgtaggctggtcagcacggcgtcgctgtgatgaagcagcgcccggcgggtccgctgtaactgctgttgtaggcggtaacaggcgcggatcagcaccgccagggcgctacgaccggtgcgttgcacggagcgtcgcgacagaactgcgtttgccgatacgggcggggggccgaattgtaagcgcgtcacctcttgggagtcatcggcggataacgcactgaatggttcgttggttatgggggagtgtggttcccgagggagtgggtcgagcgcctcggcctcggaatccgagaggaacaacgaggtggtgtcggagtcttcgtcgtcagagacatacagggtctgaagcagcgacacgggcgggggggtagcgtcaatgtgtagcgcgagggaggatgcccacgaagacaccccagacaaggagctgcccgtgcgtggatttgtggacgacgcggaagccgggacggatgggcggttttgcggtgcccggaaccgaaccgccggatactccccgggtgctacatgcccgttttggggctggggttggggctggggtggggctggggttgacgggttggggctggggctggggctggggttggggctggggttggggttggggctggggttggggttggggctggggttggggctggggggctggggcgcggacaggcggttgacgggcaaatgcccccgggggcgcgcagatgtgggggcgtggccaccggctgccgggtagtggggcggcgggaaaccgggcctccgggcgtaacaccgccctccagcgtcaagtatgtggggggcgggcctgacgtcgggggcggggtgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcgaactggggaccgagcgggaggggcacaccagacaccaaagcgtggagcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggcagaagcgcgggaggaggcgggggggcggggggcggggcggggggacggcaagcgccggaagtcgtcgcgggggcccacgggcgccggccgcggctttcggccgggacgcccggtcgtgcttcgcgagccgggactgccggcccagggggccgcggtgcacactgggacgtggggaagggggccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgtcccaacaaggtccgccgggccgggggtcgggttcgggaccgaggggctctggtcgtcgggggcgcgctggtacaccggatgccccgggatagctcccccgacaggagggaggcgtcgaacggccgcccgaggatagctcgcgcgaggaaggggtcctcgcggtggcgctggcggcgaggacgtcctcgccgcccgccacaaacgggagctcctcggtggcctcgctgccaacaaaccgcacgtcgggggggccgggggggtccgggttttcccacaacaccgcgaccggggtcatggagatgtccacgagcaccagacacggcgggccccgggcggggggggtccgggttttcccacaacaccgcgaccggggtcatggagatgtccacgagcaccagacacggcgggccccgggcgaggggccgctcggcgatgagcgcggacaggcgcgggagctgtgccgccagacacgcgttttcaatcgggttcaggtcggcgtgcaggaggcggacggcccacgtctcgatgtcggacgacacggcatcgcgcaaggcggcgtccggcccgcgagcgcgtgagtcaaacagcgtgagacacagctccagctccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatacggtagtcggcgcgccccccgaggggctcagcgtctgcccagattccagacgcctccggcgtcgaaactgtcatactctgtgtagtcgtccggaaacatgcaggtccaaagagcggccagggcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggtctataatctggcagccgcggatgcgggcgtctggatgcggggtcaggcgctgcacgaatagcatggaatctgctgcgttctgaaacgcacgggggagggtgagatgcatgtactcgtgttggcggaccagatccaggcgccaaaaggtgtaaatgtgttccggggagctggccaccagcgccaccagcacgtcgttctcgttaaaggaaacgcggtgcctagtggagctgtggggcccgagcggcggtcccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgatgacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggccgatccagcgtactggtggcctcgcgcagcaccaggggccccgggcctccgctcactcgcaggtacgcctcgccccggcggcgcagcatctgcgggtcggcctcttggccgggtggggcggacgcccgggcgcgtgcgtctcgggcgcgaagatccacgagcaggggcgcgggcgcggcggccgcgcccgcgcccgtctggcctgtggccttggcgtacgcgctatataagcccatgcggcgttggatgagctcccgcgcgccccggaactcctccaccgcccatggggccaggtccccggccaccgcgtcgaattccgccaacaggccccccagggtgtcaaagttcatctcccaggccacccttggcaccacctcgtcccgcagccgggcgctcaggtcggcgtgttgggccacgcgccccccgagctcctccacggccccggcccgctcggcgctcttggcgcccagggcgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgagagacctccgcagggcctcgacgcggcgacgggtgtcgaagagcgcctgcaggcgcgcgccctgtcgcgtcaggaggcccgggccgtcgccgctggccgcgtttagcgggtgcgtctcaaaggtacgctgggcatgttccaaccaggcgaccgcctgcacgtcgagctcgcgcgccttctccgtctggtccaacagaatttcgacctgatccgcgatctcctccgccgagcgcgcctggtccagcgtcttggccacggtcgccgggacggcgaccaccttcagcagggtcttcagattggccagaccctcggcctcgagctgggcccggcgctcgcgcgcggccagcacctcccgcagccccgccgtgacccgctcggtggcttcggcgcgcgctgtttggcgcgcaccacgcgtccttggtatcggccaggccctgtcgggtcacgaatgcgacgtagtcggcgtacgccgtgtccttcacggggctctggtccacgcgctccagcgccgccacgcacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtaggacggcctcgtggtagtcgataaacccgtcgttcgcctgggcccgctccagcagcccccccgccagccgcagaagccgcgccaggggctcggtgtccacccgaaacatgtcggcgtacgtgtcggccgcggccccgaaggccgcgctccagtcgatgcggtgaatggctgcgagcggggggagcatggggtggcgctggttctcgggggggtatgggttaaacgcaagggccgtctccagggcaagggtcaccgccttggcgttggttcccagcgcctgttcggcccgctttcggaagtcccgggggttgtagccgtgcgtgcccgccagcgcctgcaggcgacggagctcgaccacgtcaaactcggcaccgctttccacgcggtccagcacggcctccacgtcggcggcccagcgctcgtggctactgcgggcgcgctgggccgccatcttctctctcaggtcggcgatggcggcctcaagttcgtcggcgcggcgtcgcgtggcgccgatgacctttcccagctcctgcagggcgcgcccgctgggggagtggtccccggccgtcccttcggcgtgcaacggcccccgaacctgccctcgtggcccgcgaggctttcccgcgcgccggtggtcgcgcgcgtcgcggcctggatcagggaggcatgctctccctccggttggttggcggcccggcgcacctggacgacaaggtcggctgccgccgaccctaaggtcgtgagctgggcgatggccccccgcgcgtccagggccaaccgagtcgccttgacgtatcccgcggcgctgcggccatggccgctaggaaggccaggggggaggccgggtcgctggcggccgcgcccagggccgtcaccgcgtcgaccaggacgcggtgcgcccgcacggccgcatccaccgtcgacgcggggtctgccgtcgcgacggcggcgctgccggcgttgatggcgttcgagacggcgtgggctatgatcggggcgtgatcggcgaagaacgcaaggaaacggagtctctggggcgtcggcgacaggttcttcagcaccaccacgaagctgggatgcaagccagacagagccgcgccgtgcccgggacgggtgctccagggcatctcggtactgccccagcagcccccacatgtccgcccgcagcgccgccgtaacctcagggggcgccccccgaacggcctcggggaggtccgaccagcccgccggcagggaggcccgcagggtcgccaggacggccggacaggcctttagccccacaaagtcagggagggggcgcaggaccccctggagtttgtgcaagaacttctcccgggcgtcgcgggccaccttcgcccgctcccgcgctccctcgagcattgcctccagggagcgcgcgcgctcccgcaaacgggcacgcgcatcgggggcgagctctgccgtcagcttggcggcatccatggcccgcgcctgccgcagcgcttcccggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgctggtcgttaatgtcgcggatgctgtgcgccagctcgtccagcggcttgcgttctatcagcccttggttggcggcgtccgtcaggacggagagccaggccgccaggtcctcgggggcgtccagcgtctggccccgctggatcagatcccgcaacaggatggccgtggggctggtcgcgatcgggggcggggcgggcgcgccgggcgcaggaacaaaggccgcgtcgctggccagctgctggcccagggccgcatctagggcgcgaagcgccgcggcgcggccggccccgggcgggggcgcgcctgctggtcgttaatgtcgcggatgctgtgcgccagctcgtccagcggcttgcgttctatcagcccttggttggcggcgtccgtcaggacggagagccggccgccggtcctcgggggcgtccagcgtctggccccgctggatcagatcccgcaacaggatggccgtggggctggtcgcgatcgggggcggggcgggaatggcggcgcgctgcgcgatgtcccgcggtgctggtcgaagacaggcagggactcgagcagctggaccacgggcacgacggcggccgaagccacgtgaaaccggcggtcgttgttgtcgctggcctgtagagccttggcgctgtatacggccccccggtaaaagtactccttaaccgcgccctcgatcgcccgacgggcctgggtccgcacctcctccagccgaacctgaacggcctcggggcccagggggggtgggcgcggagccccctgcggggccgccccgccgggggaagtaagaagaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcttcggggtggcgggggcaaaaagggtgtccgggtgggcctgcgagcgctccagaagccactcgccgaggcgtgtatacagattggccggcggggccgcgcgaagctgcagctccagggccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccggggcagcatgctcagggtaaactccagcagggcggcggccgggccggccaccccggcctgggggtgcgtccgggccccgttctcgatgagaaaggcgaggacgcgttcaaagaaaaaaataacacagagctccagcagccccggagaagccggatacggcgaccgtaaggcgctgatggtgagccgcgaacacgcggcgccctcgcgggccagggtggcggagcacgcggtgaacttaaccgccgtggcggccacgtttgggtgggcctcgaacagctgggcgaggtctgcgcccgggggctcgggtgagcggcgagtcttcagcgcctcgagggcctgtgaggacgccggaaccatgggcccgtcgtcctcgcccgcctcggcgaccggcggcccggccgggtcggggggtgccgaggcgaggacaggctccggaacggaggcggggaccgcggccccgacgggggttttgcctttgggggtggatttcttcttggttttggcagggggggccgagcgtttcgttttctcccccgaagtcaggtcttcgacgctggaaggcggagtccaggtgggtcggcggcgcttgggaaggccggccgagtagcgtgcccggtgccgaccaaccgggacgacgcccatctccaggacccgcatgtcgtcgtcatcttcttcggccgcctctgcggcgggggtcttgggggcggagggaggcggtggtgggatcgcggagggtgggtcggcggaggggggatccgtgggtggggtaccctttagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtgtggccccggcccccctcccccctcccgggggcccacgccgacgcagggctcccccaggcccgcgatctcgccccgcagggggggcgtgatggccacgcgccgttcgctgaacgcttcgtcctgcatgtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccatgcgggtcaaaaggtaggcggtgtcgccgtccctgaacagccccatccctagggggccaatggttaggagcgtgtacgacagggggcgcagggcccacgggccggcgaagaacgtgtgtgcggggcattgtgtctccagcaggcctgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcagtcgcgcgcgtccactcgcccccctcgaccaaacatccctcgatggcctccgcggacagaacgtcgcgagggcccacatcaaatatgaggctgagaaaggcagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatattgccctgcgggccagagacccggcctgtttatggaccggacccccggggttagtgttgtttccgccacccacgcccccgtaccatggccccggttcccctgattaggctacgagtcgcggtgatcgcttcccaaaaaccgagctgcgtttgtctgtcttggtcttccccccccccagcccgcacaccataacaccgagaacaacacacgggggtgggcggaacataataaagctttattggtaactagttaacggcaagtccgtgggtggcgcgacggtgtcctccgggatcatctcgtcgtcctcgacgggggtgttggaatgaggcgcctcctcgcggtccacctggcgtgggccgtgcccataggcctccggcttctgtgcgtccatgggcgtaggcgcggggagactgtttccggcgtcgcggacctccaggtccctgggagcctccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgacagacgccgccagcgcgctagaaactgacccatgaaacaacccccgtgtacctggtctcccgacagcagcttcgacgcccgggcgtgaatgccggccacgacggacagaaacccgtgaatttcgcgccggaccacggccagcacgttgtcctcgtgcgacacctgggccgccagctcgtcgcacacccccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggcgtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtcggcgcgctcggcgaggtcctgcagcaccccccgggcagccagggcgtacatgctaatcaacaggaggctggtgcccacctcggggggcggggggggctgcagttggaccaggggccgcagctgctcgacggcacccctggagatcacgtacagctcccggagcagctgctctatgttgtcggccatctgcatagtggggccgaggccgccccgggcggccggttcgaggagagtgatcagcgcgcccagtttggtgcgatggccctcgaccgtggggagatagcccagcccaaagtcccgggcccaggccaacacacgcagggcgaactcgaccgggcggggaaggtaggccgcgctacacgtggccctcagcgcgtccccaaccaccagggccagaacgtaggggacgaagcccgggtcggcgaggacgttggggtgaatgccctcgagggcggggaagcggatctgggtcgccgcggccaggtggacagagggggcatggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggccccatgatacgcgccgcgtctggctcagaacgccccccgacaggccgccgcctcacagcgccgcccgtgcgtgtgctcgctggcgccctggcccgcctgaaagtttttacgtagttggcatagtacccgtatcccgcgccagaccaaacacgttcgcccccgcgagggcaatgccccaaagagctgctggacttcgccgagtccgtggccggcgggcgtccgcgcggggacgcccgccgccagaaacccctccagggccgaaagggagtgcgtgcagtgcgagggcgtgaacccagcgtcgatcagggtgttgatcaccacggagggcgaattggattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctccagtggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactccccccaagaaccggaggccctcttgcacgaacggggcggggaagagcaggctgtacgccggggtggtaaggttcgcgctgggctgccccaacgggaccggcgccagcttgagcgacgtctccccaagggcctcgatggaggtccgcgggctcatggccaagcagctcttggtgacggtttgccagcggtctatccactccacggcgcactgcggcgcggaccggccccagggccgccgcggtgcgcaggccggcggactccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcatgcccgcgcgtctgccggggcgatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggggggggacagcgtagaccaacgacgagaccgggcgggaatgactgtcgtgcgctgtagggagcggcgaattatcgatcccccgcggccctccaggaaccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttcggcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgaccccccccctcccccgggtcttgctaggtcgcgatctgtggtcgcaatgaagaccaatccgctacccgcaaccccttccgtgtggggcgggagtaccgtggaactcccccccaccacacgcgataccgcggggcagggcctgcttcggcgcgtcctgcgccccccgatctctcgccgcgacggcccagtgctccccagggggtcgggaccccggagggcggccagcacgctgtggttgcttggcctggacggcacagacgcgccccctggggcgctgacccccaacgcgataccgaacaggccctggacaagatcctgcggggcaccatgcgcgggggggcggccctgatcggctccccgcgccatcatctaacccgccaagtgatcctgacggatctgtgccaacccaacgcggatcgtgccgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctcgtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccccaggaggcggcgcacaccggccacccgggccggccccgctcggccgtgatcctcccggcgtgtgcgttcgtggacctggacgccgagctggggctggggggcccgggcgcggcgtttctgtacctggttcacttaccgccagcgggaccaggagctgtgttgtgtgtacgtgatcaagagccagctccccccgcgcgggttggagccggccctggagcggctgtttgggcgcctccggatcccaacacgattcacggcaccgaggacatgacgcccccggccccaaaccgaaaccccgacttccccctcgcgggcctggccgccaatccccaaaccccgcgttgctctgctggccaggtcacgaacccccagttcgccgacaggctgtaccgctggcagccggacctgcgggggcgccccaccgcacgcacctgtacgtacgccgccttcgcagagctcggcatgatgcccgaggatagtccccgctgcctgcaccgcaccgagcgctttggggcggtcagcgtccccgttgtcatcctggaaggcgtggtgtggcgccccggcgagtggcgggcctgcgcgtgagcgtagcaaacgccccgcccacacaacgctccgcccccaaccccttccccgctgtcactcgttgttcgttgacccggacgtccgccaaataaagccactgaaacccgaaacgcgagtgttgtaacgtcctttgggcgggaggaagccacaaaatgcaaatgggatacatggaaggaacacacccccgtgactcaggacatcggcgtgtccttttgggtttcactgaaactggcccgcgccccacccctgcgcgatgtggataaaaagccagcgcgggtggtttagggtaccacaggtgggtgctttggaaacttgtcggtcgccgtgctcctgtgagcttgcgtccctccccggtttcctttgcgctcccgccttccggacctgctctcgcctatcttctttggctctcggtgcgattcgtcaggcagtggccttgtcgaatctcgaccccaccactcgccggacccgccgacgtcccctctcgagcccgccgaaacccgccgcgtctgttgaaatggccagccgccccgccgcatcctctcccgtcgaagcgcgggccccggttgggggacaggaggccggcggccccagcgcagccacccagggggaggccgccggggcccctctcgcccgcggccaccacgtgtactgccagcgagtcaatggcgtgatggtgctttccgacaagacgcccgggtccgcgtcctaccgcatcagcgatagcaactttgtccaatgtggttccaactgcaccatgatcatagacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacgaccgacgtccccaccgaggcccttgggggcccccctcctcctccccgcttcaccctggggggcggctgttgttcctgtcgcgacacacggcgccgctctgcgggattcgggggggagggggatcccgtcggccccgcgttgtcgtctcggacgaccgttgctccgattccgactcggatgactcggaggacaccgactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcaccgacgccaggggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattccgcccgggtccgcgtcctaccgcatcagcgatagcaactttgtccaatgtggttccaactgcaccatgatcatagacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacgaccgacgtccccaccgaggcccttgggggcccccctcctcctccccgcttcaccctgggtggcggctgttgttcctgtcgcgacacacggcgccgctctgcggtattcgggggggagggggatcccgtcggccccgcggagttcgtctcggacgaccggtcgtccgattccgactcggatgactcggaggacaccgactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcccgacgccaggggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattctgcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggactttggcctgacggaaaggcttcgcgagcacgaagcccagctggtgatcctggcccaggctctggaccattacgactgtctgatccacagcacaccgcacacgctggtcgagcgggggctgcaatcggccctgaagtatgaggagttttacctaaagcgctttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacatcagcgccatcctcgcccgcaacgggggcatcgggctatgcgtgcaggcgtttaacgactccggccccgggaccgctagcgtcatacccgccctcaaggtcctcgactcgctggtggcggcgcacaacaaaggagcgcgcgtccaaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaatctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgtcgaatttcaggacctgaacaaacacatcgccgagggatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagctggatgaaacacggcctgcgcaacagccagtttgtcgcgctgatgcccaccgccgcctcggcgcagatctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctggtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcacgagctgcgcgctgtgaccgacaaaccccctccgcgccaggcccgccgccactgtcgtcgccgtcccacgcgctcccccgctgccatggattccgcggccccagccctctcccccgctctgacggcccatacgggccagagcgcgccggcggacctggcgatccagattccaaagtgccccgaccccgagaggtacttctacacctcccagtgtcccgacattaaccacctgcgctccctcaacatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgctttttcacaacaacgaccaggcgcgccgcgagtacgtggccggcaccatcaaccacccggccatccgcgccaaggtggactggctggaagcgcgggtgcgggaatgcgcctccgttccggaaaagttcatcctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccgtatgtcccggagaaggcggacgggaccctcccagcctccccctggtccgccttctggtccacgcatataagcgcggactaaaaacagggatgtactactgcaaggttcgcaaggcgaccaacagcggggtctttggcggcgacgacaacattgtctgcacgagctgcgcgctgtgaccgacaaacccccccgcgccaggcccgccgccactgtcgtcgccgtcccacgcgctcccccgctgccatggattccgcggccccagcccccccccgctcgacggcccatacgggccagagcgcgccggcggacctggcgatccagattccaaagtgccccgaccccgagaggtacttctacacctcccagtgtcccgacattaaccacctgcgctccctcaacatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgctttttcacaacaacgccaggcgcgccgcgagtacgtggccggcaccatcaaccacccggccatccgcgccaaggtggactggctggaagcgcgggtgcgggaatgcgcctccgttccggaaaagttcatcctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacaaccttctgcgggtcacctgccagtcaaacgacctcatcagccgggacgaggccgtgcacacgcggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagggtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccgtccagattctgagggcataggataccataattttattggtgggtcgtttgttcggggacaagcgcgctcgtctgacgtttgggctactcgtcccagaatttggccaggacgtccttgtagaacgcgggtgggggggcctgggtccgcagctgctccagaaacctgtcggcgatatcaggggccgtgatatgccgggtcacaatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgccggctggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcatctcggtgccgccggcggccggcggcaaagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtactttagtatatcgcgacttgatcgtgggaatgtagcaggcgctaatatccaacacaatatcacagcccatcaacaggaggtcagtgtctgtggtgtacacgtacgcgaccgtgttggtgtgatagaggttggcgcaggcatcgtccgcctccagctgacccgagctaatgtagggaccccagggcccggagaacgcgaatacagaacagatgcgccagacgcagggccggcttcgagggcgcggcggacggcagcgcggctccggcccggccgtcccccgggtccccgaggccagagaggtgccgcgccggcgcatgttggaaaaggcagagctgggtctggagtcggtgatgggggaaggcggtggagaggcgtccacgtcactggcctcctcgtccgtccggcattgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtagcggcgctgatatttgaccaccaacgtgtacatgacgttccacaggtccacggcgatgggggtgaagtacccggccggggccccaaggccctggcgcttgaccagatggtgtgtgtgggcaaacttcatcatcccgaacaaacccatgtcaggtcgattgtaactgcggatcggcctaactaaggcgtggttggtgcgacggtccgggacacccgagcctgtctctctgtgtatggtgacccagacaacaacaccgacacaagaggacaataatccgttaggggacgctctttataatttcgatggcccaactccacgcggattggtgcagcaccctgcatgcgccggtgtgggccaaacttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccagcaaaaccccaccctcgcagcggcatacgccccctccgcgtcccgcacccgagaccccggcccggctgccctcaccaccgaagcccacctcgtcactgtggggtgttcccagcccgcattgggatgacggattcccctggcggtgtggcccccgcctcccccgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacatttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggcgtcgacgattgcagcagcgggcggtgctccggcgactgcaggtcgccgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttcagccgggagctcggcctcccgggcctctgggagcgagccaacagacagccaggactccgcgtcggacgcggtcagccacggcgatccggaagacctcgatggcgctgcccgggcgggagaggcgggggcctcgcacgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccacagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccgggggggcccccaaaccccgtatggttttggattcccctgacggggcggggccttggcggccgcccaactctcgcaccatcccggggtaatgtaaataaacttggtattgcccaacactctcccgcgtgtcgcgtgtggttcatgtgtgtgcctggcgtcccccaccctcggggtcgtgtatttcctttccctgtccttataaaagccgtatgtggggcgctgacggaaccaccccgcgtgccatcacggccaaggcgcgggatgctccgcaacgacagccaccgggccgcgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgatgccgcgtctggggcgttcgccgtcggggcgccgtgctgggctttatgcgcgcaccccccccctcgcgcggcccaccgcgcggatatacgcctggctcaaactggcggccggtggagcggcccttgttctgtggagtctcggggagcccggaacgcagccgggggccccgggcccggccacccagtgcctggcgctgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccttggccgcggcggtgttggcgggcccgggggcgaccaccgccagggctgcttctccagggcgtgccccgaccaccgccgcgtctgcgtcatcgtcgcaggcgagtctgtttcccgccgccccccggaggacccagagcgacccggggaccccgggccaccgtcccccccgacaccccaacgatcccaggggccgccggccgatgaggtcgcaccggccggggtagcgcggcccgaaaacgtctgggtgcccgtggtcacctttctgggggcgggcgcgctcgccgtcaagacggtgcgagaacatgcccgggaaacgccgggcccgggcctgccgctgtggccccaggtgtttctcggaggccatgtggcggtggccctgacggagctgtgtcaggcgcttatgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggcgctgctgtgccctccggggtcaacgggcgggtggtcgggcgattgatatatttttcaataaaaggcattagtccgaaaccgccggtgtgtgatgatttcgccataacacccaaaccccggatggggcccgggaaaattccggaaggggacacgggctaccctcactaccgagggcgcttggtcgggaggccgcatcgaacgcacacccccatccggtggtccgtgtggaggtcgtttttcagtgcccggtctcgctttgccgggaacgctagccgatccctcgcgagggggaggcgtcgggcatggccccggggcgggtgggccttgccgtggtcctgtggagcctgttgtggctcggggcgggggtggccgggggctcggaaactgcctccaccgggcccacgatcaccgcgggagcggtgacaaacgcgagcgaggcccccacatcggggtcccccgggtcagccgccagcccggaagtcacccccacatcgaccccaaaccccaacaatgtcacacaaaacaaaaccacccccaccgagccggccagccccccaacaacccccaagcccacctccacgcccaaaagcccccccacgcccccccccgaccccaaacccaagaacaacaccccccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgaccagaggaggtcctgacgaacatcccgccccacccgggggactcctggtgtacgacagcgcccccaacctgacggacccccacgtgtctgggcggagggggccggcccgggcgccgaccctccgttgtattcgtcccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccgccaggtgtttaacccgggccagatcgacacgcagacgcacgagcaccccgacgggttcaccacagtctctaccgtgacctccgaggctgtcggcggccaggtccccccgcggaccttcacctgccagatgacgtggcaccgcgactccgtgatgttctcgcgacgcaatgccaccgggctggccctggtgctgccgcggccaaccatcaccatggaatttggggtccggcatgtggtctgcacggccggctgcgtccccgagggcaaaagagggagtgacgttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgtctagagcaccacggcagtcaccagcccccacccagggaccccaccgagcggcaggtgatcgaggcgatcgagtgggtggggattggaatcggggttctcgcggcgggggtcccggtcgtaacggcaatcgtgtacgtcgtccgcacatcacagtcgcggcagcgtcatcggcggtaacgcgagacccccccgttacctttttaatatctatatagtttggtccccctctatcccgcccaccgctgggcgctataaagccgccaccctctcttccctcaggtcatccttggtcgatcccgaacgacacacggcgtggagcaaaacgcctccccctgagccgctttcctaccaacacaacggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgcccttcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggccaacgagggttgggggggggtccggcacggacccaaaataataaacacacaatcacgtgcgataaaaagaacacgcggtcccctgtggtgtttttggttatttttattaaatctcgtcgacaaacagggggaaaggggcgtggtctagcgacggcagcacgggcggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggtccggtgcggcccgggggggaaaatagggcggatcccccccagtcgtacagggggttttccgcctcaatgtacggggaggccggcgctgcattcgccgtgttcacgcagacgttttcgtagacccgcatccatggtatttcctcgtagacacgccccccgtcctcgctccccgccgtatattgactcgtcgtcctcgtagggggcgtgccgttcgcgggccgaggcggcgtgggtggctttgcggcgggcgtcgtcgtcgtcgtcgtcggccgtcagatacgtggcttccatctggtcgggttctccctccggggcgggtccccacacccgtggccgatcgaggctccccagagacgcgcgccggacaagaagggggcacgtcgccgccggcggtcgcctgtcgggtcccgcgacgttacgggccgggaggcgcgggggcaccccccccatgtgcgtgtaatacgtggccggctgtgcggccgcagcggggggctcggcgaccgggtcgtccgcatccggaagcgggggccccgcgccgtccgcacggcgcctccggaaccgccgggtggacggcgcgggggtcgagtgtaggcgaggtcgggggaggggcgggggctcgttgtcgcgccgcgcccgctgaatcttttcccgacaggtcccaccccccgcgcgatgcccccccgggccgcgggccatgtcgtccgggggaggccccgcggaccacgtcgtccggcgagacgccacgagccgcaggatggactcgtagtggagcgacggcgccccgctgcggagcagatccgcggccagggcggccccgaaccaagccttgatgctcactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggataccacggtatccacggtcccgtacgtaccgccgctccgcttgccggtctgccacaggttggctaggcacgtcaggtggcccaggacgtcgctgaccgccgccctgagcgccatgcactgcatggagccggtcgtgccgctgggaccccggtccagatggcgcgcgaacgtttccgcgggcgcctccgggctgccgccgagcgggaggaaccggcgattggagggactcagccgggacatacgtgcttgtccgtcgtccacagcatccaggacgcccaccggtacagcacggggacgtaggccaggagctcgttgagccgcagtgcggtgtcggtgctggggcggcttgggtccgccgggcgcaagaacatgtcgctgatccgatggagggcgtcgcgcaggccggccacggtggcggcgtacttggccgccgcggccccgctcttgacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggagaggagaaaagggccgccgggcagctccacctcgcccagcgcctgggtggcggccgaagcgtagtgccggatgtaccgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgtccaccacgcggacccgcgggcccaacacggacccgccgcttacgggggcgggggggaagggctccaggtccttgagcagaaaggcggggtctgccgtcccggacacgggggcccggggcgcggaggaggcggggcgcagatccacgtgctccgcggccgcgcggacgtccgcccagaacttggcgggggtggtgcgcgcgtacaggggctgggtcgctcggaggacacacgcgtagcgcaggggggtgtacgtgcccacctcgggggccgtgaatcccccgtcaaacgcggccagtgtcacgcacgccaccacggtgtcggcaaagcccagcagccgctgcaggacgagcccggcggccagaatggcgcgcgtggtcgcagcgtcgtcccggcgccggtgcgcgtccccgcacgcccgggcgtactttaaggtcactgtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccaacacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcgaaacaccgcggcgtcggggccggattggggggacactaacccccccgcgctcagtaacggcacggccgcggccccgacgggacgcccgcctctcgcgaactgccgcctcagctcggcagccctgtcgtccaggtccgacccgcgcgcctctgcgtgaaggcgcgtcccgcacacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggaccgtcacacacggcaacctgaggggccagtcgatccaacgggaatgccgcccggagctccgggcccggcccgcgtccctccagaccctccccttgggcggggaacgggccccgccgccgtcctccggcccgacgtcttccgggtagtcgtcctcctcgtactgcagttcctctaggaacagcggcgacggcgccccccgcgaaccgccgacccgccccaaaatagcccgcgcgtcgacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgagggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccccccatcgcccgctggctcgtccgccacgggcgaggcgcgggggcgggtggatgcgcgcctcctgcgccccgcgggttcgcgagccgacatggtggcgatagacgcgggtatcggatgtccgctaccccccaaaaaagaaaaagaccccacagcgcggatggaggtcggggtaggtgccgccggaccccctcgcgatgggaatggacgggagcgacggggccggcgcaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttctttcccccaccaccaaataatcagacgacaaccgcaggtttttgtaatgtatgtgctcgtgtttattgtggatacgaaccggggacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggacccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctggttgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacagaacacgcgccagacgagcggtctctcggtagtacctgtccgcgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtcgggaaggccacgtcgccgtgggcgcgaatgtcgatttgggcgcgttcggggacgtacgcgtccccccattccaccacatcgctgggcagcgttgataggaatttacactcccggtacaggtcggcgttggtcggtaacgccgaaaacaaatcctcgttccaggtatcgagcatggtacatagcgcggggcccgcgctaaagcccaagtcgtcgaggagacggttaaagagggcggcgggggggacgggcatgggcggggagggcatgagctgggcctggctcaggcgccccgttgcgtacagcggaggggccgccggggtgtttttgggacccccggccgggcgggggggtggtggcgaagcgccgtccgcgtccatgtcggcaaacagctcgtcgaccaagaggtccattgggtggggttgatacgggaaagacgatatcgggcttttgatgcgatcgtccccgcccgccccgcgagtgtgggacgcccgacggcgcgggaagagaaaaacccccaaacgcgttagaggaccggacggaccttatggggggaagtgggcagcgggaaccccgtccgttcccgaggaatgacagcccgtggtcgccaccccgcatttaagcaacccgcacgggccgccccgtacctcgtgacttccccccacattggctcctgtcacgtgaaggcaaaccgagggcggctgtccaacccaccccccgccacccagtcacggtccccgtcggattgggaaacaaaggcacgcaacgccaacaccgaatgaacccctgttggtgctttattgtctgggtacggaagtttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaatcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccaaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtggcgtccggcccctccggacccagcgggcggcgggggcgcccgcgcaggccccggggcggacaaaaccgccccggaaacgggacgccgcgtccgggggacctccgggtgttcgtcgtcttcggatgacgagcccccgtagagggcataatccgactcgtcgtactggacgaaacggacctcgccccttgggcgcgcgcgtgtctgtagggcgccacggcgggaggtgtcaggcggactatcgggactcgccatacatgaagacgggggtagtacagatcctcgtactcatcgcgcggaacctcccgcggacccgacttcacggagcggcgagaggtcatggttccacgaacacgctagggtcggatgcgcggacaattaggcctgggttcggacggcgggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaggaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagcgttttgggtctcgcaccgcgcgcccccgatcccatcgcgcccgccctcctcgccgggcggctccccgtgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggtcagcggggactccggatgggccgtcgggcgcgtctctctggacctgcgaatggctatgccggctgacttttgtgcgattattcacgcccccgcgctatccagcccagggcaccacgtaatactgggtcttatcgactcggggtaccgcggaaccgttatggccgtggtcgtagcgcctaaaaggacgcgggaatttgcccccgggaccctgcgggtcgacgtgacgttcctggacatcctggcgacccccccggccctcaccgagccgatttccctgcggcagttcccgcaactggcgccccccctcaacggggccgggatacgcgcagatccttggttggagggggcgctcggggacccaagcgtgactcctgccctaccggcgcgacgcgagggcggtcccgcgcccatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgctatgtgctggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcctggtgtttacgaacgagtttgacgcggaggcccccccgagcgagcgcgggaccgggggttttggctctaccggtatttagcccatagcttggggttcgttccgggcaataaaaaacgtttgtatctcatctttcctgtgtgtagttgtttctgttggatgcctgtgggtctatcacacccgcccctccatcccacaaacacagaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacccgggggcggcgtgtgggggtgccccaaggagggcgcctcggtcaccccaagcccccccgagcgggtcccccggcaaccccgaaggcggagaggccaagggcccgggcggcgatggccacatcctccatgaccacgtcgctctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgagcccggggggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggagtagcccgcccctccatcccacaaacacagaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaagcccccccgagcgggttcccccggcaaccccgaaggcggagaggccaagggcccgttcggcgatggccacatcctccatgaccacgtcgctctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgagcccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccaccagggcctcctgcagccgcggctccgcctccgagggcggaccggccgcgcggatcatctcatattgttcctcggggcgcgctccccagccacatatagccccgagaagaagcatcgcgggcgggtacggcttgggcgcgcggacgcaatggggcaggaagacgggaaccgcggggagaggcgggcggccgggactcccgtggaggtgaccgcgctttatgcgaccgacgggggcgttattacctcttcgatcgccctcctcacaaactctctactgggggccgagccggtttatatattcagctacgacgcatacacgcacgatggccgtgccgacgggcccacggagcaagacaggttcgaagagagtagggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgacctggctctcaccgtggccgtcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggcgcgcgtgctggccgcgtaccgcagggcgtattatggaagcgcgcagagtcccttctggtttcttagcaaattcgggcctgacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgccgcgtccctgacctcgtttgccgccatccgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtactttccctggcccattttgagtgtttcagccccccgcgcctagccacgcatcttcgggccgtgacgacccaggaccccaaccccgcggccaacacggagcagccctcgcccctgggcagggaggccgtggaacaatttttttgccacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgaggggcgtttggcggacgccgaaaagctcctggtcccccgcgggtggcccgcgtttgcgcccgccagtcccggggaggatacggcggaggatacggcgggcggcacgccgcccccacagcctgcggaatcgcaagcgcctcctgagactggccgccacggaacaacaggacaccacgcccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatatttaacggcgcgctggcaatcacaaacatcatcctggatctcgacatcgccctgaaggagcccgtcccctttcgccggctccacgaggccctgggccactttaggcgcggggctctggcggcggttcagctcctgtttcccgcggcccgcgtggccccgacgcatatccctgttattttttcaaaagcgcatgtcggcccggcccggcgtccgtgggttccggcagcggactcggcaacgacgacgacggggactggtttccctgctacgacgccgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctacgatgcggggggtggcacgggtcatccagcaggcggtgctgttggaccgagattttgtggaggccatcgggagctacgtaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggctatgtgagcttctttgaaaggaaggcgtcccacaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttgataacggcgtacggcctcgtgctcgtgtggtacaccgtcttcggtgccagtccgctgcaccgatgtatttcgtggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccacgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtccccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatgacgtcacatccaggccggcggaaaccggaacggcatatgcaaactggaaactgtcctgtcttggggcccacccacccgacgcgtcatatgtaaatgaaaatcgttcccccgaggccatgtgtagcctggatcccaacgaccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccgccccgcccatgggtcccaattggccgtcccgttaccaagaccaacccagccagcgtatccacccccgcccgggtccccgcggaagcggaacggtgtatgtgatatgctaattaaatacatgccacgtacttatggtgtctgattggtccttgtctgtgccggaggtggggcgggggccccgcccggggggcggaactaggaggggtttgggagagccggccccggcaccacgggtataaggacatccaccacccggccgctccctatcagtgatagagatctccctatcatgatagagatcgctgcactgaggtgcaggtacatccagctgacgagtcccaaataggacgaaacgcgcttcggtgtgtcctggattccactgctatccaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcaacggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcggcacgggggcaaggtggcccgcctccaacccccaccgaccaaagcccagcctgcccgcggcggacgccgtgggcgtcgcaggggtcggggtcgcggtggtcccggggccgccgatggtttgtcggacccccgccggcgtgcccccagaaccaatcgcaacccggggggaccccgccccggggcggggtggacggacggccccggcgccccccatggcgaggcgtggcgcggaagtgagcagcccgacccacccggaggcccgcggacacggagcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagcttcggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacggccgcggcggtgctggataacctcgccacccgcctgcggccctttctccagtgctacctgaaggcgcgaggcctgtgcggcctggacgaactgtgttcgcggcggcgtctggcgggcattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgccccacgtgttgtccttctttgctcatggccggcggggcgtgggtcacggcagatggcgggggtgggcccggcgtacggcctgggtgggcggagggaactaacccaacgtataaatccgtccccgctccaaggccggtgtcatagtgcccttaggagcttcccgcccgggcgcatccccccttttgcactatgacagcgacccccctccccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacccccaccatccccctccgctgattaccgagctccgcaactttgcggacctggttaaccacccgccggtcctacgcgaactggaggataagcgcggggtgcggctgcggtgtgcgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatctccaaccccccatgaatgtgtgtaaccccccccccccaaaaaaataaagagccgtaacccaaccaaaccaggcgtggtgtgagtttgtggacccaaagccctcagagacaatgcgacaggccagtatggaccgtgatacttttatttattaactcacaggggcgcttaccgccacaggaataccagaataatgaccaccacaatcgcgaccagccctgtcgccggatggggcatgatcagacgagccgcgcgccgcgcgttgggccctgtacagctcgcgcgaattgaccctaggaggccgccacgcgcccgagttttgcgttcgtcgctggtcgtcgggcgccaaagccccggacggctgttcggtcgaacgaacggccacgacagtggcataggttggggggtggtccgacatagcctcggcgtacgtcgggaggcccgacaagaggtcccttgtgatgtcgggtggggccacaagcctggtttccggaagaaacaggggggttgccaataacccgccagggccaaaactccggcgctgcgcacgtcgttcggcgcggcgccgggcgcgccgagcggctcgctgggcggcttggcgtgagcggccccgctccgacgcctcgccctctccggaggaggttggcggaattggcacggacgacaggggcccagcagagtacggtggaggtgggtccgtgggggtgtccagatcaataacgacaaacggcccctcgttcctaccagacaagctatcgtaggggggcgggggatcagcaaacgcgttccccgcgctccatagacccgcgtcgggttgcgccgcctccgaagccatggatgcgccccaaagccacgactcccgcgcgctaggtccttggggtaagggaaaaggccctactccccatccaagccagccaagttaacgggctacgccttcggggatgggactggcaccccggcggattttgttgggctggtacgcgtcgcccaaccgggcacggacgacaggggcccagcagagtacggtggaggtgggtccgtggggggggccaggtcaataacgacaaacggcccctcgttcctaccgacaagctatcgtaggggggcgggggatcagcaaacgcgttccccgcgctccatagacccgcgtcgggttgcgccgcctccgaagccatggatgcgccccaaagccacgactcccgcgcgctaggtccttggggtaagggaaaaggccctactccccatccaagccagccaagttaacgggctacgccttcggggatgggactggcaccccggcggattttgttgggctggtacgcgtcgcccaaccgagggccgcgtccacgggacgcgccttttataaccccggggtcattcccaacgatcacatgcaatctaactggctcccctctcccctctccccccctctccccgctggggctggggagggctggggctggggaggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcaggtgtggtggatgacgtgacgtgtgacgtgcggattgcgccgtgctttgttggtgttgttttacctgtggcagcccgggccccccgcgggcgcgcgcgcgcgcaaaaaaggcgggcggcggtccgggcggcgtgcgcgcgcgcggcgggcgttgggggcggggccgcgggagcgggggaggagcgggggaggagcgggggaggagcgggggaggagcgggggggggagcgggggggggggggggggggggggggggggggggggggggggggggggggggggggggggggggggggggagggggggggggaggagcgggggaggagcggggccgcgcgcggcccccggggggtgtgttttgggggggcccgtttccggggtctggccgctcctcccccgctcctccccccgctcctccccccgctcctccccccccccccccccccccccccccccccccccccccccccccccccccccccaccccccccccccccccccccccccccccccccccgccccccccccgctccccccccgctcctcccccgctcccgcggccccgcccccaacgcccgccgcgcgcgcgcacgccgcccggaccgccgcccgccttttttgcgcgcgcgcgcgcccgcggggggcccgggctgccacaggtaaaacaacaccaacaaagcacggcgcaatccgcacgtcacacgtcacgtcatccaccacacctgcccaacaacacaactcacagcgacaactcaccgcgcaacaactcctgttcctcatccacacgtccccgcgcccctcccgctcctccagacgtaccccggcgcaacacaccgctcctgctacacaccaccgcccctccccagccccagccctccccaccccacccccccccccccccccccccccccgcccccacccccccccccccccccacccccccccccccccccccccaccccagccccccccagccccagccctccccagccccagccctccccagccgcgtcccgcgctccctcgggggggttcgggcatctctacctcagtgccgccaatctcaggtcagagatccaaaccctccgggggcgcccgcgcaccccccccccccccccccccccccccccccccccccccccccccccccccccccccccccccaccccccgggggggggggggggaggggggggggggggggggggggggggggggggggggggggggggggggggggggggggggggggtgggggtgcgcgggcgcccccggagggtttggatctctgacctgagattggcggcactgaggtagagatgcccgaacccccccgagggagcgcgggacgcggctggggagggctggggctggggagggctggggctggggagggctggggctggggggggggggggggggggagggcgggggggggggggggccccccccccgcccccctccccccccccccccccccccccccccccccgcccctccccccctcccgcccctcgaataaacaacgctactgcaaaacttaatcaggtcgttgccgtttattgcgtcttcgggtttcacaagcgccccgccccgtcccggcccgttacagcaccccgtccccctcgaacgcgccgccgtcgtcttcgtcccaggcgccttcccagtccacaacgtcccgtcgcgggggcgtggccaagcccgcctccgcccccagcacctccacggcccccgccgccgccagcacggtgccgctgcggcccgtggccgaggcccagcgaatcccgggcggcgccggcggcagggcccccgggcggtcgtcggcgcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcgcggtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcccgcgccgccggccagcgcacggcgcactgcacggcgggcagcaggtcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgcgcgcccagcgccgacacgtcgggggcgccggtccagttgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttctcgtcgtcgtcgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgggctgggcccccggggctgccgccgccagccgcccagggggtcggggccctcggcgggcgggcgcgacagcgccacggggcgcgggcgggcctgcgccgcggcgccccgggccgccgcgggctgggcgggtgtgtgctcgggcccaggccgcgtgcggcggcgacgacgacgagaacccccacccccacggggccgccggggccgcctctttggccccctgcgcgcctcgggcccgctgcgccgcatggcggcctggatgcgccagatccccgaccccgaggacgtgcgcgtggtggtgctgtctcgccgctgccgggcgaggacctggccggcggcggggcctcgggggggccgccgggggtccgccgagcgcggcgggctgtcctgcctgctggcggccctggccaaccggctgtgcgggccggacacggccgcctgggcgggcaactggaccggcgcccccgacgtgtcggcgctgggcgcgcagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgacctgctgcccgccgtgcagtgcgccgtgcgctggccggcggcgcgggacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcaccgcgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacgacggcccgggggcccgccgccggcgccgcccgggattcgctgggcctcggccacgggccgcagcggcaccgtgctggcggcggcgggggccgtggggtgctgggggcggaggcgggcttggccacgcccccgcgacgggacgttgtggactgggaaggcgcctgggacgaagacgacggcggcgcgttcgagggggacggggtgctgtaacgggccgggacggggcggggcgcttgtgaaacccgaagacgcaataaacggcaacgacctgattaaggtttgcaggagcgttgtttattcgaggggcgggagggggcgggggggggggggggggggggggggggggggggggggggggggggggggggggggggcggcggcgcccgcggccgccgccctggaggcctactgctccccgcgcgccgtggccgagctcacggaccccccgctgttccccgtcccctggcgaccggccctcatgtttgccccgcgggccctggcctcgatcgccgcgcggtgcgccgggcccgcccccgccgccctgggtgggtggggagtgggtgggtggggagtgggtgggtggggagtggcaaggaagaaacaagcccgaccaccagacagaaaatgtaaccatacccaaaccgactctgggggctgtttgtggggtcggaaccataggatgaacaaaccaccccgtacctcccgcacccaagggtgcgggtggctcatcggcatctgtccggtatgggttgttccccacccactcgcgttcggacgtcttagaatcatggcggttttctatgccgacatcggttttctcccccgcaataagacacgatgcgataaaatctgtttgtaaaatttattaagggtacaaaatgccctagcacaggggtgggggtagggccggggccccacacccaaacgcaccaaacagatgcaggcagtgggtcgagtacagccccgcgtacgaacacgtcgatgcgtgtgtcagacagcaccagaaagcacaggccatcaacaggtcgtgcatgtgtcggtgggtttggacgcggggggccatggtggtgataaagttaatggccgccgtccgccagggccacaggggcgccgtctcttggttggcccggagccactgggtgtggaccagccgcgcgtggcggcccaacatggcccctgtagccgggggcgggggatcgcgcacgtttgcagcgcacatgcgagacacctcgaccacggttcgaaagaaggcccggtggtccgcgggcaacatcaccaggtgcgcaagcgcccgggcgtccagagggtagagccctgagtcatccgaggttggctcatcgcccgggtcttgccgcaagtgcgtgtgggttgggcttccggtgggcgggacgcgaaccgcggtgtggatcccgacgcgggcccgagcgtatgctccatcttgtggggagaaggggtctgggctcgccaggggggcatacttgcccgggctatacagacccgcgagccgtacgtggttcgcggggggtgcgtggggtccggggtccctgggagaccggggttgtcgtggatccctggggtcacgcggtaccctggggtctctgggagctcgcggtactctgggtccctaggttctcggggtggtcgcggacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagacacgggggggaaccaccctccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatgttgtccaggaaggtgtccgccatttccagggcccacgacatgcttttcccgacgagcaggaagcggtccacgcaacggtcgccgccggtcgcctcgacgagggcgttcctcctgcgggaaggcacgaacgcgggtgagccccctcctccgcccccgcgtcccccctcccccgcccccgcgtcccccccctccgcccccgcgtccccccctcctccgcccccgcgtccccccccctccgcccccgcgtcccccctcctccccccccaaggtgcttacccgtgcaaaaaggcggaccggtgggtttctgtcgtcggaggcccccggggtgcgtcccctgtgtttcgtgggtggggtgggcgggtctttcccccccgcgtccgcgtgtccctttccgatgcgatcccgatcccgagccggggcgtcgcgatgccgacgccgtccgctccgacggccctctgcgactcccgctcccggtccgcgtgctccgcagccgctcccgtcgttcgtggccggcgccgtctgcgggcgtcggtcgcgccgggcctttatgtgcgccggagagacccgccccccgccgcccgggtccgcccccggggccggcgcggagtcgggcacggcgccagtgctcgcacttcgccctaataatatatatatattgggacgaagtgcgaacgcttcgcgttctcacttcttttccccggcggccccgcccccttggggcggtcccgcccgccggccaatgggggggcggcaaggcgggcggcccttgggccgcccgccgtcccgttggtcccggcgtccggcgggcgggaccgggggcccggggacggccaacgggcgcgcggggctcgatctcattaccgccgaaccgggaagtcggggcccgggccccgccccctgcccgttcctcgttagcatgcggaacggaagcggaaaccgccggatcgggcggtaatgagatgccatgcggggcggggcgcggacccacccgccctcgcgccccgtccatggcagatggcgcggatgggcggggccgggggttcgaccaacgggccgcggccacgggcccccggcgtgccggcgtcggggcggggtcgtgcataatggaattccgttcggggtgggcccgccggggggcgggggggcggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttccccacccaagcatcgaccggtccgcgctagttccgcgtcgacggcgggggtcgtcggggtccgtgggtctcgccccctcccccctcgagagtccgtaggtgacctaccgtgctacgtccgccgtcgcagccgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgccccgaccacccccacgacctcgacgacgcccggcgggacgggagggcccccgcggcgggcaccgacgccggcgaggacgccggggacgccgctcgccgcgacagctggctctgcggcctccctggtagaggaggccggccggacgatcccgacgcccgaccccgcggcctcgccgccccggacccccgcctttctagccgacgacgatgacggggacgagtacgacgacgcagccgacgccgccggcgaccgggccccggcccggggccgcgaacgggaggccccgctacgcggcgcgtatccggaccccacggaccgcctgtcccgcgcccgccggcccagccgccgcggagacgtcgtcacggccggcggcggccatcggcgcatcgacctcggcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggaagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgggctggacgccgacgcggcctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggggccgtgtggggcccgagcggggagacgccgcgcagcagacgccctgatcacgcggctgctgtacaccccggacgcggaggccagtctggggctccagacccgcgtggtccccggggacgtggcgctggaccaggcctgcttccggatctcgggcgccgcgcgcaacagcagctccttcatcccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgtcgccgccgccgcaccgggcggcgcgcgggcccgccgggtacggcgccgcggggatcctcgccgccctggggcggctgtccgccgcgcccgcctcccccgtggggggcgacgaccccgacgccgcccgccacgccgacgccgacccgggcgccgcgcccaggccggccgcgtggccgtcgagtgcctggccgcctgccgcgggtcctggcggcgctggccgagggcttcgacggcgacctggcggccgtcccggggctggccggggcccggcccgccagccccccgcgccggagggacccgcggaccccgcttccccgccgccgccgcacgccgacgcgccccgcctgcgcgcgggctgcgcggcgcggtcgtgcgcgccgcgctggtgctcatgcccctgcgcggggacctgcgcgtggccggcggcagcgggccgccgtggccgccgtgcgcgccggagcctggtcgccggggccctgggccccgcgctgccgcgggacccgcgcctgccgagctccgcggccgccgccgccgcggaccgctgtttgagaaccagagcctccgccccctgctggcggcggcggccagcgcaccggacgccgccgacgcgctggcggccgccgccgcctccgccgcccgcgggaggggcgcaagcgcaagagtcccggcccggcccggccgcccggaggcggcggcccgcgacccccgaagacgaagaagagcggcgcggacgccccccccgcgcgccccccgcccccccccccccgcccccccccccccccccccggggcccgagcccccccccgcccagcccgcggcggcccggggcgccgcggcgcaggcccgcccgcgccccgtggcgctgtcgcgccggcccgccgagggccccgaccccctgggcggctggcggcggcagccccgggggcccaaccccacagcggcggggggggggggggggggggggggggggggggcgggcggccgggccgggggcgtccgcgccgctcttcttcgtcttcgggggtcgcgggccgccgcctccgggcggccgggccgggccgggactcttgcgcttgcgcccctcccgcggcgcggcgggggcggcggcggccgccagcgcgtcggcggcgtccggtgcgctggccgccgccgccagcagggggcggaggctctggtctcaaacagcaggtccgcggcggcggcggccgcggagctcggcaggccggtcccgcggcagcgcgggacaagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggtccgcgggtccctccggccgcggggggctggcgggccgggccccggccagccccgggacggccgccaggtcgccgtcgaagccctcggccagcgcctccaggatcccgcggcaggcggccaggcactcgacggccacgcggccggcctgggcgcggcgcccggcgtcggcgtcggcgtggcgggcggcgtcggggtcgtcgccccccacgggggaggcgggcgcggcggacagccgccccagggcggcgaggatccccgcggcgccgtacccggcgggcaccgcgcgctcgcccggtgcggcggcggcgacggcggcgaccccctcgtcatctgcgccggcgccggggctccccgcggcccccgtcagcgggtgggaacaggggcgcgtaggcgcggcgcaggctggtcagcaggaagcccttctgcgcctcgtatcggcgctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccacccctggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggccgcgtcggcgtccagctcgacccgccggggctgcccggccgtgaagcggcccgtggcgtcgcggccggccaccgccgcgcgggcccggcggcgctcgatgcggcccgcggaggccgcgggggtccgcgccgccgcccggggcttgggcgcggcctcggagagggggggtggcccgggcgggggcggcgtccgcccgggggcttccggcgccgcgctcgacggaccccgcccgacggcccgcgcccgcggcgtggtcggccgcgtcgttgccgtcgtcgtcccgtcctcgtcggacgacgggacgaagcggatgcggacggcgaggacgaggacccggagtccggcgaggccgagacgccgatggccgccgccggccgtgacgacgtctccgcggcggctgggccggcgggcgcggcgacaggcggtccgtggggtccggatacgcgccgcgtagcggggcctcccgttcgcggccccgggccggggcccggtcgccggcggcgtcggctgcgtcgtcgtactcgtccccgtcatcgtcgtcggctagaaaggcgggggccggggcggcgaggccgcggggtcgggcgtcgggatcgtccggacggcctcctctaccatggaggccagcgagccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcgggggggcggcggcccgtcggtggggcccgggggccggggcgctgcttgttctccgacgccatcgccgatgcggggcgatcctccggggatacggctgcgacggcggacgtagcacggtaggtcacctacggactctcgatggggggagggggcgagacccacggaccccgacgacccccgccgtcgacgcggaactagcgcggaccggtcgatgcttgggtggggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagcccaggggccggcgggaaggaggagcagcggaggccgccggccccccgccccccggcgggcccaccccgaacggaattccattatgcacgaccccgccccgacgccggcacgccgggggcccgtggccgcggcccgttggtcgaacccccggccccgcccatccgcgccatctgccatggacggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggcccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggagggaggaggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggggggggaggaggggggacgcgggggcggaggaggggggacgcgggggcggaggaggggggacgcgggggcgggggggggggcgcgggggcggaggagggggggacgcggggggggggggggggggcgcgggggcggaggagggggctcacccgcgttcgtgccttcccgcaggaggaacgccctcgtcgaggcgaccggcggcgaccgttgcgtggaccgcttcctgctcgtcggggggggggggagccactgtggtcctccgggacgttttctggatggccgacatttccccaggcgcttttgtgccttgtgtaaaagcgcggcgtcccgctctccgatccccgcccctgggcacgcgcaagcgcaagcgccctgcccgccccctctcatcggagtctgaggtcgaatccgagacagccttggagtctgaggtcgatccgagacagcatcggattcgaccgagtctggggaccaggaggaagccccgcatcggtggccgtagggccccccggaggcttggggggcggttttttctggacatgtcggcggaatccaccacggggacggaaacggatgcgtcggtgtcggacgaccccgacgacacgtccgactggtcttgtgacgacattcccccacgacccaagcgggcccgggtaaacctgcggctcactagctctcccgatcggcgggatggggttatttttcctaagatggggcgggtccggtctacccgggaaacgcagccccgggcccccaccccgtcggccccaagcccaaatgcaatgctccggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgcttgctgcaggtgtccggcggaacctggggcatgcacctgcgcaacaccatacgggaggtggaggctcgattcgacgccaccgcagaacccgtgtgcaagcttccttgtttggaggccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgcagcagaagaccgcaagtgtctggacggctgccggaaaatgcgcttctccaccgcctgcccctatccgtgcagcgacacgtttctccggccgtgagtccggtcgccccgacccccttgtatgtccccaaaataaaagaccaaaatcaaagcgtttgtcccagcgtcttaatggcgggaagggcggagagaaacagaccacgcgtacatggggggtgtttgggggtttattgacatcggggctacagggtggtaaccggatagcagatgtgaggaagtctgggccgttcgccgcgaacggcgatcagagggtccgtttcttgcggaccacggcccggtgatgtgggttgctcgtctaaaatctcgggcatacccatacacgcacaacacggacgccgcaccgaatgggacgtcgtaagggggtgggaggtagctgggtggggtttgtgcagagcaatcagggaccgcagccagcgcatacaatcgcgctcccgtccgttggtcccgggcaggaccacgccgtactggtattcgtaccggctgagcagggtctccagggggtggttgggtgccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcccacacaggtccgccgcgcctaccacccacaagtggtagaggcgaggggggctgggttggtctcggtgcaacagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccgggcgaacgacgaccggggtacccaggggttctgatgccaaaatgcggcactgcctaagcaggaagctccacagggccgggcttgcgtcgacggaagtccggggcagggcgttgttctggtcaagggggggcattacgttgacgacaacaacgcccctgttgggatattacaggcccgtgtccggtttggggcacttgcagatttgtaaggccacgcacggcggggagacaggccgacgcgggggctgctctaaaaatttaagggccctacggtccacagacccgccttcccgggggggcccttggagcgaccggcagcggaggcgtccgggggaggggagggttatttacgggggggtaggtcagggggtgggtcgtcaaactgccgctccttaaaaccccggggcccgtcgttcggggtgctcgttggttggcactcacggggcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccggcgatgccgagcgccggcatctgcaggagaggcagatggacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgcgtggcgcatctccccccggtatacgacgacgcagccgggatgagattggggccacgggatttaccgcagaagagctggacgccatggacaggcaggcggctcgagccatcagccgcggcggcaagcccccctcgaccaatggccaagctggtgactggcatgggctttacgatccacggagcgctcaccccaggatcggaggggtgtgtctttgacagcagccacccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaagtaccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaattttttattaacacccccgaggacatttgcctgggggactttggtgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctccctcgcgctaccgctcccgcgcggccgggaacaatcgcccgccttacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgcccccggggggcggtgctgtttgcgggttggcacaataagaccccgacccgcgtctgtggtgtttttggcatcatgtcgccgggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtatcggactggaggaggaggaggaagaggaggagggggccggggatggcgaacatcttaaggggggagatgggacccgtgacaccctaccccagtccccgggtccagccgtcccgttggccggggatgacgagaaggacaaacccaaccgtcccgtagtcccaccccccggtcccaacaactcccccgcgcgccccgagaccagtcgaccgaagacaccccccaccagtatcgggccgctggcaactcgacccacgacccaactcccctcaaaggggcgacccttggttccgacgcctcaacataccccgctgttctcgttcctcactgcctcccccgccctggacaccctcttcgtcgtcagcaccgtcatccacaccttatcgtttgtgtgtattgttgctatggcgacacacctgtgtggtggttggtccagacgcgggcgacgcacacaccctagcgtgcgttacgtgtgcctgccgcccgaacgcgggtagggtatggggcggggatggggagagcccacacgcggaaagcaagaacaataaaggcggcgggatctagttgatatgcgtctctgggtgtttttggggtgtggtgggcgcggggcggtcattggacgggggtgcagttaaatacatgcccgggacccatgaagcatgcgcgacttccgggcctcggaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatctgggacctgcggccaacacaacggaccccttaacgcacgccccagtgtcccctcaccccagccccctggggggctttgccgtccccctcgtagtcggtgggctgtgtgccgtagtcctgggggcggcgtgtctgcttgagctcctgcgtcgtacgtgccgcgggtgggggcgttaccatccctacatggacccagttgtcgtataattttttcccccccccccttctccgcatgggtgatgtcgggtccaaactcccgacaccaccagctggcatggtataaatcaccggtgcgccccccaaaccatgtccggcagggggatggggggcgaatgcggagggcacccaacaacaccgggctaaccaggaaatccgtggccccggcccccaacaaagatcgcggtagcccggccgtgtgacattatcgtccataccgaccacaccgacgaatcccctaagggggaggggccattttacgaggaggaggggtataacaaagtctgtctttaaaaagcaggggttagggagttgttcggtcataagcttcagtgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgctcatccccgagaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttactagataccccccccttaatgggtgcggggggggtcaggtctgcggggttgggatgggaccttaactccatataaagcgagtctggaaggggggaaaggcggacagtcgataagtcggtagcgggggacgcgcacctgttccgcctgtcgcacccacagctttttcgcgaaccgtcccgtttcgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatggggtcgtagagctgtggcactaccccatgggacacaaatgcccacgggtcgtgcatgtcgtcacggtgaccgcgtgcccacgtcgccccgccgtggcatttgccctgtgtcgcgcgaccgacagcactcacagccccgcggtgcggggggggtcaggtctgcggggttgggatgggaccttaactccatataagcgagtcggaggggggaaaggcggacagtcgataagtcggtagcgggggacgcgcacctgttccgcctgtcgcacccacagctttttcgcgaaccgtcccgtttcgggatgccgtgccgcccgttgcagggcctggtgctcgtgggcctctgggtctgtgccaccagcctggttgtccgtggccccacggtcagtcggtatcaaactcatttgtggacgccggggccttggggcccgacggcgtagtggaggaagacctgcttattctcggggagcttcgctttgtgggggaccaggtcccccacaccacctactacgatggggtcgtagagctgtggcactaccccatgggacacaaatgcccacgggtcgtgcatgtcgtcacggtgaccgcgtgcccacgtcgccccgccgtggcatttgccctgtgtcgcgcgaccgacagcactcacagccccgcatatcccaccctggagctgaatctggcccaacagccgcttttgcgggtccggagggcgacgcgtgactatgccggggtgtacgtgttacgcgtatgggtcggggacgcaccaaacgccagcctgtttgtcctggggatggccatagccgccgaaggtactctggcgtacaacggctcggcccatggctcctgcgacccgaaactgcttccgtcttcggccccgcgtctggccccggcgagcgtataccaacccgcccctaacccggcctccaccccctccaccaccacctccccccctcgaccaccacctccaccccctcgaccaccatccccgctccccaagcatcgaccacacccttccccacgggagacccaaaaccccaacctcacggggtcaaccacgaacccccatcgaatgccacgcgagcgacccgcgactcgcgatatgcgctaacggtgacccagataatccagatagccatccccgcgtccattatagccctggtgtttctggggagctgtatttgctttatacacagatgtcaacgccgctaccgacgctcccgccgcccgatttacagcccccagatacccacgggcatctcatgcgcggtgaacgaagcggccatggcccgcctcggagccgagctcaaatcgcatccgagcaccccccccaaatcccggcgccggtcgtcacgcacgccaatgccctccctgacggccatcgccgaagagtcggagcccgcgggggcggctgggcttccgacgccccccgtggaccccacgacatccccccaacgcctcccctgttggtataggtccacggccactggccgggggcaccacataaccgaccgcagtcactgagttgggaataaaccggtattattttcctatatccgtgtatgtccatttctttcttcccccccccccccggaaaccaaagaaggaagcaaagaatggatgggaggagttcaggaagccggggagagggcccgcggcgcatttaaggcgttgttgtgttgactttggctcttctggcgggttggtgcggtgctgtttgttgggctcccattttacccgaagatcggctgctatccccgggcatggatcgcggggcggtgggggggcttcttctcggtgtttgtgttgtatcgtgcttggcgggaacgcccaaaacgtcctggagacgggtgagtgtcggcgaggacgtttcgttgcttccactcggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacgaacggacttcgtgtggcaggagcgcgcggccgtggttaaccggagtctggttattcacggggtccgagagacggacagcggcctgtataccctgtccgtgggcgacataaaggacccggctcgccaagtggcctcggtggtcctggtggtgcaaccggccccagttccgaccccacccccgaccccagccgattacgacgaggatgacaatgacgagggcgaggacgaaagtctcgccggcactcccgccagcgggaccccccggctcccgcctcccccccccccccgaggtcttggcccagcgcccccgaagtctcacatgtgcgtggggtgaccgtgcgtatggagactccggaagctatcctgttttcccccggggagacgttcagcacgaacgtctccatccatgccatcgcccacgacgaccagacctactccatggacgtcgtctggttgaggttcgacgtgccgacctcgtgtgccgagatgcgaatatacgaatcgtgtctgtatcacccgcagctcccagaatgtctgtccccggccgacgcgccgtgcgccgcgagtacgtggacgtctcgcctggccgtccgcagctacgcggggtgttccagaacaaaccccccaccgcgctgttcggccgaggctcacatggagcccgtcccggggctggcgtggcaggcggcctccgtcaatctggagttccgggacgcgtccccacaacactccggcctgtatctgtgtgtggtgtacgtcaacgaccatattcacgcctggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcgctggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatcccccgtagcgacgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccggctgtcgacgccccctccctgtttgtcgcctggctggccgctcggtggctccggggggcttccggcctgggggccgtcctgtgtgggattgcgtggtatgtgacgtcaattgcccgaggcgcacaaagggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgttgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactacattaaattgggttcgattggcaatgttgtctcccggttgatttttgggtgggtggggagtgggtgggtggggagtgggtgggggaatgggtggg

SEQ ID NO: 2 - protein, 338 aa, Human alphaherpesvirus 1
  source 1..338: note = Herpes simplex virus 1
MLAVRSLQHLSTVVLITAYGLVLVWYTVFGASPLHRCIYAVRPTGTNNDTALVWMKMNQTLLFLGAPTHPPNGGWRNHAHICYANLIAGRVVPFQVPPDATNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELMLRFVAVGLIVGTAFISRGACAITYPLFLTITTWCFVSTIGLTELYCILRRGPAPKNADKAAAPGRSKGLSGVCGRCCSIILSGIAMRLCYIAVVAGVVLVALHYEQEIQRRLFDV

SEQ ID NO: 3 - protein, 269 aa, Human alphaherpesvirus 1
  source 1..269: note = Herpes simplex virus 1
MAARTRSLVERRRVLMAGVRSHTRFYKALAKEVREFHATKICGTLLTLLSGSLQGRSVFEATRVTLICEVDLGPRRPDCICVFEFANDKTLGGVCVIIELKTCKYISSGDTASKREQRATGMKQLRHSLKLLQSLAPPGDKIVYLCPVLVFVAQRTLRVSRVTRLVPQKVSGNITAVVRMLQSLSTYTVPMEPRTQRARRRRGGAARGSASRPKRSHSGARDPPEPAARQVPPADQTPASTEGGGVLKRIAALFCVPVATKTKPRAASE

SEQ ID NO: 4 - DNA, 81 bp, synthetic construct
  misc_feature 1..81: note = Description of Artificial Sequence: Synthetic oligonucleotide
  source 1..81: mol_type = other DNA
ctgaggtgcaggtacatccagctgacgagtcccaaataggacgaaacgcgcttcggtgtgtcctggattccactgctatcc

SEQ ID NO: 5 - DNA, 465 bp, Homo sapiens
  source 1..465: mol_type = unassigned DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataa

SEQ ID NO: 6 - DNA, 763 bp, Homo sapiens
  source 1..763: mol_type = unassigned DNA
aatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 7 - DNA, 489 bp, Homo sapiens
  source 1..489: mol_type = unassigned DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 8 - DNA, 19 bp, Human alphaherpesvirus 1
  source 1..19: mol_type = unassigned DNA; note = Herpes simplex virus 1
tccctatcagtgatagaga

SEQ ID NO: 9 - DNA, 5606 bp, synthetic construct
  misc_feature 1..5606: note = Description of Artificial Sequence: Synthetic polynucleotide
  source 1..5606: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggctgcaggtcaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcatccccgcgatacatccaacacagacagggaaaagatacaaaagtaaacctttatttcccaacagacagcaaaaatcccctgagtttttttttattagggccaacacaaaagacccgctggtgtgtggtgcccgtgtctttcacttttcccctccccgacacggattggctggtgtagtgggcgcggccagagaccacccagcgcccgacccccccctccccacaaacacggggggcgtcccttattgttttccctcgtcccgggtcgaccagacatgataagatacattgatgagtttggacaaaccacaactagaatgcagtgaaaaaaatgctttatttgtgaaatttgtgatgctattgctttatttgtaaccattataagctgcaataaacaagttctgctttaataagatctgaattcccgggatccgctgtacgcggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatatcaattcgccctatagtgagtcgtattacaattctttgccaaaatgatgagacagcacaataaccagcacgttgcccaggagctgtaggaaaaagaagaaggcatgaacatggttagcagaggggcccggtttggactcagagtattttatcctcatctcaaacagtgtatatcattgtaaccataaagagaaaggcaggatgatgaccaggatgtagttgtttctaccaataagaatatttccacgccagccagaatttatatgcagaaatattctaccttatcatttaattataacaattgttctctaaaactgtgctgaagtacaatataatataccctgattgccttgaaaaaaaagtgattagagaaagtacttacaatctgacaaataaacaaaagtgaatttaaaaattcgttacaaatgcaagctaaagtttaacgaaaaagttacagaaaatgaaaagaaaataagaggagacaatggttgtcaacagagtagaaagtgaaagaaacaaaattatcatgagggtccatggtgatacaagggacatcttcccattctaaacaacaccctgaaaactttgccccctccatataacatgaattttacaatagcgaaaaagaaagaacaatcaagggtccccaaactcaccctgaagttctcaggatcgatccggagctttttgcaaaagcctaggcctccaaaaaagcctcttcactacttctggaatagctcagaggccctagaggatccccggcggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagactgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcggcccgccctgtgagggcgggctggctccaagcgtatatatgcgcggctcctgccatcgtctctccggagagcggcttggtgcggagctcgaattcggtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 810 bp, Human alphaherpesvirus 1
  source 1..810: mol_type = unassigned DNA; note = Herpes simplex virus 1
atggccgcgagaacgcgcagcctggtcgaacgcagacgcgtgttgatggcaggggtacgaagccatacgcgcttctacaaggcgcttgccaaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccatggagcctaggacccagcgagcccgtcgccgccgcggcggcgctgcccgggggtctgcgagcagaccgaaaaggtcacactctggggcgcgcgacccgcccgagccagcggcccgccaggtaccacccgccgaccaaacccccgcctccacggagggcgggggggtgcttaagaggatcgcggcgctcttctgcgtgcccgtggccaccaagaccaaaccccgagctgcctccgaatga

SEQ ID NO: 11 - DNA, 1017 bp, Human alphaherpesvirus 1
  source 1..1017: mol_type = unassigned DNA; note = Herpes simplex virus 1
atgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacccgacgccacgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggtgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgttgttccatcatcctgtcgggcatcgcaatgcgattgtgttatatcgccgtggtggccggggtggtgctcgtggcgcttcactacgagcaggagatccagaggcgcctgtttgatgtatga